(19) United States
(12) Patent Application Publication
(10) Pub. No.: US 2025/0277004 A1
(43) Pub. Date: Sep. 4, 2025

(54) HYBRID AAV CAPSIDS

(71) Applicant: REGENXBIO INC., Rockville, MD (US)

(72) Inventors: Olivier DANOS, Princeton, NJ (US); Ye LIU, Clarksville, MD (US); Andrew MERCER, Poolesville, MD (US); Samantha YOST, Clarksburg, MD (US); Randolph QIAN, Silver Spring, MD (US)

(21) Appl. No.: 18/857,586

(22) PCT Filed: Apr. 17, 2023
(86) PCT No.: PCT/US2023/065855

§ 371 Date: Oct. 17, 2024

Related U.S. Application Data
(60) Provisional application No. 63/351,796, filed on Jun. 13, 2022.
(60) Provisional application No. 63/342,567, filed on May 16, 2022.
(60) Provisional application No. 63/332,132, filed on Apr. 18, 2022.

(51) Int. Cl. C07K 14/005; A61K 48/00; A61P 21/00; C12N 15/86

(52) U.S. Cl. CPC C07K 14/005; A61P 21/00; C12N 15/86; A61K 48/00; C12N 2750/14122; C12N 2750/14143; C12N 2750/14152

(57) ABSTRACT

Provided herein are hybrid adeno-associated viruses comprising VP1u and/or VP1/2s region(s) from a first AAV and a VP3 region from a second AAV. Also provided herein are hybrid AAVs and compositions comprising hybrid AAVs that can be used for treating a subject in need thereof. Also provided herein are methods of making such hybrid AAVs.

### CROSS-REFERENCE TO RELATED APPLICATIONS

This application claims the benefit of U.S. Provisional Patent Application No. 63/332,132, filed Apr. 18, 2022, U.S. Provisional Patent Application No. 63/342,567, filed May 16, 2022, and U.S. Provisional Patent Application No. 63/351,796, filed Jun. 13, 2022, the content of each of which is incorporated herein by reference in its entirety.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application entitled “12656-168-228_SEQ_LISTING.xml”, was created on Apr. 17, 2023 and is 334,216 bytes in size.

### 1. FIELD

The field relates to the treatment of a disease or disorder in a subject (e.g., myopathy). Provided herein are methods and compositions involving hybrid adeno-associated viruses (hybrid AAVs) for the treatment of a disease or disorder in a subject (e.g., muscle related disease or disorder). Provided herein are hybrid adeno-associated viruses (AAVs) having hybrid capsid proteins engineered to maintain parental tropism while having improved propertices (e.g., endosomal escape and nuclear trafficking properties). For example, provided herein are hybrid AAVs comprising hybrid capsid proteins comprising VP1u of AAV5, VP1/2s of AAV5, and VP3 of AAV8; VP1u of AAV8, VP1/2s of AAV5, and VP3 of AAV8, or VP1u of AAVhu32, VP1/2s of AAVhu32 and VP3 of AAVrh13. Also provided herein are hybrid capsid proteins that direct the hybrid AAVs of the disclosure to target tissues (e.g., skeletal muscle tissue) or location (e.g., targeted to a muscle related tissue or cell) in the subject, while, for example, detargeting the liver. Further provided herein are AAVhu32 capsid proteins and variant AAVhu32 capsid proteins that direct AAV vectors of the disclosure to target tissues (e.g., skeletal muscle tissue) or location (e.g., targeted to a muscle related tissue or cell) in the subject, while, for example, detargeting the liver.

### 2. BACKGROUND

The use of adeno-associated viruses (AAV) as gene delivery vectors is a promising avenue for the treatment of many unmet patient needs. Dozens of naturally occurring AAV capsids have been reported, and mining the natural diversity of AAV sequences in primate tissues has identified over a hundred variants, distributed in clades. AAVs belong to the parvovirus family and are single-stranded DNA viruses with relatively small genomes and simple genetic components. Without a helper virus, AAV establishes a latent infection. An AAV genome generally has a Rep gene and a Cap gene, flanked by inverted terminal repeats (ITRs), which serve as replication and packaging signals for vector production. The capsid proteins form capsids that carry genome DNA and can determine tissue tropism to deliver DNA into target cells.

Due to low pathogenicity and the promise of long-term, targeted gene expression, recombinant AAVs (rAAVs) have been used as gene transfer vectors, in which therapeutic sequences are packaged into various capsids. Such vectors have been used in preclinical gene therapy studies and several gene therapy products are currently in clinical development. However, there remains a need for hybrid AAV vectors that achieve high transduction efficiency in a specific tissue (e.g., muscle associated tissue) with minimal toxicity. There also is a need for hybrid AAV vectors with enhanced tissue-specific targeting, cell-specific tropism, and/or enhanced tissue-specific transduction to deliver therapies.

### 3. SUMMARY

In one aspect, provided herein is a polypeptide comprising: a) the VP1/2s region of AAV5; and b) the VP3 region of AAV8. In some aspects, the polypeptide further comprises the VP1u region of AAV5 or AAV8. In some aspects, the regions are present in the following order from amino- to carboxy-terminus of the polypeptide: a) the VP1u region of AAV5 or AAV8; b) the VP1/2s region of AAV5; and c) the VP3 region of AAV8.

In one aspect, provided herein is a polypeptide comprising: a) the VP1u region of AAVhu32; b) the VP1/2s region of AAVhu32; and c) the VP3 region of AAVrh13.

In some aspects, the VP1/2s region of AAV5 comprises SEQ ID NO: 3. In some aspects, the VP1u region of AAV5 or AAV8 comprises SEQ ID NO: 7 or 17, respectively. In some aspects, the VP3 region of AAV8 comprises SEQ ID NO: 11. In some aspects, the VP1/2s region of AAV5 comprises an amino acid sequence consisting of SEQ ID NO: 3. In some aspects, the VP1u region of AAV5 or AAV8 comprises an amino acid sequence consisting of SEQ ID NO: 7 or 17, respectively. In some aspects, the VP3 region of AAV8 comprises an amino acid sequence consisting of SEQ ID NO: 11. In some aspects, the VP1/2s region of AAV5 consists of SEQ ID NO: 3. In some aspects, the VP1u region of AAV5 or AAV8 consists of SEQ ID NO: 7 or 17, respectively. In some aspects, the VP3 region of AAV8 consists of SEQ ID NO: 11. In some aspects, the polypeptide comprises SEQ ID NO: 23 or 25. In some aspects, the polypeptide consists of SEQ ID NO: 23 or 25. In some aspects, the polypeptide comprises and amino acid sequence consisting of SEQ ID NO: 23 or 25. In some aspects, the VP1u region of AAVhu32 comprises the VP1u portion of SEQ ID NO: 29, or SEQ ID NO: 146. In some aspects, the VP1/2s region of AAVhu32 comprises the VP1/2s portion of SEQ ID NO: 29, or SEQ ID NO: 148. In some aspects, the VP3 region of AAVrh13 comprises SEQ ID NO: 27. In some aspects, the VP1u region of AAVhu32 comprises the VP1u portion of an amino acid sequence consisting of SEQ ID NO: 29, or SEQ ID NO: 146. In some aspects, the VP1/2s region of AAVhu32 comprises the VP1/2s portion of an amino acid sequence consisting of SEQ ID NO: 29, or SEQ ID NO: 148. In some aspects, the VP3 region of AAVrh13 comprises an amino acid sequence consisting of SEQ ID NO: 27. In some aspects, the VP1u region of AAVhu32 consists of the VP1u portion of SEQ ID NO: 29, or SEQ ID NO: 146. In some aspects, the VP1/2s region of AAVhu32 consists of the VP1/2s portion of SEQ ID NO: 29, or SEQ ID NO: 148. In some aspects, the VP3 region of AAVrh13 consists of SEQ ID NO: 27. In some aspects, the polypeptide comprises an amino acid sequence of SEQ ID NO: 29. In some aspects, the polypeptide consists of an amino acid sequence of SEQ ID NO: 29. In some aspects, the polypeptide comprises an amino acid sequence consisting of SEQ ID NO: 29. In some aspects, provided herein is a nucleotide sequence encoding the amino acid sequence of the polypeptide. In some aspects, an expression vector comprises the nucleotide sequence. In some aspects, a host cell comprises the nucleotide sequence or the expression vector. In some aspects, a recombinant AAV comprises the polypeptide.

In one aspect, provided herein is a recombinant adeno-associated virus (AAV) comprising a capsid protein, wherein the capsid protein comprises: (i) an amino acid sequence that is at least about 95% sequence identical to VP1/2s region of AAV5, and (ii) an amino acid sequence that is at least about 95% sequence identical to VP3 region of AAV8, and wherein the rAAV comprises an expression cassette.

In some aspects, the capsid protein further comprises an amino acid sequence that is at least about 95% sequence identical to VP1u region of AAV5 or AAV8. In some aspects, the capsid protein further comprises an amino acid sequence that is at least about 98% sequence identical to VP1u region of AAV5 or AAV8. In some aspects, the capsid protein further comprises an amino acid sequence of the VP1u region of AAV5 or AAV8. In some aspects, the capsid protein comprises: (i) an amino acid sequence that is at least about 98% sequence identical to VP1/2s region of AAV5, and (ii) an amino acid sequence that is at least about 98% sequence identical to VP3 region of AAV8. In some aspects, the capsid protein comprises: (i) an amino acid sequence of the VP1/2s region of AAV5, and (ii) an amino acid sequence of the VP3 region of AAV8. In some aspects, the VP1/2s region of AAV5 comprises SEQ ID NO: 3. In some aspects, the VP1u region of AAV5 or AAV8 comprises SEQ ID NO: 7 or 17, respectively. In some aspects, the VP3 region of AAV8 comprises SEQ ID NO: 11. In some aspects, the VP1/2s region of AAV5 comprises an amino acid sequence consisting of SEQ ID NO: 3. In some aspects, the VP1u region of AAV5 or AAV8 comprises an amino acid sequence consisting of SEQ ID NO: 7 or 17, respectively. In some aspects, the VP3 region of AAV8 comprises an amino acid sequence consisting of SEQ ID NO: 11. In some aspects, the VP1/2s region of AAV5 consists of SEQ ID NO: 3. In some aspects, the VP1u region of AAV5 or AAV8 consists of SEQ ID NO: 7 or 17, respectively. In some aspects, the VP3 region of AAV8 consists of SEQ ID NO: 11. In some aspects, the expression cassette comprises an open reading frame (ORF) encoding a transgene. In some aspects, the expression cassette further comprises a promoter.

In one aspect, provided herein is a recombinant adeno-associated virus (AAV) comprising a capsid protein, wherein the capsid protein comprises: (i) an amino acid sequence that is at least about 95% sequence identical to VP1u region of AAVhu32, (ii) an amino acid sequence that is at least about 95% sequence identical to VP1/2s region of AAVhu32, and (iii) an amino acid sequence that is at least about 95% sequence identical to VP3 region of AAVrh13, and an expression cassette.

In some aspects, the capsid protein comprises: (i) an amino acid sequence that is at least about 98% sequence identical to VP1u region of AAVhu32, (ii) an amino acid sequence that is at least about 98% sequence identical to VP1/2s region of AAVhu32, and (iii) an amino acid sequence that is at least about 98% sequence identical to VP3 region of AAVrh13. In some aspects, the capsid protein comprises: (i) an amino acid sequence of the VP1u region of AAVhu32, (ii) an amino acid sequence of the VP1/2s region of AAVhu32, and (iii) an amino acid sequence of the VP3 region of AAVrh13. In some aspects, the expression cassette comprises an open reading frame (ORF) encoding a transgene. In some aspects, the expression cassette further comprises a promoter.

In some aspects, the VP1u region of AAVhu32 comprises the VP1u portion of SEQ ID NO: 29, or SEQ ID NO: 146. In some aspects, the VP1u region of AAVhu32 consists of the VP1u portion of SEQ ID NO: 29, or SEQ ID NO: 146. In some aspects, the VP1u region of AAVhu32 comprises the VP1u portion of an amino acid sequence consisting of SEQ ID NO: 29, or SEQ ID NO: 146. In some aspects, the VP1/2s region of AAVhu32 comprises the VP1/2s portion of SEQ ID NO: 29, or SEQ ID NO: 148. In some aspects, the VP1/2s region of AAVhu32 consists the VP1/2s portion of SEQ ID NO: 29, or SEQ ID NO: 148. In some aspects, the VP1/2s region of AAVhu32 comprises an amino acid sequence consisting of the VP1/2s portion of SEQ ID NO: 29, or SEQ ID NO: 148. In some aspects, the VP3 region of AAVrh13 comprises an amino acid sequence of SEQ ID NO: 27. In some aspects, the VP3 region of AAVrh13 consists of an amino acid sequence of SEQ ID NO: 27. In some aspects, the VP3 region of AAVrh13 comprises an amino acid sequence consisting of SEQ ID NO: 27. In some aspects, the capsid protein comprises SEQ ID NO: 21 or 23. In some aspects, the capsid protein consists of SEQ ID NO: 21 or 23. In some aspects, the capsid protein comprises an amino acid sequence consisting of SEQ ID NO: 21 or 23. In some aspects, the capsid protein comprises an amino acid sequence of SEQ ID NO: 29. In some aspects, the capsid protein consists of an amino acid sequence of SEQ ID NO: 29. In some aspects, the capsid protein comprises an amino acid sequence consisting of SEQ ID NO: 29. In some aspects, the rAAV further comprises an AAV inverted terminal repeat.

In some aspects, a composition comprises the rAAV and a physiologically acceptable carrier. In some aspects, the rAAV further comprises a transgene. In some aspects, provided herein is a method of delivering the transgene to a cell, comprising contacting the cell with the rAAV. In some aspects, the cell is a muscle cell. In some aspects, the transgene comprises a heterologous gene associated with a muscle related disease or disorder. In some aspects, the heterologous gene is operably linked to a regulatory sequence that controls expression of the heterologous gene in a host cell. In some aspects, the host cell is a muscle cell. In some aspects, the transgene encodes a therapeutic protein. In some aspects, the therapeutic protein is associated with a muscle related disease or disorder.

In some aspects, the muscle related disease or disorder is at least one of Duchenne muscular dystrophy, Becker muscular dystrophy, Bethlem congenital muscular dystrophy (CMD), Fukuyama CMD, muscle-eye-brain disease, rigid spine syndrome, Ullrich CMD, walker-warburg syndrome, Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), amyotrophic lateral sclerosis (ALS), Spinal-bulbar muscular atrophy (SBMA), Spinal muscular atrophy (SMA), Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia, Friedreich's ataxia (FA), Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO), Congenital myasthenic syndromes (CMS), Lambert-Eaton myasthenic syndrome (LEMS), Myasthenia gravis (MG), Charcot-Marie-Tooth disease (CMT), Giant axonal neuropathy (GAN), Myofibrillar myopathy (MFM), Scapuloperoneal myopathy, Metabolic myopathy, inflammatory myopathy, endocrine myopathy, distal myopathy, and congenital myopathy. In some aspects, the muscle related disease or disorder is Duchenne muscular dystrophy. In some aspects, the transgene encodes a microdystrophin protein.

In some aspects, provided herein is a vector comprising a nucleic acid sequence encoding the capsid protein. In some aspects, the nucleic acid sequence is operably linked to a heterologous regulatory element that controls expression of the capsid protein in a host cell. In some aspects, provided herein is an in vitro cell comprising the vector.

In one aspect provided herein is a method of producing a recombinant adeno-associated virus (rAAV), the method comprising the steps of: a) culturing a cell in a cell culture to produce the rAAV, the cell comprising a nucleotide sequence encoding a capsid protein, and a nucleotide sequence comprising a transgene, and wherein the capsid protein comprises: (i) an amino acid sequence that is at least about 95% sequence identical to VP1/2s region of AAV5, and (ii) an amino acid sequence that is at least about 95% sequence identical to VP3 region of AAV8; and b) collecting the rAAV from the cell culture.

In some aspects, the capsid protein further comprises an amino acid sequence that is at least about 95% sequence identical to VP1u region of AAV5 or AAV8. In some aspects, the capsid protein further comprises an amino acid sequence that is at least about 98% sequence identical to VP1u region of AAV5 or AAV8. In some aspects, the capsid protein further comprises an amino acid sequence of VP1u region of AAV5 or AAV8. In some aspects, the capsid protein comprises: (i) an amino acid sequence that is at least about 98% sequence identical to VP1/2s region of AAV5, and (ii) an amino acid sequence that is at least about 98% sequence identical to VP3 region of AAV8. In some aspects, the capsid protein comprises: (i) an amino acid sequence of VP1/2s region of AAV5, and (ii) an amino acid sequence of VP3 region of AAV8.

In one aspect provided herein is a method of producing a recombinant adeno-associated virus (rAAV), the method comprising the steps of: a) culturing a cell in a cell culture to produce the rAAV, the cell comprising a nucleotide sequence encoding a capsid protein, and a nucleotide sequence comprising a transgene, and wherein the capsid protein comprises: (i) an amino acid sequence that is at least about 95% sequence identical to VP1u region of AAVhu32, (ii) an amino acid sequence that is at least about 95% sequence identical to VP1/2s region of AAVhu32, and (iii) an amino acid sequence that is at least about 95% sequence identical to VP3 region of AAVrh13; and b) collecting the rAAV from the cell culture.

In some aspects, the capsid protein comprises: (i) an amino acid sequence that is at least about 98% sequence identical to VP1u region of AAVhu32, (ii) an amino acid sequence that is at least about 98% sequence identical to VP1/2s region of AAVhu32, and (iii) an amino acid sequence that is at least about 98% sequence identical to VP3 region of AAVrh13. In some aspects, the capsid protein comprises: (i) an amino acid sequence of VP1u region of AAVhu32, (ii) an amino acid sequence of VP1/2s region of AAVhu32, and (iii) an amino acid sequence of VP3 region of AAVrh13.

In some aspects, the cell comprises a nucleotide sequence encoding an AAV rep protein. In some aspects, the transgene is flanked by AAV inverted terminal repeats. In some aspects, the transgene comprises a heterologous gene associated with a muscle related disease or disorder. In some aspects, the heterologous gene is operably linked to a regulatory sequence that controls expression of the heterologous gene in a host cell. In some aspects, the transgene encodes a therapeutic protein. In some aspects, the therapeutic protein is associated with a muscle related disease or disorder. In some aspects, the therapeutic protein encodes microdystrophin protein. In some aspects, the transgene encodes a functional gene product. In some aspects, the cell is selected from an invertebrate cell, an insect cell, or a mammalian cell. In some aspects, the mammalian cell is selected from HEK293, HeLa, CHO, NS0, SP2/0, PER.C6, Vero, RD, BHK, HT-1080, A549, Cos-7, ARPE-19, MRC-5, or any combination thereof. In some aspects, the insect cell is selected from High Five, Sf9, Se301, SeIZD2109, SeUCR1, Sf900+, Sf21, Bti-tn-5b1-4, MG-1, Tn368, HzAm1, BM-N, Ha2302, Hz2E5, Ao38, or any combination thereof.

In some aspects, provided herein is a method of treating a muscle related disease or disorder in a subject in need thereof, the method comprising administering the rAAV or the composition to the subject. In some aspects, the muscle related disease or disorder is at least one of Duchenne muscular dystrophy, Becker muscular dystrophy, Bethlem congenital muscular dystrophy (CMD), Fukuyama CMD, muscle-eye-brain disease, rigid spine syndrome, Ullrich CMD, walker-warburg syndrome, Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), amyotrophic lateral sclerosis (ALS), Spinal-bulbar muscular atrophy (SBMA), Spinal muscular atrophy (SMA), Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia, Friedreich's ataxia (FA), Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO), Congenital myasthenic syndromes (CMS), Lambert-Eaton myasthenic syndrome (LEMS), Myasthenia gravis (MG), Charcot-Marie-Tooth disease (CMT), Giant axonal neuropathy (GAN), Myofibrillar myopathy (MFM), Scapuloperoneal myopathy, Metabolic myopathy, inflammatory myopathy, endocrine myopathy, distal myopathy, and congenital myopathy. In some aspects, the muscle related disease or disorder is muscular dystrophy. In some aspects, the muscular dystrophy is Duchenne muscular dystrophy. In some aspects, the rAAV provides at least one improvement of packaging efficiency, yield, titer, infectivity, transduction efficiency, and transfection efficiency, as compared to a reference AAV. In some aspects, the reference AAV is AAV8. In some aspects, the reference AAV comprises VP1u of AAV8. In some aspects, the reference AAV is AAV Rh13. In some aspects, the improvement is by about or at least about 1-fold, 1.5-fold, 2-fold, 2.5-fold, 3-fold, 3.5-fold, 4-fold, 4.5-fold, 5 fold, 5.5-fold, 6-fold, 6.5-fold, 7-fold, 7.5-fold, 8-fold, 8.5-fold, 9-fold, 9.5-fold, 10-fold, or higher than 10-fold. In some aspects, the improvement is by about or at least about 2.5-fold. In some aspects, the at least one improvement is an improvement in packaging efficiency. In some aspects, the at least one improvement is an improvement in titer. In some aspects, the rAAV transduces muscle and/or liver tissues at lower levels as compared to a reference AAV. In some aspects, the rAAV results in lower RNA expression in liver as compared to a reference AAV. In some aspects, the RNA expression is lower by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100% as compared to the RNA expression from a reference AAV. In some aspects, the rAAV results in higher RNA/DNA ratio in muscle tissue as compared to a reference AAV. In some aspects, the RNA/DNA ratio is higher by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100%. In some aspects, the rAAV results in lower DNA levels in muscle tissue as compared to a reference AAV. In some aspects, the DNA levels is lower by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100% as compared to the DNA levels from a reference AAV. In some aspects, the rAAV results in a lower level of liver toxicity as compared to the level of liver toxicity from a reference AAV. In some aspects, the level of liver toxicity is lower by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100% as compared to the level of liver toxicity from a reference AAV. In some aspects, the rAAV results in transcription-specific liver de-targeting as compared to a reference AAV. In some aspects, the administering comprises administering a lower amount of vector genome of the rAAV as compared to the amount of vector genome of a reference AAV necessary to obtain the same therapeutic effect after the administering. In some aspects, the reference AAV is AAV8. In some aspects, the reference AAV comprises VP1u of AAV8. In some aspects, the reference AAV is AAVrh13.

In one aspect, provided herein is a polypeptide comprising one or more of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71. In one aspect, provided herein is a polypeptide consisting of one or more of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71. In one aspect, provided herein is a polypeptide comprising one or more of any one of SEQ ID NOs: 81, 83, 85, 87, 89, 91, 93, 95, 97, 99, 101, 103, 105, 107, 109, 111, 113, 115, 117, and 119. In one aspect, provided herein is a polypeptide consisting of one or more of any one of SEQ ID NOs: 81, 83, 85, 87, 89, 91, 93, 95, 97, 99, 101, 103, 105, 107, 109, 111, 113, 115, 117, and 119. In one aspect, provided herein is a polynucleotide encoding an AAV capsid protein comprising the amino acid sequence of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71. In one aspect, provided herein is a polynucleotide encoding an AAV capsid protein consisting of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71. In one aspect, provided herein is a polynucleotide comprising any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72, wherein the polynucleotide encodes an AAV capsid protein. In one aspect, provided herein is a polynucleotide consisting of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72, wherein the polynucleotide encodes an AAV capsid protein. In one aspect, provided herein is a polynucleotide encoding an AAV capsid protein comprising the amino acid sequence of any one of SEQ ID NOs: 81, 83, 85, 87, 89, 91, 93, 95, 97, 99, 101, 103, 105, 107, 109, 111, 113, 115, 117, and 119. In one aspect, provided herein is a polynucleotide encoding an AAV capsid protein consisting of any one of SEQ ID NOs: 81, 83, 85, 87, 89, 91, 93, 95, 97, 99, 101, 103, 105, 107, 109, 111, 113, 115, 117, and 119. In one aspect, provided herein is a polynucleotide comprising any one of SEQ ID NOs: 82, 84, 86, 88, 90, 92, 94, 96, 98, 100, 102, 104, 106, 108, 110, 112, 114, 116, 118, and 120, wherein the polynucleotide encodes an AAV capsid protein. In one aspect, provided herein is a polynucleotide consisting of any one of SEQ ID NOs: 82, 84, 86, 88, 90, 92, 94, 96, 98, 100, 102, 104, 106, 108, 110, 112, 114, 116, 118, and 120, wherein the polynucleotide encodes an AAV capsid protein.

In one aspect, provided herein is a method of treating a muscle related disease or disorder in a subject in need thereof, the method comprising administering an rAAV comprising a polypeptide comprising an amino acid sequence of SEQ ID NO:31 or an rAAV encoded by SEQ ID NO:32. In some aspects, the muscle related disease or disorder is at least one of Duchenne muscular dystrophy, Becker muscular dystrophy, Bethlem congenital muscular dystrophy (CMD), Fukuyama CMD, muscle-eye-brain disease, rigid spine syndrome, Ullrich CMD, walker-warburg syndrome, Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), amyotrophic lateral sclerosis (ALS), Spinal-bulbar muscular atrophy (SBMA), Spinal muscular atrophy (SMA), Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia, Friedreich's ataxia (FA), Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO), Congenital myasthenic syndromes (CMS), Lambert-Eaton myasthenic syndrome (LEMS), Myasthenia gravis (MG), Charcot-Marie-Tooth disease (CMT), Giant axonal neuropathy (GAN), Myofibrillar myopathy (MFM), Scapuloperoneal myopathy, Metabolic myopathy, inflammatory myopathy, endocrine myopathy, distal myopathy, and congenital myopathy. In some aspects, the muscle related disease or disorder is muscular dystrophy. In some aspects, the muscular dystrophy is Duchenne muscular dystrophy. In some aspects, the rAAV further comprises a transgene encoding a functional dystrophin, a minidystrophin, a microdystrophin, and/or dystrophin exon-skipping snRNA. In some aspects, the transgene comprises a polynucleotide sequence encoding any one of SEQ ID NOs: 73, 74, 75, 76, 77, 78, 79, or 80. In some aspects, the rAAV provides at least one improvement of packaging efficiency, yield, titer, infectivity, transduction efficiency, and transfection efficiency, as compared to a reference AAV, wherein the reference AAV is AAV9

In some aspects, the rAAV provides a higher vector yield after the rAAV is administered to the subject, as compared to a reference AAV. In some aspects, the vector yield is higher by about or at least about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10 times, or more than 10 times. In some aspects, the vector yield is higher by between about 1.5 to about 3 times. In some aspects, the RNA level is increased after the rAAV is administered to the subject, as compared to a reference AAV. In some aspects, the RNA level is increased by about or at least about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10 times, or more than 10 times. In some aspects, the RNA level is increased by between about 2.5 to about 3.5 times. In some aspects, the ratio of RNA to DNA is increased after the rAAV is administered to the subject. In some aspects, the ratio of RNA to DNA is increased by about or at least about 0.5, 1, 1.5, 2, 2.5, 3, 3.5, 4, 4.5, 5, 5.5, 6, 6.5, 7, 7.5, 8, 8.5, 9, 9.5, 10 times, or more than 10 times. In some aspects, the ratio of RNA to DNA is increased by between about 2.5 to about 3.5 times. In some aspects, the reference AAV is AAV5. In some aspects, the reference AAV is AAV8. In some aspects, the reference AAV comprises the expression cassette.

In one aspect, provided herein is a recombinant adeno-associated virus (rAAV) hu.32 capsid protein comprising SEQ ID NO: 31, or an rAAV capsid protein comprising an amino acid sequence that is about or at least about 90% identical to SEQ ID NO: 31, comprising a peptide insertion of at least 4 and up to 12 contiguous amino acids from a heterologous protein that is not an AAV protein, wherein the peptide insertion is inserted immediately after an amino acid residue corresponding to one of any one of amino acids 451 to 461 of AAVhu32 capsid protein of SEQ ID NO: 31.

In one aspect, provided herein is a recombinant adeno-associated virus (rAAV) hu.32 capsid protein comprising SEQ ID NO: 31, or an rAAV capsid protein comprising an amino acid sequence that is about or at least about 90% identical to SEQ ID NO: 31, comprising a peptide insertion of at least 4 and up to 12 contiguous amino acids from a heterologous protein that is not an AAV protein, wherein the peptide insertion is inserted immediately after an amino acid residue corresponding to one of any one of amino acids 570 to 595 of AAVhu32 capsid protein of SEQ ID NO: 31.

In some aspects, the peptide insertion is between amino acid 454 and amino acid 455 of SEQ ID NO: 31. In some aspects, the peptide insertion is between amino acid 589 and amino acid 590 of SEQ ID NO: 31. In some aspects, the peptide insertion is a muscle-homing peptide. In some aspects the muscle-homing peptide comprises an integrin receptor-binding domain or an integrin-binding domain.

In one aspect, provided herein is a recombinant adeno-associated virus (rAAV) capsid protein comprising an amino acid sequence that is about or at least about 90% identical to any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119. In some aspects, the rAAV capsid protein comprises an amino acid sequence that is about or at least about 98% identical to any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119. In some aspects, the rAAV capsid protein comprises an amino acid sequence of any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119. In some aspects, provided herein is a recombinant adeno-associated virus (rAAV) vector comprising a nucleic acid sequence encoding the rAAV capsid protein.

In one aspect, provided herein is a recombinant adeno-associated virus (rAAV) vector comprising a nucleic acid sequence of any one of SEQ ID NOs: 22, 24, 26, 30, 34, 36, 38, 40, 42, 44, 46, 48, 50, 52, 54, 56, 58, 60, 62, 64, 66, 68, 70, 72, 82, 84, 86, 88, 90, 92, 94, 96, 98, 100, 102, 104, 106, 108, 110, 112, 114, 116, 118, and 120. In some aspects, provided herein is a cell comprising the rAAV vector. In some aspects, provided herein is a cell expressing the rAAV capsid protein. In some aspects, provided herein is a recombinant AAV viral particle comprising the rAAV capsid protein. In some aspects, a first AAV viral particle comprising the rAAV capsid protein results in increased transduction of muscle cells or heart cells relative to a second AAV viral particle comprising a capsid protein that comprises any one of SEQ ID NOs: 19, 31, and 124. In some aspects, the first AAV viral particle has at least about 1.5 fold increase in transduction of muscle cells or heart cells relative to the second AAV viral particle. In some aspects, a first AAV viral particle comprising the rAAV capsid protein has reduced transduction of liver cells relative to a second AAV viral particle comprising a capsid protein that comprises any one of SEQ ID NOs: 11, 15, 19, 31, and 124. In some aspects, the first AAV viral particle has about or at least about 1 fold decrease in transduction of liver cells relative to the second AAV viral particle. In some aspects, the second AAV viral particle comprises an AAVhu32, AAV8, and/or AAV9 capsid protein.

In one aspect provided herein is a pharmaceutical composition comprising a recombinant adeno-associated virus (rAAV) vector comprising a recombinant AAV capsid protein, wherein the recombinant AAV capsid protein comprises an amino acid sequence that is at least 90% identical to any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119.

In some aspects provided herein is a method of treating a muscle related disease or disorder in a subject in need thereof, the method comprising administering the rAAV viral particle, the cell, the rAAV vector, or the pharmaceutical composition to the subject. In some aspects, the muscle related disease or disorder is at least one of Duchenne muscular dystrophy, Becker muscular dystrophy, Bethlem congenital muscular dystrophy (CMD), Fukuyama CMD, muscle-eye-brain disease, rigid spine syndrome, Ullrich CMD, walker-warburg syndrome, Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), amyotrophic lateral sclerosis (ALS), Spinal-bulbar muscular atrophy (SBMA), Spinal muscular atrophy (SMA), Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia, Friedreich's ataxia (FA), Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO), Congenital myasthenic syndromes (CMS), Lambert-Eaton myasthenic syndrome (LEMS), Myasthenia gravis (MG), Charcot-Marie-Tooth disease (CMT), Giant axonal neuropathy (GAN), Myofibrillar myopathy (MFM), Scapuloperoneal myopathy, Metabolic myopathy, inflammatory myopathy, endocrine myopathy, distal myopathy, and congenital myopathy. In some aspects, the muscle related disease or disorder is muscular dystrophy. In some aspects, the muscular dystrophy is Duchenne muscular dystrophy.

In some aspects, the rAAV further comprises a transgene encoding a functional dystrophin, a minidystrophin, a microdystrophin, or a dystrophin exon-skipping snRNA.

In one aspect, provided herein is an isolated nucleic acid molecule comprising a nucleic acid sequence encoding the rAAV capsid protein. In one aspect, provided herein is an isolated nucleic acid molecule comprising a nucleic acid sequence of any one of SEQ ID NOs: 22, 24, 26, 30, 34, 36, 38, 40, 42, 44, 46, 48, 50, 52, 54, 56, 58, 60, 62, 64, 66, 68, 70, 72, 82, 84, 86, 88, 90, 92, 94, 96, 98, 100, 102, 104, 106, 108, 110, 112, 114, 116, 118, and 120. In some aspects, provided herein is a cultured cell comprising the nucleic acid molecule.

### 4. BRIEF DESCRIPTION OF THE FIGURES

The foregoing and other objects, features and advantages will be apparent from the following description of particular embodiments of the invention, as illustrated in the accompanying drawings. The drawings are not necessarily to scale, emphasis instead being placed upon illustrating the principles of various embodiments of the invention.

FIG. 1 shows an illustration of the design of hybrid capsids. Hybrid 1 features a hybrid capsid protein having a VP1u and a VP1/2s of AAV5 and a VP3 of AAV8. Hybrid 2 features a hybrid capsid protein having a VP1u of AAV8, a VP1/2s of AAV5, and a VP3 of AAV8.

FIG. 2 shows packaging efficiency studies of hybrid AAVs comprising hybrid capsid proteins (Hybrid 1 and Hybrid 2). Packaging efficiency was measured by titer, expressed as genome copies per mL (GC/mL), and observed on Day 3 (left) and Day 5 (right).

FIGS. 3A-3C show packaging efficiency studies of hybrid AAVs expressing various transgenes. GFP transgene (FIG. 3A) or a large muscle protein-encoding transgene (FIG. 3B) were packaged in either wtAAV5, wtAAV8, Hybrid 1, or Hybrid 2, and transgene titers were measured from a 1 mL scale cell culture at 5 days post transfection. FIG. 3C shows the fold change over AAV8.

FIG. 4 illustrates the relative abundance of input vector pool compiled for the NHP study. The dotted line indicates target input for each vector, showing that each vector is well represented in the pool.

FIG. 5 shows biodistribution of hybrid and parental AAV vector pool assessed in NHP by obtaining DNA from various tissues harvested 21 days after dosing. FIG. 5 represents relative abundance of the vector pool in the liver by DNA vector genome copies. Bars in the graph are grouped together by shading according to same parental VP3 sequence as indicated in Table 1. Capsids containing AAV5 VP1u and/or VP1/2s region exhibit lower transduction (genome copy) in liver, e.g. Hybrid 1 (2171), Hybrid 2 (2172), 2092, and 2095, compared to their parental capsids.

FIG. 6 shows biodistribution of hybrid and parental AAV vector pool assessed in NHP by obtaining RNA quantitated from various tissues harvested 21 days after dosing. FIG. 6 represents relative abundance of the vector pool transgene in the liver by mRNA (cDNA) transcript copies. Bars in the graph are grouped together by shading according to same parental VP3 sequence as indicated in Table 1. Capsids containing AAV5 VP1u and/or VP1/2s region exhibit lower expression (RNA transcripts) in liver, e.g. Hybrid 1 (2171), Hybrid 2 (2172), 2092, and 2095, compared to their parental capsids.

FIGS. 7A-7C show biodistribution of hybrid and parental AAV vector pool assessed in NHP, by obtaining DNA and RNA quantitated in various tissues harvested 21 days after dosing. AAV5/AAV8 hybrid capsids Hybrid 1 (white bar) and Hybrid 2 (gray bar) transduce less efficiently than AAV8 (black bar; FIG. 7A) but are liver de-targeted on the transcriptional level (FIG. 7B) and express RNA more efficiently than wild type AAV8 in muscle, leading to an improved RNA/DNA ratio (2.5-3.5× increase over AAV8; FIG. 7C). Data represented is from one neutralizing antibody negative animal.

FIGS. 8A-8C show graphs depicting AAV hybrids assessed for productivity. AAV5/AAV8 hybrids, Hybrid 1 (white bar) and Hybrid 2 (gray bar), produce 1.5-3× more vector than their respective parental capsid, AAV8 (dotted line), in standard triple transfection of HEK293 suspension culture, regardless of the packaged genome size (FIG. 8A; 1 mL culture) or culture scale (FIG. 8B and FIG. 8C; 10 mL and 1 L culture, respectively). Lysate titers shown as a ratio over AAV8.

FIG. 9 illustrates an alignment of N-terminal VP1 amino acids (1-58) of AAVhu32 and AAV9 capsid proteins. The consensus sequence depicted (SEQ ID NO: 113) has the following variable amino acids (represented by X in the sequence listing and in the sequence Table at Section 7 of the disclosure): 14X=N or T; 21X=E or Q; 24X=A or K; 29X=A or P; 31X=Q or P; 33X=A or P; 34X=A or P; 35X=N or A; 36X=Q or E; 37X=Q or R; 39X-Q or K; 41X=N or D; and 42X=A or S.

FIGS. 10A-10B show the relative abundance of vector genome copies (DNA;

FIG. 10A) and transcript copies (RNA; FIG. 10B) of and AAVhu32 and AAV9 in a pooled vector NHP study.

FIGS. 11A-11B show biodistribution of AAVhu32 and AAV9 in mdx mouse tissues after intravenous administration. AAVhu32 transduction of muscle tissues are compared to AAV9, in terms of genome copy numbers (GC/cell; FIG. 11A) and RNA transcript copies (FIG. 11B).

FIGS. 12A-12B show graphs of engineered AAVhu32 capsids tested for transduction of skeletal muscle in wild type (FIG. 12A) and mdx (FIG. 12B) mice. Relative abundance of transcript copies was adjusted based on the vector input and presented as a fold change over AAVhu32.

FIG. 13 shows sequence alignment for different AAV serotypes

FIG. 14 shows AAVhu32 capsid variants following administration in the capsid pool and analyzed for the fold difference in transduction in muscle tissues versus liver compared to wild-type AAVhu32 (transcript levels). All skeletal muscle transcript data represent all bicep, gas, quad, TA, and diaphragm tissue; heart transcript data represents samples taken from aorta, apex, left and right atrium, left and right ventricle; and liver transcript data was from the left lobe sample from 2 animals.

FIGS. 15A-15B illustrate AA Vhu32 capsid variants #1 to 21 as analyzed compared to AAV8, AAV9, and wild-type AAVhu32 and the relative abundance of mRNA transcript copies (of transgene) for transduced capsid as detected in gastrocnemius (gas) (FIG. 15A) and quadriceps (quad) (FIG. 15B) tissues of wt and mdx mice.

FIGS. 16A-16B illustrate AA Vhu32 capsid variants #1 to 21 as analyzed compared to AAV8, AAV9, and wild-type AAVhu32 and the relative abundance of mRNA transcript copies (of transgene) for transduced capsid as detected in tibialis anterior (TA) (FIG. 16A) and heart (FIG. 16B) tissues of wt and mdx mice.

FIG. 17 illustrates AAVhu32 capsid variants #1 to 21 as analyzed compared to AAV8, AAV9, and wild-type AAVhu32 and the relative abundance of mRNA transcript copies (of transgene) for transduced capsid as detected in livers of wt and mdx mice.

FIGS. 18A-18B show the biodistribution of AAVhu32 and AAV9 in mdx mouse tissues after intravenous administration of AAVhu32, AAV9, or AAV8 (heart, FIG. 18A) or AAVhu32 or AAV9 (gastrocnemius/gas, FIG. 18B). Protein quantification of the transgene and a representative muscle protein, actin, is represented from a Western Blot.

FIG. 19 shows the immunofluorescent staining of gastrocnemius (gas) tissues from AAVhu32, AAV8 or AAV9-injected mdx mice expressing microdydtrophin protein.

FIGS. 20A-20B RNASCOPE experiments were performed on mdx mouse tissues following AAVhu32-microdystrophin vector systemic administration. DNA and RNA probes detected vector or transgene in gastrocnemius (gas) tissues, and scoring (semi-quantification) was based on the number of green (AAV-microdystrophin DNA), red (AAV-microdystrophin RNA/DNA), or blue (DAPI) signals (dots) per cell, for semi-quantification of the transduction events. AAVhu32-microdystrophin DNA detection was observed at about 3.73 GC/diploid cell (FIG. 20A) and RNA detection at about 82.3 microidystrophin RNA copies/TBP (TBP=TATA-box binding protein control) (FIG. 20B).

FIGS. 21A-21B illustrate RNASCOPE experiments performed analogously to that described for FIGS. 20A and 20B. AAV8-microdystrophin DNA detection was observed at about 1 copy per cell (FIG. 21A), and AAV9-microdystrophin DNA detection was observed at about 1.4 copies per cell (FIG. 21B).

### 5. DETAILED DESCRIPTION

Described herein are hybrid AAV capsid polypeptides (e.g., VP1u and/or VP1/2s from a first AAV and VP3 from a second AAV) as described in Section 5.1.1. Described herein are recombinant adeno-associated viruses (rAAVs) comprising hybrid AAV capsid proteins as described in Section 5.2.1. In some embodiments, such rAAVs further comprise a transgene (refer to Section 5.2.4) and have enhanced functional properties as compared to a reference AAV (e.g., an AAV that does not comprise a hybrid AAV capsid of the disclosure), as described in Section 5.2.2. In some embodiments, these rAAVs can be used to treat a disease or disorder in a subject. In some embodiments, an rAAV of the disclosure or compositions comprising the same can be used to treat or prevent a muscle related disease or disorder in a subject as described in Section 5.4. Also provided herein are rAAV vectors as described in Section 5.2.3, and pharmaceutical compositions comprising the rAAVs of the disclosure, such as described in Section 5.2.5. Also provided herein are methods of manufacturing the rAAVs of the disclosure, such as described in Section 5.3. Non-limiting illustrative examples are provided in Section 6.

### 5.1 Hybrid AAV Capsid Polypeptides

### 5.1.1 Polypeptides

Provided herein are polypeptides comprising hybrid adeno-associated virus capsid proteins. In various aspects of the disclosure, a polypeptide comprises a VP1u region and/or a VP1/2s region of a first adeno-associated virus serotype, and a VP3 region of a second adeno-associated virus serotype. In some embodiments, a polypeptide comprises a VP1u region from a first AAV serotype, a VP1/2s region of a second AAV serotype, and a VP3 region of a first AAV serotype. In some embodiments, a polypeptide comprises a VP1u region from a first AAV serotype, a VP1/2s region of a second AAV serotype, and a VP3 region of a third AAV serotype. In some embodiments, a polypeptide comprises a VP1/2s region of a second AAV serotype and a VP3 region of a first AAV serotype. In some embodiments, a polypeptide comprises a VP1u region of a second AAV serotype, a VP1/2s region of the second AAV serotype, and a VP3 region of a first AAV serotype. In some embodiments, a polypeptide comprises a VP1u region of a first AAV serotype, a VP1/2s region of a second AAV serotype, and a VP3 region of the first AAV serotype.

In some embodiments, the first AAV is an AAV serotype 1 (AAV1), serotype 2 (AAV2), serotype 2yYF (AAV2tYF), serotype 3 (AAV3), serotype 3 (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 10 (AAV10), serotype 11 (AAV11), serotype 12 (AAV12), serotype 13 (AAV13), serotype rh10 (AAVrh10), serotype rh20 (AAVrh20), serotype rh39 (AAVrh39), serotype hu.37 (AAVhu.37), serotype hu32 (AAVhu32), serotype rh13 (AAVrh13), serotype rh15 (AAVrh15), serotype rh73 (AAVrh73) and serotype rh74 (AAVrh74). In some embodiments, the second AAV is an AAV serotype 1 (AAV1), serotype 2 (AAV2), AAV2tYF, serotype 3 (AAV3), serotype 3 (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 10 (AAV10), serotype 11 (AAV11), serotype 12 (AAV12), serotype 13 (AAV13), serotype rh10 (AAVrh10), serotype rh20 (AAVrh20), serotype rh39 (AAVrh39), serotype hu.37 (AAVhu.37), serotype hu32 (AAVhu32), serotype rh13 (AAVrh13), serotype rh15 (AAVrh15), serotype rh73 (AAVrh73) and serotype rh74 (AAVrh74). In some embodiments, the first AAV is an AAV1, AAV2, AAV3, AAV3B, AAV4, AAV5, AAV6, AAV7, AAV8, AAV9, AAVrh10, AAV11, AAV12, AAV13, AAVrh20, AAVhu.37, AAVrh39, AAVhu32, AAVrh21, AAVrh13, AAVrh15, AAVrh73, or AAVrh74. In some embodiments, the second AAV is an AAV1, AAV2, AAV3, AAV3B, AAV4, AAV5, AAV6, AAV7, AAV8, AAV9, AAVrh10, AAV11, AAV12, AAV13, AAVrh20, AAVhu.37, AAVrh39, AAV hu32, AAVrh21, AAVrh13, AAVrh15, AAVrh73, or AAVrh74.

In some embodiments, one AAV (e.g., first AAV) is AAV5 and one AAV (e.g., second AAV) is AAV8. In some embodiments, one AAV (e.g., first AAV) is AAV8 and one AAV (e.g., second AAV) is AAV5. In some embodiments, one AAV (e.g., first AAV) is AAVhu32 and one AAV (e.g., second AAV) is AAVrh13. In some embodiments, one AAV (e.g., first AAV) is AAVrh13 and one AAV (e.g., second AAV) is AAVhu32. In some embodiments, the first AAV serotype is AAV8. In some embodiments, the first AAV serotype is AAVrh13. In some embodiments, the second AAV serotype is AAV5. In some embodiments, the second AAV serotype is AAVhu32. In some embodiments, the second AAV serotype is AAV8. In some embodiments, the second AAV serotype is AAVrh13. In some embodiments, the first AAV serotype is AAV5. In some embodiments, the first AAV serotype is AAVhu32. In some embodiments, the first AAV serotype is AAV8 and the second AAV serotype is AAV5. In specific embodiments, the first AAV serotype is AAVrh13 and the second AAV serotype is AAVhu32. In some embodiments, the second AAV serotype is AAV8 and the first AAV serotype is AAV5. In some embodiments, the second AAV serotype is AAVrh13 and the first AAV serotype is AAVhu32.

In some embodiments, provided herein is a polypeptide comprising: a) the VP1/2s region of a first AAV (e.g., AAV5); and the VP3 region of a second AAV (e.g., AAV8). In some embodiments, the polypeptide further comprises the VP1u region of a first AAV (e.g., AAV5) or a second AAV (e.g., AAV8). In some embodiments, the regions are present in the following order from amino- to carboxy-terminus of the polypeptide: VP1u followed by VP1/2s followed by VP3. In some embodiments, the regions are present in the following order from amino- to carboxy-terminus of the polypeptide: the VP1u region of a first AAV (e.g., AAV5) or a second AAV (e.g., AAV8); the VP1/2s region of a first AAV (e.g., AAV5); and the VP3 region of a second AAV (e.g., AAV8). In some embodiments, a polypeptide of the disclosure comprises the VP1u region of a first AAV (e.g., AAVhu32), the VP1/2s region of a first AAV (e.g., AAVhu32), and the VP3 region of a second AAV (e.g., AAVrh.13).

In certain embodiments, a polypeptide of the disclosure comprises a VP3 region of AAV5. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:1. In some embodiments, a polypeptide comprises the amino acid sequence of SEQ ID NO:1. In some embodiments, a polypeptide consists of the amino acid sequence of SEQ ID NO:1. In some embodiments, a polypeptide comprises the amino acid sequence consisting of SEQ ID NO:1.

In certain embodiments, a polypeptide of the disclosure comprises a VP1/2s region of AAV5. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:3. In some embodiments, a polypeptide comprises the amino acid sequence of SEQ ID NO:3. In some embodiments, a polypeptide consists of the amino acid sequence of SEQ ID NO:3. In some embodiments, a polypeptide comprises the amino acid sequence consisting of SEQ ID NO:3.

In certain embodiments, a polypeptide of the disclosure comprises a VP1/2s region and VP3 region of AAV5. In certain embodiments, a polypeptide of the disclosure comprises a VP2 of AAV5. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:3 and/or an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:1. In some embodiments, a polypeptide comprises the amino acid sequences of SEQ ID NO:3 and/or SEQ ID NO:1. In some embodiments, a polypeptide consists of SEQ ID NO:3 and/or SEQ ID NO:1. In some embodiments, a polypeptide comprises an amino acid sequence consisting of SEQ ID NO:3 and/or SEQ ID NO:1.

In some embodiments, a polypeptide comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:5. In some embodiments, a polypeptide comprises the amino acid sequence of SEQ ID NO:5. In some embodiments, a polypeptide consists of the amino acid sequence of SEQ ID NO:5. In some embodiments, a polypeptide comprises the amino acid sequence consisting of SEQ ID NO:5.

In certain embodiments, a polypeptide of the disclosure comprises a VP1u region of AAV5. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:7. In some embodiments, a polypeptide comprises the amino acid sequence of SEQ ID NO:7. In some embodiments, a polypeptide consists of the amino acid sequence of SEQ ID NO:7. In some embodiments, a polypeptide comprises the amino acid sequence consisting of SEQ ID NO:7.

In certain embodiments, a polypeptide of the disclosure comprises a VP1u and the VP1/2s region of AAV5. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:3 and/or an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:7. In some embodiments, a polypeptide comprises the amino acid sequences of SEQ ID NO:3 and/or SEQ ID NO:7. In some embodiments, a polypeptide consists of SEQ ID NO:3 and/or SEQ ID NO:7. In some embodiments, a polypeptide comprises an amino acid sequence consisting of SEQ ID NO:3 and/or SEQ ID NO:7.

In certain embodiments, a polypeptide of the disclosure comprises a VP3 region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, a polypeptide comprises the amino acid sequence of SEQ ID NO:11. In some embodiments, a polypeptide consists of the amino acid sequence of SEQ ID NO:11. In some embodiments, a polypeptide comprises the amino acid sequence consisting of SEQ ID NO:11.

In certain embodiments, a polypeptide of the disclosure comprises a VP1/2s region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:13. In some embodiments, a polypeptide comprises the amino acid sequence of SEQ ID NO:13. In some embodiments, a polypeptide consists of the amino acid sequence of SEQ ID NO:13. In some embodiments, a polypeptide comprises the amino acid sequence consisting of SEQ ID NO: 13.

In certain embodiments, a polypeptide of the disclosure comprises a VP1u region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:17. In some embodiments, a polypeptide comprises the amino acid sequence of SEQ ID NO:17. In some embodiments, a polypeptide consists of the amino acid sequence of SEQ ID NO:17. In some embodiments, a polypeptide comprises the amino acid sequence consisting of SEQ ID NO:17.

In some embodiments, a polypeptide of the disclosure comprises a VP1u region of AAV8 and a VP3 region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:17 and/or 11. In some embodiments, a polypeptide comprises the amino acid sequence of SEQ ID NO: 17 and/or 11. In some embodiments, a polypeptide consists of the amino acid sequence of SEQ ID NO:17 and/or 11. In some embodiments, a polypeptide comprises the amino acid sequence consisting of SEQ ID NO:17 and/or 11.

In certain embodiments, a polypeptide of the disclosure comprises a VP1/2s region and VP3 region of AAV8. In certain embodiments, a polypeptide of the disclosure comprises a VP2 of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:13 and/or an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, a polypeptide comprises the amino acid sequences of SEQ ID NO:13 and/or SEQ ID NO:11. In some embodiments, a polypeptide consists of the amino acid sequence of SEQ ID NO:13 and/or 11. In some embodiments, a polypeptide comprises the amino acid sequence consisting of SEQ ID NO:13 and/or 11. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:15. In some embodiments, a polypeptide comprises the amino acid sequence of SEQ ID NO:15. In some embodiments, a polypeptide consists of the amino acid sequence of SEQ ID NO:15. In some embodiments, a polypeptide comprises the amino acid sequence consisting of SEQ ID NO:15.

In certain embodiments, a polypeptide of the disclosure comprises a VP1u region, a VP1/2s region, and/or VP3 region of AAV8. In certain embodiments, a polypeptide of the disclosure comprises a VP1 of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:17, an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:13, and/or an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, a polypeptide comprises the amino acid sequences of SEQ ID NO:17, SEQ ID NO:13, and/or SEQ ID NO:11. In some embodiments, a polypeptide consists of the amino acid sequence of SEQ ID NO:17, 13, and/or 11. In some embodiments, a polypeptide comprises the amino acid sequence consisting of SEQ ID NO:17, 13, and/or 11. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:19. In some embodiments, a polypeptide comprises the amino acid sequence of SEQ ID NO:19. In some embodiments, a polypeptide consists of the amino acid sequence of SEQ ID NO:19. In some embodiments, a polypeptide comprises the amino acid sequence consisting of SEQ ID NO: 19.

In certain embodiments, a polypeptide of the disclosure comprises a VP 1/2s region of AAV5, and a VP3 region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:3, and an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, a polypeptide comprises the amino acid sequences of SEQ ID NO:3 and SEQ ID NO:11. In some embodiments, a polypeptide consists of the amino acid sequence of SEQ ID NO:3 and 11. In some embodiments, a polypeptide comprises the amino acid sequence consisting of SEQ ID NO:3 and/or 11. In some embodiments, a polypeptide comprises a an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:21. In some embodiments, a polypeptide comprises the amino acid sequence of SEQ ID NO:21. In some embodiments, a polypeptide consists of the amino acid sequence of SEQ ID NO:21. In some embodiments, a polypeptide comprises the amino acid sequence consisting of SEQ ID NO:21.

In certain embodiments, a polypeptide of the disclosure comprises a VP1u region of AAV5, a VP 1/2s region of AAV5, and a VP3 region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:7, an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:3, and an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, a polypeptide comprises the amino acid sequences of SEQ ID NO:7, SEQ ID NO:3, and SEQ ID NO:11. In some embodiments, a polypeptide consists of the amino acid sequence of SEQ ID NO:7, 3 and 11. In some embodiments, a polypeptide comprises the amino acid sequence consisting of SEQ ID NO:7, 3 and 11. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:23. In some embodiments, a polypeptide comprises the amino acid sequence of SEQ ID NO:23. In some embodiments, a polypeptide consists of the amino acid sequence of SEQ ID NO:23. In some embodiments, a polypeptide comprises the amino acid sequence consisting of SEQ ID NO:23.

In certain embodiments, a polypeptide of the disclosure comprises a VP1u region of AAV8, a VP 1/2s region of AAV5, and a VP3 region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:17, an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:3, and an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, a polypeptide comprises the amino acid sequences of SEQ ID NO:17, SEQ ID NO:3, and SEQ ID NO:11. In some embodiments, a polypeptide consists of the amino acid sequence of SEQ ID NO:17, 3, and 11. In some embodiments, a polypeptide comprises the amino acid sequence consisting of SEQ ID NO:17, 3, and 11. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:25. In some embodiments, a polypeptide comprises the amino acid sequence of SEQ ID NO:25. In some embodiments, a polypeptide consists of the amino acid sequence of SEQ ID NO:25. In some embodiments, a polypeptide comprises the amino acid sequence consisting of SEQ ID NO:25.

In certain embodiments, a polypeptide of the disclosure comprises a VP3 region of AAVrh13. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:27. In some embodiments, a polypeptide comprises the amino acid sequence of SEQ ID NO:27. In some embodiments, a polypeptide consists of the amino acid sequence of SEQ ID NO:27. In some embodiments, a polypeptide comprises the amino acid sequence consisting of SEQ ID NO:27.

In certain embodiments, a polypeptide of the disclosure comprises a VP1u region of AAVhu32, a VP1/2s region of AAVhu32, and a VP3 region of AAVrh13. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:29. In some embodiments, a polypeptide comprises the amino acid sequence of SEQ ID NO:29. In some embodiments, a polypeptide consists of the amino acid sequence of SEQ ID NO:29. In some embodiments, a polypeptide comprises the amino acid sequence consisting of SEQ ID NO:29. In some embodiments, a polypeptide comprises the VP1u portion (e.g., AAVhu32 VP1u) of SEQ ID NO:29 or SEQ ID NO: 146. In some embodiments, a polypeptide comprises the VP1/2s portion (e.g., AAVhu32 VP1/2) of SEQ ID NO: 29 or SEQ ID NO: 148. In some embodiments, a polypeptide comprises the VP3 portion (e.g., AAVrh13 VP3) of SEQ ID NO:29. In some embodiments, a polypeptide consists of the VP1u portion (e.g., AAVhu32 VP1u) of SEQ ID NO:29 or SEQ ID NO: 146. In some embodiments, a polypeptide consists of the VP1/2s portion (e.g., AAVhu32 VP1/2) of SEQ ID NO:29 or SEQ ID NO: 148. In some embodiments, a polypeptide consists of the VP3 portion (e.g., AAVrh13 VP3) of SEQ ID NO:29. In some embodiments, a polypeptide comprises the VP1u portion (e.g., AAVhu32 VP1u) of an amino acid sequence consisting of SEQ ID NO:29 or SEQ ID NO: 146. In some embodiments, a polypeptide comprises the VP1/2s portion (e.g., AAVhu32 VP1/2) of an amino acid sequence consisting of SEQ ID NO: 29 or SEQ ID NO: 148. In some embodiments, a polypeptide comprises the VP3 portion (e.g., AAVrh13 VP3) of an amino acid sequence consisting of SEQ ID NO:29. In some embodiments a nucleic acid sequence encoding VP1u portion (e.g., AAVhu32 VP1/2) of the rAAV is SEQ ID NO: 147. In some embodiments a nucleic acid sequence encoding VP1/2s portion (e.g., AAVhu32 VP1/2) of the rAAV is SEQ ID NO: 149.

In certain embodiments, a polypeptide of the disclosure comprises a VP1 region of AAVhu32. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:31. In some embodiments, a polypeptide comprises the amino acid sequence of SEQ ID NO:31. In some embodiments, a polypeptide consists of the amino acid sequence of SEQ ID NO:31. In some embodiments, a polypeptide comprises the amino acid sequence consisting of SEQ ID NO:31.

In certain embodiments, a polypeptide of the disclosure comprises a VP1u region and/or VP1/2s region of AAV6, and a VP3 region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:33. In some embodiments, a polypeptide comprises the amino acid sequence of SEQ ID NO:33. In some embodiments, a polypeptide consists of the amino acid sequence of SEQ ID NO:33. In some embodiments, a polypeptide comprises the amino acid sequence consisting of SEQ ID NO:33. In some embodiments, a polypeptide comprises the VP1u portion (e.g., AAV6 VP1u) of SEQ ID NO:33. In some embodiments, a polypeptide comprises the VP1/2s portion (e.g., AAV6 VP1/2) of SEQ ID NO:33. In some embodiments, a polypeptide comprises the VP3 portion (e.g., AAV8 VP3) of SEQ ID NO:33. In some embodiments, a polypeptide consists of the VP1u portion (e.g., AAV6 VP1u) of SEQ ID NO: 33. In some embodiments, a polypeptide consists of the VP1/2s portion (e.g., AAV6 VP1/2) of SEQ ID NO:33. In some embodiments, a polypeptide consists of the VP3 portion (e.g., AAV8 VP3) of SEQ ID NO:33. In some embodiments, a polypeptide comprises the VP1u portion (e.g., AAV6 VP1u) of an amino acid sequence consisting of SEQ ID NO:33. In some embodiments, a polypeptide comprises the VP1/2s portion (e.g., AAV6 VP1/2) of an amino acid sequence consisting of SEQ ID NO:33. In some embodiments, a polypeptide comprises the VP3 portion (e.g., AAV8 VP3) of an amino acid sequence consisting of SEQ ID NO: 33.

In certain embodiments, a polypeptide of the disclosure comprises a VP1u region and/or VP1/2s region of AAV6, and a VP3 region of AAV rh.21. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:35. In some embodiments, a polypeptide comprises the amino acid sequence of SEQ ID NO:35. In some embodiments, a polypeptide consists of the amino acid sequence of SEQ ID NO:35. In some embodiments, a polypeptide comprises the amino acid sequence consisting of SEQ ID NO:35. In some embodiments, a polypeptide comprises the VP1u portion (e.g., AAV6 VP1u) of SEQ ID NO:35. In some embodiments, a polypeptide comprises the VP1/2s portion (e.g., AAV6 VP1/2) of SEQ ID NO:35. In some embodiments, a polypeptide comprises the VP3 portion (e.g., AAV rh.21 VP3) of SEQ ID NO:35. In some embodiments, a polypeptide consists of the VP1u portion (e.g., AAV6 VP1u) of SEQ ID NO: 35. In some embodiments, a polypeptide consists of the VP1/2s portion (e.g., AAV6 VP1/2) of SEQ ID NO:35. In some embodiments, a polypeptide consists of the VP3 portion (e.g., AAV rh.21 VP3) of SEQ ID NO:35. In some embodiments, a polypeptide comprises the VP1u portion (e.g., AAV6 VP1u) of an amino acid sequence consisting of SEQ ID NO: 35. In some embodiments, a polypeptide comprises the VP1/2s portion (e.g., AAV6 VP1/2) of an amino acid sequence consisting of SEQ ID NO:35. In some embodiments, a polypeptide comprises the VP3 portion (e.g., AAV rh.21 VP3) of an amino acid sequence consisting of SEQ ID NO:35.

In certain embodiments, a polypeptide of the disclosure comprises a VP1u region and/or VP1/2s region of AAV6, and a VP3 region of AAV rh.73. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:37. In some embodiments, a polypeptide comprises the amino acid sequence of SEQ ID NO:37. In some embodiments, a polypeptide consists of the amino acid sequence of SEQ ID NO:37. In some embodiments, a polypeptide comprises the amino acid sequence consisting of SEQ ID NO:37. In some embodiments, a polypeptide comprises the VP1u portion (e.g., AAV6 VP1u) of SEQ ID NO:37. In some embodiments, a polypeptide comprises the VP1/2s portion (e.g., AAV6 VP1/2) of SEQ ID NO:37. In some embodiments, a polypeptide comprises the VP3 portion (e.g., AAV rh.73 VP3) of SEQ ID NO:37. In some embodiments, a polypeptide consists of the VP1u portion (e.g., AAV6 VP1u) of SEQ ID NO: 37. In some embodiments, a polypeptide consists of the VP1/2s portion (e.g., AAV6 VP1/2) of SEQ ID NO:37. In some embodiments, a polypeptide consists of the VP3 portion (e.g., AAV rh.73 VP3) of SEQ ID NO:37. In some embodiments, a polypeptide comprises the VP1u portion (e.g., AAV6 VP1u) of an amino acid sequence consisting of SEQ ID NO: 37. In some embodiments, a polypeptide comprises the VP1/2s portion (e.g., AAV6 VP1/2) of an amino acid sequence consisting of SEQ ID NO:37. In some embodiments, a polypeptide comprises the VP3 portion (e.g., AAV rh.73 VP3) of an amino acid sequence consisting of SEQ ID NO:37.

In certain embodiments, a polypeptide of the disclosure comprises a VP1u region and/or VP1/2s region of AAV8, and a VP3 region of AAV6. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:39. In some embodiments, a polypeptide comprises the amino acid sequence of SEQ ID NO:39. In some embodiments, a polypeptide consists of the amino acid sequence of SEQ ID NO:39. In some embodiments, a polypeptide comprises the amino acid sequence consisting of SEQ ID NO:39. In some embodiments, a polypeptide comprises the VP1u portion (e.g., AAV8 VP1u) of SEQ ID NO:39. In some embodiments, a polypeptide comprises the VP1/2s portion (e.g., AAV8 VP1/2) of SEQ ID NO:39. In some embodiments, a polypeptide comprises the VP3 portion (e.g., AAV6 VP3) of SEQ ID NO:39. In some embodiments, a polypeptide consists of the VP1u portion (e.g., AAV8 VP1u) of SEQ ID NO: 39. In some embodiments, a polypeptide consists of the VP1/2s portion (e.g., AAV8 VP1/2) of SEQ ID NO:39. In some embodiments, a polypeptide consists of the VP3 portion (e.g., AAV6 VP3) of SEQ ID NO:39. In some embodiments, a polypeptide comprises the VP1u portion (e.g., AAV8 VP1u) of an amino acid sequence consisting of SEQ ID NO:39. In some embodiments, a polypeptide comprises the VP1/2s portion (e.g., AAV8 VP1/2) of an amino acid sequence consisting of SEQ ID NO:39. In some embodiments, a polypeptide comprises the VP3 portion (e.g., AAV6 VP3) of an amino acid sequence consisting of SEQ ID NO: 39.

In certain embodiments, a polypeptide of the disclosure comprises a VP1u region and/or VP1/2s region of AAV8, and a VP3 region of AAV rh.21. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:41. In some embodiments, a polypeptide comprises the amino acid sequence of SEQ ID NO:41. In some embodiments, a polypeptide consists of the amino acid sequence of SEQ ID NO:41. In some embodiments, a polypeptide comprises the amino acid sequence consisting of SEQ ID NO:41. In some embodiments, a polypeptide comprises the VP1u portion (e.g., AAV8 VP1u) of SEQ ID NO:41. In some embodiments, a polypeptide comprises the VP1/2s portion (e.g., AAV8 VP1/2) of SEQ ID NO:41. In some embodiments, a polypeptide comprises the VP3 portion (e.g., AAV rh.21 VP3) of SEQ ID NO:41. In some embodiments, a polypeptide consists of the VP1u portion (e.g., AAV8 VP1u) of SEQ ID NO: 41. In some embodiments, a polypeptide consists of the VP1/2s portion (e.g., AAV8 VP1/2) of SEQ ID NO:41. In some embodiments, a polypeptide consists of the VP3 portion (e.g., AAV rh.21 VP3) of SEQ ID NO:41. In some embodiments, a polypeptide comprises the VP1u portion (e.g., AAV8 VP1u) of an amino acid sequence consisting of SEQ ID NO: 41. In some embodiments, a polypeptide comprises the VP1/2s portion (e.g., AAV8 VP1/2) of an amino acid sequence consisting of SEQ ID NO:41. In some embodiments, a polypeptide comprises the VP3 portion (e.g., AAV rh.21 VP3) of an amino acid sequence consisting of SEQ ID NO:41.

In certain embodiments, a polypeptide of the disclosure comprises a VP1u region and/or VP1/2s region of AAV8, and a VP3 region of AAV rh.73. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:43. In some embodiments, a polypeptide comprises the amino acid sequence of SEQ ID NO:43. In some embodiments, a polypeptide consists of the amino acid sequence of SEQ ID NO:43. In some embodiments, a polypeptide comprises the amino acid sequence consisting of SEQ ID NO:43. In some embodiments, a polypeptide comprises the VP1u portion (e.g., AAV8 VP1u) of SEQ ID NO:43. In some embodiments, a polypeptide comprises the VP1/2s portion (e.g., AAV8 VP1/2) of SEQ ID NO:43. In some embodiments, a polypeptide comprises the VP3 portion (e.g., AAV rh.73 VP3) of SEQ ID NO:43. In some embodiments, a polypeptide consists of the VP1u portion (e.g., AAV8 VP1u) of SEQ ID NO: 43. In some embodiments, a polypeptide consists of the VP1/2s portion (e.g., AAV8 VP1/2) of SEQ ID NO:43. In some embodiments, a polypeptide consists of the VP3 portion (e.g., AAV rh.73 VP3) of SEQ ID NO:43. In some embodiments, a polypeptide comprises the VP1u portion (e.g., AAV8 VP1u) of an amino acid sequence consisting of SEQ ID NO: 43. In some embodiments, a polypeptide comprises the VP1/2s portion (e.g., AAV8 VP1/2) of an amino acid sequence consisting of SEQ ID NO:43. In some embodiments, a polypeptide comprises the VP3 portion (e.g., AAV rh.73 VP3) of an amino acid sequence consisting of SEQ ID NO:43.

In certain embodiments, a polypeptide of the disclosure comprises a VP1u region and/or VP1/2s region of AAV9, and a VP3 region of AAV4. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:45. In some embodiments, a polypeptide comprises the amino acid sequence of SEQ ID NO:45. In some embodiments, a polypeptide consists of the amino acid sequence of SEQ ID NO:45. In some embodiments, a polypeptide comprises the amino acid sequence consisting of SEQ ID NO:45. In some embodiments, a polypeptide comprises the VP1u portion (e.g., AAV9 VP1u) of SEQ ID NO:45. In some embodiments, a polypeptide comprises the VP1/2s portion (e.g., AAV9 VP1/2) of SEQ ID NO:45. In some embodiments, a polypeptide comprises the VP3 portion (e.g., AAV4 VP3) of SEQ ID NO:45. In some embodiments, a polypeptide consists of the VP1u portion (e.g., AAV9 VP1u) of SEQ ID NO: 45. In some embodiments, a polypeptide consists of the VP1/2s portion (e.g., AAV9 VP1/2) of SEQ ID NO:45. In some embodiments, a polypeptide consists of the VP3 portion (e.g., AAV4 VP3) of SEQ ID NO:45. In some embodiments, a polypeptide comprises the VP1u portion (e.g., AAV9 VP1u) of an amino acid sequence consisting of SEQ ID NO:45. In some embodiments, a polypeptide comprises the VP1/2s portion (e.g., AAV9 VP1/2) of an amino acid sequence consisting of SEQ ID NO:45. In some embodiments, a polypeptide comprises the VP3 portion (e.g., AAV4 VP3) of an amino acid sequence consisting of SEQ ID NO: 45.

In certain embodiments, a polypeptide of the disclosure comprises a VP1u region and/or VP1/2s region of AAV9, and a VP3 region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:47. In some embodiments, a polypeptide comprises the amino acid sequence of SEQ ID NO:47. In some embodiments, a polypeptide consists of the amino acid sequence of SEQ ID NO:47. In some embodiments, a polypeptide comprises the amino acid sequence consisting of SEQ ID NO:47. In some embodiments, a polypeptide comprises the VP1u portion (e.g., AAV9 VP1u) of SEQ ID NO:47. In some embodiments, a polypeptide comprises the VP1/2s portion (e.g., AAV9 VP1/2) of SEQ ID NO:47. In some embodiments, a polypeptide comprises the VP3 portion (e.g., AAV8 VP3) of SEQ ID NO:47. In some embodiments, a polypeptide consists of the VP1u portion (e.g., AAV9 VP1u) of SEQ ID NO: 47. In some embodiments, a polypeptide consists of the VP1/2s portion (e.g., AAV9 VP1/2) of SEQ ID NO:47. In some embodiments, a polypeptide consists of the VP3 portion (e.g., AAV8 VP3) of SEQ ID NO:47. In some embodiments, a polypeptide comprises the VP1u portion (e.g., AAV9 VP1u) of an amino acid sequence consisting of SEQ ID NO:47. In some embodiments, a polypeptide comprises the VP1/2s portion (e.g., AAV9 VP1/2) of an amino acid sequence consisting of SEQ ID NO:47. In some embodiments, a polypeptide comprises the VP3 portion (e.g., AAV8 VP3) of an amino acid sequence consisting of SEQ ID NO: 47.

In certain embodiments, a polypeptide of the disclosure comprises a VP1u region and/or VP1/2s region of AAV9, and a VP3 region of AAV rh18. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:49. In some embodiments, a polypeptide comprises the amino acid sequence of SEQ ID NO:49. In some embodiments, a polypeptide consists of the amino acid sequence of SEQ ID NO:49. In some embodiments, a polypeptide comprises the amino acid sequence consisting of SEQ ID NO:49. In some embodiments, a polypeptide comprises the VP1u portion (e.g., AAV9 VP1u) of SEQ ID NO:49. In some embodiments, a polypeptide comprises the VP1/2s portion (e.g., AAV9 VP1/2) of SEQ ID NO:49. In some embodiments, a polypeptide comprises the VP3 portion (e.g., AAV rh18 VP3) of SEQ ID NO:49. In some embodiments, a polypeptide consists of the VP1u portion (e.g., AAV9 VP1u) of SEQ ID NO: 49. In some embodiments, a polypeptide consists of the VP1/2s portion (e.g., AAV9 VP1/2) of SEQ ID NO:49. In some embodiments, a polypeptide consists of the VP3 portion (e.g., AAV rh18 VP3) of SEQ ID NO:49. In some embodiments, a polypeptide comprises the VP1u portion (e.g., AAV9 VP1u) of an amino acid sequence consisting of SEQ ID NO:49. In some embodiments, a polypeptide comprises the VP1/2s portion (e.g., AAV9 VP1/2) of an amino acid sequence consisting of SEQ ID NO:49. In some embodiments, a polypeptide comprises the VP3 portion (e.g., AAV rh18 VP3) of an amino acid sequence consisting of SEQ ID NO:49.

In certain embodiments, a polypeptide of the disclosure comprises a VP1u region and/or VP1/2s region of AAV rh.21, and a VP3 region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:51. In some embodiments, a polypeptide comprises the amino acid sequence of SEQ ID NO:51. In some embodiments, a polypeptide consists of the amino acid sequence of SEQ ID NO:51. In some embodiments, a polypeptide comprises the amino acid sequence consisting of SEQ ID NO:51. In some embodiments, a polypeptide comprises the VP1u portion (e.g., AAV rh.21 VP1u) of SEQ ID NO:51. In some embodiments, a polypeptide comprises the VP1/2s portion (e.g., AAV rh.21 VP1/2) of SEQ ID NO:51. In some embodiments, a polypeptide comprises the VP3 portion (e.g., AAV8 VP3) of SEQ ID NO:51. In some embodiments, a polypeptide consists of the VP1u portion (e.g., AAV rh.21 VP1u) of SEQ ID NO:51. In some embodiments, a polypeptide consists of the VP1/2s portion (e.g., AAV rh.21 VP1/2) of SEQ ID NO:51. In some embodiments, a polypeptide consists of the VP3 portion (e.g., AAV8 VP3) of SEQ ID NO:51. In some embodiments, a polypeptide comprises the VP1u portion (e.g., AAV rh.21 VP1u) of an amino acid sequence consisting of SEQ ID NO:51. In some embodiments, a polypeptide comprises the VP1/2s portion (e.g., AAV rh.21 VP1/2) of an amino acid sequence consisting of SEQ ID NO:51. In some embodiments, a polypeptide comprises the VP3 portion (e.g., AAV8 VP3) of an amino acid sequence consisting of SEQ ID NO:51.

In certain embodiments, a polypeptide of the disclosure comprises a VP1u region and/or VP1/2s region of AAV rh73, and a VP3 region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:53. In some embodiments, a polypeptide comprises the amino acid sequence of SEQ ID NO:53. In some embodiments, a polypeptide consists of the amino acid sequence of SEQ ID NO:53. In some embodiments, a polypeptide comprises the amino acid sequence consisting of SEQ ID NO:53. In some embodiments, a polypeptide comprises the VP1u portion (e.g., AAV rh73 VP1u) of SEQ ID NO:53. In some embodiments, a polypeptide comprises the VP1/2s portion (e.g., AAV rh73 VP1/2) of SEQ ID NO:53. In some embodiments, a polypeptide comprises the VP3 portion (e.g., AAV8 VP3) of SEQ ID NO:53. In some embodiments, a polypeptide consists of the VP1u portion (e.g., AAV rh73 VP1u) of SEQ ID NO:53. In some embodiments, a polypeptide consists of the VP1/2s portion (e.g., AAV rh73 VP1/2) of SEQ ID NO:53. In some embodiments, a polypeptide consists of the VP3 portion (e.g., AAV8 VP3) of SEQ ID NO:53. In some embodiments, a polypeptide comprises the VP1u portion (e.g., AAV rh 73 VP1u) of an amino acid sequence consisting of SEQ ID NO:53. In some embodiments, a polypeptide comprises the VP1/2s portion (e.g., AAV rh73 VP1/2) of an amino acid sequence consisting of SEQ ID NO:53. In some embodiments, a polypeptide comprises the VP3 portion (e.g., AAV8 VP3) of an amino acid sequence consisting of SEQ ID NO:53.

In certain embodiments, a polypeptide of the disclosure comprises a VP1u region and/or VP1/2s region of AAV3B, and a VP3 region of AAV8. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:55. In some embodiments, a polypeptide comprises the amino acid sequence of SEQ ID NO:55. In some embodiments, a polypeptide consists of the amino acid sequence of SEQ ID NO:55. In some embodiments, a polypeptide comprises the amino acid sequence consisting of SEQ ID NO:55. In some embodiments, a polypeptide comprises the VP1u portion (e.g., AAV3B VP1u) of SEQ ID NO:55. In some embodiments, a polypeptide comprises the VP1/2s portion (e.g., AAV3B VP1/2) of SEQ ID NO:55. In some embodiments, a polypeptide comprises the VP3 portion (e.g., AAV8 VP3) of SEQ ID NO:55. In some embodiments, a polypeptide consists of the VP1u portion (e.g., AAV3B VP1u) of SEQ ID NO:55. In some embodiments, a polypeptide consists of the VP1/2s portion (e.g., AAV3B VP1/2) of SEQ ID NO:55. In some embodiments, a polypeptide consists of the VP3 portion (e.g., AAV8 VP3) of SEQ ID NO:55. In some embodiments, a polypeptide comprises the VP1u portion (e.g., AAV3B VP1u) of an amino acid sequence consisting of SEQ ID NO: 55. In some embodiments, a polypeptide comprises the VP1/2s portion (e.g., AAV3B VP1/2) of an amino acid sequence consisting of SEQ ID NO:55. In some embodiments, a polypeptide comprises the VP3 portion (e.g., AAV8 VP3) of an amino acid sequence consisting of SEQ ID NO:55.

In certain embodiments, a polypeptide of the disclosure comprises a VP1u region and/or VP1/2s region of AAV rh. 15, and a VP3 region of AAVhu32. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:57. In some embodiments, a polypeptide comprises the amino acid sequence of SEQ ID NO:57. In some embodiments, a polypeptide consists of the amino acid sequence of SEQ ID NO:57. In some embodiments, a polypeptide comprises the amino acid sequence consisting of SEQ ID NO:57. In some embodiments, a polypeptide comprises the VP1u portion (e.g., AAV rh.15 VP1u) of SEQ ID NO:57. In some embodiments, a polypeptide comprises the VP1/2s portion (e.g., AAV rh. 15 VP1/2) of SEQ ID NO:57. In some embodiments, a polypeptide comprises the VP3 portion (e.g., AAVhu32 VP3) of SEQ ID NO: 57. In some embodiments, a polypeptide consists of the VP1u portion (e.g., AAV rh. 15 VP1u) of SEQ ID NO:57. In some embodiments, a polypeptide consists of the VP1/2s portion (e.g., AAV rh.15 VP1/2) of SEQ ID NO:57. In some embodiments, a polypeptide consists of the VP3 portion (e.g., AAVhu32 VP3) of SEQ ID NO:57. In some embodiments, a polypeptide comprises the VP1u portion (e.g., AAV rh.15 VP1u) of an amino acid sequence consisting of SEQ ID NO:57. In some embodiments, a polypeptide comprises the VP1/2s portion (e.g., AAV rh. 15 VP1/2) of an amino acid sequence consisting of SEQ ID NO:57. In some embodiments, a polypeptide comprises the VP3 portion (e.g., AAVhu32 VP3) of an amino acid sequence consisting of SEQ ID NO:57.

In certain embodiments, a polypeptide of the disclosure a VP1u region and/or VP1/2s region of AAV rh.13, and a VP3 region of AAVhu32. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:59. In some embodiments, a polypeptide comprises the amino acid sequence of SEQ ID NO:59. In some embodiments, a polypeptide consists of the amino acid sequence of SEQ ID NO:59. In some embodiments, a polypeptide comprises the amino acid sequence consisting of SEQ ID NO:59. In some embodiments, a polypeptide comprises the VP1u portion (e.g., AAV rh.13 VP1u) of SEQ ID NO:59. In some embodiments, a polypeptide comprises the VP1/2s portion (e.g., AAV rh. 13 VP1/2) of SEQ ID NO:59. In some embodiments, a polypeptide comprises the VP3 portion (e.g., AAVhu32 VP3) of SEQ ID NO: 59. In some embodiments, a polypeptide consists of the VP1u portion (e.g., AAV rh.13 VP1u) of SEQ ID NO:59. In some embodiments, a polypeptide consists of the VP1/2s portion (e.g., AAV rh.13 VP1/2) of SEQ ID NO:59. In some embodiments, a polypeptide consists of the VP3 portion (e.g., AAVhu32 VP3) of SEQ ID NO:59. In some embodiments, a polypeptide comprises the VP1u portion (e.g., AAV rh.13 VP1u) of an amino acid sequence consisting of SEQ ID NO:59. In some embodiments, a polypeptide comprises the VP1/2s portion (e.g., AAV rh. 13 VP1/2) of an amino acid sequence consisting of SEQ ID NO:59. In some embodiments, a polypeptide comprises the VP3 portion (e.g., AAVhu32 VP3) of an amino acid sequence consisting of SEQ ID NO:59.

In certain embodiments, a polypeptide of the disclosure comprises a VP1u region and/or VP1/2s region of AAV6, and a VP3 region of AAVhu32. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:61. In some embodiments, a polypeptide comprises the amino acid sequence of SEQ ID NO:61. In some embodiments, a polypeptide consists of the amino acid sequence of SEQ ID NO:61. In some embodiments, a polypeptide comprises the amino acid sequence consisting of SEQ ID NO:61. In some embodiments, a polypeptide comprises the VP1u portion (e.g., AAV6 VP1u) of SEQ ID NO:61. In some embodiments, a polypeptide comprises the VP1/2s portion (e.g., AAV6 VP1/2) of SEQ ID NO:61. In some embodiments, a polypeptide comprises the VP3 portion (e.g., AAVhu32 VP3) of SEQ ID NO:61. In some embodiments, a polypeptide consists of the VP1u portion (e.g., AAV6 VP1u) of SEQ ID NO: 61. In some embodiments, a polypeptide consists of the VP1/2s portion (e.g., AAV6 VP1/2) of SEQ ID NO:61. In some embodiments, a polypeptide consists of the VP3 portion (e.g., AAVhu32 VP3) of SEQ ID NO:61. In some embodiments, a polypeptide comprises the VP1u portion (e.g., AAV6 VP1u) of an amino acid sequence consisting of SEQ ID NO:61. In some embodiments, a polypeptide comprises the VP1/2s portion (e.g., AAV6 VP1/2) of an amino acid sequence consisting of SEQ ID NO:61. In some embodiments, a polypeptide comprises the VP3 portion (e.g., AAVhu32 VP3) of an amino acid sequence consisting of SEQ ID NO:61.

In certain embodiments, a polypeptide of the disclosure comprises a VP1u region and/or VP1/2s region of AAV5, and a VP3 region of AAVhu32. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:63. In some embodiments, a polypeptide comprises the amino acid sequence of SEQ ID NO:63. In some embodiments, a polypeptide consists of the amino acid sequence of SEQ ID NO:63. In some embodiments, a polypeptide comprises the amino acid sequence consisting of SEQ ID NO:63. In some embodiments, a polypeptide comprises the VP1u portion (e.g., AAV5 VP1u) of SEQ ID NO:63. In some embodiments, a polypeptide comprises the VP1/2s portion (e.g., AAV5 VP1/2) of SEQ ID NO:63. In some embodiments, a polypeptide comprises the VP3 portion (e.g., AAVhu32 VP3) of SEQ ID NO:63. In some embodiments, a polypeptide consists of the VP1u portion (e.g., AAV5 VP1u) of SEQ ID NO: 63. In some embodiments, a polypeptide consists of the VP1/2s portion (e.g., AAV5 VP1/2) of SEQ ID NO:63. In some embodiments, a polypeptide consists of the VP3 portion (e.g., AAVhu32 VP3) of SEQ ID NO:63. In some embodiments, a polypeptide comprises the VP1u portion (e.g., AAV5 VP1u) of an amino acid sequence consisting of SEQ ID NO:63. In some embodiments, a polypeptide comprises the VP1/2s portion (e.g., AAV5 VP1/2) of an amino acid sequence consisting of SEQ ID NO:63. In some embodiments, a polypeptide comprises the VP3 portion (e.g., AAVhu32 VP3) of an amino acid sequence consisting of SEQ ID NO:63.

In certain embodiments, a polypeptide of the disclosure comprises a VP1u regio and/or VP1/2s region of AAV hu.32, and a VP3 region of AAV rh.15. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:65. In some embodiments, a polypeptide comprises the amino acid sequence of SEQ ID NO:65. In some embodiments, a polypeptide consists of the amino acid sequence of SEQ ID NO:65. In some embodiments, a polypeptide comprises the amino acid sequence consisting of SEQ ID NO:65. In some embodiments, a polypeptide comprises the VP1u portion (e.g., AAV hu.32 VP1u) of SEQ ID NO:65. In some embodiments, a polypeptide comprises the VP1/2s portion (e.g., AAV hu.32 VP1/2) of SEQ ID NO:65. In some embodiments, a polypeptide comprises the VP3 portion (e.g., AAV rh.15 VP3) of SEQ ID NO: 65. In some embodiments, a polypeptide consists of the VP1u portion (e.g., AAV hu.32 VP1u) of SEQ ID NO:65. In some embodiments, a polypeptide consists of the VP1/2s portion (e.g., AAV hu.32 VP1/2) of SEQ ID NO:65. In some embodiments, a polypeptide consists of the VP3 portion (e.g., AAV rh. 15 VP3) of SEQ ID NO:65. In some embodiments, a polypeptide comprises the VP1u portion (e.g., AAV hu.32 VP1u) of an amino acid sequence consisting of SEQ ID NO:65. In some embodiments, a polypeptide comprises the VP1/2s portion (e.g., AAV hu.32 VP1/2) of an amino acid sequence consisting of SEQ ID NO: 65. In some embodiments, a polypeptide comprises the VP3 portion (e.g., AAV rh.15 VP3) of an amino acid sequence consisting of SEQ ID NO:65.

In certain embodiments, a polypeptide of the disclosure comprises a VP1u region and/or VP1/2s region of AAV6, and a VP3 region of AAV ru.15. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:67. In some embodiments, a polypeptide comprises the amino acid sequence of SEQ ID NO:67. In some embodiments, a polypeptide consists of the amino acid sequence of SEQ ID NO:67. In some embodiments, a polypeptide comprises the amino acid sequence consisting of SEQ ID NO:67. In some embodiments, a polypeptide comprises the VP1u portion (e.g., AAV6 VP1u) of SEQ ID NO:67. In some embodiments, a polypeptide comprises the VP1/2s portion (e.g., AAV6 VP1/2) of SEQ ID NO:67. In some embodiments, a polypeptide comprises the VP3 portion (e.g., AAV ru. 15 VP3) of SEQ ID NO:67. In some embodiments, a polypeptide consists of the VP1u portion (e.g., AAV6 VP1u) of SEQ ID NO: 67. In some embodiments, a polypeptide consists of the VP1/2s portion (e.g., AAV6 VP1/2) of SEQ ID NO:67. In some embodiments, a polypeptide consists of the VP3 portion (e.g., AAV ru. 15 VP3) of SEQ ID NO:67. In some embodiments, a polypeptide comprises the VP1u portion (e.g., AAV6 VP1u) of an amino acid sequence consisting of SEQ ID NO: 67. In some embodiments, a polypeptide comprises the VP1/2s portion (e.g., AAV6 VP1/2) of an amino acid sequence consisting of SEQ ID NO:67. In some embodiments, a polypeptide comprises the VP3 portion (e.g., AAV ru.15 VP3) of an amino acid sequence consisting of SEQ ID NO:67.

In certain embodiments, a polypeptide of the disclosure a VP1u region and/or VP1/2s region of AAV5, and a VP3 region of AAV rh.15. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:69. In some embodiments, a polypeptide comprises the amino acid sequence of SEQ ID NO:69. In some embodiments, a polypeptide consists of the amino acid sequence of SEQ ID NO:69. In some embodiments, a polypeptide comprises the amino acid sequence consisting of SEQ ID NO:69. In some embodiments, a polypeptide comprises the VP1u portion (e.g., AAV5 VP1u) of SEQ ID NO:69. In some embodiments, a polypeptide comprises the VP1/2s portion (e.g., AAV5 VP1/2) of SEQ ID NO:69. In some embodiments, a polypeptide comprises the VP3 portion (e.g., AAV rh.15 VP3) of SEQ ID NO:69. In some embodiments, a polypeptide consists of the VP1u portion (e.g., AAV5 VP1u) of SEQ ID NO: 69. In some embodiments, a polypeptide consists of the VP1/2s portion (e.g., AAV5 VP1/2) of SEQ ID NO:69. In some embodiments, a polypeptide consists of the VP3 portion (e.g., AAV rh. 15 VP3) of SEQ ID NO:69. In some embodiments, a polypeptide comprises the VP1u portion (e.g., AAV5 VP1u) of an amino acid sequence consisting of SEQ ID NO: 69. In some embodiments, a polypeptide comprises the VP1/2s portion (e.g., AAV5 VP1/2) of an amino acid sequence consisting of SEQ ID NO:69. In some embodiments, a polypeptide comprises the VP3 portion (e.g., AAV rh.15 VP3) of an amino acid sequence consisting of SEQ ID NO:69.

In certain embodiments, a polypeptide of the disclosure comprises a VP1u region and/or VP1/2s region of AAV6, and a VP3 region of AAV rh.13. In some embodiments, a polypeptide comprises an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:71. In some embodiments, a polypeptide comprises the amino acid sequence of SEQ ID NO:71. In some embodiments, a polypeptide consists of the amino acid sequence of SEQ ID NO:71. In some embodiments, a polypeptide comprises the amino acid sequence consisting of SEQ ID NO:71. In some embodiments, a polypeptide comprises the VP1u portion (e.g., AAV6 VP1u) of SEQ ID NO:71. In some embodiments, a polypeptide comprises the VP1/2s portion (e.g., AAV6 VP1/2) of SEQ ID NO:71. In some embodiments, a polypeptide comprises the VP3 portion (e.g., AAV rh.13 VP3) of SEQ ID NO:71. In some embodiments, a polypeptide consists of the VP1u portion (e.g., AAV6 VP1u) of SEQ ID NO: 71. In some embodiments, a polypeptide consists of the VP1/2s portion (e.g., AAV6 VP1/2) of SEQ ID NO:71. In some embodiments, a polypeptide consists of the VP3 portion (e.g., AAV rh. 13 VP3) of SEQ ID NO:71. In some embodiments, a polypeptide comprises the VP1u portion (e.g., AAV6 VP1u) of an amino acid sequence consisting of SEQ ID NO: 71. In some embodiments, a polypeptide comprises the VP1/2s portion (e.g., AAV6 VP1/2) of an amino acid sequence consisting of SEQ ID NO:71. In some embodiments, a polypeptide comprises the VP3 portion (e.g., AAV rh.13 VP3) of an amino acid sequence consisting of SEQ ID NO:71.

In some embodiments, the polypeptide of the disclosure comprises about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and/or 71. In some embodiments, a polypeptide comprises the amino acid sequence of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and/or 71. In some embodiments, a polypeptide consists of the amino acid sequence of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and/or 71. In some embodiments, a polypeptide comprises the amino acid sequence consisting of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and/or 71.

In some embodiments, a polypeptide comprises one or more of any one of SEQ ID NOs: 81, 83, 85, 87, 89, 91, 93, 95, 97, 99, 101, 103, 105, 107, 109, 111, 113, 115, 117, and 119. In some embodiments, a polypeptide comprises about or at least about 80%, 85%, 90%, 91%, 92%, 93%, 94%, 95%, 96%, 97%, 98%, 99%, or 100% identical to any one of SEQ ID NOs: 81, 83, 85, 87, 89, 91, 93, 95, 97, 99, 101, 103, 105, 107, 109, 111, 113, 115, 117, and 119. In some embodiments, a polypeptide consists of any one of SEQ ID NOs: 81, 83, 85, 87, 89, 91, 93, 95, 97, 99, 101, 103, 105, 107, 109, 111, 113, 115, 117, and 119.

In various aspects of the polypeptide, the percent identity differences between amino acid sequences is from truncation of amino acids, addition of amino acids, or one or more amino acid substitutions of the polypeptide. In some embodiments, an amino acid substitution is a conservative substitution. Illustrative examples for conserved amino acid exchanges are amino acid substitutions that maintain structural and/or functional properties of the amino acids' side-chains, e.g., an aromatic amino acid is substituted for another aromatic amino acid, an acidic amino acid is substituted for another acidic amino acid, a basic amino acid is substituted for another basic amino acid, and an aliphatic amino acid is substituted for another aliphatic amino acid. In some embodiments, a conservative amino acid substitution is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In contrast, examples of non-conserved amino acid exchanges are amino acid substitutions that do not maintain structural and/or functional properties of the amino acids' side-chains, e.g., an aromatic amino acid is substituted for a basic, acidic, or aliphatic amino acid, an acidic amino acid is substituted for an aromatic, basic, or aliphatic amino acid, a basic amino acid is substituted for an acidic, aromatic or aliphatic amino acid, and an aliphatic amino acid is substituted for an aromatic, acidic or basic amino acid.

In one aspect, provided herein are vectors comprising a nucleotide sequence encoding a polypeptide as described herein. In some embodiments, the vector is an expression vector. In some embodiments, a vector comprises an expression cassette for expression of a polypeptide as described herein. In some embodiments, the vector is a viral vector.

Also provided are nucleic acids encoding the polypeptides as described herein. In some embodiments, the polypeptide is encoded by a nucleotide sequence comprising nucleotides that encode an AAV VP3 polypeptide comprising a sequence that shares about or at least about 80%, 85%, 90%, 95%, 96%, 97%, 98%, 99%, 99.5%, or 100% identity to SEQ ID NO:2, 12, or 28. In some embodiments, a polypeptide comprises the amino acid sequence of SEQ ID NO: 2, 12, and/or 28. In some embodiments, a polypeptide consists of the amino acid sequence of SEQ ID NO:2, 12, and/or 28. In some embodiments, a polypeptide comprises the amino acid sequence consisting of SEQ ID NO: 2, 12, and/or 28. In some embodiments, the polypeptide is encoded by a nucleotide sequence comprising nucleotides that encode an AAV VP1/2s comprises a sequence that shares about or at least about 80%, 85%, 90%, 95%, 96%, 97%, 98%, 99%, 99.5%, or 100% identity to SEQ ID NO:4 or 14. In some embodiments, a polypeptide comprises the amino acid sequence of SEQ ID NO: 4 and/or 14. In some embodiments, a polypeptide consists of the amino acid sequence of SEQ ID NO:4 and/or 14. In some embodiments, a polypeptide comprises the amino acid sequence consisting of SEQ ID NO: 4 and/or 14. In some embodiments, the polypeptide is encoded by a nucleotide sequence comprising nucleotides that encode an AAV VP2 comprises a sequence that shares about or at least about 80%, 85%, 90%, 95%, 96%, 97%, 98%, 99%, 99.5%, or 100% identity to SEQ ID NO:6 or 16. In some embodiments, a polypeptide comprises the amino acid sequence of SEQ ID NO: 6 and/or 16. In some embodiments, a polypeptide consists of the amino acid sequence of SEQ ID NO:6 and/or 16. In some embodiments, a polypeptide comprises the amino acid sequence consisting of SEQ ID NO: 6 and/or 16. In some embodiments, the polypeptide is encoded by a nucleotide sequence comprising nucleotides that encode an AAV VP1u comprises a sequence that shares about or at least about 80%, 85%, 90%, 95%, 96%, 97%, 98%, 99%, 99.5%, or 100% identity to SEQ ID NO:8 or 18. In some embodiments, a polypeptide comprises the amino acid sequence of SEQ ID NO: 8 and/or 18. In some embodiments, a polypeptide consists of the amino acid sequence of SEQ ID NO:8 and/or 18. In some embodiments, a polypeptide comprises the amino acid sequence consisting of SEQ ID NO: 8 and/or 18. In some embodiments, the polypeptide is encoded by a nucleotide sequence comprising nucleotides that encode an AAV VP1 and the nucleotide sequence comprises a sequence that shares about or at least about 80%, 85%, 90%, 95%, 96%, 97%, 98%, 99%, 99.5%, or 100% identity to SEQ ID NO:10, 20, 30, 32, 34, 36, 38, 40, 42, 44, 46, 48, 50, 52, 54, 56, 58, 60, 62, 64, 66, 68, 70, or 72. In some embodiments, a polypeptide comprises the amino acid sequence of SEQ ID NO: 10, 20, 30, 32, 34, 36, 38, 40, 42, 44, 46, 48, 50, 52, 54, 56, 58, 60, 62, 64, 66, 68, 70, and/or 72. In some embodiments, a polypeptide consists of the amino acid sequence of SEQ ID NO: 10, 20, 30, 32, 34, 36, 38, 40, 42, 44, 46, 48, 50, 52, 54, 56, 58, 60, 62, 64, 66, 68, 70, and/or 72. In some embodiments, a polypeptide comprises the amino acid sequence consisting of SEQ ID NO: 10, 20, 30, 32, 34, 36, 38, 40, 42, 44, 46, 48, 50, 52, 54, 56, 58, 60, 62, 64, 66, 68, 70, and/or 72. In some embodiments, the polypeptide is encoded by a nucleotide sequence comprising about or at least about 80%, 85%, 90%, 95%, 96%, 97%, 98%, 99%, 99.5%, or 100% identity to any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and/or 72. In some embodiments, a nucleotide sequence comprises the amino acid sequence of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and/or 72. In some embodiments, a nucleotide sequence consists of the amino acid sequence of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and/or 72. In some embodiments, a nucleotide sequence comprises the amino acid sequence consisting of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and/or 72. In some embodiments, provided herein is a host cell (e.g., in vitro cell) comprising a nucleotide sequence of the disclosure (e.g., a nucleotide sequence that encodes the polypeptide or hybrid AAV of the disclosure). In some embodiments, provided herein is a host cell (e.g., in vitro cell) comprising an expression vector of the disclosure.

As a skilled artisan would recognize, a polypeptide corresponding to a VP1 capsid protein may be truncated in vivo into a VP2 or VP3 capsid protein.

5.2 Recombinant Adeno-Associated Viruses (rAAVs) and Viral Vectors

### 5.2.1 Hybrid AAV Capsids

Provided herein is a recombinant adeno-associated virus (rAAV) comprising hybrid capsid proteins engineered to maintain or improve parental tropism while having improved endosomal escape and nuclear trafficking. In various aspects of the invention, an rAAV comprises a hybrid capsid protein comprising a VP1u and/or VP1/2s of a different serotype than the serotype of the VP3 capsid protein. In some embodiments, an rAAV comprises a hybrid capsid protein comprising a VP1/2s of a second AAV serotype and a VP3 of a first AAV serotype. In some embodiments, an rAAV comprises a hybrid capsid protein comprising a VP1u of a second AAV serotype, a VP1/2s of the second AAV serotype, and a VP3 of a first AAV serotype. In some embodiments, an rAAV comprises a hybrid capsid protein comprising a VP1u of a first AAV serotype, a VP1/2s of a second AAV serotype, and a VP3 of the first AAV serotype. In some embodiments, the first AAV serotype is AAV8 or AAV Rh13. In some embodiments, the second AAV serotype is AAV5 or AAVhu32. In specific embodiments, the first AAV serotype is AAV8 and the second AAV serotype is AAV5. In specific embodiments, the first AAV serotype is AAVrh13 and the second AAV serotype is AAVhu32. In some embodiments, a hybrid AAV of the disclosure comprises one or more than one of AAV1, AAV2, AAV3, AAV3B, AAV4, AAV5, AAV6, AAV7, AAV8, AAV9, AAVrh10, AAV11, AAV12, AAV13, AAVrh20, AAVhu.37, AAVrh39, AAVhu32, AAVrh21, AAVrh13, AAVrh15, AAVrh73, and/or AAVrh74.

In some embodiments, an rAAV (hybrid AAV) of the disclosure comprises one or more polypeptide of the disclosure (e.g., as described in Section 5.1.1). In some embodiments, provided herein is a recombinant adeno-associated virus (AAV) comprising a capsid protein (e.g., hybrid capsid protein) of the disclosure. In some embodiments, the rAAV comprises an expression cassette. In some embodiments, the expression cassette comprises an open reading frame (ORF) encoding a transgene (e.g., a transgene of the disclosure). In some embodiments, the expression cassette further comprises a promoter. In some embodiments, an rAAV comprises a capsid protein comprising: (i) an amino acid sequence that is at least about 95% sequence identical to VP1/2s region of a first AAV (e.g., AAV5, AAVhu32, or VP1/2s region of any wild-type AAV, or a VP1/2s region of any AAV of the disclosure or identified in Section 7), and (ii) an amino acid sequence that is at least about 95% sequence identical to VP3 region of a second AAV (e.g., AAV8, AAVrh13, or VP3 region of any wild-type AAV y AAV, or a VP3 region of any AAV of the disclosure or identified in Section 7). In some embodiments, an rAAV comprises a capsid protein comprising: (i) an amino acid sequence that is about or at least about 90%, 91%, 92%, 93%, 94%, 95%, 96%, 97%, 98%, 99% or 100% sequence identical to VP1/2s region of a first AAV (e.g., AAV5, AAVhu32, or VP1/2s region of any wild-type AAV, or a VP1/2s region of any AAV of the disclosure or identified in Section 7), and (ii) an amino acid sequence that is about or at least about 90%, 91%, 92%, 93%, 94%, 95%, 96%, 97%, 98%, 99% or 100% sequence identical to VP3 region of a second AAV (e.g., AAV8, AAVrh13, or VP3 region of any wild-type AAV, or a VP3 region of any AAV of the disclosure or identified in Section 7). In some embodiments, the capsid protein further comprises an amino acid sequence that is about or at least about 90%, 91%, 92%, 93%, 94%, 95%, 96%, 97%, 98%, 99% or 100% sequence identical to VP1u region of a first AAV or a second AAV (e.g., AAV5, AAVhu32, AAV8, AAVrh13, or VP1u of any wild-type AAV, or a VP1u region of any AAV of the disclosure or identified in Section 7). In some embodiments, the capsid protein comprises an amino acid sequence that is about or at least about 95% identical to VP1u, VP1/2s, and/or VP3 of a first or second AAV. In some embodiments, the capsid protein comprises an amino acid sequence that is about or at least about 98% identical to VP1u, VP1/2s, and/or VP3 of a first or second AAV. In some embodiments, the capsid protein comprises an amino acid sequence that is about or at least about 99% identical to VP1u, VP1/2s, and/or VP3 of a first or second AAV. In some embodiments, provided herein is a recombinant adeno-associated virus (AAV) comprising a capsid protein, comprising: (i) an amino acid sequence that is about or at least about 90%, 91%, 92%, 93%, 94%, 95%, 96%, 97%, 98%, 99% or 100% sequence identical to VP1u region of a first AAV (e.g., AAV5, AAVhu32, or VP1/2s region of any wild-type AAV, or a VP1/2s region of any AAV of the disclosure or identified in Section 7), (ii) an amino acid sequence that is about or at least about 90%, 91%, 92%, 93%, 94%, 95%, 96%, 97%, 98%, 99% or 100% sequence identical to VP1/2s region of a first AAV (e.g., AAV5, AAVhu32, or VP1/2s region of any wild-type AAV, or a VP1/2s region of any AAV of the disclosure or identified in Section 7), and (iii) an amino acid sequence that is about or at least about 90%, 91%, 92%, 93%, 94%, 95%, 96%, 97%, 98%, 99% or 100% sequence identical to VP3 region of a second AAV (e.g., AAV8, AAVrh13, or VP3 region of any wild-type AAV, or a VP3 region of any AAV of the disclosure or identified in Section 7).

In certain embodiments, an rAAV of the disclosure comprises a VP3 of AAV5. In some embodiments, an rAAV comprises a hybrid capsid protein comprising a VP3 of AAV5. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:1. In some embodiments, an rAAV comprises a hybrid capsid protein comprising the amino acid sequence of SEQ ID NO:1.

In certain embodiments, an rAAV of the disclosure comprises a VP1/2s of AAV5. In some embodiments, an rAAV comprises a hybrid capsid protein comprising a VP1/2s of AAV5. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:3. In some embodiments, an rAAV comprises a hybrid capsid protein comprising the amino acid sequence of SEQ ID NO:3.

In certain embodiments, an rAAV of the disclosure comprises a VP1/2s and VP3 of AAV5. In certain embodiments, an rAAV of the disclosure comprises a VP2 of AAV5. In some embodiments, an rAAV comprises a hybrid capsid protein comprising a VP1/2s and VP3 of AAV5. In some embodiments, an rAAV comprises a hybrid capsid protein comprising a VP2 of AAV5. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:3 and an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:1. In some embodiments, an rAAV comprises a hybrid capsid protein comprising the amino acid sequences of SEQ ID NO:3 and SEQ ID NO:1. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:5. In some embodiments, an rAAV comprises a hybrid capsid protein comprising the amino acid sequence of SEQ ID NO:5.

In certain embodiments, an rAAV of the disclosure comprises a VP1u of AAV5. In some embodiments, an rAAV comprises a hybrid capsid protein comprising a VP1u of AAV5. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:7. In some embodiments, an rAAV comprises a hybrid capsid protein comprising the amino acid sequence of SEQ ID NO:7.

In certain embodiments, an rAAV of the disclosure comprises a VP3 of AAV8. In some embodiments, an rAAV comprises a hybrid capsid protein comprising a VP3 of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, an rAAV comprises a hybrid capsid protein comprising the amino acid sequence of SEQ ID NO:11.

In certain embodiments, an rAAV of the disclosure comprises a VP1/2s of AAV8. In some embodiments, an rAAV comprises a hybrid capsid protein comprising a VP1/2s of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:13. In some embodiments, an rAAV comprises a hybrid capsid protein comprising the amino acid sequence of SEQ ID NO: 13.

In certain embodiments, an rAAV of the disclosure comprises a VP1/2s and VP3 of AAV8. In certain embodiments, an rAAV of the disclosure comprises a VP2 of AAV8. In some embodiments, an rAAV comprises a hybrid capsid protein comprising a VP1/2s and VP3 of AAV8. In some embodiments, an rAAV comprises a hybrid capsid protein comprising a VP2 of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:13 and an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, an rAAV comprises a hybrid capsid protein comprising the amino acid sequence of SEQ ID NO:3 and SEQ ID NO:1. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:15. In some embodiments, an rAAV comprises a hybrid capsid protein comprising the amino acid sequence of SEQ ID NO:15.

In certain embodiments, an rAAV of the disclosure comprises a VP1u of AAV8. In some embodiments, an rAAV comprises a hybrid capsid protein comprising a VP1u of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:17. In some embodiments, an rAAV comprises a hybrid capsid protein comprising the amino acid sequence of SEQ ID NO:17.

In certain embodiments, an rAAV comprises a VP1u, VP1/2s, and VP3 of AAV8. In certain embodiments, an rAAV comprises a VP1 of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:17, an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:13, and an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, an rAAV comprises a hybrid capsid protein comprising the amino acid sequences of SEQ ID NO:17, SEQ ID NO:13, and SEQ ID NO:11. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO: 19. In some embodiments, an rAAV comprises a hybrid capsid protein comprising the amino acid sequence of SEQ ID NO: 19.

In certain embodiments, an rAAV of the disclosure comprises a VP 1/2s of AAV5, and a VP3 of AAV8. In some embodiments, an rAAV comprises a hybrid capsid protein comprising a VP 1/2s of AAV5 and a VP3 of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:3, and an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, an rAAV comprises a hybrid capsid protein comprising the amino acid sequences of SEQ ID NO:3 and SEQ ID NO:11. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:21. In some embodiments, an rAAV comprises a hybrid capsid protein comprising the amino acid sequence of SEQ ID NO:21.

In certain embodiments, an rAAV of the disclosure comprises a VP1u of AAV5, a VP 1/2s of AAV5, and a VP3 of AAV8. In some embodiments, an rAAV comprises a hybrid capsid protein comprising a VP1u of AAV5, a VP 1/2s of AAV5, and a VP3 of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:7, an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:3, and an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, an rAAV comprises a hybrid capsid protein comprising the amino acid sequences of SEQ ID NO:7, SEQ ID NO:3, and SEQ ID NO:11. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:23. In some embodiments, an rAAV comprises a hybrid capsid protein comprising the amino acid sequence of SEQ ID NO:23.

In certain embodiments, an rAAV of the disclosure comprises a VP1u of AAV8, a VP 1/2s of AAV5, and a VP3 of AAV8. In some embodiments, an rAAV comprises a hybrid capsid protein comprising a VP1u of AAV8, a VP 1/2s of AAV5, and a VP3 of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:17, an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:3, and an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:11. In some embodiments, an rAAV comprises a hybrid capsid protein comprising the amino acid sequences of SEQ ID NO:17, SEQ ID NO:3, and SEQ ID NO:11. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:25. In some embodiments, an rAAV comprises a hybrid capsid protein comprising the amino acid sequence of SEQ ID NO:25.

In certain embodiments, an rAAV of the disclosure comprises a VP3 of AAV Rh13. In some embodiments, an rAAV comprises a hybrid capsid protein comprising a VP3 of AAV Rh13. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:27. In some embodiments, an rAAV comprises a hybrid capsid protein comprising the amino acid sequence of SEQ ID NO:27.

In certain embodiments, an rAAV of the disclosure comprises a VP1u of AAVhu32, a VP1/2s of AAVhu32, and a VP3 of AAVrh13. In some embodiments, an rAAV comprises a hybrid capsid protein comprising VP1u of AAVhu32, a VP1/2s of AAVhu32, and a VP3 of AAVrh13. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:29. In some embodiments, an rAAV comprises a hybrid capsid protein comprising the amino acid sequence of SEQ ID NO:29.

In certain embodiments, an rAAV of the disclosure comprises a VP1 of AAVhu32. In some embodiments, an rAAV comprises a capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:31. In some embodiments, an rAAV comprises a capsid protein comprising the amino acid sequence of SEQ ID NO:31.

In certain embodiments, an rAAV of the disclosure comprises a hybrid capsid protein comprising a VP1u and/or VP1/2s of AAV6, and a VP3 of AAVhu32. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:33. In some embodiments, an rAAV comprises a hybrid capsid protein comprising the amino acid sequence of SEQ ID NO:33. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:61. In some embodiments, an rAAV comprises a hybrid capsid protein comprising the amino acid sequence of SEQ ID NO:61

In certain embodiments, an rAAV of the disclosure comprises a hybrid capsid protein comprising a VP1u and/or VP1/2s of AAV6, and a VP3 of AAV rh.21. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:35. In some embodiments, an rAAV comprises a hybrid capsid protein comprising the amino acid sequence of SEQ ID NO:35.

In certain embodiments, an rAAV of the disclosure comprises a hybrid capsid protein comprising a VP1u and/or VP1/2s of AAV6, and a VP3 of AAV rh.73. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:37. In some embodiments, an rAAV comprises a hybrid capsid protein comprising the amino acid sequence of SEQ ID NO:37.

In certain embodiments, an rAAV of the disclosure comprises a hybrid capsid protein comprising a VP1u and/or VP1/2s of AAV8, and a VP3 of AAV6. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:39. In some embodiments, an rAAV comprises a hybrid capsid protein comprising the amino acid sequence of SEQ ID NO:39.

In certain embodiments, an rAAV of the disclosure comprises a hybrid capsid protein comprising a VP1u and/or VP1/2s of AAV8, and a VP3 of AAV rh.21. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:41. In some embodiments, an rAAV comprises a hybrid capsid protein comprising the amino acid sequence of SEQ ID NO:41.

In certain embodiments, an rAAV of the disclosure comprises a hybrid capsid protein comprising a VP1u and/or VP1/2s of AAV8, and a VP3 of AAV rh.73. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:43. In some embodiments, an rAAV comprises a hybrid capsid protein comprising the amino acid sequence of SEQ ID NO:43.

In certain embodiments, an rAAV of the disclosure comprises a hybrid capsid protein comprising a VP1u and/or VP1/2s of AAV9, and a VP3 of AAV4. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:45. In some embodiments, an rAAV comprises a hybrid capsid protein comprising the amino acid sequence of SEQ ID NO:45.

In certain embodiments, an rAAV of the disclosure comprises a hybrid capsid protein comprising a VP1u and/or VP1/2s of AAV9, and a VP3 of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:47. In some embodiments, an rAAV comprises a hybrid capsid protein comprising the amino acid sequence of SEQ ID NO:47.

In certain embodiments, an rAAV of the disclosure comprises a hybrid capsid protein comprising a VP1u and/or VP1/2s of AAV9, and a VP3 of AAV rh18. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:49. In some embodiments, an rAAV comprises a hybrid capsid protein comprising the amino acid sequence of SEQ ID NO:49.

In certain embodiments, an rAAV of the disclosure comprises a hybrid capsid protein comprising a VP1u and/or VP1/2s of AAV rh.21, and a VP3 of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:51. In some embodiments, an rAAV comprises a hybrid capsid protein comprising the amino acid sequence of SEQ ID NO:51.

In certain embodiments, an rAAV of the disclosure comprises a hybrid capsid protein comprising a VP1u and/or VP1/2s of AAV rh73, and a VP3 of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:53. In some embodiments, an rAAV comprises a hybrid capsid protein comprising the amino acid sequence of SEQ ID NO:53.

In certain embodiments, an rAAV of the disclosure comprises a hybrid capsid protein comprising a VP1u and/or VP1/2s of AAV3B, and a VP3 of AAV8. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:55. In some embodiments, an rAAV comprises a hybrid capsid protein comprising the amino acid sequence of SEQ ID NO:55.

In certain embodiments, an rAAV of the disclosure comprises a hybrid capsid protein comprising a VP1u and/or VP1/2s of AAV rh. 15, and a VP3 of AAVhu32. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:57. In some embodiments, an rAAV comprises a hybrid capsid protein comprising the amino acid sequence of SEQ ID NO:57.

In certain embodiments, an rAAV of the disclosure comprises a hybrid capsid protein comprising a VP1u and/or VP1/2s of AAVrh13, and a VP3 of AAVhu32. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:59. In some embodiments, an rAAV comprises a hybrid capsid protein comprising the amino acid sequence of SEQ ID NO:59.

In certain embodiments, an rAAV of the disclosure comprises a hybrid capsid protein comprising a VP1u and/or VP1/2s of AAV6, and a VP3 of AAVhu32. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:61. In some embodiments, an rAAV comprises a hybrid capsid protein comprising the amino acid sequence of SEQ ID NO:61.

In certain embodiments, an rAAV of the disclosure comprises a hybrid capsid protein comprising a VP1u and/or VP1/2s of AAV5, and a VP3 of AAVhu32. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:63. In some embodiments, an rAAV comprises a hybrid capsid protein comprising the amino acid sequence of SEQ ID NO:63.

In certain embodiments, an rAAV of the disclosure comprises a hybrid capsid protein comprising a VP1u and/or VP1/2s of AAV hu.32, and a VP3 of AAV rh.15. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:65. In some embodiments, an rAAV comprises a hybrid capsid protein comprising the amino acid sequence of SEQ ID NO:65.

In certain embodiments, an rAAV of the disclosure comprises a hybrid capsid protein comprising a VP1u and/or VP1/2s of AAV6, and a VP3 of AAV ru.15. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:67. In some embodiments, an rAAV comprises a hybrid capsid protein comprising the amino acid sequence of SEQ ID NO:67.

In certain embodiments, an rAAV of the disclosure comprises a hybrid capsid protein comprising a VP1u and/or VP1/2s of AAV5, and a VP3 of AAV rh.15. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:69. In some embodiments, an rAAV comprises a hybrid capsid protein comprising the amino acid sequence of SEQ ID NO:69.

In certain embodiments, an rAAV of the disclosure comprises a hybrid capsid protein comprising a VP1u and/or VP1/2s of AAV6, and a VP3 of AAV rh.13. In some embodiments, an rAAV comprises a hybrid capsid protein comprising an amino acid sequence having about or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 96%, 97%, 98%, or 99% sequence identity to SEQ ID NO:71. In some embodiments, an rAAV comprises a hybrid capsid protein comprising the amino acid sequence of SEQ ID NO:71.

In some embodiments, an rAAV of the disclosure comprises a capsid protein comprising an amino acid sequence that is about or at least about 80%, 85%, 90%, 91%, 92%, 93%, 94%, 95%, 96%, 97%, 98%, 99%, or 100% identical to any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119. In some embodiments, an rAAV of the disclosure comprises a capsid protein consisting of any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119.

In various aspects of the invention, the percent identity differences between amino acid sequences is from truncation of amino acids, addition of amino acids, or one or more amino acid substitutions. In some embodiments, an amino acid substitution is a conservative substitution. Illustrative examples for conserved amino acid exchanges are amino acid substitutions that maintain structural and/or functional properties of the amino acids' side-chains, e.g., an aromatic amino acid is substituted for another aromatic amino acid, an acidic amino acid is substituted for another acidic amino acid, a basic amino acid is substituted for another basic amino acid, and an aliphatic amino acid is substituted for another aliphatic amino acid. In some embodiments, a conservative amino acid substitution is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In contrast, examples of non-conserved amino acid exchanges are amino acid substitutions that do not maintain structural and/or functional properties of the amino acids' side-chains, e.g., an aromatic amino acid is substituted for a basic, acidic, or aliphatic amino acid, an acidic amino acid is substituted for an aromatic, basic, or aliphatic amino acid, a basic amino acid is substituted for an acidic, aromatic or aliphatic amino acid, and an aliphatic amino acid is substituted for an aromatic, acidic or basic amino acid.

In some embodiments, provided herein are AAV vectors comprising a viral genome comprising an expression cassette for expression of a therapeutic product, under the control of regulatory elements and flanked by ITRs, and a hybrid viral capsid as described herein.

In some embodiments, an AAV of the disclosure can be an AAV derived from a naturally occurring “wild-type” virus, an AAV derived from an rAAV genome packaged into a capsid comprising capsid proteins encoded by a naturally occurring cap gene and/or from an rAAV genome packaged into a capsid comprising capsid proteins encoded by a non-naturally occurring capsid cap gene. An example of the latter includes an rAAV having a hybrid capsid protein as described herein.

In some embodiments, an AAV capsid is characterized by DNAse-resistant particle which is an assembly of about 60 variable proteins (VP) which are typically expressed as alternative splice variants resulting in capsid proteins of different length of any one of SEQ ID NOS: 9, 19, or 35. The largest protein, VP1, is generally the full-length of the amino acid sequence of any one of SEQ ID NOS: 9, 19, 29, 31, 33, 35, 37, 39, 41, 43, 45, 47, 49, 51, 53, 55, 57, 59, 61, 63, 65, 67, 69, or 71. In certain embodiments, an AAV hybrid VP1 capsid protein has the amino acid sequence of 1 to 736 of SEQ ID NOS: 9, 19, 29, 31, 33, 35, 37, 39, 41, 43, 45, 47, 49, 51, 53, 55, 57, 59, 61, 63, 65, 67, 69, or 71. In certain embodiments, an AAV hybrid VP2 capsid protein has the amino acid sequence of 138 to 736 of SEQ ID NOS: 9, 19, 29, 31, 33, 35, 37, 39, 41, 43, 45, 47, 49, 51, 53, 55, 57, 59, 61, 63, 65, 67, 69, or 71. VP1u is the N-terminal region unique to VP1. VP1/2s is the common region shared by VP1 and VP2. In some embodiments, a VP1 region or capsid protein or fragment thereof includes VP1u, such as amino acids 1-137 of SEQ ID NO: 31, corresponding to SEQ ID NO: 146, and/or functional fragments thereof. In some embodiments, a VP1/2s includes about amino acids 138-202 of SEQ ID NO: 31, corresponding to SEQ ID NO: 148, and/or functional fragments thereof. In some embodiments, a VP1 capsid protein or fragment thereof includes VP1u region of an AAV disclosed in Section 7 of the disclosure. In some embodiments, a VP1/2s region includes VP1/2s region of an AAV disclosed in Section 7 of the disclosure. In certain embodiments, an AAV hybrid VP3 capsid protein has the amino acid sequence of 203 to 736 of SEQ ID NOS: 9, 19, 29, 31, 33, 35, 37, 39, 41, 43, 45, 47, 49, 51, 53, 55, 57, 59, 61, 63, 65, 67, 69, or 71. In certain embodiments, the VP1, VP2 or VP3 proteins may have truncations (e.g., 1 or more amino acids at the N-terminus or C-terminus). An assembled AAV capsid is composed of about 60 VP proteins, in which VP1, VP2 and VP3 are present in a ratio of about one VP1, to about one VP2, to about 10 to 20 VP3 proteins. This ratio may vary depending upon the production system used. In certain embodiments, an engineered AAV hybrid capsid may be generated in which VP2 is absent.

Also provided are nucleic acids encoding an engineered capsid protein and variants thereof, packaging cells for expressing the nucleic acids to produce an rAAV vector, an rAAV vector further comprising a therapeutic product (e.g., transgene), and pharmaceutical compositions comprising an rAAV vector; as well as methods of using an rAAV vector to deliver a therapeutic protein to a cell or target tissue of a subject in need thereof. In some embodiments, the nucleotide sequence that encodes an AAV VP3 comprises a sequence that shares about or at least about 80%, 85%, 90%, 95%, 96%, 97%, 98%, 99%, 99.5%, or 100% identity to SEQ ID NO:2, 12, or 28. In some embodiments, the nucleotide sequence that encodes an AAV VP1/2s comprises a sequence that shares about or at least about 80%, 85%, 90%, 95%, 96%, 97%, 98%, 99%, 99.5%, or 100% identity to SEQ ID NO:4 or 14. In some embodiments, the nucleotide sequence that encodes an AAV VP2 comprises a sequence that shares about or at least about 80%, 85%, 90%, 95%, 96%, 97%, 98%, 99%, 99.5%, or 100% identity to SEQ ID NO:6 or 16. In some embodiments, the nucleotide sequence that encodes an AAV VP1u comprises a sequence that shares about or at least about 80%, 85%, 90%, 95%, 96%, 97%, 98%, 99%, 99.5%, or 100% identity to SEQ ID NO:8 or 18. In some embodiments, the nucleotide sequence that encodes an AAV VP1 comprises a sequence that shares about or at least about 80%, 85%, 90%, 95%, 96%, 97%, 98%, 99%, 99.5%, or 100% identity to SEQ ID NO:10, 20, 30, 32, 34, 36, 38, 40, 42, 44, 46, 48, 50, 52, 54, 56, 58. 60, 62, 64, 66, 68, 70 or 72.

It is within the skill in the art to design nucleic acid sequences encoding an AAV hybrid capsid protein, including DNA (genomic or cDNA), or RNA (e.g., mRNA). In certain embodiments, the nucleic acid sequence encoding the AAV hybrid VP1 capsid protein is provided in SEQ ID NO:10, 20, 30, 32, 34, 36, 38, 40, 42, 44, 46, 48, 50, 52, 54, 56, 58. 60, 62, 64, 66, 68, 70 or 72. In other embodiments, a nucleic acid sequence of 70% to 99.9% identity to SEQ ID NO:10, 20, 30, 32, 34, 36, 38, 40, 42, 44, 46, 48, 50, 52, 54, 56, 58. 60, 62, 64, 66, 68, 70 or 72 may be selected to express the AAV hybrid VP1 capsid protein. In certain other embodiments, the nucleic acid sequence is at least about 75% identical, at least 80% identical, at least 85%, at least 90%, at least 95%, at least 97% identical, or at least 99% to 99.9% identical to SEQ ID NO:10, 20, 30, 32, 34, 36, 38, 40, 42, 44, 46, 48, 50, 52, 54, 56, 58. 60, 62, 64, 66, 68, 70 or 72.

In some embodiments, a nucleic acid sequence comprises a nucleic acid sequence that is about or at least about 80%, 85%, 90%, 91%, 92%, 93%, 94%, 95%, 96%, 97%, 98%, 99%, or 100% identical to any one of SEQ ID NOs: 22, 24, 26, 30, 34, 36, 38, 40, 42, 44, 46, 48, 50, 52, 54, 56, 58, 60, 62, 64, 66, 68, 70, 72, 82, 84, 86, 88, 90, 92, 94, 96, 98, 100, 102, 104, 106, 108, 110, 112, 114, 116, 118, and 120. In some embodiments, a nucleic acid sequence comprises the nucleic acid sequence of any one of SEQ ID NOs: 22, 24, 26, 30, 34, 36, 38, 40, 42, 44, 46, 48, 50, 52, 54, 56, 58, 60, 62, 64, 66, 68, 70, 72, 82, 84, 86, 88, 90, 92, 94, 96, 98, 100, 102, 104, 106, 108, 110, 112, 114, 116, 118, and 120. In some embodiments, a nucleic acid sequence consists of the nucleic acid sequence of any one of SEQ ID NOs: 22, 24, 26, 30, 34, 36, 38, 40, 42, 44, 46, 48, 50, 52, 54, 56, 58, 60, 62, 64, 66, 68, 70, 72, 82, 84, 86, 88, 90, 92, 94, 96, 98, 100, 102, 104, 106, 108, 110, 112, 114, 116, 118, and 120.

In some embodiments, an rAAV comprising the hybrid capsid protein of the disclosure (e.g., an AAV capsid comprising a VP1u and/or VP1/2s of a different serotype than the serotype of the VP3 capsid protein) has enhanced cell or tissue tropism (e.g., cell or tissue associated with the musculature) as compared to an AAV comprising an unmodified capsid or a capsid without hybrid engineering of the disclosure; and/or is homogeneously expressed or uniformly distributed at a desired location (e.g, homogeneously expressed or uniformly widespread at the musculature or an area within the musculature a subject) after the rAAV is administered to a subject. In some embodiments, the rAAV comprising the hybrid capsid protein of the disclosure is detargeted from the liver.

In some embodiments, an rAAV vector comprises AAVhu32 having muscle-homing properties that are enhanced compared to other Clade F capsids, for example AAV9. In some embodiments, the muscle-specific rAAV vector, such as AAVhu32, further comprises a peptide insertion within the capsid protein, which peptide includes amino acid sequences that confer and/or enhance desired muscle-homing properties, or muscle cell tropism. In particular, the rAAV vector comprises an engineered capsid protein comprising a peptide insertion from a heterologous protein inserted within or near variable region IV (VR-IV) or, alternatively, within or near variable region VIII (VR-VIII) of the virus capsid, such that the insertion is surface exposed on the AAV particle. In some embodiments, the peptide insertion is inserted after amino acid residue 454 or 589. In some embodiments, the peptide insertion is inserted between amino acids 454 and 455. In some embodiments, the peptide insertion is inserted between amino acids 589 and 590. In some embodiments, the peptide insertion is inserted between any two consecutive amino acids between amino acid at position 451 and amino acid at position 461. In some embodiments, the peptide insertion is inserted between any two consecutive amino acids between amino acid at position 570 and amino acid at position 589.

In some embodiments, a peptide insertion (e.g., muscle-homing peptide) is inserted before or after at least one amino acid at position: 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600 of an AAV (e.g., AAVhu32 or a corresponding position in another AAV). In some embodiments, the peptide insertion occurs immediately after one of the amino acid residues within: 450-456, 451-461, 454-456, 450-460, 440-470, 430-480, 420-500, 510-600, 520-590, 520-540, 530-540, 530-550, 530-535, 532-535, 560-590, 580-590, 570-600, 585-589, 585-590 of AAVhu32 or a corresponding position in another AAV. In some embodiments, a peptide insertion is inserted at or after position 454, particularly S454 in AAVhu32 or a corresponding position in another AAV. In some embodiments, a peptide insertion is inserted at or after position 589, particularly A589 in AAVhu32 or a corresponding position in another AAV.

In some embodiments, a peptide insertion (or an insertion of a heterologous amino acid sequence) is of about 4 to about or up to about 15 (e.g., 4, 5, 6, 7, 8, 9, 10, 11, 12, 13, 14, or 15) amino acids, within or near a variable region (e,g., VR-VIII, VR-IV, and/or VR-VI of AAVhu32 or a corresponding variable region in another AAV) of a virus capsid. In some embodiments, the peptide insertion is between two amino acids without deleting any capsid amino acid(s) of an AAV capsid (e.g., AAVhu32). In some embodiments, the peptide insertion occurs within (i.e., between two amino acids without deleting any capsid amino acids) variable region VIII (VR-VIII), VR-IV, and/or VR-VI of AAVhu32 capsid, or a corresponding region in another AAV serotype.

In some embodiments, the muscle-homing peptide comprises an integrin receptor-binding domain or an integrin-binding domain, such as RGD and other recognition sequences for integrin (Ruoslahti E. Annu Rev Cell Dev Biol. 1996; 12:697-715. doi: 10.1146/annurev.cellbio.12.1.697. PMID: 8970741; e.g. RGD-peptide RGDLRVS (SEQ ID NO: 151), or peptide SLRSPPS (SEQ ID NO: 140), as described in Varadi, et al. 2012 Gene Therapy, 19, 800-809; e.g. RGD-peptide RGDLGLS (SEQ ID NO: 141), as described in Michelfelder S, et al., 2009, PLOS ONE 4 (4): e5122. doi: 10.1371/journal.pone.0005122; e.g. 4C-RGD peptide CDCRGDCFC (SEQ ID NO: 142) or CDCRGDCFCGLS (SEQ ID NO: 143), as described in Shi and Bartlett 2003 Mol. Ther. 2003, 7, 515-525; and other RGD-containing peptide sequences described in Tabebordbar et al., 2021, Cell 184, 4919-4938, including e.g. RGDLTTP (SEQ ID NO: 144) and RGDLSTP (SEQ ID NO: 145), each of which is incorporated herein by reference in its entirety). In certain embodiments, the peptide insertion may be a sequence of consecutive amino acids from a domain that targets muscle, or an analog, or a conformational analog designed to mimic the three-dimensional structure of said domain.

In some embodiments, a muscle-homing peptide is RRQPPRSISSHP (M12; SEQ ID NO: 134) or a portion thereof. In some embodiments, a muscle-homing peptide comprises about or at least 4, 5, 6, 7, or more than 7 contiguous amino acids of RRQPPRSISSHP (M12; SEQ ID NO: 134). In some embodiments, a muscle-homing peptide comprises about or at least about 80%, 85%, 90%, 95%, or 100% sequence identity to RRQPPRSISSHP (M12; SEQ ID NO: 134). In some embodiments, a muscle-homing peptide is ASSLNIA (a muscle-specific peptide (MSP); SEQ ID NO: 135) or a portion thereof. In some embodiments, a muscle-homing peptide comprises about or at least 4, 5, 6, or 7 contiguous amino acids of ASSLNIA (a muscle-specific peptide (MSP); SEQ ID NO: 135). In some embodiments, a muscle-homing peptide comprises about or at least about 80%, 85%, 90%, 95%, or 100% sequence identity to ASSLNIA (a muscle-specific peptide (MSP); SEQ ID NO: 135). In some embodiments, a muscle-homing peptide is AGSTSAGSAAGSSGDRRQPPRSISSHP (SEQ ID NO: 136) or a portion thereof. In some embodiments, a muscle-homing peptide comprises about or at least 4, 5, 6, 7, or more than 7 contiguous amino acids of AGSTSAGSAAGSSGDRRQPPRSISSHP (SEQ ID NO: 136). In some embodiments, a muscle-homing peptide comprises about or at least about 80%, 85%, 90%, 95%, or 100% sequence identity to AGSTSAGSAAGSSGDRRQPPRSISSHP (SEQ ID NO: 136). In some embodiments, a muscle-homing peptide is CYAIGSFDC (SEQ ID NO: 137) or a portion thereof. In some embodiments, a muscle-homing peptide comprises about or at least 4, 5, 6, 7, or more than 7 contiguous amino acids of CYAIGSFDC (SEQ ID NO: 137). In some embodiments, a muscle-homing peptide comprises about or at least about 80%, 85%, 90%, 95%, or 100% sequence identity to CYAIGSFDC (SEQ ID NO: 137). In some embodiments, a muscle-homing peptide is SGASAV (SEQ ID NO: 138) or a portion thereof. In some embodiments, a muscle-homing peptide comprises about or at least 4 contiguous amino acids of SGASAV (SEQ ID NO: 138). In some embodiments, a muscle-homing peptide comprises about or at least about 80%, 85%, 90%, 95%, or 100% sequence identity to SGASAV (SEQ ID NO: 138). In some embodiments, a muscle-homing peptide comprises the motif GRSGXR (SEQ ID NO: 139; wherein X can be any naturally occurring amino acid). In some embodiments, a muscle-homing peptide comprises the motif DFSGIAX (SEQ ID NO: 150; wherein X can be any naturally occurring amino acid). A muscle-homing peptide of the disclosure can be any known muscle-homing peptide or any predicted muscle-homing peptide.

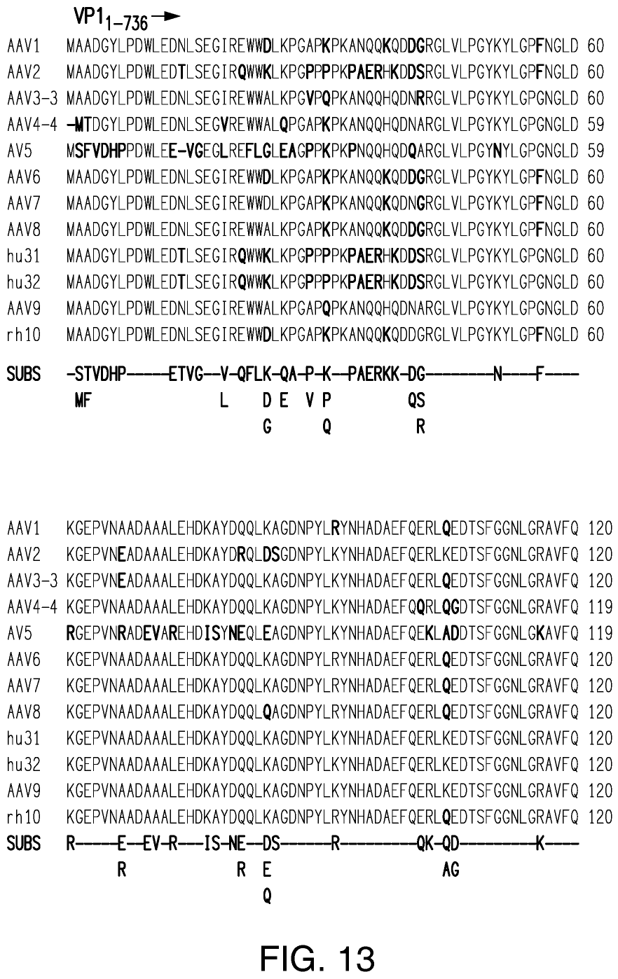

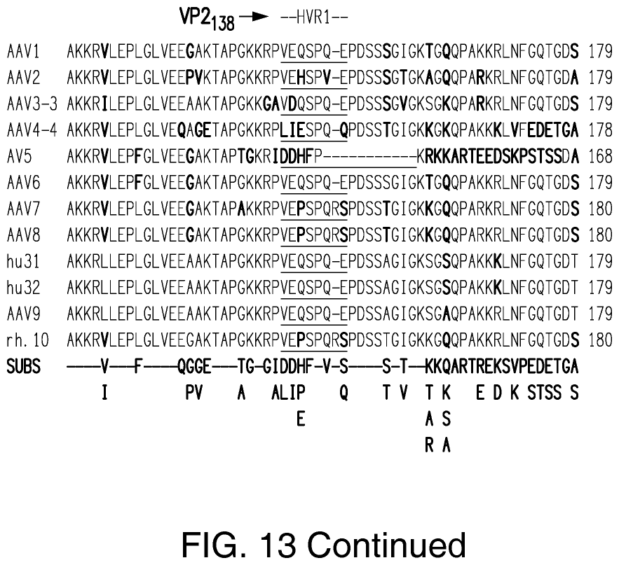

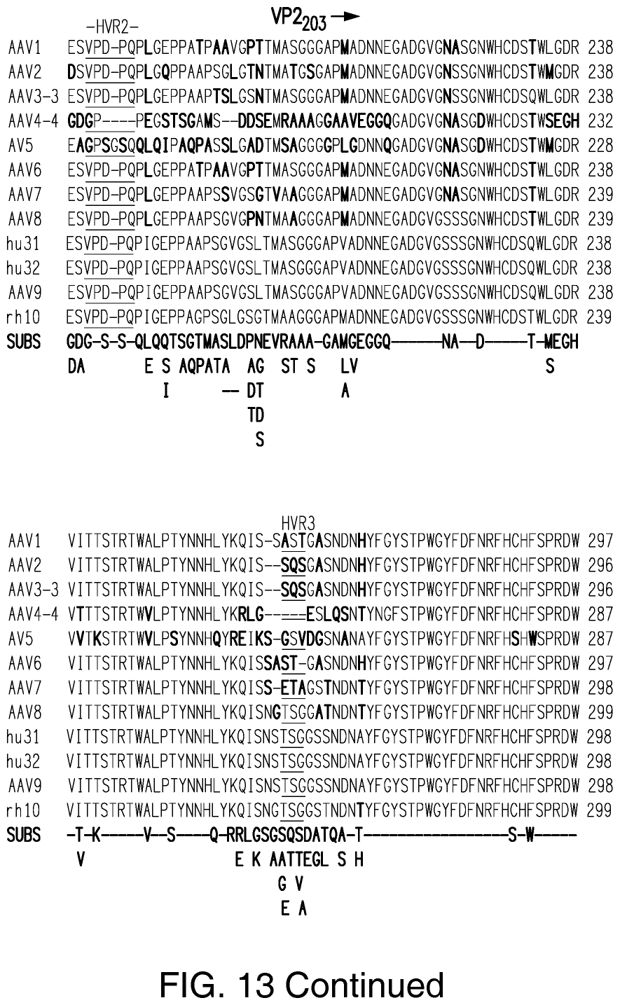

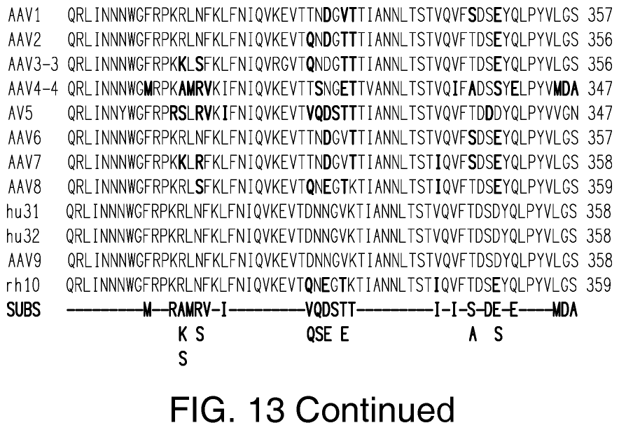

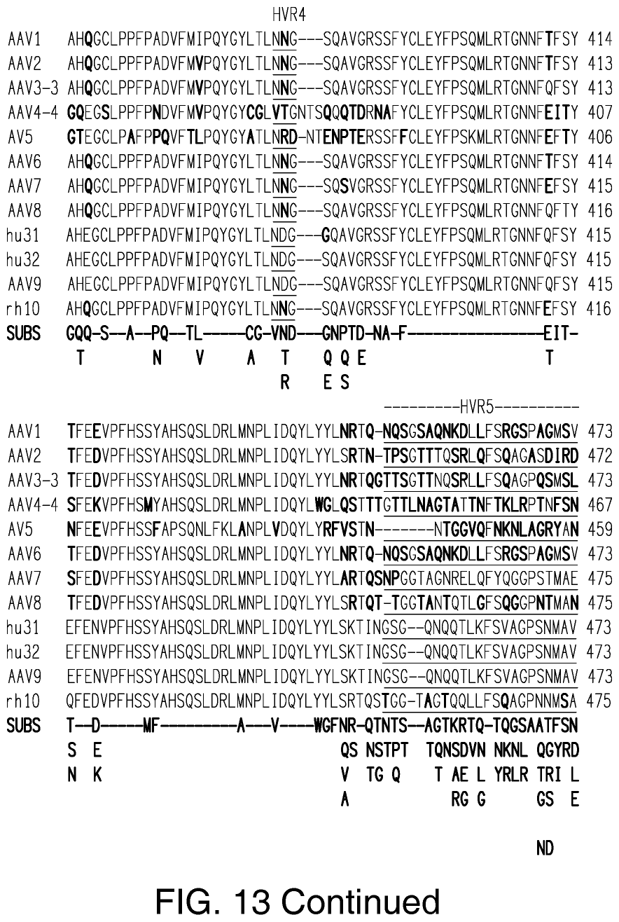

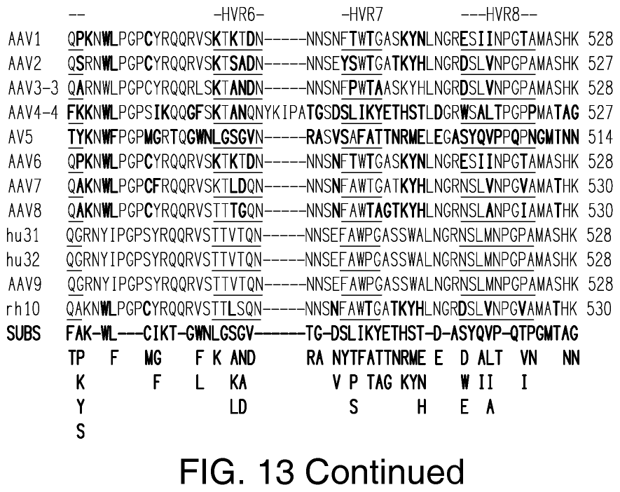

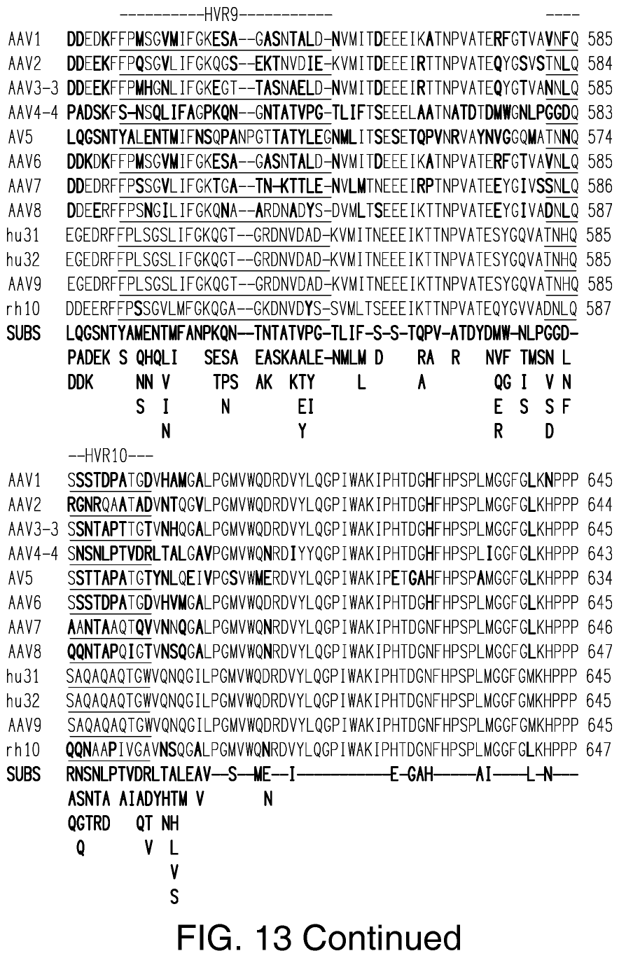

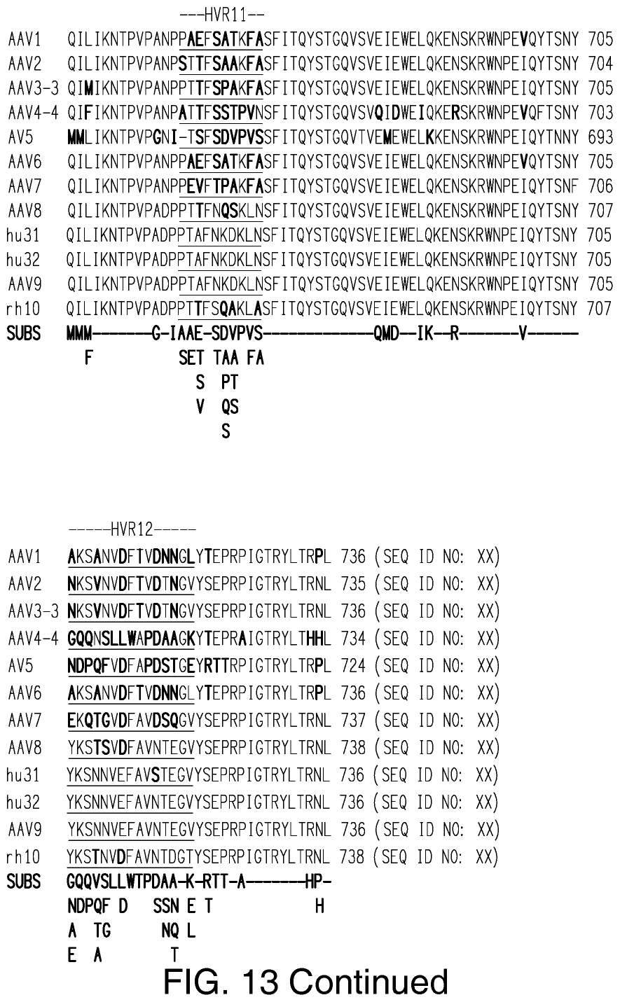

The muscle-homing peptide can be inserted into an AAV capsid, for example, at sites that allow surface exposure of the peptide, such as within variable surface-exposed loops, and, in more examples, sites described herein corresponding to VR-I, VR-IV, or VR-VIII of the capsid protein or may be inserted after the first amino acid of VP2, e.g. after amino acid 137 (e.g. as in AAV4, AAV4-4, and AAV5) or at amino acid 138 (e.g. as in AAV1, AAV2, AAV3, AAV3-3, AAV6, AAV7, AAV8, AAV9, AAVhu.31, AAVhu32, and rh.10) (FIG. 13). Recombinant AAV vectors comprising one or more muscle-homing peptides, e.g., inserted into a surface-exposed loop of an AAV capsid, are referred to herein as “rAAV muscle-homing vectors.” In some embodiments, the capsid protein is an AAVhu32 capsid protein and the muscle homing peptide insertion occurs immediately after at least one of the amino acid residues 451 to 461 or 570 to 590 of the AAVhu32 capsid (e.g., SEQ ID NO: 31). In other embodiments, the rAAV vector is an AAVhu32 variant, and the muscle-homing peptide insertion occurs immediately after an amino acid residue corresponding to at least one of the amino acid residues 451 to 461 or 570 to 590 of AAVhu32 capsid protein (e.g., SEQ ID NO: 31). The alignments of several different AAV serotypes, as shown in FIG. 13, indicates corresponding amino acid residues in these different capsid protein sequences.

Liver detargeting has also been associated with substitution of particular amino acids in capsid proteins. In some embodiments, the liver detargeting mutation and/or peptide insertion comprises any one of the capsid mutations or peptides described in PCT Publication No. WO2020206189A1 (the content of which is herein incorporated by reference in its entirety).

5.2.2 Properties of rAAV Comprising a Variant AAV Capsid

Provided herein are recombinant adeno-associated viruses (rAAVs) comprising a hybrid AAV capsid as described in Section 5.2.1 or a hybrid AAV capsid comprising a polypeptide as described in Section 5.1.1. In some embodiments, a hybrid AAV of the disclosure provides for at least one enhanced property (e.g., enhanced cell or tissue tropism, homogeneous distribution of a transgene at a specific location after such rAAVs are administered to a subject, packaging efficiency, yield, titer, infectivity, transduction efficiency, or transfection efficiency) as compared to a reference AAV, a wild-type AAV or an AAV that does not comprise a hybrid AAV capsid of the disclosure. In some embodiments, the rAAV provides at least one improvement of packaging efficiency, yield, titer, infectivity, transduction efficiency, and transfection efficiency, as compared to a reference AAV. In some embodiments, a hybrid capsid promotes transduction and/or tissue tropism as described herein (i.e., muscle tropism and/or liver detargeting). In some embodiments, an rAAV vector comprising a hybrid capsid of the disclosure enhances targeted delivery, improves transduction and/or treatment of disorders associated with the target tissue as compared to a reference AAV, a wild-type AAV vector or an AAV vector that does not comprise a hybrid capsid of the disclosure. In some embodiments, an rAAV vector comprising a hybrid capsid of the disclosure enhances packaging efficiency as compared to a reference AAV, a wild-type AAV vector or an AAV vector that does not comprise a hybrid capsid of the disclosure. In some embodiments, an rAAV vector comprising a hybrid capsid of the disclosure enhances viral yield or titer as compared to a reference AAV, a wild-type AAV vector or an AAV vector that does not comprise a hybrid capsid of the disclosure. In some embodiments, an rAAV vector comprising a hybrid capsid of the disclosure enhances infectivity, transduction efficiency, or transfection efficiency as compared to a reference AAV, a wild-type AAV vector or an AAV vector that does not comprise a hybrid capsid of the disclosure. In some embodiments, a reference AAV is AAV8. In some embodiments, a reference AAV is wild-type AAV. In some embodiments, a reference AAV comprises VP1u of AAV8. In some embodiments, a reference AAV is AAVrh13. In some embodiments, a reference AAV is AAVhu32. In some embodiments, a reference AAV is AAV5. In some embodiments, reference AAV is AAV9.

In some embodiments, the rAAV transduces muscle and/or liver tissues at lower levels as compared to a reference AAV. In some embodiments, the rAAV results in lower RNA expression in liver as compared to a reference AAV. In some embodiments, an improved property as compared to a reference AAV is an improvement by about or at least about 1-fold, 1.5-fold, 2-fold, 2.5-fold, 3-fold, 3.5-fold, 4-fold, 4.5-fold, 5 fold, 5.5-fold, 6-fold, 6.5-fold, 7-fold, 7.5-fold, 8-fold, 8.5-fold, 9-fold, 9.5-fold, 10-fold, or higher than 10-fold. In some embodiments, the improvement is by about or at least about 2 fold. In some embodiments, the improvement is by about or at least about 2.5 fold. In some embodiments, the improvement is by about or at least about 3 fold. In some embodiments, the RNA expression is lower by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100% as compared to the RNA expression from a reference AAV. In some embodiments, the rAAV results in higher RNA/DNA ratio in muscle tissue as compared to a reference AAV. In some embodiments, the RNA/DNA ratio is higher by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100%. In some embodiments, the rAAV results in lower DNA levels in muscle tissue as compared to a reference AAV. In some embodiments, the DNA levels is lower by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100% as compared to the DNA levels from a reference AAV.

In some embodiments, an rAAV of the disclosure results in a higher relative abundance of mRNA transcript copies (of transgene) in a muscle cell or tissue (e.g., gastrocnemius, quadriceps, and/or tibialis anterior) and/or in a heart cell or tissue compared to a reference AAV. In some embodiments, the relative abundance of mRNA transcript copies (of transgene) is higher by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100%. In some embodiments, the relative abundance of mRNA transcript copies (of transgene) is higher by about or at least about 1-fold, 1.5-fold, 2-fold, 2.5-fold, 3-fold, 3.5-fold, 4-fold, 4.5-fold, 5 fold, 5.5-fold, 6-fold, 6.5-fold, 7-fold, 7.5-fold, 8-fold, 8.5-fold, 9-fold, 9.5-fold, 10-fold, or higher than 10-fold. In some embodiments, an rAAV of the disclosure results in a higher relative abundance of cDNA in a muscle cell or tissue (e.g., gastrocnemius, quadriceps, and/or tibialis anterior) and/or in a heart cell or tissue compared to a reference AAV. In some embodiments, the relative abundance of cDNA is higher by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100%. In some embodiments, the relative abundance of cDNA is higher by about or at least about 1-fold, 1.5-fold, 2-fold, 2.5-fold, 3-fold, 3.5-fold, 4-fold, 4.5-fold, 5 fold, 5.5-fold, 6-fold, 6.5-fold, 7-fold, 7.5-fold, 8-fold, 8.5-fold, 9-fold, 9.5-fold, 10-fold, or higher than 10-fold.

In some embodiments, an rAAV of the disclosure results in a higher biodistribution or transgene expression (e.g., microdystrophin) in a muscle cell or tissue (e.g., gastrocnemius, quadriceps, and/or tibialis anterior) and/or in a heart cell or tissue compared to a reference AAV. In some embodiments, the biodistribution or transgene expression (e.g., microdystrophin) is higher by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100%. In some embodiments, the biodistribution or transgene expression (e.g., microdystrophin) is higher by about or at least about 1-fold, 1.5-fold, 2-fold, 2.5-fold, 3-fold, 3.5-fold, 4-fold, 4.5-fold, 5 fold, 5.5-fold, 6-fold, 6.5-fold, 7-fold, 7.5-fold, 8-fold, 8.5-fold, 9-fold, 9.5-fold, 10-fold, or higher than 10-fold. In some embodiments, an rAAV viral particle of the disclosure has increased tropism for a particular cell or tissue (e.g., a muscle cell or tissue; and/or a heart cell or tissue) as compared to a reference AAV. In some embodiments, the increase is about 5%, 10%, 15%, 20%, 25%, 30%, 35%, 40%, 45%, or 50% increase in tropism for a particular cell type or tissue (e.g., a muscle cell or tissue; and/or a heart cell or tissue) as compared to a reference AAV. In some embodiments, the increase is about 5% to about 20%, about 25% to about 50% increase in tropism for a particular cell type or tissue (e.g., a muscle cell or tissue; and/or a heart cell or tissue) as compared to a reference AAV. In some embodiments, the increase is about 0.1 fold, about 0.2 fold, about 0.3 fold, about 0.4 fold, about 0.5 fold, about 0.6 fold, about 0.7 fold, about 0.8 fold, about 0.9 fold, or about 1 fold increase in tropism for a particular cell type or tissue (e.g., a muscle cell or tissue; and/or a heart cell or tissue) as compared to a reference AAV. In some embodiments, the increase is about 0.1 to 1 fold increase in tropism for a particular cell type or tissue (e.g., a muscle cell or tissue; and/or a heart cell or tissue) as compared to a reference AAV. In some embodiments, an rAAV viral particle of the disclosure has increased transduction of muscle and/or heart cells relative to a second AAV viral particle comprising a capsid protein that comprises any one of SEQ ID NOs: 19, 31, and 124 and/or comprises AAV5, AAV8, AAV-9, AAVrh13, or AAVhu32 capsid protein. In some embodiments, the rAAV viral particle has about a 0.10 fold to about a 1 fold increase in transduction of muscle and/or heart cells relative to the second AAV viral particle. In some embodiments, the rAAV viral particle has about 0.10 fold, about 0.2 fold, about 0.3 fold, about 0.4 fold, about 0.5 fold, about 0.6 fold, about 0.7 fold, about 0.8 fold, about 0.9 fold, or about 1 fold increase in transduction of muscle and/or heart cells relative to the second AAV viral particle (e.g., a reference AAV). In some embodiments, an AAV of the disclosure has increased muscle and/or heart tropism as compared to a reference or second AAV (e.g., AAV5, AAV8, AAV-9, AAVrh13, or AAVhu32).

In some embodiments, the rAAV results in a lower level of liver toxicity as compared to the level of liver toxicity from a reference AAV. In some embodiments, the level of liver toxicity is lower by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100% as compared to the level of liver toxicity from a reference AAV. In some embodiments, an rAAV viral particle of the disclosure has decreased tropism for a particular cell or tissue (e.g., a liver cell or the liver) as compared to a reference AAV. In some embodiments, the decrease is about 5%, 10%, 15%, 20%, 25%, 30%, 35%, 40%, 45%, or 50% decrease in tropism for a particular cell type or tissue (e.g., a liver cell or the liver) as compared to a reference AAV. In some embodiments, the decrease is about 5% to about 20%, about 25% to about 50% decrease in tropism for a particular cell type or tissue (e.g., a liver cell or the liver) as compared to a reference AAV. In some embodiments, the decrease is about 0.1 fold, about 0.2 fold, about 0.3 fold, about 0.4 fold, about 0.5 fold, about 0.6 fold, about 0.7 fold, about 0.8 fold, about 0.9 fold, or about 1 fold decrease in tropism for a particular cell type or tissue (e.g., a liver cell or the liver) as compared to a reference AAV. In some embodiments, the decrease is about 0.1 to 1 fold decrease in tropism for a particular cell type or tissue (e.g., a liver cell or the liver) as compared to a reference AAV. In some embodiments, an rAAV viral particle of the disclosure has reduced transduction of liver cells relative to a second AAV viral particle comprising a capsid protein that comprises any one of SEQ ID NOs: 19, 31, and 124 and/or AAV5, AAV8, AAV-9, AAVrh13, or AAVhu32 capsid protein. In some embodiments, the rAAV viral particle has about a 0.10 fold to about a 1 fold decrease in transduction of liver cells relative to the second AAV viral particle. In some embodiments, the rAAV viral particle has about 0.10 fold, about 0.2 fold, about 0.3 fold, about 0.4 fold, about 0.5 fold, about 0.6 fold, about 0.7 fold, about 0.8 fold, about 0.9 fold, or about 1 fold decrease in transduction of liver cells relative to the second AAV viral particle (e.g., a reference AAV). In some embodiments, the rAAV results in transcription-specific liver de-targeting as compared to a reference AAV. In some embodiments, an AAV of the disclosure has decreased liver tropism as compared to a reference AAV (e.g., AAV5, AAV8, AAV-9, AAVrh13, or AAVhu32). In some embodiments, a reference AAV is AAV8. In some embodiments, a reference AAV is wild-type AAV. In some embodiments, reference AAV comprises VP1u of AAV8. In some embodiments, reference AAV is AAVrh13. In some embodiments, reference AAV is AAVhu32. In some embodiments, reference AAV is AAV5. In some embodiments, reference AAV is AAV9. In some embodiments, a reference AAV is the same AAV as the AAV of the VP3 region of a hybrid AAV. In some embodiments, a reference AAV is the same AAV as the AAV of the VP1/2s region of a hybrid AAV. In some embodiments, a reference AAV is the same AAV as the AAV of the VP1u region of a hybrid AAV.

In some embodiments a lower amount of vector genome of the rAAV is sufficient to obtain a comparable therapeutic effect as compared to the amount of vector genome of a reference AAV necessary to obtain the same therapeutic effect after administration in a subject. In some embodiments, the amount is lower by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100%. In some embodiments, the amount is lower by 1-fold, 1.5-fold, 2-fold, 2.5-fold, 3-fold, 3.5-fold, 4-fold, 4.5-fold, 5 fold, 5.5-fold, 6-fold, 6.5-fold, 7-fold, 7.5-fold, 8-fold, 8.5-fold, 9-fold, 9.5-fold, 10-fold, or higher than 10-fold.

In some embodiments, the rAAV provides a higher vector yield after the rAAV is administered to the subject, as compared to a reference AAV. In some embodiments, the vector yield is higher by about or at least about: 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10 times, or more than 10 times. In some embodiments, the vector yield is increased by about or at least about: 5%, 10%, 15%, 20%, 25%, 30%, 35%, 40%, 45%, 50%, 55%, 60%, 65%, 70%, 75%, 80%, 85%, 90%, 95%, 100%, or more than about 100%. In some embodiments, the vector yield is higher by between about 1.5 to about 3 times. In some embodiments, the vector yield is higher from about 2.5 to about 3.5, from about 1 to about 4 times, from about 1 to about 5 times, from about 1 to about 7 times, from about 1 to about 10 times, from about 2 to about 5 times, from about 2 to about 7 times, from about 2 to about 10 times, from about 3 to about 5 times, from about 3 to about 7 times, from about 3 to about 10 times, from about 4 to about 5 times, from about 4 to about 7 times, from about 4 to about 10 times, from about 5 to about 7 times, from about 5 to about 10 times, from about 6 to about 8 times, from about 6 to about 7 times, from about 6 to about 10 times, from about 7 to about 10, or more than from about 7 to about 10. In some embodiments, the RNA level is increased after the rAAV is administered to the subject, as compared to a reference AAV. In some embodiments, the RNA level is increased by about or at least about: 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10 times, or more than 10 times. In some embodiments, the RNA level is increased by about or at least about: 5%, 10%, 15%, 20%, 25%, 30%, 35%, 40%, 45%, 50%, 55%, 60%, 65%, 70%, 75%, 80%, 85%, 90%, 95%, 100%, or more than about 100%. In some embodiments, the RNA level is increased by between about 2.5 to about 3.5 times. In some embodiments, the RNA level is increased from about 1.5 to about 3, from about 2.5 to about 3.5, from about 1 to about 4 times, from about 1 to about 5 times, from about 1 to about 7 times, from about 1 to about 10 times, from about 2 to about 5 times, from about 2 to about 7 times, from about 2 to about 10 times, from about 3 to about 5 times, from about 3 to about 7 times, from about 3 to about 10 times, from about 4 to about 5 times, from about 4 to about 7 times, from about 4 to about 10 times, from about 5 to about 7 times, from about 5 to about 10 times, from about 6 to about 8 times, from about 6 to about 7 times, from about 6 to about 10 times, from about 7 to about 10, or more than from about 7 to about 10. In some embodiments, the ratio of RNA to DNA is increased after the rAAV is administered to the subject. In some embodiments, the ratio of RNA to DNA is increased by about or at least about: 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10 times, or more than 10 times. In some embodiments, the ratio of RNA to DNA is increased by about or at least about: 5%, 10%, 15%, 20%, 25%, 30%, 35%, 40%, 45%, 50%, 55%, 60%, 65%, 70%, 75%, 80%, 85%, 90%, 95%, 100%, or more than about 100%. In some embodiments, the ratio of RNA to DNA is increased by between about 2.5 to about 3.5 times. In some embodiments, the ratio of RNA to DNA is increased from about 1.5 to about 3, from about 2.5 to about 3.5, from about 1 to about 4 times, from about 1 to about 5 times, from about 1 to about 7 times, from about 1 to about 10 times, from about 2 to about 5 times, from about 2 to about 7 times, from about 2 to about 10 times, from about 3 to about 5 times, from about 3 to about 7 times, from about 3 to about 10 times, from about 4 to about 5 times, from about 4 to about 7 times, from about 4 to about 10 times, from about 5 to about 7 times, from about 5 to about 10 times, from about 6 to about 8 times, from about 6 to about 7 times, from about 6 to about 10 times, from about 7 to about 10, or more than from about 7 to about 10.

In some embodiments, a reference AAV is AAV5. In some embodiments, a reference AAV is AAV8. In some embodiments, a reference AAV is AAV9. In some embodiments, a reference AAV is AAVhu32. In some embodiments, a reference AAV comprises a capsid protein that comprises or consists of any one of SEQ ID NOs: 19, 31, and 124. In some embodiments, a reference AAV comprises an expression cassette. In some embodiments, a reference AAV comprises or encodes a transgene.

In some embodiments, an rAAV vector comprising a hybrid capsid of the disclosure targets a tissue or cell associated with the musculature. In some embodiments, the rAAV comprising a hybrid capsid of the disclosure facilitates delivery of therapeutic agents or transgenes for treating diseases or disorders of the musculature. In some embodiments, a hybrid capsid increases muscular tropism, or directs the rAAV to target the musculature or a muscle cell of the subject. The term “muscule cell” or “muscle cell” refers to one or more of the cell types of skeletal muscle cells, cardiac muscle cells, smooth muscle cells, or any other cell associated with a muscle, and the like.

In some embodiments, the rAAV vector comprises a peptide insertion for muscle cell-homing, the vector is administered by in vivo injection, such as subcutaneous injection, intradermal injection, or intramuscular injection. In some embodiments, injection is directly into a muscle. For example, the rAAV comprising a hybrid capsid as described herein has enhanced muscle tropism and is injected intramuscularly. In some embodiments, the rAAV for increasing muscle tropism is administered to deltoid, dorsogluteal, rectus femoris, vastus lateralis, or ventrogluteal muscular injection.

Provided herein are recombinant adeno-associated viruses (rAAVs) having hybrid capsid proteins engineered to confer and/or enhance desired properties (e.g., enhanced tissue or cell specific targeting, cell-specific tropism, and/or enhanced transduction efficacy). In some embodiments, the tissue or cell specific targeting or cell-specific tropism is related to a tissue or cell associated with the musculature of a subject (e.g., a myopathy or dystrophy). In some embodiments, an rAAV of the disclosure has about 5%, 10%, 15%, 20%, 25%, 30%, 35%, 40%, 45%, 50%, 55%, 60%, 65%, 70%, 75%, 80%, 85%, 90%, 95%, 99%, or 100% greater tropism for a particular muscle cell type or tissue as compared to a reference AAV (e.g., an AAV that does not comprise a hybrid capsid of the disclosure). In some embodiments, an rAAV of the disclosure has about 125%, 150%, 175%, 200%, 250%, 300%, 350%, 400%, or 500% greater tropism for a particular muscle cell type or tissue compared to a reference AAV. In some embodiments, an rAAV of the disclosure has about 5%, 10%, 15%, 20%, 25%, 30%, 35%, 40%, 45%, 50%, 55%, 60%, 65%, 70%, 75%, 80%, 85%, 90%, 95%, 99%, or 100% greater tropism for the heart or a heart cell or tissue as compared to a reference AAV (e.g., an AAV that does not comprise a hybrid capsid of the disclosure). In some embodiments, an rAAV of the disclosure has about 125%, 150%, 175%, 200%, 250%, 300%, 350%, 400%, or 500% greater tropism for the heart or a heart cell or tissue compared to a reference AAV. In some embodiments, the reference AAV is a wild-type AAV8 or another wild-type AAV. In some embodiments, the reference AAV is a wild-type AAV9 or another wild-type AAV. In some embodiments, the reference AAV is a wild-type AAVhu32 or another wild-type AAV. In some embodiments, the reference AAV is any AAV that does not comprise a hybrid capsid of the disclosure (e.g., does not comprise a hybrid capsid comprising a polypeptide described in Section 5.1.1 or a hybrid capsid as disclosed in Section 5.2.1).

In some embodiments, immunohistochemistry analysis (e.g., myocyte immunohistochemistry) can be performed to determine the cellular tropism of an rAAV vector of the disclosure.

In some embodiments, administration of an rAAV of the disclosure comprising a hybrid capsid comprising a polypeptide described in Section 5.1.1 or a hybrid capsid as disclosed in Section 5.2.1 and a transgene (or therapeutic product) to a subject, results in detectable expression levels of the transgene in a subject (e.g., at injection site, throughout the musculature or muscle type of the subject, in a specific area of a muscle of the subject, and/or homogeneous expression of the transgene in an area of a muscle. In some embodiments, expression levels of a transgene can be monitored in the muscle of a subject to whom an rAAV of the disclosure has been administered. Transgene expression may be measured by any suitable assay known in the art, including, without limitation, Western Blotting, electrochemiluminescent (ECL) immunoassays implemented using the Meso Scale Discovery (MSD) platform, and ELISA. In some embodiments, expression levels of the transgene in the eye may vary between different areas of the musculature.

In some embodiments, expression levels of the transgene may vary between different areas of the musculature. In some embodiments, administration of an rAAV of the disclosure to a subject results in detectable (e.g., detectable using a method described herein, or a method known in the art) transgene expression levels in an area of a muscle of the subject within about 1 hour, about 3 hours, about 5 hours, about 10 hours, about 15 hours, about 20 hours, about 24 hours, about 48 hours, about 72 hours, about 1 week, about 2 weeks, about 3 weeks, about 4 weeks, about 2 months, about 3 months, about 6 months, about 9 months, about 12 months, about 15 months, about 18 months, about 21 months, or about 24 months after administration of the rAAV to the subject, wherein the level of the transgene expression in the area of the muscle was undetectable prior to administration of the rAAV. In some embodiments, administration of an rAAV of the disclosure to a subject results in detectable (e.g., detectable using a method described herein, or a method known in the art) transgene expression levels in an area of a muscle of the subject within about 3 months, about 6 months, about 9 months, about 12 months, about 15 months, about 18 months, about 21 months, or about 24 months of administration of the rAAV to the subject, wherein the level of the transgene expression in the area of the muscle was undetectable prior to administration of the rAAV, and wherein the transgene expression levels in serum of the subject remain undetectable (e.g., undetectable using a method described herein, or a method known in the art). In some embodiments, an increased expression level of a transgene is detectable after an rAAV of the disclosure is administered to a subject (e.g., detectable using a method described herein, or a method known in the art) in an area of a muscle of the subject as compared an expression level of a transgene after a wild-type AAV or an AAV that does not comprise a hybrid capsid of the disclosure is administered to a subject. In some embodiments, a transgene is detectable or undetectable using an immunofluorescence assay. In some embodiments, a transgene is detectable or undetectable using an immunostaining assay. In some embodiments, administration of an rAAV of the disclosure to a subject results in an increase in the transgene expression levels (e.g., an increase of about or at least about 100-fold to about 500-fold, about 500-fold to about 1000-fold, about 1000-fold to about 1500-fold, about 1500-fold to about 2000-fold, about 2000-fold to about 2500-fold, about 2500-fold to about 3000-fold, about 3000-fold to about 4000-fold, about 4000-fold to about 5000-fold, about 5000-fold to about 10,000-fold, about 10,000-fold to about 15,000-fold, about 15,000-fold to about 20,000-fold, or more than about 20,000-fold) in an area of a muscle of the subject within about 1 hour, about 3 hours, about 5 hours, about 10 hours, about 15 hours, about 20 hours, about 24 hours, about 48 hours, about 72 hours, about 1 week, about 2 weeks, about 3 weeks, about 4 weeks, about 2 months, about 3 months, about 6 months, about 9 months, about 12 months, about 15 months, about 18 months, about 21 months, or about 24 months of administration of the rAAV to the subject compared to the transgene expression levels prior to administration of the rAAV or compared to the transgene expression levels after a reference AAV (e.g. AAV that does not comprise a hybrid capsid of the disclosure) is administered to a subject. In some embodiments, administration of an rAAV provided herein to a subject results in an increase in the transgene expression levels (e.g., an increase of about or at least about 100-fold to about 500-fold, about 500-fold to about 1000-fold, about 1000-fold to about 1500-fold, about 1500-fold to about 2000-fold, about 2000-fold to about 2500-fold, about 2500-fold to about 3000-fold, about 3000-fold to about 4000-fold, about 4000-fold to about 5000-fold, about 5000-fold to about 10,000-fold, about 10,000-fold to about 15,000-fold, about 15,000-fold to about 20,000-fold, or more than about 20,000-fold) in an area of a muscle of the eye of the subject within about 1 hour, about 3 hours, about 5 hours, about 10 hours, about 15 hours, about 20 hours, about 24 hours, about 48 hours, about 72 hours, about 1 week, about 2 weeks, about 3 weeks, about 4 weeks, about 2 months, about 3 months, about 6 months, about 9 months, about 12 months, about 15 months, about 18 months, about 21 months, or about 24 months of administration of the rAAV to the subject compared to the transgene expression levels prior to administration of the rAAV or compared to the transgene expression levels after a reference AAV (e.g. AAV that does not comprise a hybrid capsid of the disclosure) is administered to a subject, wherein the transgene expression levels in serum of the subject remain about constant (e.g., remain within about 10%) compared to the transgene expression levels prior to administration.

In some embodiments, administration of an rAAV provided herein to a subject results in the transgene expression in the musculature of the subject. In some embodiments, the transgene is homogeneously expressed in a muscle or throughout a muscle of the subject. In some embodiments, homogeneous expression is measured by detecting transgene expression level at injection site. In some embodiments, homogeneous expression is measured by detecting transgene expression level throughout an area of the muscle. In some embodiments, homogeneous expression is measured by detecting transgene expression level at injection site and comparing such expression level with the level of transgene expression within ¼ radius from injection site. In some embodiments, homogeneous expression is measured by detecting transgene expression level at injection site and comparing such expression level with the level of transgene expression within ⅓ radius from injection site. In some embodiments, homogeneous expression is measured by detecting transgene expression level at injection site and comparing such expression level with the level of transgene expression within ½ radius from injection site. In some embodiments, homogeneous expression is measured by detecting transgene expression level at injection site and comparing such expression level with the level of transgene expression within ⅛ radius from injection site. In some embodiments, homogeneous expression is measured by detecting transgene expression level at injection site and comparing such expression level with the level of transgene expression within 1/16 radius from injection site. In some embodiments, homogeneous expression is measured by detecting transgene expression level at injection site and comparing such expression level with the level of transgene expression within ½ radius from injection site, within ⅓ radius from injection site, within ¼ radius from injection site, within ⅛ radius from injection site, and/or within 1/16 radius from injection site. In some embodiments, homogenous expression of the transgene means that the level of expression level of the transgene at injection site is about the same as the expression level of the transgene within ½ radius from injection site, within ⅓ radius from injection site, within ¼ radius from injection site, within ⅛ radius from injection site, and/or within 1/16 radius from injection site. In some embodiments, “about the same” refers to an expression level (e.g., at injection site) that is about or at most about 1%, 2%, 3%, 4%, 5%, 6%, 7%, 8%, 9%, 10%, 11%, 12%, 13%, 14%, 15%, 16%, 17%, 18%, 19%, or 20% of the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is about or at most about 5% of the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is about or at most about 10% of the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is about or at most about 15% of the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is about or at most about 20% of the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is within 1 standard deviation (SD), 2SD, 3SD, or 4 SD from the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is within 1 standard deviation (SD) from the expression level at the comparison location (e.g., within ½ radius from injection site, within ⅓ radius from injection site, within ¼ radius from injection site, within ⅛ radius from injection site, and/or within 1/16 radius from injection site). In some embodiments, “about the same” refers to an expression level (e.g., at injection site) that is within 2SD from the expression level at the comparison location (e.g., within ½ radius from injection site, within ⅓ radius from injection site, within ¼ radius from injection site, within ⅛ radius from injection site, and/or within 1/16 radius from injection site).

In some embodiments, the transgene is more homogeneously expressed in a muscle of a subject after an rAAV of the disclosure is administered to the subject by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100% as compared to after a corresponding wild-type AAV is administered to the same subject or a different subject (e.g., an AAV that does not comprise a variant capsid comprising a peptide insertion of the disclosure). In some embodiments, homogeneity is measured using a fluorescence assay (e.g., fluorescence resonance energy transfer (FRET)). In some embodiments, transgene expression is detected and characterized by immunohistochemistry. In some embodiments, the transgene is more uniformly expressed in a muscle of the subject after an rAAV of the disclosure is administered to the subject as compared to after a reference AAV is administered to the same subject or a different subject (e.g., an AAV that does not comprise a variant capsid of the disclosure). In some embodiments, the transgene is expressed in a wider area of the muscle of the subject after an rAAV of the disclosure is administered to the subject as compared to after a corresponding wild type AAV is administered to the same subject or a different subject (e.g., an AAV that does not comprise a hybrid capsid of the disclosure). In some embodiments, the rAAV of the disclosure provides a widespread transgene expression in the musculature of the subject. Transgene product levels can be measured in patient samples of the treated muscle, or non-treated muscles.

5.2.3 rAAV Vectors

Provided herein are rAAV vectors comprising a hybrid AAV capsid as disclosed in Section 5.1.1. In some embodiments, a recombinant viral vector of the disclosure comprises a capsid region from one or more than one of: an AAV1, AAV2, AAV3, AAV3B, AAV4, AAV5, AAV6, AAV7, AAV8, AAV9, AAVrh10, AAV11, AAV12, AAV13, AAVrh20, AAVhu.37, AAVrh39, AAVhu32, AAVrh21, AAVrh13, AAVrh15, AAVrh73, or AAVrh74. In some embodiments, a recombinant viral vector of the disclosure comprises a capsid region from one or more than one of: AAV serotype 1 (AAV1), serotype 2 (AAV2), serotype 2tYF (AAV2tYF), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 10 (AAV10), serotype 11 (AAV11), serotype 12 (AAV12), serotype 13 (AAV13), serotype rh10 (AAVrh10), serotype rh20 (AAVrh20), serotype rh39 (AAVrh39), serotype hu.37 (AAVhu.37), serotype hu32 (AAVhu32), serotype rh13 (AAVrh13), rh15 (AAVrh15), serotype rh73 (AAVrh73), and serotype rh74 (AAVrh74). In some embodiments, the second AAV is an AAV serotype 1 (AAV1), serotype 2 (AAV2), serotype 2tYF (AAV2tYF), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 10 (AAV10), serotype 11 (AAV11), serotype 12 (AAV12), serotype 13 (AAV13), serotype rh10 (AAVrh10), serotype rh20 (AAVrh20), serotype rh39 (AAVrh39), serotype hu.37 (AAVhu.37), serotype hu32 (AAVhu32), serotype rh13 (AAVrh13), serotype rh15 (AAVrh15), serotype rh73 (AAVrh73), and serotype rh74 (AAVrh74).

In some embodiments, the AAV vector of the disclosure is a hybrid AAV. In some embodiments, the AAV vector of the disclosure is a chimeric AAV. In certain embodiments, the recombinant viral vector is a hybrid vector, e.g., an AAV vector placed into a “helpless” adenoviral vector. In some embodiments, the rAAV of the disclosure is useful for the treatment of a subject having, suspected of having, or experiencing a symptom associate with a disease (e.g., a human subject having, suspected of having, or experiencing a symptom associated with a disease associated with muscle), which rAAV comprises a hybrid AAV capsid as disclosed in Section 5.1.1 and 5.2.1. In certain embodiments, the AAV-based viral vector (e.g., hybrid AAV) provided herein retains tropism for a specific cell or tissue (e.g., muscle cell or tissue). In certain embodiments, the AAV-based vector provided herein encodes the AAV rep gene (required for replication) and/or the AAV cap gene (required for synthesis of the capsid protein). In some embodiments, the rAAV vector of the disclosure comprises a viral genome comprising an expression cassette for expression of a transgene or therapeutic product, under the control of regulatory elements, flanked by ITRs, and a variant capsid of the disclosure (e.g., a hybrid AAV capsid). In some embodiments, the AAV-based viral vector is a targeted vector, e.g., a vector targeted to muscle cells. In some embodiments, an rAAV vector of the disclosure comprises a nucleic acid sequence that is about or at least about 80%, 85%, 90%, 91%, 92%, 93%, 94%, 95%, 96%, 97%, 98%, 99%, or 100% identical to any one of SEQ ID NOs: 22, 24, 26, 30, 34, 36, 38, 40, 42, 44, 46, 48, 50, 52, 54, 56, 58, 60, 62, 64, 66, 68, 70, 72, 82, 84, 86, 88, 90, 92, 94, 96, 98, 100, 102, 104, 106, 108, 110, 112, 114, 116, 118, and 120. In some embodiments, an rAAV vector of the disclosure comprises a nucleic acid sequence of any one of SEQ ID NOs: 22, 24, 26, 30, 34, 36, 38, 40, 42, 44, 46, 48, 50, 52, 54, 56, 58, 60, 62, 64, 66, 68, 70, 72, 82, 84, 86, 88, 90, 92, 94, 96, 98, 100, 102, 104, 106, 108, 110, 112, 114, 116, 118, and 120. In some embodiments, an rAAV vector of the disclosure consists of a nucleic acid sequence of any one of SEQ ID NOs: 22, 24, 26, 30, 34, 36, 38, 40, 42, 44, 46, 48, 50, 52, 54, 56, 58, 60, 62, 64, 66, 68, 70, 72, 82, 84, 86, 88, 90, 92, 94, 96, 98, 100, 102, 104, 106, 108, 110, 112, 114, 116, 118, and 120. In some embodiments, an rAAV vector of the disclosure comprises a nucleic acid sequence encoding an rAAV capsid protein of the disclosure (Section 5.1.1 or 5.2.1). In some embodiments, an rAAV vector of the disclosure comprises a nucleic acid sequence encoding a polypeptide of the disclosure (Section 5.1.1 or 5.2.1). In some embodiments, an rAAV vector of the disclosure comprises a nucleic acid sequence encoding an rAAV capsid protein comprising an amino acid sequence that is about or at least about 80%, 85%, 90%, 91%, 92%, 93%, 94%, 95%, 96%, 97%, 98%, 995, or 100% identical to any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119. In some embodiments, an rAAV vector of the disclosure comprises a nucleic acid sequence encoding an rAAV capsid protein consisting of any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119.

Hybrid AAV-based viral vectors are used in certain embodiments of the methods described herein. Nucleic acid sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV-based viral vectors encoding a therapeutic product.

Provided in some embodiments are hybrid AAV vectors comprising a viral genome comprising an expression cassette for expression of a transgene, under the control of regulatory elements, and flanked by ITRs and an engineered viral capsid as described herein (refer to Section 5.1.1 or 5.2.1) or is at least 95%, 96%, 97%, 98%, 99% or 99.9% identical to the amino acid sequence of a hybrid AAV capsid protein or a wild-type AAV capsid protein, while retaining the biological function of the engineered hybrid AAVcapsid or wild-type AAV capsid.

In certain embodiments, a single-stranded AAV (ssAAV) may be used. In certain embodiments, a self-complementary vector, e.g., scAAV, may be used (see, e.g., Wu, 2007, Human Gene Therapy, 18 (2): 171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In some embodiments, a viral vector may be replication-deficient. A “replication-defective virus” or “viral vector” refers to a synthetic or recombinant viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In some embodiments, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

Fragments of AAV may be readily utilized in a variety of vector systems and host cells. Among desirable AAV fragments are the cap proteins, including the vp1, vp2, vp3 and hypervariable regions, the rep proteins, including rep 78, rep 68, rep 52, and rep 40, and the sequences encoding these proteins. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. An artificial AAV serotype may be, without limitation, a chimeric AAV capsid, a recombinant AAV capsid, or a “humanized” AAV capsid. In one embodiment, a vector contains AAV cap and/or rep sequences (e.g., the cap and/or rep sequences from a first AAV or a second AAV of the disclosure). See, U.S. Pat. No. 7,906,111, which is incorporated by reference herein.

### A. Promoters and Modifiers of Gene Expression

In certain embodiments, the recombinant vectors provided herein comprise components that modulate delivery or expression of the therapeutic product (e.g., “expression control elements”). In certain embodiments, the recombinant vectors provided herein comprise components that modulate expression of the transgene or therapeutic product. In certain embodiments, the recombinant vectors provided herein comprise components that influence binding or targeting to cells. In certain embodiments, the recombinant vectors provided herein comprise components that influence the localization of the polynucleotide encoding the therapeutic product within the cell after uptake. In certain embodiments, the recombinant vectors provided herein comprise components that can be used as detectable or selectable markers, e.g., to detect or select for cells that have taken up the polynucleotide encoding the therapeutic product.

In certain embodiments, the recombinant vectors provided herein comprise one or more promoters. In certain embodiments, the promoter is a constitutive promoter. In certain embodiments, the promoter is an inducible promoter. Inducible promoters may be preferred so that expression of the therapeutic product may be turned on and off as desired for therapeutic efficacy. Such promoters include, for example, hypoxia-induced promoters and drug inducible promoters, such as promoters induced by rapamycin and related agents. Hypoxia-inducible promoters include promoters with HIF binding sites, see, for example, Schödel, et al., 2011, Blood 117 (23): e207-e217 and Kenneth and Rocha, 2008, Biochem J. 414:19-29, each of which is incorporated by reference for teachings of hypoxia-inducible promoters. In addition, hypoxia-inducible promoters that may be used in the constructs include the erythropoietin promoter and N-WASP promoter (see, Tsuchiya, 1993, J. Biochem. 113:395 for disclosure of the erythropoietin promoter and Salvi, 2017, Biochemistry and Biophysics Reports 9:13-21 for disclosure of N-WASP promoter, both of which are incorporated by reference for the teachings of hypoxia-induced promoters). Alternatively, the recombinant vectors may contain drug inducible promoters, for example promoters inducible by administration of rapamycin and related analogs (see, for example, International Patent Application Publication Nos. WO94/18317, WO 96/20951, WO 96/41865, WO 99/10508, WO 99/10510, WO 99/36553, and WO 99/41258, and U.S. Pat. No. 7,067,526 (disclosing rapamycin analogs), which are incorporated by reference herein for their disclosure of drug inducible promoters). The inducible promoter may also be selected from known promoters including the ecdysone promoter, the estrogen-responsive promoter, and the tetracycline-responsive promoter, or heterodimeric repressor switch. See, Sochor et al, An Autogenously Regulated Expression System for Gene Therapeutic Ocular Applications. Scientific Reports, 2015 Nov. 24; 5:17105 and Daber R, Lewis M., A novel molecular switch. J Mol Biol. 2009 Aug. 28; 391 (4): 661-70, Epub 2009 Jun. 21 which are both incorporated herein by reference in their entirety.

In certain embodiments the promoter is a hypoxia-inducible promoter. In certain embodiments, the promoter comprises a hypoxia-inducible factor (HIF) binding site. In certain embodiments, the promoter comprises a HIF-1α binding site. In certain embodiments, the promoter comprises a HIF-2α binding site. In certain embodiments, the HIF binding site comprises an RCGTG motif. For details regarding the location and sequence of HIF binding sites, see, e.g., Schödel, et al., Blood, 2011, 117 (23): e207-e217, which is incorporated by reference herein in its entirety. In certain embodiments, the promoter comprises a binding site for a hypoxia induced transcription factor other than a HIF transcription factor. In certain embodiments, the recombinant vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety.

In another embodiment, the promoter is cell-specific. The term “cell-specific” means that the particular promoter selected for the recombinant vector can direct expression of the optimized transgene coding sequence in a particular cell or tissue type. In some embodiments, the promoter is a ubiquitous or constitutive promoter.

In some embodiments, a promoter is a muscle specific promoter. In some embodiments, a muscle-specific promoter may be operably linked to a transgene and then the artificial genome delivered via any of the AAV capsids described herein. In some embodiments, the muscle-specific promoter is selected from an SPc5-12 promoter, a muscle creatine kinase myosin light chain (MLC) promoter, a myosin heavy chain (MHC) promoter, a desmin promoter, a MHCK7 promoter, a CK6 promoter, a CK8 promoter, a MCK promoter, an alpha actin promoter, an beta actin promoter, an gamma actin promoter, an E-syn promoter, a cardiac troponin C promoter, a troponin I promoter, a myoD gene family promoter, or a muscle-selective promoter residing within intron 1 of the ocular form of Pitx3, or a truncated form or variant of any of the foregoing promoters.

In one example, synthetic promoter c5-12 (Li, X. et al. Nature Biotechnology Vol. 17, pp. 241-245, March 1999), known as the SPc5-12 promoter, has been shown to have cell type restricted expression, specifically muscle-cell specific expression. At less than 350 bp in length, the SPc5-12 promoter is smaller in length than most endogenous promoters, which can be advantageous when the length of the nucleic acid encoding the therapeutic protein is relatively long. Muscle-specific promoters may be combined with other cell- or tissue-specific promoters, for example liver-specific promoters as described in International Publication No. WO2021021661A1, which is hereby incorporated by reference.

In certain embodiments, the promoter is a CB7 promoter (see Dinculescu et al., 2005, Hum Gene Ther 16:649-663, incorporated by reference herein in its entirety). In certain embodiments, the CB7 promoter includes other expression control elements that enhance expression of the therapeutic product driven by the vector, e.g. (1) a CAG promoter; (2) a CBA promoter; (3) a CMV promoter; (4) a 1.7-kb red cone opsin promoter (PR1.7 promoter); (5) a Rhodopsin Kinase (GRK1) photoreceptor-specific enhancer-promoter (Young et al., 2003, Retinal Cell Biology; 44:4076-4085); (6) an hCARp promoter, which is a human cone arrestin promoter; (7) an hRKp, which is a rhodopsin kinase promoter; (8) a cone photoreceptor specific human arrestin 3 (ARR3) promoter; (9) a rhodopsin promoter; and (10) a U6 promoter (in particular when the therapeutic product is a small RNA such as shRNA or siRNA).

In certain embodiments, the promoter is a hybrid chicken β-actin (CBA) promoter with cytomegalovirus (CMV) enhancer elements. In another embodiment, the promoter is the CB7 promoter. Other suitable promoters include the human β-actin promoter, the human elongation factor-1α promoter, the cytomegalovirus (CMV) promoter, the simian virus 40 promoter, and the herpes simplex virus thymidine kinase promoter. See, e.g., Damdindorj et al, (August 2014) A Comparative Analysis of Constitutive Promoters Located in Adeno-Associated Viral Vectors. PLOS ONE 9 (8): e106472. Still other suitable promoters include viral promoters, constitutive promoters, regulatable promoters (see, e.g., WO 2011/126808 and WO 2013/04943). Alternatively a promoter responsive to physiologic cues may be utilized in the expression cassette, rAAV genomes, vectors, plasmids and viruses described herein. In one embodiment, the promoter is of a small size, under 1000 bp, due to the size limitations of the AAV vector. In another embodiment, the promoter is under 400 bp. Other promoters may be selected by one of skill in the art.

In a further embodiment, the promoter is selected from SV40 promoter, the dihydrofolate reductase promoter, a phage lambda (PL) promoter, a herpes simplex viral (HSV) promoter, a tetracycline-controlled trans-activator-responsive promoter (tet) system, a long terminal repeat (LTR) promoter, such as a RSV LTR, MoMLV LTR, BIV LTR or an HIV LTR, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter. The promoter sequences thereof are known to one of skill in the art or available publically, such as in the literature or in databases, e.g., GenBank, PubMed, or the like.

In certain embodiments, the other expression control elements include chicken β-actin intron and/or rabbit β-globin polA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the recombinant vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the recombinant vectors provided herein comprise one or more long terminal repeat (LTR) promoters selected from the group consisting of AAV, MLV, MMTV, SV40, RSV, HIV-1, and HIV-2 LTRs. In certain embodiments, the recombinant vectors provided herein comprise one or more tissue specific promoters (e.g., a retinal pigment epithelial cell-specific promoter). In certain embodiments, the recombinant vectors provided herein comprise a RPE65 promoter. In certain embodiments, the recombinant vectors provided herein comprise a VMD2 promoter.

In certain embodiments, the recombinant vectors provided herein comprise one or more regulatory elements other than a promoter. In certain embodiments, the recombinant vectors provided herein comprise an enhancer. In certain embodiments, the recombinant vectors provided herein comprise a repressor. In certain embodiments, the recombinant vectors provided herein comprise an intron or a chimeric intron. In certain embodiments, the recombinant vectors provided herein comprise a polyadenylation sequence.

### B. Untranslated Regions

In certain embodiments wherein the therapeutic product is a therapeutic protein, the recombinant vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the half-life of the mRNA encoding the therapeutic protein. In certain embodiments, the UTRs are optimized for the stability of the mRNA encoding the therapeutic protein. In certain embodiments, the UTRs are optimized for the secondary structure of the mRNA encoding the therapeutic protein.

### C. Inverted Terminal Repeats

In certain embodiments, the recombinant viral vectors provided herein comprise one or more inverted terminal repeat (ITR) sequences. ITR sequences may be used for packaging the recombinant therapeutic product expression cassette into the virion of the recombinant viral vector. In certain embodiments, the ITR is from an AAV, e.g., AAV9, AAV8 or AAV2 (see, e.g., Yan et al., 2005, J. Virol., 79 (1): 364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety.

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, VA). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

### 5.2.4 Transgene and Therapeutic Product

In some embodiments, provided herein is a method of delivering a therapeutic product or transgene to a subject (e.g., to treat a muscle related disease or disorder). In some embodiments, the rAAV of the disclosure mediates delivery of a transgene to a muscle tissue or cell of a subject. In some embodiments, provided herein is a method for delivering a transgene to a muscle tissue, e.g., for treating or preventing a disease or disorder associated with a muscle. In some embodiments, methods provided herein include delivering to a muscle of a subject an effective amount of rAAV, wherein the rAAV comprises (i) a hybrid capsid protein (e.g., from Section 5.1.1 or 5.2.1) and (ii) a nucleic acid comprising a promoter operably linked to a transgene or a nucleic acid encoding a transgene or therapeutic product. In some embodiments, the rAAV of the disclosure further comprises two AAV inverted terminal repeats (ITRs), wherein the ITRs flank the transgene. In some embodiments, the transgene encodes a gene or at least part of a gene associated with a muscle disease or disorder. A transgene incorporated into an rAAV of the disclosure is not limited and may be any heterologous nucleotide sequence of interest (e.g., a heterologous gene of interest). In some embodiments, a transgene is a nucleic acid sequence, heterologous to the vector genome sequences flanking the transgene, which encodes a polypeptide, protein, or other product, of interest. The nucleic acid coding sequence is operatively linked to one or more regulatory components (e.g., promoter, enhancer, poly-A, 3′UTR, Woodchuck Hepatitis Virus Posttranscriptional Regulatory Element (WPRE)) in a manner which permits transgene transcription, translation, and/or expression in a host cell. The composition of the heterologous transgene sequence will depend upon the application (e.g., the therapeutic application or indication to be treated). In some embodiments, an rAAV of the disclosure comprises two or more heterologous transgenes, for example, two, three, four or five heterologous transgenes. In other embodiments, an rAAV of the disclosure comprises one heterologous transgene incorporated into the rAAV viral particle. In some embodiments, the transgene comprises a heterologous gene associated with a disease or disorder (e.g., muscle related disease or disorder). In some embodiments, the heterologous gene is operably linked to a regulatory sequence that controls expression of the heterologous gene in a host cell. In some embodiments, a host cell is a muscle cell. In some embodiments, a transgene encodes a therapeutic protein. In some embodiments, a therapeutic protein is associated with a muscle related disease or disorder. In some embodiments, a transgene encodes a functional dystrophin, a minidystrophin, a microdystrophin, and/or a dystrophin exon-skipping snRNA. In some embodiments, a transgene comprises a polynucleotide sequence encoding one or more of any one of SEQ ID NOs: 73, 74, 75, 76, 77, 78, 79, and/or 80. In some embodiments, a transgene comprises a polynucleotide sequence encoding at least a portion (e.g., about or at least about: 10 amino acids, 15 amino acids, 20 amino acids, 25 amino acids, 30 amino acids, 35 amino acids, 40 amino acids, 45 amino acids, 50 amino acids, 60 amino acids, 65 amino acids, 70 amino acids, 75 amino acids, 80 amino acids, 85 amino acids, 90 amino acids, 95 amino acids, 100 amino acids, or more than 100 amino acids) of one or more of any one of SEQ ID NOs: 73, 74, 75, 76, 77, 78, 79, and/or 80. In some embodiments, a transgene consists of a polynucleotide sequence encoding one or more of any one of SEQ ID NOs: 73, 74, 75, 76, 77, 78, 79, and/or 80.

The size of the nucleotide sequence of a transgene can vary. For example, the nucleotide sequence of a transgene encoding a therapeutic protein can be at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length. In some embodiments, the nucleotide sequence of a transgene encoding a therapeutic protein is at least about 1.4 kb in length. In certain embodiments, the nucleotide sequence of a transgene encoding a therapeutic protein is about 1.4 kb to 5 kb in length. In some embodiments, the nucleotide sequences of a transgene encoding a therapeutic protein is 1.4 kb to 5 kb or 5 kb to 10 kb. Alternatively, the nucleotide sequence of a transgene is at least about 30 nucleotides, at least about 40 nucleotides, at least about 50 nucleotides, at least about 75 nucleotides in length, at least about 100 nucleotides in length, at least about 150 nucleotides in length, at least about 200 nucleotides in length, at least about 250 nucleotides in length, at least about 300 nucleotides in length, at least about 350 nucleotides in length, at least about 400 nucleotides in length, at least about 500 nucleotides in length, at least about 600 nucleotides in length, at least about 700 nucleotides in length, at least about 800 nucleotides in length, at least about 900 nucleotides in length, at least about 1000 nucleotides in length, or at least about 1200 nucleotides in length. In some embodiments, the nucleotide sequence of a transgene is about 30 to 150 nucleotides in length or about 150 to 500 nucleotides in length. In certain embodiments, the nucleotide sequence of a transgene is about 100 to 500 nucleotides in length or 500 to 1000 nucleotides in length. In some embodiments, the nucleotide sequence of a transgene is 500 nucleotides to 1200 nucleotides in length.

In some embodiments, an rAAV of the disclosure comprises a therapeutic transgene. A therapeutic transgene of the disclosure can be a sequence that encodes a biomolecule (e.g., a therapeutic biomolecule) which is useful in biology and treatment of a disease, such as a protein (e.g., an enzyme), polypeptide, peptide, RNA (e.g., tRNA, dsRNA, ribosomal RNA, catalytic RNAs, siRNA, miRNA, pre-miRNA, lncRNA, snoRNA, small hairpin RNA, trans-splicing RNA, and antisense RNA), one or more components of a gene or base editing system, e.g., CRISPR gene editing system, antisense oligonucleotides (AONs), antisense oligonucleotide (AON)-mediated exon skipping, a poison exon(s) that triggers nonsense mediated decay (NMD), or a dominant negative mutant. In some embodiments, a transgene comprises a nucleic acid sequence encoding a sequence useful for gene therapy applications. For example, certain diseases come about when one or more loss-of-function mutations within a gene reduce or abolish the amount or activity of the protein encoded by the gene. In certain embodiments, a transgene utilized herein encodes a functional version of the protein. In other embodiments, an rAAV of the disclosure comprises a transgene comprising a nucleic acid sequence encoding a sequence useful for gene therapy applications that benefit from gene silencing. For example, certain diseases come about when gain-of-function mutations within a gene result in an aberrant amount or activity of the protein encoded by the gene. In certain embodiments, a transgene utilized herein encodes an inhibitory polynucleotide, e.g., an inhibitory RNA such as an miRNA or siRNA, or one or more components of gene editing system, e.g., a CRISPR gene editing system. In some embodiments, a transgene comprises a nucleic acid encoding a CRISPR-Cas system for targeted gene disruption or correction. In other embodiments, a transgene comprising a nucleic acid sequence encodes a sequence useful for gene therapy applications that benefit from gene addition. In certain embodiments, a transgene utilized herein encodes a gene product, e.g., a protein, not present in a recipient, e.g., a human subject, of the gene therapy.

In some embodiments, a transgene comprises a nucleic acid sequence encoding an RNA sequence useful in biology and medicine, such as, e.g., tRNA, dsRNA, ribosomal RNA, catalytic RNA, siRNA, miRNA, pre-miRNA, lncRNA, snoRNA, small hairpin RNA, trans-splicing RNA, and antisense RNA. One example of a useful RNA sequence is a sequence which inhibits or extinguishes expression of a targeted nucleic acid sequence in a treated subject. Suitable target nucleic acid sequences may include oncologic sequences and viral sequences. In some embodiments, a transgene comprises a nucleic acid sequence encoding a small nuclear RNA (snRNA) construct which induces exon skipping. In certain embodiments, an RNAi agent targets a gene of interest at a location of a single-nucleotide polymorphism (SNP) or a variant within the nucleotide sequence.

In some embodiments, an RNAi agent is an siRNA duplex, wherein the siRNA duplex contains an antisense strand (guide strand) and a sense strand (passenger strand) hybridized together forming a duplex structure, wherein the antisense strand is at least partially complementary to the nucleic acid sequence of the targeted gene, and wherein the sense strand is at least partially homologous to the nucleic acid sequence of the targeted gene. In some embodiments, the 5′ end of the antisense strand has a 5′phosphate group and the 3′end of the sense strand contains a 3′ hydroxyl group. In some embodiments, there are none, one or 2 nucleotide overhangs at the 3′ end of one or both strands. In some embodiments, one or more than one nucleotide of an antisense strand and/or a sense strand is modified. Non-limiting examples of nucleotide modifications include 2′deoxy, 2′-fluoro, 2′ O-methyl, 2′deoxy-2′fluoro, a phosphorothioate, 5′-morpholinno, a universal base modified nucleotide, a terminal cap molecule at the 3′-end, the 5′-end, or both 3′ and 5′-ends, an inverted abasic, or an inverted abasic locked nucleic acid modification at the 5′-end and/or 3′ end.

In some embodiments, each strand of an siRNA duplex targeting a gene of interest is about 19 to 25, 19 to 24 or 19 to 21 nucleotides in length. In some embodiments, an siRNA or dsRNA includes at least two sequences that are complementary to each other. The dsRNA includes a sense strand having a first sequence and an antisense strand having a second sequence. In some embodiments, the antisense strand includes a nucleotide sequence that is substantially complementary to at least part of an mRNA encoding the target gene, and the region of complementarity is 30 nucleotides or less, and at least 15 nucleotides in length. In some embodiments, the dsRNA is 19 to 25, 19 to 24 or 19 to 21 nucleotides in length. In some embodiments, the dsRNA is from about 15 to about 25 nucleotides in length. In some embodiments, the dsRNA is from about 25 to about 30 nucleotides in length. In some embodiments, the dsRNA is about, at least about, or at most about 15 nucleotides in length, 16 nucleotides in length, 17 nucleotides in length, 18 nucleotides in length, 19 nucleotides, 20 nucleotides, 21 nucleotides, 22 nucleotides, 23 nucleotides, 24 nucleotides, 25 nucleotides in length, 26 nucleotides in length, 27 nucleotides in length, 28 nucleotides in length, 29 nucleotides in length, or 30 nucleotides in length.

In some embodiments, an rAAV of the disclosure comprises a transgene comprising a nucleic acid sequence encoding a protein, peptide or other product that corrects or ameliorates a genetic deficiency or other abnormality in a subject. Such genetic deficiencies may include deficiencies in which gene products are expressed at less than levels considered normal for a particular subject (e.g., a human subject) or deficiencies in which a functional gene product is not expressed. In some embodiments, an rAAV of the disclosure comprises multiple transgenes to, e.g., correct or ameliorate a genetic defect caused by a multi-subunit protein. In some instances, a different transgene may be used to encode each subunit of a protein, or to encode different peptides or proteins. This may be desirable when the size of the nucleic acid sequence encoding the protein subunit is large, non-limiting examples include e.g., for an immunoglobulin, the platelet-derived growth factor, or a dystrophin protein. A host cell may be infected with an rAAV of the disclosure containing transgenes, wherein each transgene comprises a nucleic acid sequence encoding a different subunit of a multi-subunit protein, in order to produce the multi-subunit protein. Alternatively, an rAAV of the disclosure may comprise a single transgene comprising nucleic acid sequences encoding different subunits of a multi-subunit protein. In this case, a single transgene comprises nucleic acid sequences encoding each of the subunits and the nucleic acid sequence encoding each subunit may be separated by an internal ribozyme entry site (IRES). This may be desirable when the size of the nucleic acid sequence encoding each of the subunits is small, e.g., the total size of the nucleic acid sequences encoding the subunits and the IRES is less than five kilobases. As an alternative to an IRES, the nucleic acid sequence may be separated by sequences encoding a peptide, such as, e.g., 2A peptide, which self-cleaves in a post-translational event. See, e.g., Donnelly et al, J. Gen. Virol., 78 (Pt 1): 13-21 (January 1997); Furler, et al, Gene Ther., 8 (11): 864-873 (June 2001); Klump et al., Gene Ther., 8 (10): 811-817 (May 2001). A 2A peptide is significantly smaller than an IRES, making it well suited for use when space is a limiting factor. More often, when a transgene is large, consists of multi-subunits, or both, two or more AAV viral particles (including an rAAV of the disclosure) each carrying a desired transgene may be co-administered to allow them to concatamerize in vitro or in vivo to form a single vector genome. See, e.g., Yang et al., J Virol. 1999 November; 73 (11): 9468-9477 for information regarding the concatamerization of AAV. For example, a first AAV viral particle may comprise a single transgene and a second AAV viral particle may comprise a different transgene for co-expression in a host cell.

In some embodiments, a transgene comprises a nucleic acid sequence encoding a protein heterologous to AAV (e.g., a therapeutic protein). In certain embodiments, a transgene comprises a nucleic acid sequence encoding a therapeutic protein that is endogenously expressed in, for example, a muscle tissue of a subject.

In some embodiments, a transgene comprises a nucleic acid sequence, which upon expression produces a detectable signal. In some instances, such a nucleic acid sequence encodes an enzyme (such as, e.g., β-lactamase, β-galactosidase (LacZ), alkaline phosphatase, thymidine kinase, chloramphenicol acetyltransferase (CAT), and luciferase), a fluorescent protein (such as, e.g., green fluorescent protein (GFP), yellow fluorescent protein, and red fluorescent protein), a membrane bound protein (such as, e.g., CD2, CD4, CD8, the influenza hemagglutinin protein, and others well known in the art, to which high affinity antibodies directed thereto exist or can be produced by conventional means) or a fusion protein comprising a membrane bound protein appropriately fused to an antigen tag domain. These nucleic acid sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry (IHC). For example, where the nucleic acid sequence encodes LacZ, the presence of an AAV vector genome expressing LacZ may be detected by assays for beta-galactosidase activity. In another example, where the transgene comprises a nucleic acid sequence encoding green fluorescent protein or luciferase, an AAV vector genome expressing the green fluorescent protein or luciferase may be detected visually by color or light production in a luminometer. An AAV viral particle comprising a transgene that comprises a nucleotide sequence encoding a product with a detectable signal may be used a selectable marker or may be used to trace the virus.

A gene associated with a muscle related disease or a disease can be a protein, polypeptide, antibody or fragment thereof (e.g., ScFv), toxin, or interfering RNA (e.g., siRNA, dsRNA, miRNA, artificial miRNA (ami-RNA), antagomir). Examples of genes associated with a muscle related disease or a disease include, but are not limited to DYS1, DYS3, DYS5, human MD1 (R4-R23/ΔCT), human microdystrophin, Dys3978, human MD3, and/or human MD4. In some embodiments, the rAAV comprises a transgene comprising at least a portion of a DYS1 sequence. In some embodiments, the DYS1 sequence comprises an amino acid sequence of SEQ ID NO: 73. In some embodiments, the DYS1 sequence consists of SEQ ID NO: 73. In some embodiments, the DYS1 sequence comprises an amino acid sequence consisting of SEQ ID NO: 73. In some embodiments, the rAAV comprises a transgene comprising at least a portion of DYS3 sequence. In some embodiments, the DYS3 sequence comprises the amino acid sequence of SEQ ID NO: 74. In some embodiments, the DYS3 sequence consists of SEQ ID NO: 74. In some embodiments, the DYS3 sequence comprises an amino acid sequence consisting of SEQ ID NO: 74. In some embodiments, the rAAV comprises a transgene comprising at least a portion of DYS5 sequence. In some embodiments, the DYS5 sequence comprises an amino acid sequence of SEQ ID NO: 75. In some embodiments, the DYS5 sequence consists of SEQ ID NO: 75. In some embodiments, the DYS5 sequence comprises an amino acid sequence consisting of SEQ ID NO: 75. In some embodiments, the rAAV comprises a transgene comprising at least a portion of a human MD1 (R4-R23/ΔCT) sequence. In some embodiments, the human MD1 (R4-R23/ΔCT) sequence comprises an amino acid sequence of SEQ ID NO: 76. In some embodiments, the human MD1 (R4-R23/ΔCT) sequence consists of SEQ ID NO: 76. In some embodiments, the human MD1 (R4-R23/ΔCT) sequence comprises an amino acid sequence consisting of SEQ ID NO: 76. In some embodiments, the rAAV comprises a transgene comprising at least a portion of a human microdystrophin sequence. In some embodiments, the human microdystrophin sequence comprises the amino acid sequence of SEQ ID NO: 77. In some embodiments, the human microdystrophin sequence consists of SEQ ID NO: 77. In some embodiments, the human microdystrophin sequence comprises an amino acid sequence consisting of SEQ ID NO: 77. In some embodiments, the rAAV comprises a transgene comprising at least a portion of Dys3978 sequence. In some embodiments, the Dys3978 sequence comprises the amino acid sequence of SEQ ID NO:78. In some embodiments, the Dys3978 sequence consists of SEQ ID NO: 78. In some embodiments, the Dys3978 sequence comprises an amino acid sequence consisting of SEQ ID NO: 78. In some embodiments, the rAAV comprises a transgene comprising at least a portion of human MD3 sequence. In some embodiments, the human MD3 sequence comprises the amino acid sequence of SEQ ID NO:79. In some embodiments, the human MD3 sequence consists of SEQ ID NO: 79. In some embodiments, the human MD3 sequence comprises an amino acid sequence consisting of SEQ ID NO: 79. In some embodiments, the rAAV comprises a transgene comprising a human MD4 sequence. In some embodiments, the human MD4 sequence comprises the amino acid sequence of SEQ ID NO:80. In some embodiments, the human MD4 sequence consists of SEQ ID NO: 80. In some embodiments, the human MD4 sequence comprises an amino acid sequence consisting of SEQ ID NO: 80. In some embodiments, the transgene is selected from SMN, mini/micro dystrophin gene, human-alpha-sarcoglycan, gamma-sarcoglycan, huFollistatin344, GALGT2, and/or hSGCB. In some embodiments, the transgene comprises or consists of SMN. In certain embodiments, the rAAV comprising a SMN transgene is for treating Spinal Muscular Atrophy (SMA) in the subject. In some embodiments, the transgene comprises or consists of a mini/micro dystrophin gene. In certain embodiments, the rAAV comprising a mini/micro dystrophin gene transgene is for treating Duchenne Muscular Dystrophy in the subject. In some embodiments, the transgene comprises or consists of a human-alpha-sarcoglycan. In certain embodiments, the rAAV comprising a human-alpha-sarcoglycan transgene is for treating Duchenne Muscular Dystrophy in the subject. In certain embodiments, the rAAV comprising a human-alpha-sarcoglycan transgene is for treating Limb Girdle Muscular Dystrophy Type 2C or Gamma-sarcoglycanopathy in the subject. In some embodiments, the transgene comprises or consists of a gamma-sarcoglycan. In certain embodiments, the rAAV comprising a gamma-sarcoglycan transgene is for treating Limb Girdle Muscular Dystrophy Type 2C or Gamma-sarcoglycanopathy in the subject. In some embodiments, the transgene comprises or consists of a huFollistatin344. In certain embodiments, the rAAV comprising a huFollistatin344 transgene is for treating Becker Muscular Dystrophy or Sporadic Inclusion Body Myositis in the subject. In some embodiments, the transgene comprises or consists of GALGT2. In certain embodiments, the rAAV comprising a GALGT2 transgene is for treating Duchenne Muscular Dystrophy in the subject In some embodiments, the transgene comprises or consists of hSGCB. In certain embodiments, the rAAV comprising a GALGT2 transgene is for treating Limb-Girdle Muscular Dystrophy Type 2E in the subject.

### 5.2.5 Pharmaceutical Compositions and Kits

In certain embodiments, provided herein are pharmaceutical compositions comprising an rAAV of the disclosure and a pharmaceutically acceptable carrier. In some embodiments, a pharmaceutical composition comprises a recombinant adeno-associated virus (rAAV) vector comprising a recombinant AAV capsid protein, wherein the recombinant AAV capsid protein comprises an amino acid sequence that is about or at least about 80%, 85%, 90%, 91%, 92%, 93%, 94%, 95%, 96%, 97%, 98%, 99%, or 100% identical to any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119. In some embodiments, a pharmaceutical composition comprises a recombinant adeno-associated virus (rAAV) vector comprising a nucleic acid sequence that is about or at least about 80%, 85%, 90%, 91%, 92%, 93%, 94%, 95%, 96%, 97%, 98%, 99%, or 100% identical to any one of SEQ ID NOs: 22, 24, 26, 30, 34, 36, 38, 40, 42, 44, 46, 48, 50, 52, 54, 56, 58, 60, 62, 64, 66, 68, 70, 72, 82, 84, 86, 88, 90, 92, 94, 96, 98, 100, 102, 104, 106, 108, 110, 112, 114, 116, 118, and 120. The pharmaceutical composition may be prepared as individual, single unit dosage forms. The pharmaceutical compositions provided herein can be formulated for, for example, parenteral, subcutaneous, intramuscular, intravenous, intraperitoneal, intranasal, intrathecal, transdermal, suprachoroidal, retinal, subretinal, juxtascleral, intravitreal, subconjunctival, and/or intraretinal administration.

Compositions are described comprising a recombinant vector encoding a therapeutic product described herein and a suitable carrier. A suitable carrier (e.g., for suprachoroidal, subretinal, juxtascleral, intravitreal, subconjunctival, and/or intraretinal administration) would be readily selected by one of skill in the art.

The disclosure provides a pharmaceutical composition comprising a recombinant adeno-associated virus (AAV), potassium phosphate monobasic, sodium chloride, sodium phosphate dibasic anhydrous, sucrose, and surfactant.

In some embodiments, the disclosure provides a pharmaceutical composition comprising a recombinant adeno-associated virus (AAV), ionic salt excipient or buffering agent, sucrose, and poloxamer 188. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, calcium citrate.

In certain embodiments, the pharmaceutical composition has a ionic strength about 60 mM to 115 mM. In certain embodiments, the pharmaceutical composition has a ionic strength about 60 mM to 100 mM. In certain embodiments, the pharmaceutical composition has a ionic strength about 65 mM to 95 mM. In certain embodiments, the pharmaceutical composition has a ionic strength about 70 mM to 90 mM. In certain embodiments, the pharmaceutical composition has a ionic strength about 75 mM to 85 mM.

In certain embodiments, the pharmaceutical composition has a ionic strength about 30 mM to 100 mM. In certain embodiments, the pharmaceutical composition has a ionic strength about 35 mM to 95 mM. In certain embodiments, the pharmaceutical composition has a ionic strength about 40 mM to 90 mM. In certain embodiments, the pharmaceutical composition has a ionic strength about 45 mM to 85 mM. In certain embodiments, the pharmaceutical composition has a ionic strength about 50 mM to 80 mM. In certain embodiments, the pharmaceutical composition has a ionic strength about 55 mM to 75 mM. In certain embodiments, the pharmaceutical composition has a ionic strength about 60 mM to 70 mM.

In certain embodiments, the pharmaceutical composition has a ionic strength ranging from 60 mM to 115 mM. In certain embodiments, the pharmaceutical composition has a ionic strength ranging from 60 mM to 100 mM. In certain embodiments, the pharmaceutical composition has a ionic strength ranging from 65 mM to 95 mM. In certain embodiments, the pharmaceutical composition has a ionic strength ranging from 70 mM to 90 mM. In certain embodiments, the pharmaceutical composition has a ionic strength ranging from 75 mM to 85 mM.

In certain embodiments, the pharmaceutical composition has a ionic strength range from 30 mM to 100 mM. In certain embodiments, the pharmaceutical composition has a ionic strength range from 35 mM to 95 mM. In certain embodiments, the pharmaceutical composition has a ionic strength range from 40 mM to 90 mM. In certain embodiments, the pharmaceutical composition has a ionic strength range from 45 mM to 85 mM. In certain embodiments, the pharmaceutical composition has a ionic strength range from 50 mM to 80 mM. In certain embodiments, the pharmaceutical composition has a ionic strength range from 55 mM to 75 mM. In certain embodiments, the pharmaceutical composition has a ionic strength range from 60 mM to 70 mM.

In certain embodiments, the pharmaceutical composition comprises potassium chloride at a concentration of 0.2 g/L.

In certain embodiments, the pharmaceutical composition comprises potassium phosphate monobasic at a concentration of 0.2 g/L.

In certain embodiments, the pharmaceutical composition comprises sodium chloride at a concentration of 5.84 g/L, and

In certain embodiments, the pharmaceutical composition comprises sodium phosphate dibasic anhydrous at a concentration of 1.15 g/L.

In certain embodiments, the pharmaceutical composition comprises sucrose at a concentration of 3% (weight/volume, 30 g/L) to 18% (weight/volume, 180 g/L). In certain embodiments, the pharmaceutical composition comprises sucrose at a concentration of 4% (weight/volume, 40 g/L).

In certain embodiments, the pharmaceutical composition comprises poloxamer 188 at a concentration of 0.001% (weight/volume, 0.01 g/L).

In certain embodiments, the pharmaceutical composition comprises poloxamer 188 at a concentration of 0.0005% (weight/volume, 0.005 g/L) to 0.05% (weight/volume, 0.5 g/L). In certain embodiments, the pharmaceutical composition comprises poloxamer 188 at a concentration of 0.001% (weight/volume, 0.01 g/L).

In some embodiments, the disclosure provides a pharmaceutical composition comprises a recombinant adeno-associated virus (AAV), ionic salt excipient or buffering agent, sucrose, and surfactant. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, calcium citrate. In some embodiments, the surfactant can be one or more components from the group consisting of poloxamer 188, polysorbate 20, and polysorbate 80.

In certain embodiments, the pharmaceutical composition comprises polysorbate 20 at a concentration of 0.0005% (weight/volume, 0.05 g/L) to 0.05% (weight/volume, 0.5 g/L).

In certain embodiments, the pharmaceutical composition comprises polysorbate 80 at a concentration of 0.0005% (weight/volume, 0.05 g/L) to 0.05% (weight/volume, 0.5 g/L).

In certain embodiments, the pH of the pharmaceutical composition is about 7.4.

In certain embodiments, the pH of the pharmaceutical composition is about 6.0 to 9.0.

In certain embodiments, the pH of the pharmaceutical composition is 7.4.

In certain embodiments, the pH of the pharmaceutical composition is 6.0 to 9.0.

As used herein and unless otherwise specified, the term “about” means within plus or minus 10% of a given value or range.

In certain embodiments, the pharmaceutical composition is in a hydrophobically-coated glass vial.

In certain embodiments, the pharmaceutical composition is in a Cyclo Olefin Polymer (COP) vial.

In certain embodiments, the pharmaceutical composition is in a Daikyo Crystal Zenith® (CZ) vial.

In certain embodiments, the pharmaceutical composition is in a TopLyo coated vial.

In certain embodiments, disclosed herein is a pharmaceutical composition consists of: (a) the recombinant AAV, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the recombinant AAV is AAV9.

In certain embodiments, the vector genome concentration (VGC) of the pharmaceutical composition is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 1× 10¹¹ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 3×10¹² GC/mL, about 2×10¹³ GC/mL or about 3×10¹³ GC/mL. In some embodiments, 2×10⁹ and 1×10¹⁰ of vector genome is administered to a subject.

In certain embodiments, the disclosure provides a pharmaceutical composition or formulation comprising a recombinant adeno-associated virus (AAV) of the disclosure, potassium phosphate monobasic, sodium chloride, sodium phosphate dibasic anhydrous, sucrose, and poloxamer 188. In some embodiments, a pharmaceutical composition or formulation comprises a polypeptide of the disclosure (e.g., from Section 5.1.1), a hybrid AAV of the disclosure (e.g., from Section 5.2.1), a nucleotide sequence of the disclosure, a vector of the disclosure, or any other component of the disclosure.

In some embodiments, the pharmaceutical composition consists of: (a) an AAV capsid packaging vector encoding a transgene of interest, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water.

In some embodiments, the pharmaceutical composition is a liquid composition. In some embodiments, the pharmaceutical composition is a frozen composition. In some embodiments, the pharmaceutical composition is a lyophilized composition from a liquid composition disclosed herein. In some embodiments, the pharmaceutical composition is a reconstituted lyophilized formulation.

In some embodiments, the pharmaceutical composition is a lyophilized composition comprising a residual moisture content between about 1% and about 7%. In some embodiments, the pharmaceutical composition is a lyophilized composition comprising a residual moisture content between about 2% and about 6%. In some embodiments, the pharmaceutical composition is a lyophilized composition comprising a residual moisture content between about 3% and about 4%. In some embodiments, the pharmaceutical composition is a lyophilized composition comprising a residual moisture content about 5%.

In certain aspects, disclosed herein is a method of treating or preventing a disease in a subject, comprising administering to the subject the pharmaceutical composition. In some embodiments, a pharmaceutical composition provided herein is suitable for administration by one, two or more routes of administration.

In certain aspects, disclosed herein is a method of treating or preventing a disease in a subject, comprising administering to the subject the pharmaceutical composition by intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface).

In certain embodiments, the pharmaceutical composition provided herein is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired viscosity that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired density that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired osmolality that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)). In specific embodiments, the desired osmolality for subretinal administration is 160 to 430 mOsm/kg H₂O. In other specific embodiments, the desired osmolality of suprachoroidal administration is less than 600 mOsm/kg H₂O.

In certain embodiments, the pharmaceutical composition has a osmolality of about 100 to 500 mOsm/kg H₂O. In certain embodiments, the pharmaceutical composition has a osmolality of about 130 to 470 mOsm/kg H₂O. In certain embodiments, the pharmaceutical composition has a osmolality of about 160 to 430 mOsm/kg H₂O. In certain embodiments, the pharmaceutical composition has a osmolality of about 200 to 400 mOsm/kg H₂O. In certain embodiments, the pharmaceutical composition has a osmolality of about 240 to 340 mOsm/kg H₂O. In certain embodiments, the pharmaceutical composition has a osmolality of about 280 to 300 mOsm/kg H₂O. In certain embodiments, the pharmaceutical composition has a osmolality of about 295 to 395 mOsm/kg H₂O. In certain embodiments, the pharmaceutical composition has a osmolality of less than 600 mOsm/kg H₂O. In certain embodiments, the pharmaceutical composition has a osmolality range of 200 mOsm/L to 660 mOsm/L. In certain embodiments, the pharmaceutical composition has a osmolality of about 200 mOsm/L. In certain embodiments, the pharmaceutical composition has a osmolality of about 250 mOsm/L. In certain embodiments, the pharmaceutical composition has a osmolality of about 300 mOsm/L. In certain embodiments, the pharmaceutical composition has a osmolality of about 350 mOsm/L. In certain embodiments, the pharmaceutical composition has a osmolality of about 400 mOsm/L. In certain embodiments, the pharmaceutical composition has a osmolality of about 450 mOsm/L. In certain embodiments, the pharmaceutical composition has a osmolality of about 500 mOsm/L. In certain embodiments, the pharmaceutical composition has a osmolality of about 550 mOsm/L. In certain embodiments, the pharmaceutical composition has a osmolality of about 600 mOsm/L. In certain embodiments, the pharmaceutical composition has a osmolality of about 650 mOsm/L. In certain embodiments, the pharmaceutical composition has a osmolality of about 660 mOsm/L.

In some embodiments, the recombinant vector of the disclosure is used for delivering the transgene to a cell. Such vectors can include non-replicating recombinant adeno-associated virus vectors (“rAAV”), however, other viral vectors may be used, including but not limited to lentiviral vectors, vaccinia viral vectors, or non-viral expression vectors referred to as “naked DNA” constructs.

In certain embodiments, gene therapy constructs are supplied as a frozen sterile, single use solution of the AAV vector active ingredient in a formulation buffer. In a specific embodiment, the pharmaceutical compositions suitable for subretinal administration comprise a suspension of the recombinant vector in a formulation buffer comprising a physiologically compatible aqueous buffer, a surfactant and optional excipients. In a specific embodiment, the construct is formulated in Dulbecco's phosphate buffered saline and 0.001% poloxamer 188, pH=7.4.

The disclosure provides a pharmaceutical composition comprising a pharmaceutically acceptable carrier and an agent of the disclosure, said agent comprising a rAAV of the disclosure. In some embodiments, the pharmaceutical composition comprises rAAV combined with a pharmaceutically acceptable carrier for administration to a subject. In some embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present disclosure can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

In certain embodiments of the disclosure, pharmaceutical compositions are provided for use in accordance with the methods of the disclosure, said pharmaceutical compositions comprising a therapeutically and/or prophylactically effective amount of an agent of the disclosure along with a pharmaceutically acceptable carrier.

In certain embodiments, the agent of the disclosure is substantially purified (i.e., substantially free from substances that limit its effect or produce undesired side-effects). In one embodiment, the host or subject is an animal, e.g., a mammal such as non-primate (e.g., cows, pigs, horses, cats, dogs, rats etc.) and a primate (e.g., monkey such as, a cynomolgus monkey and a human). In a certain embodiment, the host is a human.

Provided herein are kits comprising a pharmaceutical composition described herein, contained in one or more containers. The containers that the pharmaceutical composition can be packaged in can include, but are not limited to, bottles, packets, ampoules, tubes, inhalers, bags, vials, and containers. In certain embodiments, the kit comprises instructions for administering the pharmaceutical administration. In certain embodiments, the kit comprises devices that can be used to administer (e.g., to musculature tissue) the pharmaceutical composition, including, but not limited to, syringes, catheters, needle-less injectors, drip bags, patches and inhalers. In another embodiment, a kit further comprises one or more other prophylactic or therapeutic agents useful for the treatment of a condition, in one or more containers.

The disclosure also provides agents of the disclosure packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the disclosure is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The compositions of the disclosure include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The disclosure further provides a pharmaceutical pack or kit comprising one or more containers filled with one or more of the agents of the disclosure. Additionally, one or more other prophylactic or therapeutic agents useful for the treatment of the target disease or disorder can also be included in the pharmaceutical pack or kit. The disclosure also provides a pharmaceutical pack or kit comprising one or more containers filled with one or more of the ingredients of the pharmaceutical compositions of the disclosure. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

5.3 Manufacture of rAAVS

### 5.3.1 Polynucleotides, Plasmids and Cells

In some embodiments, the disclosure provides for an isolated nucleic acid comprising a nucleotide sequence encoding a variant adeno-associated virus (AAV) capsid protein of the disclosure (e.g., a hybrid AAV capsid comprising a heterologous amino acid sequence or a peptide insert). In some aspects, the disclosure provides for a nucleic acid for use, wherein the nucleic acid encodes a therapeutic product operatively linked to a promoter or enhancer-promoter described herein.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence encoding the therapeutic product of interest, untranslated regions, and termination sequences. In certain embodiments, recombinant vectors provided herein comprise a promoter operably linked to the sequence encoding the therapeutic product of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161). In some embodiments, the polynucleotide is in the form of a ssDNA. In another embodiment, the polynucleotide is in the form of a dsDNA.

Also provided herein are plasmids comprising a polynucleotide provided herein (hereinafter “rAAV plasmids”). In one embodiment, the rAAV plasmid is a ssDNA plasmid. In another embodiment, the rAAV plasmid is a dsDNA plasmid. In some embodiments, the rAAV plasmid is in a circular form. In other embodiments, the rAAV plasmid is in a linear form.

Further provided herein are cells (preferably ex vivo cells) expressing (e.g., recombinantly) an rAAV provided herein. In certain embodiments, the cell (e.g., ex vivo cell) comprises a polynucleotide provided herein or an rAAV plasmid provided herein. In certain embodiments, the cell (e.g., ex vivo cell) further comprises helper polynucleotide(s) or helper plasmids providing the AAV Rep, Cap, and Ad5 functions. The cell (e.g., ex vivo cells) can be a mammalian host cell, for example, a cell associated with a muscle, HEK293, HEK293-T, A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The mammalian host cell can be derived from, for example, human, monkey, mouse, rat, rabbit, or hamster. In some embodiments, the mammalian host cell is a muscle related cell. In certain embodiments, the mammalian host cell is a human embryonic kidney 293 (HEK293) cell or HEK293-T cell. Provided are polynucleotides encoding the recombinant AAV hybrid capsid proteins, as an rAAV plasmid, and such Cap or “RepCap” constructs comprise the Cap gene encoding an hybrid capsid described herein. Also provided are host cells, such as bacterial host cells, for replication and production of these plasmid vectors. In other embodiments, the polynucleotides described herein comprise an rAAV plasmid which is replicable in a bacterial cell. In some embodiments, the host cell is a bacterial host cell comprising the rAAV plasmid.

5.3.2 Methods of Making rAAVs and Assessment of Efficacy

In some embodiments, provided herein is a method of producing a recombinant adeno-associated virus (rAAV) of the disclosure. In some embodiments, the method comprises culturing a cell in a cell culture to produce the rAAV. In some embodiments, a cell is a muscle cell. In some embodiments, a cell (e.g., muscle cell) comprises a nucleotide sequence encoding a capsid protein. In some embodiments, a cell further comprises a nucleotide sequence comprising or encoding a transgene (e.g., a transgene of the disclosure; Section 5.2.4 and 5.4). In some embodiments, the capsid protein comprises: (i) an amino acid sequence that is about or at least about 90%, 91%, 92%, 93%, 94%, 95%, 96%, 97%, 98%, 99% or 100% sequence identical to VP1/2s region of a first AAV (e.g., AAV5, AAVhu32, or VP1/2s region of any wild-type AAV, or a VP1/2s region of any AAV of the disclosure or identified in Section 7), and (ii) an amino acid sequence that is about or at least about 90%, 91%, 92%, 93%, 94%, 95%, 96%, 97%, 98%, 99% or 100% sequence identical to VP3 region of a second AAV (e.g., AAV8, AAVrh13, or VP3 region of any wild-type AAV, or a VP3 region of any AAV of the disclosure or identified in Section 7). In some embodiments, the method further comprises collecting the rAAV from the cell culture. In some embodiments, the capsid protein further comprises an amino acid sequence that is about or at least about 90%, 91%, 92%, 93%, 94%, 95%, 96%, 97%, 98%, 99% or 100% sequence identical to VP1u region of a first AAV or a second AAV (e.g., AAV5, AAVhu32, AAV8, AAVrh13, or VP1u of any wild-type AAV, or a VP1u region of any AAV of the disclosure or identified in Section 7). In some embodiments, the capsid protein comprises an amino acid sequence that is about or at least about 95% identical to VP1u, VP1/2s, and/or VP3 of a first or second AAV. In some embodiments, the capsid protein comprises an amino acid sequence that is about or at least about 98% identical to VP1u, VP1/2s, and/or VP3 of a first or second AAV. In some embodiments, the capsid protein comprises an amino acid sequence that is about or at least about 99% identical to VP1u, VP1/2s, and/or VP3 of a first or second AAV. In some embodiments, the capsid protein, comprises: (i) an amino acid sequence that is about or at least about 90%, 91%, 92%, 93%, 94%, 95%, 96%, 97%, 98%, 99% or 100% sequence identical to VP1u region of a first AAV (e.g., AAV5, AAVhu32, or VP1/2s region of any wild-type AAV, or a VP1/2s region of any AAV of the disclosure or identified in Section 7), (ii) an amino acid sequence that is about or at least about 90%, 91%, 92%, 93%, 94%, 95%, 96%, 97%, 98%, 99% or 100% sequence identical to VP1/2s region of a first AAV (e.g., AAV5, AAVhu32, or VP1/2s region of any wild-type AAV, or a VP1/2s region of any AAV of the disclosure or identified in Section 7), and (iii) an amino acid sequence that is about or at least about 90%, 91%, 92%, 93%, 94%, 95%, 96%, 97%, 98%, 99% or 100% sequence identical to VP3 region of a second AAV (e.g., AAV8, AAVrh13, or VP3 region of any wild-type AAV, or a VP3 region of any AAV of the disclosure or identified in Section 7).

In some embodiments, the cell is selected from an invertebrate cell, an insect cell, or a mammalian cell. In some embodiments, the mammalian cell is selected from HEK293, HeLa, CHO, NS0, SP2/0, PER.C6, Vero, RD, BHK, HT-1080, A549, Cos-7, ARPE-19, MRC-5, or any combination thereof. In some embodiments, the insect cell is selected from High Five, Sf9, Se301, SeIZD2109, SeUCR1, Sf900+, Sf21, Bti-tn-5b1-4, MG-1, Tn368, HzAm1, BM-N, Ha2302, Hz2E5, Ao38, or any combination thereof.

Provided herein are methods of making an rAAV of the disclosure. In certain embodiments, the method comprises transfecting a cell (e.g., an ex vivo cell) with an rAAV plasmid of the disclosure and one or more helper plasmids collectively providing the AAV Rep, Cap, and Ad5 functions. In certain embodiments, the one or more helper plasmids collectively comprises the nucleotide sequences of AAV genes Rep, Cap, VA, E2a and E4.

In some embodiments, the recombinant viral vector used in the methods described herein is or is derived from a recombinant/hybrid adenovirus vector. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). In some embodiments, the transgene or therapeutic product is inserted between the packaging signal and the 3′ITR, with or without stuffer sequences to keep the genome close to wild-type size of approx. 36 kb. An exemplary protocol for production of adenoviral vectors may be found in Alba et al., 2005, “Gutless adenovirus: last generation adenovirus for gene therapy,” Gene Therapy 12: S18-S27, which is incorporated by reference herein in its entirety.

The manufacture of an rAAV provided herein for gene therapy applications can use methods known in the art, for example, as described in Clement et al., 2016, Molecular Therapy-Methods & Clinical Development, 27:16002, which is incorporated by reference herein in its entirety. In certain embodiments, transfection of the plasmid DNA is performed using calcium phosphate plasmid precipitation on human embryonic kidney 293 cells (HEK293) or HEK293-T with the rAAV plasmid and the helper plasmid(s) that provide the AAV Rep and Cap functions as well as the Ad5 genes (VA RNAs, E2a, and E4) as is described in the art. In certain embodiments, the Rep, Cap, and Ad5 genes can be on the same helper plasmid. In certain embodiments, a two-helper method (or triple transfection) is utilized where AAV Rep, Cap, and Ad5 functions are provided from separate plasmids. In certain embodiments, the HEK293 cells can be adapted to grow in suspension in an animal component and antibiotic-free media. In some embodiments, a hybrid AAV can be produced with one or more than one plasmid or vector. In some embodiments, one or more plasmid or vector can provide or encode a capsid component from one AAV (e.g., VP1u and/or VP1/2s region) and another plasmid or vector can provide a capsid component from another AAV (e.g., VP3 region).

In certain embodiments, rAAV can be manufactured using packaging and producer cell lines. The rAAV provided herein can be manufactured using mammalian host cells, for example, A549, WEHI, 10T1/2, BHK, MDCK, COS1, COS7, BSC 1, BSC 40, BMT 10, VERO, W138, HeLa, HEK293, HEK293-T, Saos, C2C12, L, HT1080, HepG2, primary fibroblast, hepatocyte, myoblast cells, or any muscle related cell. The rAAV provided herein may be manufactured using host cells from human, monkey, mouse, rat, rabbit, or hamster. In certain embodiments, stable cell lines can be engineered by introducing the means of producing viruses in the host cells, for example, the replication and capsid genes (e.g., the rep and cap genes of AAV) and the rAAV plasmid provided herein. In some embodiments, the rAAV can be manufactured using HEK293 cells. In some embodiments, rAAV can be produced in Sf9 insect cells by coinfecting three recombinant baculovirus plasmids with genes encoding the rep gene, the cap gene, and the rAAV genome.

The cells can be cultured, transfected, and harvested according to appropriate protocols which would be readily selected by one of skill in the art. In certain embodiments, the cells can be cultured in standard Dulbecco's modified Eagle medium (DMEM), including, but not limited to, fetal calf serum, glucose, penicillin, streptomycin, and 1-glutamine (McClure et al., J Vis Exp. 2011, (57): 3348; Shin et al., Methods Mol Biol. 2012, 798:267-284). Cells can be transfected in components which would be readily selected by one of skill in the art. In certain embodiments, transfection can take place in media solutions including, but not limited to, DMEM and Iscove's modified Dulbecco's medium (IMDM). In certain embodiments, the transfection time can take 46 hr, 47 hr, 48 hr, 49 hr, 50 hr, 51 hr, 52 hr, 53 hr, 54 hr, 55 hr, 56 hr, 57 hr, 58 hr, 59 hr, 60 hr, 61 hr, 62 hr, 63 hr, 64 hr, 65 hr, 66 hr, 67 hr, 68 hr, 69 hr, 70 hr, 50-55 hr, 55-60 hr, 60-65 hr, or 65-70 hr. After transfection, the cells can be harvested by scraping cells to remove them from the culture wells and washing the wells to collect all of the transfected cells.

For a method of producing rAAV, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation. In a specific embodiment, the rAAV described herein is an isolated or purified rAAV.

Multiple AAV serotypes have been identified. In certain embodiments, rAAVs or polynucleotides provided herein comprise one or more components derived from one or more serotypes of AAV. In certain embodiments, rAAVs or polynucleotides provided herein comprise one or more components derived from one or more of AAV1, AAV2, AAV3, AAV3B, AAV4, AAV5, AAV6, AAV7, AAV8, AAV9, AAVrh10, AAV11, AAV12, AAV13, AAVrh20, AAVhu.37, AAVrh39, AAVhu32, AAVrh21, AAVrh13, AAVrh15, AAVrh73, or AAVrh74. Nucleic acid sequences of AAV components and methods of making recombinant AAV and AAV capsids are described, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety.

In vitro assays, e.g., cell culture assays, can be used to measure therapeutic product expression from a vector described herein, thus indicating, e.g., potency of the vector. Cells utilized for the assay can include, but are not limited to, A549, WEHI, 10T1/2, BHK, MDCK, COS1, COS7, BSC 1, BSC 40, BMT 10, VERO, W138, HeLa, HEK293, HEK293-T, HuH7, Saos, C2C12, L, HT1080, HepG2, primary fibroblast, hepatocyte, and myoblast cells. In some embodiments, the cells utilized in the cell culture assay comprise HuH7 cells. In some embodiments, a Cell Line can be used to assess therapeutic product expression. Once expressed, characteristics of the expressed therapeutic product can be determined, including determination of the post-translational modification patterns. In addition, benefits resulting from post-translational modification of the cell-expressed therapeutic product can be determined using assays known in the art.

### 5.4 Therapeutic and Prophylactic Uses

Provided herein are hybrid AAVs of the disclosure comprising a transgene for therapy use in a subject (e.g., therapeutic or prophylactic therapy). In some embodiments, the therapy of the disclosure (e.g., hybrid AAV comprising a transgene) delays, prevents, treats, manages a disease or disorder, and/or ameliorates one or more symptoms associated with a disease or disorder in a subject (e.g., a muscle associated disease or disorder). In some embodiments a therapeutic transgene is a component of an artificial genome encapsidated by an AAV capsid protein to produce the rAAV vector of the disclosure. In some embodiments, an rAAV vector comprises a hybrid AAV capsid (see Sections 5.1.1 and 5.2.1). In some embodiments, a subject in need of treatment includes a subject suffering from the disease or disorder, or a subject pre-disposed thereto, e.g., a subject at risk of developing or having a recurrence of the disease or disorder (e.g., a muscle related disease or disorder). In some embodiments, a rAAV carrying a particular transgene finds use with respect to a given disease or disorder in a subject where the subject's native gene, corresponding to the transgene, is defective in providing the correct gene product, or correct amounts of the gene product. The transgene then can provide a copy of a gene that is defective in the subject.

In some embodiments, the transgene comprises cDNA that restores protein function to a subject having a genetic mutation(s) in the corresponding native gene. In some embodiments, the cDNA comprises associated RNA for performing genomic engineering, such as genome editing via homologous recombination. In some embodiments, the transgene encodes a therapeutic RNA, such as a shRNA, artificial miRNA, or element that influences splicing.

In some embodiments, the therapeutic transgene is a microdystrophin protein (e.g., such as those disclosed herein). Microdystrophins include, but are not limited to, those described in Table 3. In some embodiments, microdystrophins comprises or consists of an amino acid sequence of at least one of SEQ ID NO: 73-80 or a nucleotide sequence encoding one or more of any one of SEQ ID NOS: 73-80. Also provided herein are methods of treating human subjects for a muscular dystrophy disease that can be treated, for example, by providing a functional dystrophin. In some embodiments, the functional dystrophin is one or more of the microdystrophins disclosed herein (e.g., one or more of SEQ ID NOS: 73-80). In some embodiments, the disease to be treated is Duchenne Muscular Dystrophy (DMD). In some embodiments, the disease to be treated includes, but it is not limited to, Becker muscular dystrophy (BMD), myotonic muscular dystrophy (Steinert's disease), Facioscapulohumeral disease (FSHD), limb-girdle muscular dystrophy, X-linked dilated cardiomyopathy, and/or oculopharyngeal muscular dystrophy.

In some embodiments, provided herein is a method of treating a muscle related disease or disorder in a subject in need thereof. In some embodiments, the method comprises administering an rAAV comprising a polypeptide comprising an amino acid sequence of SEQ ID NO: 31 or an rAAV encoded by SEQ ID NO:32.

In some embodiments, the transgene is a muscle-specific disease therapeutic. In some embodiments, one or more of the transgene listed in Tables 1-2 is a muscle-specific transgene and can be used as a muscle specific disease therapeutics (e.g., a hybrid AAV of the disclosure comprising at least a portion of a transgene identified in Tables 1-2). A muscle-specific disease therapeutic can be a therapeutic that treats a muscle-specific disease. A muscle-specific disease can be diseases such as myopathies. In some aspects, a myopathy includes, but it is not limited to, muscular dystrophies (e.g. Duchenne Muscular Dystrophy (DMD), Becker muscular dystrophy (DMD)), human limb girdle muscular dystrophy (LGMD)), X-linked dilated cardiomyopathy, Myasthenia Gravis, Rhabdomyolysis, Amyotrophic Lateral Sclerosis (ALS), and/or Sarcopenia.

In some embodiments, the rAAVs of the disclosure is used in delivery to target tissues associated with the disorder or disease to be treated/prevented. In some embodiments, a disease or disorder associated with a particular tissue or cell type is one that largely affects the particular tissue or cell type, in comparison to other tissue of cell types of the body, or one where the effects or symptoms of the disorder appear in the particular tissue or cell type. Methods of delivering a transgene to a target tissue of a subject in need thereof involve administering to the subject an rAAV wherein the AAV capsid is an AAV hybrid serotype described herein (e.g., Section 5.1.1 and 5.2.1).

In some embodiments, the rAAV vector is administered systemically, and following transduction, the vector's production of the protein product may be further enhanced by an expression cassette employing tissue-specific promoter, for example, a muscle-specific promoter operably linked to a transgene. In some embodiments, the rAAV vector is administered by intravenous, intramuscular, and/or intra-peritoneal administration.

In some embodiments, expression of transgene from muscle tissue provides secretion of the transgene into the serum of the subject. In some embodiments, with respect to the therapeutic antibodies shown in Table 2, expression (of transgene) in muscle, or both muscle and liver may be desirable to deliver the expressed therapeutic antibody systemically.

Tables 1-2 below provide a list of transgenes that can be used in any of the rAAV vectors comprising a hybrid AAV capsid of the disclosure. In some embodiments, the disease or disorder to be treated with an rAAV of the disclosure comprising a transgene is a disease or disorder listed in Tables 1-2. The AAV hybrid serotype utilized to produce the rAAV vector is specifically engineered to optimize the tissue tropism and transduction of the vector.

TABLE 1
list of diseases associated with a transgene

| Disease | Transgene |
|---|---|
| Spinal Muscular | SMN |
| Atrophy (SMA) | |
| Duchenne Muscular | Mini-/Micro-Dystrophin Gene |
| Dystrophy | |
| Limb Girdle | human-alpha-sarcoglycan |
| Muscular Dystrophy | |
| Type 2C\|Gamma- | |
| sarcoglycanopathy | |
| Limb Girdle | gamma-sarcoglycan |
| Muscular Dystrophy | |
| Type 2C\|Gamma- | |
| sarcoglycanopathy | |
| Becker Muscular | huFollistatin344 |
| Dystrophy and | |
| Sporadic Inclusion | |
| Body Myositis | |
| Duchenne Muscular | GALGT2 |
| Dystrophy | |
| Limb-Girdle | hSGCB |
| Muscular Dystrophy, | |
| Type 2E | |

TABLE 2
list of antigens, transgene/antibodies, and indications

| ANTIGENS | | TRANSGENE/ANTIBODIES | INDICATIONS |
|---|---|---|---|
| Repulsive guidance molecule-A | | elezanumab | multiple sclerosis |
| Complement Component 5 | | ravulizumab | Myasthenia Gravis |
| Connective tissue growth factor | | pamrevlumab | fibrotic diseases, e.g. |
| (CTGF) | | | diabetic nephropathy, |
| | | | liver fibrosis, idiopathic |
| | | | pulmonary fibrosis |
| Integrin beta 7 | | etrolizumab | ulcerative colitis, |
| | | | Crohn's disease |
| Sclerostin | | romosozumab | Osteoporosis, |
| | | (EVENITY ®) | abnormal bone loss or |
| | | | weakness |
| Interleukin receptor 6 (IL6R) or | | Satralizumab | Adverse immune |
| Interleukin 6 (IL6) | | Sarilumab | responses (e.g. cytokine |
| | | Tocilizumab | storm, CAR-T therapy) |
| | | siltuximab, | |
| | | clazakizumab | |
| | | sirukumab | |
| | | olokizumab | |
| | | gerilimzumab | |
| Immunoglobin E (IgE) | | omolizumab | Asthma, COPD, |
| | | | eosinophilic asthma, |
| | | | chronic idiopathic |
| | | | urticaria |
| Thymic stromal lymphopoietin | | tezelipumab | Asthma, COPD |
| (TSLP) | | | |
| Interleukin 5 (IL5) | | benralizumab | Asthma, COPD |
| Interleukin 5 receptor (IL5R) | | reslizumab | Asthma, COPD, |
| | | | eosinophilic asthma |
| Interleukin 13 (IL13) | | tralokinumab | Atopic dermatitis |
| Interleukin 31 recptor alpha | | nemolizumab | Atopic dermatitis |
| (IL31RA) | | | |
| IL17A | | Ixekizumab | Plaque psoriasis, |
| | | secukinumab | psoriatic arthritis, |
| | | | ankylosing sponylitis |
| Interleukins or | IL-17A | ixekizumab (TALTZ ®) | Plaque psoriasis, |
| interleukin | | secukinumab | psoriatic arthritis, |
| receptors | | (COSENTYX ®) | ankylosing sponylitis |
| | IL-5 | mepolizumab | Asthma |
| | | (NUCALA ®) | |
| | IL-12/IL-23 | ustekinumab | Psoriasis & Crohn's |
| | | (STELARA ®) | disease |
| | IL-4R | dupilumab | Atopic dermatitis |
| Integrin | | vedolizumab | Ulcerative colitis & |
| | | (ENTYVIO ®) | Crohn's disease |
| | | Natalizumab (anti-integrin | Multiple sclerosis & |
| | | alpha 4) | Crohn's disease |
| Cardiovascular | PCSK9 | alirocumab | HeFH & HoFH |
| Targets | | (PRALUENT ®) | |
| | | evolucomab (REPATHA ®) | |
| | ANGPTL3 | evinacumab | HoFH & severe forms |
| | | | of dyslipidema |
| | Proinflammatory/ | E06-scFv | Cardiovascular diseases |
| | proatherogenic | | such as atherosclerosis |
| | phospholipids | | |
| RANKL | | denosumab (XGEVA ® and | Osteoporosis, |
| | | PROLIA ®) | increasing bone mass in |
| | | | breast and prostate |
| | | | cancer patients, & |
| | | | preventing skeletal- |
| | | | related events due to |
| | | | bone metastasis |
| PD-1, or PD-L1 or PD-L2 | | nivolumab (OPDIVO ®) | Metastatic melanoma, |
| | | pembrolizumab | lymphoma, non-small |
| | | (KEYTRUDA ®) | cell lung carcinoma |
| BLyS (B-lymphocyte stimulator, | | belimumab | Systemic lupus |
| also known as B-cell activating | | (BENLYSTA ®) | erythromatosis |
| factor (BAFF)) | | | |
| TNF-alpha | | adalimumab (HUMIRA ®) | Rheumatoid arthritis, |
| | | and | psoriatic arthritis, |
| | | infliximab (REMICADE ®) | askylosing spondylitis, |
| | | | Crohn's disease, plaque |
| | | | psoriasis, ulcerative |
| | | | colitis |
| Plasma Protein | C5, C5a | eculizumab (SOLIRIS ®) | Paroxysmal nocturnal |
| targets | | ravulizumab, or | hemoglobinuria, |
| | | crovalimuab | atypical hemolytic |
| | | | uremic syndrome, |
| | | | complement-mediated |
| | | | thrombotic |
| | | | microangiopathy |
| | Plasma kallikrein | lanadelumab | Hereditary angioedema |
| | | | (HAE) |

TABLE 3
amino acid sequences of microdystrophin proteins

| | SEQ | |
|---|---|---|
| | ID | |
| Structure | NO: | Amino Acid Sequence |
| DYS1 | 73 | MLWWEEVEDCYEREDVQKKTFTKWVNAQFSKFGKQHIENLFSDL |
| | | QDGRRLLDLLEGLTGQKLPKEKGSTRVHALNNVNKALRVLQNNN |
| | | VDLVNIGSTDIVDGNHKLTLGLIWNIILHWQVKNVMKNIMAGLQ |
| | | QTNSEKILLSWVRQSTRNYPQVNVINFTTSWSDGLALNALIHSH |
| | | RPDLFDWNSVVCQQSATQRLEHAFNIARYQLGIEKLLDPEDVDT |
| | | TYPDKKSILMYITSLFQVLPQQVSIEAIQEVEMLPRPPKVTKEE |
| | | HFQLHHQMHYSQQITVSLAQGYERTSSPKPRFKSYAYTQAAYVT |
| | | TSDPTRSPFPSQHLEAPEDKSFGSSLMESEVNLDRYQTALEEVL |
| | | SWLLSAEDTLQAQGEISNDVEVVKDQFHTHEGYMMDLTAHQGRV |
| | | GNILQLGSKLIGTGKLSEDEETEVQEQMNLLNSRWECLRVASME |
| | | KQSNLHRVLMDLQNQKLKELNDWLTKTEERTRKMEEEPLGPDLE |
| | | DLKRQVQQHKVLQEDLEQEQVRVNSLTHMVVVVDESSGDHATAA |
| | | LEEQLKVLGDRWANICRWTEDRWVLLQDILLKWQRLTEEQCLFS |
| | | AWLSEKEDAVNKIHTTGFKDQNEMLSSLQKLAVLKADLEKKKQS |
| | | MGKLYSLKQDLLSTLKNKSVTQKTEAWLDNFARCWDNLVQKLEK |
| | | STAQISQQPDLAPGLTTIGASPTQTVTLVTQPVVTKETAISKLE |
| | | MPSSLMLEVPTLERLQELQEATDELDLKLRQAEVIKGSWQPVGD |
| | | LLIDSLQDHLEKVKALRGEIAPLKENVSHVNDLARQLTTLGIQL |
| | | SPYNLSTLEDLNTRWKLLQVAVEDRVRQLHEAHRDFGPASQHFL |
| | | STSVQGPWERAISPNKVPYYINHETQTTCWDHPKMTELYQSLAD |
| | | LNNVRFSAYRTAMKLRRLQKALCLDLLSLSAACDALDQHNLKQN |
| | | DQPMDILQIINCLTTIYDRLEQEHNNLVNVPLCVDMCLNWLLNV |
| | | YDTGRTGRIRVLSFKTGIISLCKAHLEDKYRYLFKQVASSTGFC |
| | | DQRRLGLLLHDSIQIPRQLGEVASFGGSNIEPSVRSCFQFANNK |
| | | PEIEAALFLDWMRLEPQSMVWLPVLHRVAAAETAKHQAKCNICK |
| | | ECPIIGFRYRSLKHFNYDICQSCFFSGRVAKGHKMHYPMVEYCT |
| | | PTTSGEDVRDFAKVLKNKFRTKRYFAKHPRMGYLPVQTVLEGDN |
| | | METPVTLINFWPVDSAPASSPQLSHDDTHSRIEHYASRLAEMEN |
| | | SNGSYLNDSISPNESIDDEHLLIQHYCQSLNQDSPLSQPRSPAQ |
| | | ILISLESEERGELERILADLEEENRNLQAEYDRLKQQHEHKGLS |
| | | PLPSPPEMMPTSPQSPR |
| DYS3 | 74 | MLWWEEVEDCYEREDVQKKTFTKWVNAQFSKFGKQHIENLFSDL |
| | | QDGRRLLDLLEGLTGQKLPKEKGSTRVHALNNVNKALRVLQNNN |
| | | VDLVNIGSTDIVDGNHKLTLGLIWNIILHWQVKNVMKNIMAGLQ |
| | | QTNSEKILLSWVRQSTRNYPQVNVINFTTSWSDGLALNALIHSH |
| | | RPDLFDWNSVVCQQSATQRLEHAFNIARYQLGIEKLLDPEDVDT |
| | | TYPDKKSILMYITSLFQVLPQQVSIEAIQEVEMLPRPPKVTKEE |
| | | HFQLHHQMHYSQQITVSLAQGYERTSSPKPRFKSYAYTQAAYVT |
| | | TSDPTRSPFPSQHLEAPEDKSFGSSLMESEVNLDRYQTALEEVL |
| | | SWLLSAEDTLQAQGEISNDVEVVKDQFHTHEGYMMDLTAHQGRV |
| | | GNILQLGSKLIGTGKLSEDEETEVQEQMNLLNSRWECLRVASME |
| | | KQSNLHRVLMDLQNQKLKELNDWLTKTEERTRKMEEEPLGPDLE |
| | | DLKRQVQQHKVLQEDLEQEQVRVNSLTHMVVVVDESSGDHATAA |
| | | LEEQLKVLGDRWANICRWTEDRWVLLQDILLKWQRLTEEQCLFS |
| | | AWLSEKEDAVNKIHTTGFKDQNEMLSSLQKLAVLKADLEKKKQS |
| | | MGKLYSLKQDLLSTLKNKSVTQKTEAWLDNFARCWDNLVQKLEK |
| | | STAQISQQPDLAPGLTTIGASPTQTVTLVTQPVVTKETAISKLE |
| | | MPSSLMLEVPTLERLQELQEATDELDLKLRQAEVIKGSWQPVGD |
| | | LLIDSLQDHLEKVKALRGEIAPLKENVSHVNDLARQLTTLGIQL |
| | | SPYNLSTLEDLNTRWKLLQVAVEDRVRQLHEAHRDFGPASQHFL |
| | | STSVQGPWERAISPNKVPYYINHETQTTCWDHPKMTELYQSLAD |
| | | LNNVRFSAYRTAMKLRRLQKALCLDLLSLSAACDALDQHNLKQN |
| | | DQPMDILQIINCLTTIYDRLEQEHNNLVNVPLCVDMCLNWLLNV |
| | | YDTGRTGRIRVLSFKTGIISLCKAHLEDKYRYLFKQVASSTGFC |
| | | DQRRLGLLLHDSIQIPRQLGEVASFGGSNIEPSVRSCFQFANNK |
| | | PEIEAALFLDWMRLEPQSMVWLPVLHRVAAAETAKHQAKCNICK |
| | | ECPIIGFRYRSLKHFNYDICQSCFFSGRVAKGHKMHYPMVEYCT |
| | | PTTSGEDVRDFAKVLKNKFRTKRYFAKHPRMGYLPVQTVLEGDN |
| | | MET |
| DYS5 | 75 | MLWWEEVEDCYEREDVQKKTFTKWVNAQFSKFGKQHIENLFSDL |
| | | QDGRRLLDLLEGLTGQKLPKEKGSTRVHALNNVNKALRVLQNNN |
| | | VDLVNIGSTDIVDGNHKLTLGLIWNIILHWQVKNVMKNIMAGLQ |
| | | QTNSEKILLSWVRQSTRNYPQVNVINFTTSWSDGLALNALIHSH |
| | | RPDLFDWNSVVCQQSATQRLEHAFNIARYQLGIEKLLDPEDVDT |
| | | TYPDKKSILMYITSLFQVLPQQVSIEAIQEVEMLPRPPKVTKEE |
| | | HFQLHHQMHYSQQITVSLAQGYERTSSPKPRFKSYAYTQAAYVT |
| | | TSDPTRSPFPSQHLEAPEDKSFGSSLMESEVNLDRYQTALEEVL |
| | | SWLLSAEDTLQAQGEISNDVEVVKDQFHTHEGYMMDLTAHQGRV |
| | | GNILQLGSKLIGTGKLSEDEETEVQEQMNLLNSRWECLRVASME |
| | | KQSNLHRVLMDLQNQKLKELNDWLTKTEERTRKMEEEPLGPDLE |
| | | DLKRQVQQHKVLQEDLEQEQVRVNSLTHMVVVVDESSGDHATAA |
| | | LEEQLKVLGDRWANICRWTEDRWVLLQDILLKWQRLTEEQCLFS |
| | | AWLSEKEDAVNKIHTTGFKDQNEMLSSLQKLAVLKADLEKKKQS |
| | | MGKLYSLKQDLLSTLKNKSVTQKTEAWLDNFARCWDNLVQKLEK |
| | | STAQISQQPDLAPGLTTIGASPTQTVTLVTQPVVTKETAISKLE |
| | | MPSSLMLEVPTLERLQELQEATDELDLKLRQAEVIKGSWQPVGD |
| | | LLIDSLQDHLEKVKALRGEIAPLKENVSHVNDLARQLTTLGIQL |
| | | SPYNLSTLEDLNTRWKLLQVAVEDRVRQLHEAHRDFGPASQHFL |
| | | STSVQGPWERAISPNKVPYYINHETQTTCWDHPKMTELYQSLAD |
| | | LNNVRFSAYRTAMKLRRLQKALCLDLLSLSAACDALDQHNLKQN |
| | | DQPMDILQIINCLTTIYDRLEQEHNNLVNVPLCVDMCLNWLLNV |
| | | YDTGRTGRIRVLSFKTGIISLCKAHLEDKYRYLFKQVASSTGFC |
| | | DQRRLGLLLHDSIQIPRQLGEVASFGGSNIEPSVRSCFQFANNK |
| | | PEIEAALFLDWMRLEPQSMVWLPVLHRVAAAETAKHQAKCNICK |
| | | ECPIIGFRYRSLKHFNYDICQSCFFSGRVAKGHKMHYPMVEYCT |
| | | PTTSGEDVRDFAKVLKNKFRTKRYFAKHPRMGYLPVQTVLEGDN |
| | | METPVTLINFWPVDSAPASSPQLSHDDTHSRIEHYASRLAEMEN |
| | | SNGSYLNDSISPNESIDDEHLLIQHYCQSLNQDSPLSQPRSPAQ |
| | | ILISLES |
| human | 76 | MLWWEEVEDCYEREDVQKKTFTKWVNAQFSKFGKQHIENLFSDL |
| MD1 | | QDGRRLLDLLEGLTGQKLPKEKGSTRVHALNNVNKALRVLQNNN |
| (R4- | | VDLVNIGSTDIVDGNHKLTLGLIWNIILHWQVKNVMKNIMAGLQ |
| R23/ACT) | | QTNSEKILLSWVRQSTRNYPQVNVINFTTSWSDGLALNALIHSH |
| | | RPDLFDWNSVVCQQSATQRLEHAFNIARYQLGIEKLLDPEDVDT |
| | | TYPDKKSILMYITSLFQVLPQQVSIEAIQEVEMLPRPPKVTKEE |
| | | HFQLHHQMHYSQQITVSLAQGYERTSSPKPRFKSYAYTQAAYVT |
| | | TSDPTRSPFPSQHLEAPEDKSFGSSLMESEVNLDRYQTALEEVL |
| | | SWLLSAEDTLQAQGEISNDVEVVKDQFHTHEGYMMDLTAHQGRV |
| | | GNILQLGSKLIGTGKLSEDEETEVQEQMNLLNSRWECLRVASME |
| | | KQSNLHRVLMDLQNQKLKELNDWLTKTEERTRKMEEEPLGPDLE |
| | | DLKRQVQQHKVLQEDLEQEQVRVNSLTHMVVVVDESSGDHATAA |
| | | LEEQLKVLGDRWANICRWTEDRWVLLQDILLKWQRLTEEQCLFS |
| | | AWLSEKEDAVNKIHTTGFKDQNEMLSSLQKLAVLKADLEKKKQS |
| | | MGKLYSLKQDLLSTLKNKSVTQKTEAWLDNFARCWDNLVQKLEK |
| | | STAQISQAVTTTQPSLTQTTVMETVTTVTTREQILVKHAQEELP |
| | | PPPPQKKRQITVDTLERLQELQEATDELDLKLRQAEVIKGSWQP |
| | | VGDLLIDSLQDHLEKVKALRGEIAPLKENVSHVNDLARQLTTLG |
| | | IQLSPYNLSTLEDLNTRWKLLQVAVEDRVRQLHEAHRDFGPASQ |
| | | HELSTSVQGPWERAISPNKVPYYINHETQTTCWDHPKMTELYQS |
| | | LADLNNVRFSAYRTAMKLRRLQKALCLDLLSLSAACDALDQHNL |
| | | KQNDQPMDILQIINCLTTIYDRLEQEHNNLVNVPLCVDMCLNWL |
| | | LNVYDTGRTGRIRVLSFKTGIISLCKAHLEDKYRYLFKQVASST |
| | | GFCDQRRLGLLLHDSIQIPRQLGEVASFGGSNIEPSVRSCFQFA |
| | | NNKPEIEAALFLDWMRLEPQSMVWLPVLHRVAAAETAKHQAKCN |
| | | ICKECPIIGFRYRSLKHFNYDICQSCFFSGRVAKGHKMHYPMVE |
| | | YCTPTTSGEDVRDFAKVLKNKFRTKRYFAKHPRMGYLPVQTVLE |
| | | GDNMETDTM |
| Human | 77 | MLWWEEVEDCYEREDVQKKTFTKWVNAQFSKFGKQHIENLFSDL |
| microdystrophin | | QDGRRLLDLLEGLTGQKLPKEKGSTRVHALNNVNKALRVLQNNN |
| | | VDLVNIGSTDIVDGNHKLTLGLIWNIILHWQVKNVMKNIMAGLQ |
| | | QTNSEKILLSWVRQSTRNYPQVNVINFTTSWSDGLALNALIHSH |
| | | RPDLFDWNSVVCQQSATQRLEHAFNIARYQLGIEKLLDPEDVDT |
| | | TYPDKKSILMYITSLFQVLPQQVSIEAIQEVEMLPRPPKVTKEE |
| | | HFQLHHQMHYSQQITVSLAQGYERTSSPKPRFKSYAYTQAAYVT |
| | | TSDPTRSPFPSQHLEAPEDKSFGSSLMESEVNLDRYQTALEEVL |
| | | SWLLSAEDTLQAQGEISNDVEVVKDQFHTHEGYMMDLTAHQGRV |
| | | GNILQLGSKLIGTGKLSEDEETEVQEQMNLLNSRWECLRVASME |
| | | KQSNLHRVLMDLQNQKLKELNDWLTKTEERTRKMEEEPLGPDLE |
| | | DLKRQVQQHKVLQEDLEQEQVRVNSLTHMVVVVDESSGDHATAA |
| | | LEEQLKVLGDRWANICRWTEDRWVLLQDILLKWQRLTEEQCLFS |
| | | AWLSEKEDAVNKIHTTGFKDQNEMLSSLQKLAVLKADLEKKKQS |
| | | MGKLYSLKQDLLSTLKNKSVTQKTEAWLDNFARCWDNLVQKLEK |
| | | STAQISQAVTTTQPSLTQTTVMETVTTVTTREQILVKHAQEELP |
| | | PPPPQKKRTLERLQELQEATDELDLKLRQAEVIKGSWQPVGDLL |
| | | IDSLQDHLEKVKALRGEIAPLKENVSHVNDLARQLTTLGIQLSP |
| | | YNLSTLEDLNTRWKLLQVAVEDRVRQLHEAHRDFGPASQHELST |
| | | SVQGPWERAISPNKVPYYINHETQTTCWDHPKMTELYQSLADLN |
| | | NVRFSAYRTAMKLRRLQKALCLDLLSLSAACDALDQHNLKQNDQ |
| | | PMDILQIINCLTTIYDRLEQEHNNLVNVPLCVDMCLNWLLNVYD |
| | | TGRTGRIRVLSFKTGIISLCKAHLEDKYRYLFKQVASSTGFCDQ |
| | | RRLGLLLHDSIQIPRQLGEVASFGGSNIEPSVRSCFQFANNKPE |
| | | IEAALFLDWMRLEPQSMVWLPVLHRVAAAETAKHQAKCNICKEC |
| | | PIIGFRYRSLKHFNYDICQSCFFSGRVAKGHKMHYPMVEYCTPT |
| | | TSGEDVRDFAKVLKNKFRTKRYFAKHPRMGYLPVQTVLEGDNME |
| | | TDTM |
| Dys3978 | 78 | MLWWEEVEDCYEREDVQKKTFTKWVNAQFSKFGKQHIENLFSDL |
| | | QDGRRLLDLLEGLTGQKLPKEKGSTRVHALNNVNKALRVLQNNN |
| | | VDLVNIGSTDIVDGNHKLTLGLIWNIILHWQVKNVMKNIMAGLQ |
| | | QTNSEKILLSWVRQSTRNYPQVNVINFTTSWSDGLALNALIHSH |
| | | RPDLFDWNSVVCQQSATQRLEHAFNIARYQLGIEKLLDPEDVDT |
| | | TYPDKKSILMYITSLFQVLPQQVSIEAIQEVEMLPRPPKVTKEE |
| | | HFQLHHQMHYSQQITVSLAQGYERTSSPKPRFKSYAYTQAAYVT |
| | | TSDPTRSPFPSQHLEAPEDKSFGSSLMESEVNLDRYQTALEEVL |
| | | SWLLSAEDTLQAQGEISNDVEVVKDQFHTHEGYMMDLTAHQGRV |
| | | GNILQLGSKLIGTGKLSEDEETEVQEQMNLLNSRWECLRVASME |
| | | KQSNLHRVLMDLQNQKLKELNDWLTKTEERTRKMEEEPLGPDLE |
| | | DLKRQVQQHKVLQEDLEQEQVRVNSLTHMVVVVDESSGDHATAA |
| | | LEEQLKVLGDRWANICRWTEDRWVLLQDQPDLAPGLTTIGASPT |
| | | QTVTLVTQPVVTKETAISKLEMPSSLMLEVPTHRLLQQFPLDLE |
| | | KFLAWLTEAETTANVLQDATRKERLLEDSKGVKELMKQWQDLQG |
| | | EIEAHTDVYHNLDENSQKILRSLEGSDDAVLLQRRLDNMNFKWS |
| | | ELRKKSLNIRSHLEASSDQWKRLHLSLQELLVWLQLKDDELSRQ |
| | | APIGGDFPAVQKQNDVHRAFKRELKTKEPVIMSTLETVRIFLTE |
| | | QPLEGLEKLYQEPRELPPEERAQNVTRLLRKQAEEVNTEWEKLN |
| | | LHSADWQRKIDETLERLQELQEATDELDLKLRQAEVIKGSWQPV |
| | | GDLLIDSLQDHLEKVKALRGEIAPLKENVSHVNDLARQLTTLGI |
| | | QLSPYNLSTLEDLNTRWKLLQVAVEDRVRQLHEAHRDFGPASQH |
| | | FLSTSVQGPWERAISPNKVPYYINHETQTTCWDHPKMTELYQSL |
| | | ADLNNVRFSAYRTAMKLRRLQKALCLDLLSLSAACDALDQHNLK |
| | | QNDQPMDILQIINCLTTIYDRLEQEHNNLVNVPLCVDMCLNWLL |
| | | NVYDTGRTGRIRVLSFKTGIISLCKAHLEDKYRYLFKQVASSTG |
| | | FCDQRRLGLLLHDSIQIPRQLGEVASFGGSNIEPSVRSCFQFAN |
| | | NKPEIEAALFLDWMRLEPQSMVWLPVLHRVAAAETAKHQAKCNI |
| | | CKECPIIGFRYRSLKHFNYDICQSCFFSGRVAKGHKMHYPMVEY |
| | | CTPTTSGEDVRDFAKVLKNKFRTKRYFAKHPRMGYLPVQTVLEG |
| | | DNMET |
| Human | 79 | MLWWEEVEDCYEREDVQKKTFTKWVNAQFSKFGKQHIENLFSDL |
| MD3 | | QDGRRLLDLLEGLTGQKLPKEKGSTRVHALNNVNKALRVLQNNN |
| | | VDLVNIGSTDIVDGNHKLTLGLIWNIILHWQVKNVMKNIMAGLQ |
| | | QTNSEKILLSWVRQSTRNYPQVNVINFTTSWSDGLALNALIHSH |
| | | RPDLFDWNSVVCQQSATQRLEHAFNIARYQLGIEKLLDPEDVDT |
| | | TYPDKKSILMYITSLFQVLPQQVSIEAIQEVEMLPRPPKVTKEE |
| | | HFQLHHQMHYSQQITVSLAQGYERTSSPKPRFKSYAYTQAAYVT |
| | | TSDPTRSPFPSQHLEAPEDKSFGSSLMESEVNLDRYQTALEEVL |
| | | SWLLSAEDTLQAQGEISNDVEVVKDQFHTHEGYMMDLTAHQGRV |
| | | GNILQLGSKLIGTGKLSEDEETEVQEQMNLLNSRWECLRVASME |
| | | KQSNLHRVLMDLQNQKLKELNDWLTKTEERTRKMEEEPLGPDLE |
| | | DLKRQVQQHKVLQEDLEQEQVRVNSLTHMVVVVDESSGDHATAA |
| | | LEEQLKVLGDRWANICRWTEDRWVLLQDILLKWQRLTEEQCLFS |
| | | AWLSEKEDAVNKIHTTGFKDQNEMLSSLQKLAVLKADLEKKKQS |
| | | MGKLYSLKQDLLSTLKNKSVTQKTEAWLDNFARCWDNLVQKLEK |
| | | STAQISQAVTTTQPSLTQTTVMETVTTVTTREQILVKHAQEELP |
| | | PPPPQKKRQITVDTLERLQELQEATDELDLKLRQAEVIKGSWQP |
| | | VGDLLIDSLQDHLEKVKALRGEIAPLKENVSHVNDLARQLTTLG |
| | | IQLSPYNLSTLEDLNTRWKLLQVAVEDRVRQLHEAHRDFGPASQ |
| | | HFLSTSVQGPWERAISPNKVPYYINHETQTTCWDHPKMTELYQS |
| | | LADLNNVRFSAYRTAMKLRRLQKALCLDLLSLSAACDALDQHNL |
| | | KQNDQPMDILQIINCLTTIYDRLEQEHNNLVNVPLCVDMCLNWL |
| | | LNVYDTGRTGRIRVLSFKTGIISLCKAHLEDKYRYLFKQVASST |
| | | GFCDQRRLGLLLHDSIQIPRQLGEVASFGGSNIEPSVRSCFQFA |
| | | NNKPEIEAALFLDWMRLEPQSMVWLPVLHRVAAAETAKHQAKCN |
| | | ICKECPIIGFRYRSLKHFNYDICQSCFFSGRVAKGHKMHYPMVE |
| | | YCTPTTSGEDVRDFAKVLKNKFRTKRYFAKHPRMGYLPVQTVLE |
| | | GDNMETPVTLINFWPVDSAPASSPQLSHDDTHSRIEHYASRLAE |
| | | MENSNGSYLNDSISPNESIDDEHLLIQHYCQSLNQDSPLSQPRS |
| | | PAQILISLESEERGELERILADLEEENRNLQAEYDRLKQQHEHK |
| | | GLSPLPSPPEMMPTSPQSPRDAELIAEAKLLRQHKGRLEARMQI |
| | | LEDHNKQLESQLHRLRQLLEQPQAEDTM |
| Human | 80 | MLWWEEVEDCYEREDVQKKTFTKWVNAQFSKFGKQHIENLFSDL |
| MD4 | | QDGRRLLDLLEGLTGQKLPKEKGSTRVHALNNVNKALRVLQNNN |
| | | VDLVNIGSTDIVDGNHKLTLGLIWNIILHWQVKNVMKNIMAGLQ |
| | | QTNSEKILLSWVRQSTRNYPQVNVINFTTSWSDGLALNALIHSH |
| | | RPDLFDWNSVVCQQSATQRLEHAFNIARYQLGIEKLLDPEDVDT |
| | | TYPDKKSILMYITSLFQVLPQQVSIEAIQEVEMLPRPPKVTKEE |
| | | HFQLHHQMHYSQQITVSLAQGYERTSSPKPRFKSYAYTQAAYVT |
| | | TSDPTRSPFPSQHLEAPEDKSFGSSLMESEVNLDRYQTALEEVL |
| | | SWLLSAEDTLQAQGEISNDVEVVKDQFHTHEGYMMDLTAHQGRV |
| | | GNILQLGSKLIGTGKLSEDEETEVQEQMNLLNSRWECLRVASME |
| | | KQSNLHRVLMDLQNQKLKELNDWLTKTEERTRKMEEEPLGPDLE |
| | | DLKRQVQQHKVLQEDLEQEQVRVNSLTHMVVVVDESSGDHATAA |
| | | LEEQLKVLGDRWANICRWTEDRWVLLQDILLKWQRLTEEQCLES |
| | | AWLSEKEDAVNKIHTTGFKDQNEMLSSLQKLAVLKADLEKKKQS |
| | | MGKLYSLKQDLLSTLKNKSVTQKTEAWLDNFARCWDNLVQKLEK |
| | | STAQISQAVTTTQPSLTQTTVMETVTTVTTREQILVKHAQEELP |
| | | PPPPQKKRQITVDTLERLQELQEATDELDLKLRQAEVIKGSWQP |
| | | VGDLLIDSLQDHLEKVKALRGEIAPLKENVSHVNDLARQLTTLG |
| | | IQLSPYNLSTLEDLNTRWKLLQVAVEDRVRQLHEAHRDFGPASQ |
| | | HFLSTSVQGPWERAISPNKVPYYINHETQTTCWDHPKMTELYQS |
| | | LADLNNVRFSAYRTAMKLRRLQKALCLDLLSLSAACDALDQHNL |
| | | KQNDQPMDILQIINCLTTIYDRLEQEHNNLVNVPLCVDMCLNWL |
| | | LNVYDTGRTGRIRVLSFKTGIISLCKAHLEDKYRYLFKQVASST |
| | | GFCDQRRLGLLLHDSIQIPRQLGEVASFGGSNIEPSVRSCFQFA |
| | | NNKPEIEAALFLDWMRLEPQSMVWLPVLHRVAAAETAKHQAKCN |
| | | ICKECPIIGFRYRSLKHFNYDICQSCFFSGRVAKGHKMHYPMVE |
| | | YCTPTTSGEDVRDFAKVLKNKFRTKRYFAKHPRMGYLPVQTVLE |
| | | GDNMETPVTLINFWPVDSAPASSPQLSHDDTHSRIEHYASRLAE |
| | | MENSNGSYLNDSISPNESIDDEHLLIQHYCQSLNQDSPLSQPRS |
| | | PAQILISLESEERGELERILADLEEENRNLQAEYDRLKQQHEHK |
| | | GLSPLPSPPEMMPTSPQSPRDAELIAEAKLLRQHKGRLEARMQI |
| | | LEDHNKQLESQLHRLRQLLEQPQAEAKVNGTTVSSPSTSLQRSD |
| | | SSQPMLLRVVGSQTSDSMGEEDLLSPPQDTSTGLEEVMEQLNNS |
| | | FPSSRGRNTPGKPMREDTM |

### 5.4.1 Target Patient Populations

In some embodiments, a subject is a subject with a disease or disorder associated with a muscle or is at risk of having a disease or disorder associated with a muscle. In some embodiments, a subject is a subject with a muscle related disease (e.g., muscular dystrophy) or is at risk of having a muscle related disease. In some embodiments, a subject is a subject with at least one symptom associated with a muscle related disease or disorder. Non-limiting examples of muscle related disease or disorder that a subject may have been diagnosed with or is at risk of developing, is a disease or disorder including at least one of Duchenne muscular dystrophy, Becker muscular dystrophy, Bethlem congenital muscular dystrophy (CMD), Fukuyama CMD, muscle-eye-brain disease, rigid spine syndrome, Ullrich CMD, walker-warburg syndrome, Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), amyotrophic lateral sclerosis (ALS), Spinal-bulbar muscular atrophy (SBMA), Spinal muscular atrophy (SMA), Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia, Friedreich's ataxia (FA), Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO), Congenital myasthenic syndromes (CMS), Lambert-Eaton myasthenic syndrome (LEMS), Myasthenia gravis (MG), Charcot-Marie-Tooth disease (CMT), Giant axonal neuropathy (GAN), Myofibrillar myopathy (MFM), Scapuloperoneal myopathy, Metabolic myopathy, inflammatory myopathy, endocrine myopathy, distal myopathy, and congenital myopathy. In some embodiments, the subject has been diagnosed with or is suspected of having Duchenne muscular dystrophy. In some embodiments, a subject has at least one mutation of at least one gene associated with a muscle related disease or disorder. In some embodiments, a subject has overexpression or downexpression of a gene associated with a muscle related disease or disorder.

In certain embodiments, the subject treated in accordance with the methods described herein is female. In certain embodiments, the subject treated in accordance with the methods described herein is male. In certain embodiments, the subject treated in accordance with the methods described herein is a child. In certain embodiments, the subject treated in accordance with the methods described herein is a juvenile subject (e.g., 18 years or younger). In certain embodiments, the subject treated in accordance with the methods described herein is 1 month old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or 5 years old. In certain embodiments, the subject treated in accordance with the methods described herein is less than 1.5 months old, 2 months old, 3 months old, 4 months old, 5 months old, 6 months old, 7 months old, 8 months old, 9 months old, 10 months old, 11 months old, 1 year old, 1.5 years old, 2 years old, 2.5 years old, 3 years old, 3.5 years old, 4 years old, 4.5 years old, or less than 5 years old. In another specific embodiment, the subject treated in accordance with the methods described herein is 1-2 months old, 2-3 months old, 3-4 months old, 4-5 months old, 5-6 months old, 6-7 months old, 7-8 months old, 8-9 months old, 9-10 months old, 10-11 months old, 11 months to 1 year old, 1-1.5 years old, 1.5-2 years old, 2-2.5 years old, 2.5-3 years old, 3-3.5 years old, 3.5-4 years old, 4-4.5 years old, or 4.5-5 years old. In another specific embodiment, the subject treated in accordance with the methods described herein is 6 months to 5 years old. In another embodiment, the subject treated in accordance with the methods described herein is a human adult over 18 years old. In some embodiments, the subject treated in accordance with the methods described herein is a human child under 18 years. In some embodiments, the subject treated in accordance with the methods described herein is a human child under 84 months of age.

In a specific embodiment, the subject is an adult (at least age 16). In another specific embodiment, the subject is an adolescent (age 12-15). In another specific embodiment, the subject is a child (under age 12). In some embodiments, the subject is under age 6, under age 10, under age 15, under age 18, under age 21, under age 25, under age 30, under age 35, under age 40, under age 45, under age 50, under age 55, under age 60, under age 65, under age 70, under age 75, under age 80, under age 85, under age 90, or under age 95. In some embodiments, the subject is over age 6, over age 10, over age 15, over age 18, over age 21, over age 25, over age 30, over age 35, over age 40, over age 45, over age 50, over age 55, over age 60, over age 65, over age 70, over age 75, over age 80, over age 85, over age 90, or over age 95. In some embodiments, the subject is a subject who is not responsive to a previous treatment. In some embodiments, the subject is a subject who has not received a treatment for a disease or disorder associated with a muscle. In some embodiments, the subject is a subject who is currently undergoing treatment for a disease or disorder associated with a muscle. In some embodiments, the subject is a subject who has undergone surgery. In some embodiments, the subject is a subject who has received chemotherapy.

### 5.4.2 Administration and Dosage

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56th ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the disclosure that is effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some embodiments, animal model systems for a CNS condition are used that are based on rats, mice, or other small mammal other than a primate.

Once the prophylactic and/or therapeutic agents of the invention have been tested in an animal model, they can be tested in clinical trials to establish their efficacy. Establishing clinical trials can be done in accordance with common methodologies known to one skilled in the art, and the optimal dosages and routes of administration as well as toxicity profiles of agents of the disclosure can be established. For example, a clinical trial can be designed to test an rAAV molecule of the disclosure for efficacy and toxicity in human patients.

Toxicity and efficacy of the prophylactic and/or therapeutic agents of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, prophylactic and/or therapeutic agents exhibit large therapeutic indices. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

An rAAV molecule of the disclosure generally is administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies within a range of circulating concentrations that include the ED50 with little or no toxicity. The dosage may vary within this range depending upon the dosage form employed and the route of administration utilized.

In some embodiments, a therapeutically effective dosage of an rAAV vector is from about 0.1 ml to about 100 ml of solution containing concentrations of from about 1×10⁹ to about 1×10¹⁶ genomes rAAV vector, or about 1×10¹⁰ to about 1×10¹⁵, about 1×10¹² to about 1×10¹⁶, or about 1×10¹⁴ to about 1×10¹⁶ AAV genomes. Levels of expression of the transgene can be monitored to determine/adjust dosage amounts, frequency, scheduling, and the like.

Treatment of a subject with a therapeutically or prophylactically effective amount of the agents of the disclosure can include a single treatment or can include a series of treatments. For example, pharmaceutical compositions comprising an agent of the disclosure may be administered once a day, twice a day, or three times a day. In some embodiments, the agent may be administered once a day, every other day, once a week, twice a week, once every two weeks, once a month, once every six weeks, once every two months, twice a year, or once per year. In some embodiments, an rAAV of the disclosure increase or decrease over the course of treatment.

In some embodiments, ongoing treatment is indicated, e.g., on a long-term basis, such as in the ongoing treatment and/or management of a muscle related disease or disorder. For example, in particular embodiments, an rAAV of the disclosure is administered over a period of time, e.g., for at least 6 months, at least one year, at least two years, at least five years, at least ten years, at least fifteen years, at least twenty years, or for the rest of the lifetime of a subject in need thereof.

The rAAV molecules of the disclosure may be administered alone or in combination with other prophylactic and/or therapeutic agents (e.g., an agent for treating a muscle related disease or disorder, or an agent for reducing pain or inflammation or any side effect from the rAAV treatment). Each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In some embodiments, the rAAV of the disclosure, a polypeptide of the disclosure, or any pharmaceutical composition or formulation of the disclosure is administered in combination with an immune suppression therapy. Immune suppression therapies involving a regimen of tacrolimus or rapamycin (sirolimus) in combination with mycophenolic acid, or other immune suppression regimens procedures can be employed. Such immune suppression treatment may be administered during the course of therapy, and in certain embodiments, pre-treatment and/or post treatment with immune suppression therapy can be preferred. Immune suppression therapy can be continued subsequent to the therapy treatment of the disclosure, based on the judgment of the treating physician, and may thereafter be withdrawn when immune tolerance is induced; e.g., after 180 days.

In various embodiments, the different prophylactic and/or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart, or no more than 48 hours apart. In certain embodiments, two or more agents are administered within the same patient visit.

In another specific embodiment, agents of the disclosure may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the molecules of the disclosure are enclosed within a device such that release of the molecules is controlled by permeation through a diffusion barrier. Common reservoir devices include, for example, membranes, capsules, microcapsules, liposomes, and hollow fibers. Monolithic (matrix) device are a second type of diffusion controlled system, wherein the dual antigen-binding molecules are dispersed or dissolved in an rate-controlling matrix (e.g., a polymer matrix). Agents of the disclosure can be homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

Any technique known to one of skill in the art can be used to produce sustained release formulations comprising one or more agents described herein. See, e.g. U.S. Pat. No. 4,526,938; PCT publication WO 91/05548; PCT publication WO 96/20698; Ning et al., “Intratumoral Radioimmunotheraphy of a Human Colon Cancer Xenograft Using a Sustained-Release Gel,” Radiotherapy & Oncology, 39:179 189, 1996; Song et al., “Antibody Mediated Lung Targeting of Long-Circulating Emulsions,” PDA Journal of Pharmaceutical Science & Technology, 50:372 397, 1995; Cleek et al., “Biodegradable Polymeric Carriers for a bFGF Antibody for Cardiovascular Application,” Pro. Intl. Symp. Control. Rel. Bioact. Mater., 24:853 854, 1997; and Lam et al., “Microencapsulation of Recombinant Humanized Monoclonal Antibody for Local Delivery,” Proc. Int'l. Symp. Control Rel. Bioact. Mater., 24:759 760, 1997, each of which is incorporated herein by reference in its entirety. In one embodiment, a pump may be used in a controlled release system (see Langer, supra; Sefton, CRC Crit. Ref. Biomed. Eng., 14:20, 1987; Buchwald et al., Surgery, 88:507, 1980; and Saudek et al., N. Engl. J. Med., 321:574, 1989). In another embodiment, polymeric materials can be used to achieve controlled release of agents comprising dual antigen-binding molecule, or antigen-binding fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem., 23:61, 1983; see also Levy et al., Science, 228:190, 1985; During et al., Ann. Neurol., 25:351, 1989; Howard et al., J. Neurosurg., 7 1:105, 1989); U.S. Pat. Nos. 5,679,377; 5,916,597; 5,912,015; 5,989,463; 5,128,326; PCT Publication No. WO 99/15154; and PCT Publication No. WO 99/20253). In yet another embodiment, a controlled release system can be placed in proximity of the therapeutic target (e.g., an affected joint), thus requiring only a fraction of the systemic dose (see, e.g., Goodson, in Medical Applications of Controlled Release, supra, vol. 2, pp. 115 138 (1984)). Other controlled release systems are discussed in the review by Langer, Science, 249:1527 1533, 1990.

In certain embodiments, dosages are measured by genome copies per ml, the number of genome copies, or vg/kg administered to a location in the subject. In certain embodiments, 1×10⁹ genome copies per ml or vg/kg to 1×10¹⁵ genome copies per ml or vg/kg are administered. In a specific embodiment, 1×10⁹ genome copies per ml or vg/kg to 1×10¹⁰ genome copies per ml or vg/kg are administered. In another specific embodiment, 1×10¹⁰ genome copies per ml or vg/kg to 1×10¹¹ genome copies per ml or vg/kg are administered. In another specific embodiment, 1×10¹⁰ to 5×10¹¹ genome copies or vg/kg are administered. In another specific embodiment, 1×10¹¹ genome copies per ml or vg/kg to 1×10¹² genome copies per ml or vg/kg are administered. In another specific embodiment, 1×10¹² genome copies per ml or vg/kg to 1×10¹³ genome copies per ml or vg/kg are administered. In another specific embodiment, 1×10¹³ genome copies per ml or vg/kg to 1×10¹⁴ genome copies per ml or vg/kg are administered. In another specific embodiment, 1×10¹⁴ genome copies per ml or vg/kg to 1×10¹⁵ genome copies per ml or vg/kg are administered. In another specific embodiment, about, at least about, or at most about 1×10⁹ genome copies per ml or vg/kg are administered. In another specific embodiment, about, at least about, or at most about 1×10¹⁰ genome copies per ml or vg/kg are administered. In another specific embodiment, about, at least about, or at most about 1×10¹¹ genome copies per ml or vg/kg are administered. In another specific embodiment, about, at least about, or at most about 1×10¹² genome copies per ml or vg/kg are administered. In another specific embodiment, about, at least about, or at most about 1×10¹³ genome copies per ml or vg/kg are administered. In another specific embodiment, about, at least about, or at most about 1×10¹⁴ genome copies per ml or vg/kg are administered. In another specific embodiment, about, at least about, or at most about 1×10¹⁵ genome copies per ml or vg/kg are administered. In certain embodiments, 1×10⁹ to 1×10¹⁵ genome copies or vg/kg are administered. In a specific embodiment, 1×10⁹ to 1×10¹⁰ genome copies or vg/kg are administered. In another specific embodiment, 1×10¹⁰ to 1×10¹¹ genome copies or vg/kg are administered. In another specific embodiment, 1×10¹⁰ to 5×10¹¹ genome copies or vg/kg are administered. In another specific embodiment, 1×10¹¹ to 1×10¹² genome copies or vg/kg are administered. In another specific embodiment, 1×10¹² to 1×10¹³ genome copies or vg/kg are administered. In another specific embodiment, 1×10¹³ to 1×10¹⁴ genome copies or vg/kg are administered. In another specific embodiment, 1×10¹³ to 1×10¹⁴ genome copies or vg/kg are administered. In another specific embodiment, 1×10¹⁴ to 1×10¹⁵ genome copies or vg/kg are administered. In another specific embodiment, about 1×10⁹ genome copies or vg/kg are administered. In another specific embodiment, about 1×10¹⁰ genome copies or vg/kg are administered. In another specific embodiment, about 1×10¹¹ genome copies or vg/kg are administered. In another specific embodiment, about 1×10¹² genome copies or vg/kg are administered. In another specific embodiment, about 1×10¹³ genome copies or vg/kg are administered. In another specific embodiment, about 1×10¹⁴ genome copies or vg/kg are administered. In another specific embodiment, about 1×10¹⁵ genome copies or vg/kg are administered. In certain embodiments, about 3.0×10¹³ genome copies or vg/kg are administered. In certain embodiments, up to 3.0×10¹³ genome copies or vg/kg are administered.

In certain embodiments, about, at least about, or at most about 2.0×10¹⁰ genome copies or vg/kg are administered. In certain embodiments, about, at least about, or at most about 6.0×10¹⁰ genome copies or vg/kg are administered. In certain embodiments, about, at least about, or at most about 1.0×10¹⁰ to 2.0×10¹⁰ genome copies are or vg/kg administered. In certain embodiments, about, at least about, or at most about 2.0×10¹⁰ to 3.0×10¹⁰ genome copies or vg/kg are administered. In certain embodiments, about, at least about, or at most about 3.0×10¹⁰ to 4.0×10¹⁰ genome copies or vg/kg are administered. In certain embodiments, about, at least about, or at most about 4.0×10¹⁰ to 5.0×10¹⁰ genome copies or vg/kg are administered. In certain embodiments, about, at least about, or at most about 5.0×10¹⁰ to 6.0×10¹⁰ genome copies or vg/kg are administered. In certain embodiments, about, at least about, or at most about 6.0×10¹⁰ to 7.0×10¹⁰ genome copies or vg/kg are administered. In certain embodiments, about, at least about, or at most about 7.0×10¹⁰ to 8.0×10¹⁰ genome copies or vg/kg are administered. In certain embodiments, about, at least about, or at most about 8.0×10¹⁰ to 9.0×10¹⁰ genome copies or vg/kg are administered. In certain embodiments, about, at least about, or at most about 9.0×10¹⁰ to 1.0×10¹¹ genome copies or vg/kg are administered. In certain embodiments, about, at least about, or at most about 1.0×10¹¹ to 2.0×10¹¹ genome copies or vg/kg are administered. In certain specific embodiments, about, at least about, or at most about 2.0×10¹⁰ genome copies or vg/kg are administered. In certain specific embodiments, about, at least about, or at most about 6.0×10¹⁰ genome copies or vg/kg are administered.

### 6. EXAMPLES

Certain embodiments provided herein are illustrated by the following non-limiting examples.

### 6.1 Example 1: Construction of AAV Hybrid Capsids and Assessment of Packaging Efficiency

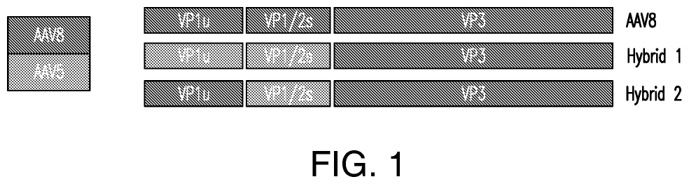

AAV-based gene therapies have the potential to treat many severe diseases through a wide range of modalities, from gene addition to gene editing and modulation of gene expression. For successful delivery of the DNA payload, AAV vectors must efficiently bind to the intended target tissue, enter the cell, and traffic to the nucleus. Cell entry depends on endocytosis followed by endosomal escape, which requires the action of the PLA2 domain located in the VP1-unique (VP1u) region of the AAV capsid, and trafficking to the nucleus, mediated by basic residues located in VP1u and the VP1/2s shared (VP1/2s) regions. With the goal of engineering capsids that maintained parental tropism with improved endosomal escape and nuclear trafficking, hybrid capsids were designed by swapping VP1u and VP1/2s (FIG. 1 and Table 4) from different serotypes selected based on data gathered from previous study (Giles et al., ASGCT 2021). Criteria for selection included several factors such as RNA/DNA ratio and tissue-specificity of gene expression. Parental capsid sequences were aligned, and regions swapped as desired, then plasmids were synthesized and constructed at GenScript. Efforts were made to avoid interruption in the assembly-activating protein (AAP) reading frame of each capsid gene. Examples shown in Table 4 were synthesized and constructed at GenScript. Overall productivity of vectors was not significantly negatively impacted by the introduction of hybrid domains as measured by small scale production by triple transfection in suspension HEK293 cells. Without ascribing to any particular theory, it was hypothesized that VP3 is the primary driver of tropism and therefore genomic DNA biodistribution would be largely unchanged by swapping VP1u and VP1/2s regions. It was anticipated that the primary differences would be seen at the RNA expression level, indicating altered endosomal escape and trafficking properties.

TABLE 4

| Capsid | Capsid Composition | | |
|---|---|---|---|
| Number | VP1u | VP1/2s | VP3 |
| 2048 | AAV6 | | |
| 2020 | AAV8 | | AAV6 |
| 1035 | AAV8 | | |
| 2017 | AAV6 | | AAV8 |
| 2024 | AAV9 | | AAV8 |
| 2117 | AAVrh.21 | | AAV8 |
| 2118 | AAVrh73 | | AAV8 |
| 2171 | AAV5 | | AAV8 |
| (Hybrid 1) | | | |
| 2172 | AAV8 | AAV5 | AAV8 |
| (Hybrid 2) | | | |

| 2040 | AAV3B | AAV8 |
|---|---|---|
| 1036 | AAV9 | |
| 2089 | AAVrh.15 | AAV9 |
| 2090 | AAVrh.13 | AAV9 |
| 2091 | AAV6 | AAV9 |
| 2092 | AAV5 | AAV9 |
| 1463 | AAVrh.13 | |
| 2096 | AAVhu32 | AAVrh.13 |
| 2097 | AAV6 | AAVrh.13 |
| 1465 | AAVrh.15 | |
| 2093 | AAVhu32 | AAVrh.15 |
| 2094 | AAV6 | AAVrh.15 |
| 2095 | AAV5 | AAVrh.15 |
| 1469 | AAVrh.21 | |
| 2018 | AAV6 | AAVrh.21 |
| 2021 | AAV8 | AAVrh.21 |
| 1513 | AAVrh.73 | |
| 2019 | AAV6 | AAVrh.73 |
| 2022 | AAV8 | AAVrh.73 |

A barcoded, AAV vector pool containing these hybrids and their parental capsids was created and administered intravenously to non-human primates. Biodistribution of vector genomes was similar to their respective parental VP3 serotypes for most of the hybrid capsids, except for those containing VP1u and VP1/2s of AAV5. The AAV5 VP1u and VP1/2s-containing hybrids had lower vector genome copy numbers in all tested tissues compared to their parental, wild type VP3 serotypes (AAV8, AAV9, and AAVrh.15); however, relative abundance of RNA transcripts from each capsid showed a liver-specific transcriptional down-regulation of AAV5-containing hybrids. For example, AAV5/AAV8 hybrid capsids had lower genome copy numbers than AAV8 in all tissues tested, but at the transcriptional level were liver de-targeted and expressed RNA more efficiently than wild type AAV8 in skeletal muscle, leading to an improved RNA/DNA ratio. These results suggest increased endosomal escape or nucleolar trafficking abilities of AAV5 VP1u and VP1/2s-containing capsids. Furthermore, AAV5/AAV8 hybrids produced 2.5-3× more vector than their parental capsid, AAV8, in standard triple transfection of HEK293 suspension culture. Ultimately, AAV5 hybrid capsids amongst other hybrid capsids described herein present an important new technique for liver de-targeting at the transcriptional level and offer improved production.

Several AAV hybrid capsid nucleotide sequences were designed and plasmids comprising the polynucleotides were constructed, such that each plasmid (trans plasmid) could be utilized to make recombinant AAV (rAAV) vectors. Briefly, the rAAV vectors were produced by triple transfection of three plasmids: 1) a plasmid containing a polynucleotide encoding a functional rep gene and a polynucleotide encoding a capsid gene as described herein, 2) a plasmid containing a genome to be packaged into a capsid (comprises at least one AAV inverted terminal repeat (ITR) and a non-AAV nucleic acid sequence encoding a transgene (e.g. fluorescent protein or muscle protein) operably linked to sequences which direct expression of the gene product in a target cell, such as a CAG promoter, and 3) a polynucleotide comprising sufficient helper functions to permit packaging of the genome into the AAV capsid protein under conditions which permit packaging of the genome into the AAV capsid. Following transfection in HEK293 cells, the cells were maintained in cell culture under conditions that allowed production of the rAAV particles.

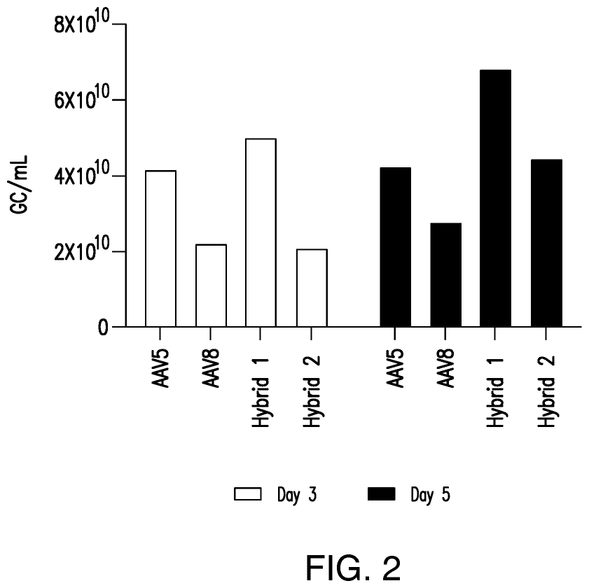

FIG. 2 depicts high packaging efficiency in terms of titer, expressed as genome copies per mL (GC/mL) of wild type AAV5 or AAV8 compared to two hybrid vectors having hybrid capsids as illustrated in FIG. 1 (e.g., Hybrid 1 and Hybrid 2), and having the sequences of SEQ ID NO: 23 (Hybrid 1 in FIG. 2) and 25 (Hybrid 2 in FIG. 2). All vectors were packaged with a muscle protein-encoding transgene and were titered following transfection and harvest from crude lysate of small flask cell cultures. Titer was determined by polyA qPCR. Assessment of titer from cells infected with viral vectors facilitated determining which insertion points did not interrupt capsid packaging and function; error bars represent standard error of the mean. As shown in FIG. 2, Hybrid 1 packages GFP with high efficiency greater than wtAAV5 or wt AAV8, and Hybrid 2 packages GFP equal to wt AAV8 at day 3, and equal to wtAAV5 but greater than wtAAV8 at day 5. It was observed that inclusion of the VP1u region of AAV5 (e.g. Hybrid 1) specifically increases the packaging efficiency of AAV8 by ˜2.5× on day 5.

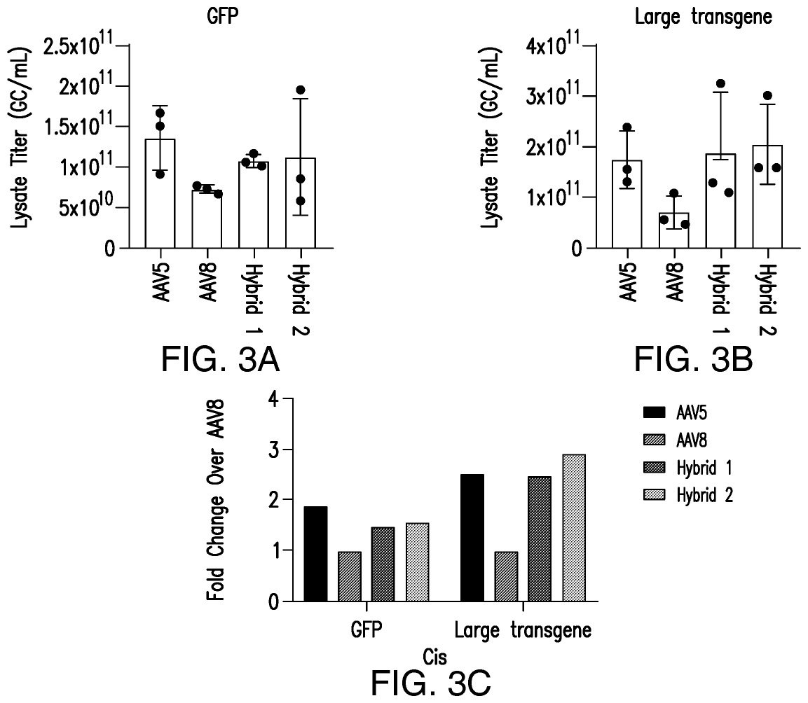

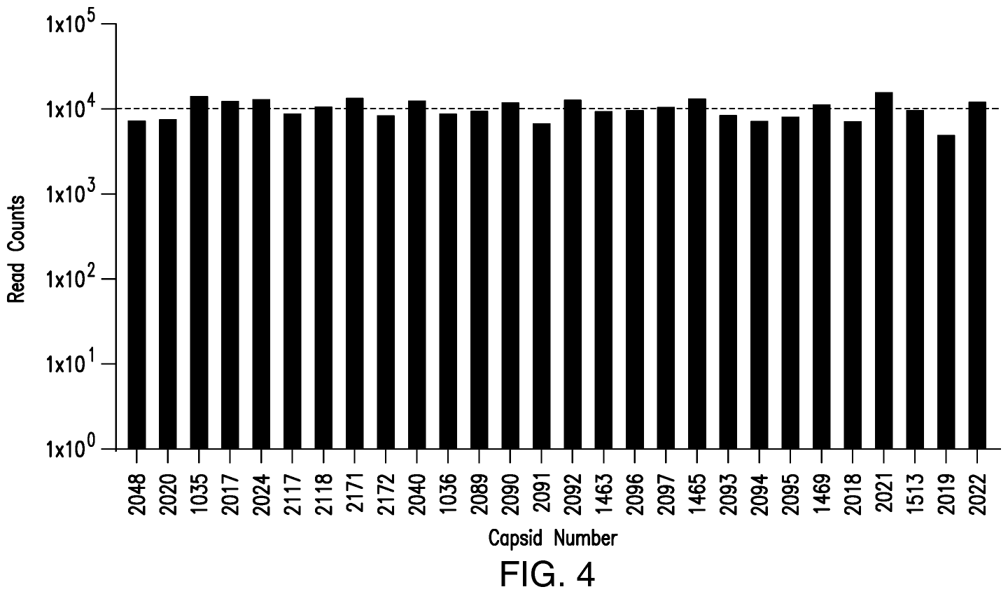

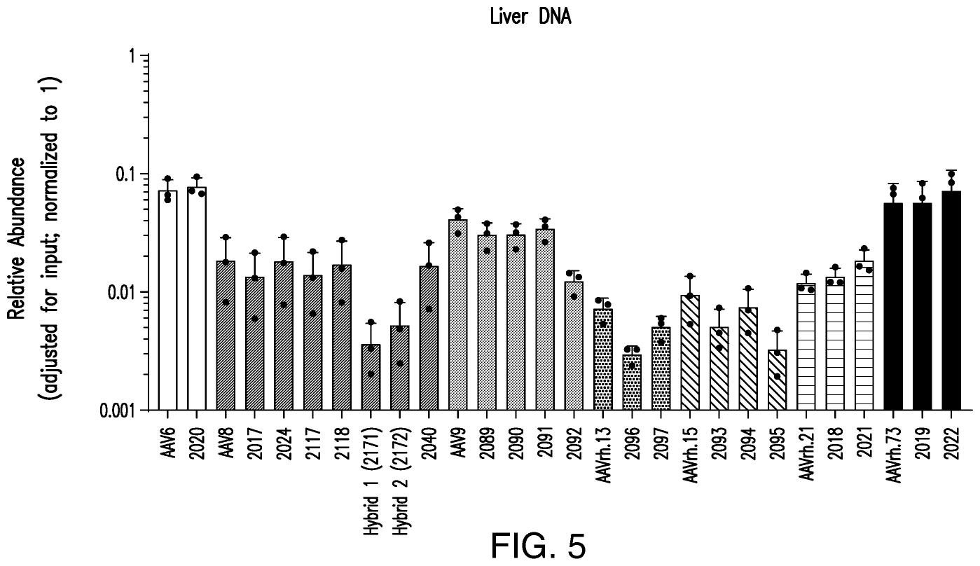

Different transgenes were compared, such that a GFP transgene or the muscle protein-encoding transgene were packaged in either wtAAV5, wtAAV8, Hybrid 1 or Hybrid 2, and transgene titers were measured from a 1 mL scale cell culture at 5 days post transfection. The muscle protein featured a large coding sequence (total expression cassette including ITRs is greater than 4 kb, “large transgene”) which is near the limit of AAV packaging capacity. The large transgene exhibited a larger difference in packaging efficiency between the capsids than GFP, as shown in FIGS. 3A and 3B. The AAV5/8 hybrids remain ˜2.5× higher producers than AAV8 of the large transgene (FIG. 3C).

### 6.2 Example 2: Evaluation of AAV Hybrid Capsids in a Non-Human Primate (NHP) Study

A barcoded, AAV vector pool was created containing 44 capsids including the hybrids described herein and several parental capsids, and administered intravenously to non-human primates, as follows.

Vector Production. Vector particles were produced by triple transfection of suspension-adapted HEK293 cells. Vector packaging a TdTomato transgene with CAG promoter and 15 base pair, unique barcode were harvested via freeze-thaw and purified via an iodixanol gradient. The final product was resuspended in phosphate-buffered saline (PBS) and 0.001% Pluronic F-68 to 500 μL.

Vector Pooling. A total of 44 different iodixanol-purified, endotoxin-removed vector preparations, including vectors comprising the capsids of Table 4, were pooled in approximately equal particle numbers and quantified by ddPCR.

Dosing. Two female and one male cynomolgus macaques of approximately two to four years old were screened for AAV2, AAV8, and AAV9 neutralizing antibody status prior to injection, with any positive titers being below 1:10. Included animals were injected intravenously at 2×10¹³ GC/kg total and subsequently sacrificed and tissues harvested 21 days after dosing.

NGS and Data Analysis. DNA extracted from NHP tissue was used as a template for amplification of the barcode region in each AAV transgene. Secondary amplification was performed to add Illumina Nextera adapters and indices to the amplicons, which were then pooled and purified. The resultant amplicon library was denatured and sequenced on a MiSeq instrument (Illumina) using the MiSeq v3 chemistry reagent kit. Barcode counts were quantified from forward reads using a custom Galaxy workflow and converted to relative abundance, adjusted for the input pool distribution, and normalized to 1. Extracted RNA was used for cDNA generation and subjected to a similar process as above to prepare for NGS analysis.

Vector Production Analysis. Vector particles were produced by triple transfection of suspension HEK293 cells packaging a small (3 kb) or large (4.7 kb) transgene at a variety of culture scales ranging from 1 mL to 1 L. Cell lysates were clarified by centrifugation and subjected to DNase digestion before ddPCR analysis.

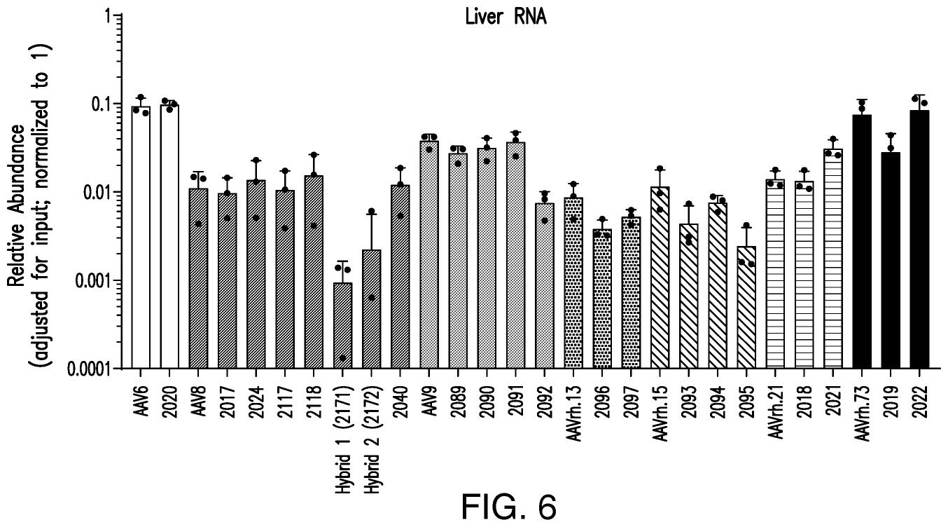

Biodistribution of vector genomes was similar to their respective parental VP3 serotypes for most of the hybrid capsids, except for those containing VP1u and VP1/2s of AAV5. Relative abundance of vector pool in the liver by DNA vector genome copies (FIG. 5) and mRNA (cDNA) transcript copies (FIG. 6) is shown as a representative data set. Capsids containing AAV5 VP1u and/or VP1/2s region, e.g., Hybrid 1 (2171), Hybrid 2 (2172), 2092, and 2095, exhibit reduced lower transduction (genome copy, FIG. 5), as well as lower expression (RNA transcripts, FIG. 6) in liver compared to their parental capsids. It was observed that AAV5-containing capsids decrease the transduction of all NHP tissues surveyed in this study, as measured by DNA genome copy number. Transcription of AAV-containing capsids in the liver was especially decreased, leading to a liver de-targeted phenotype.

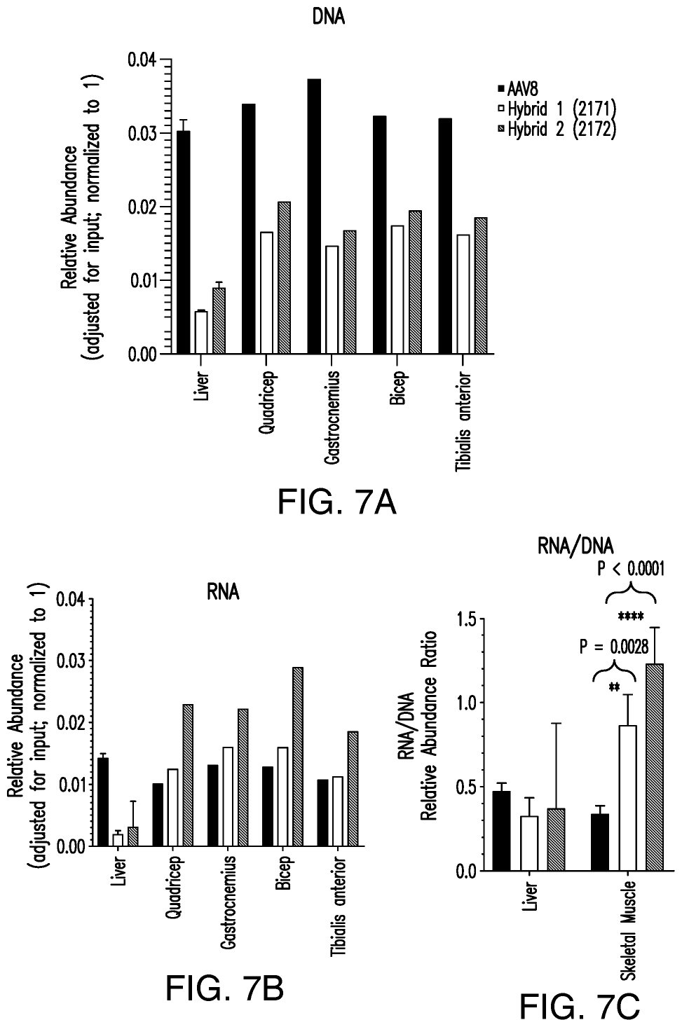

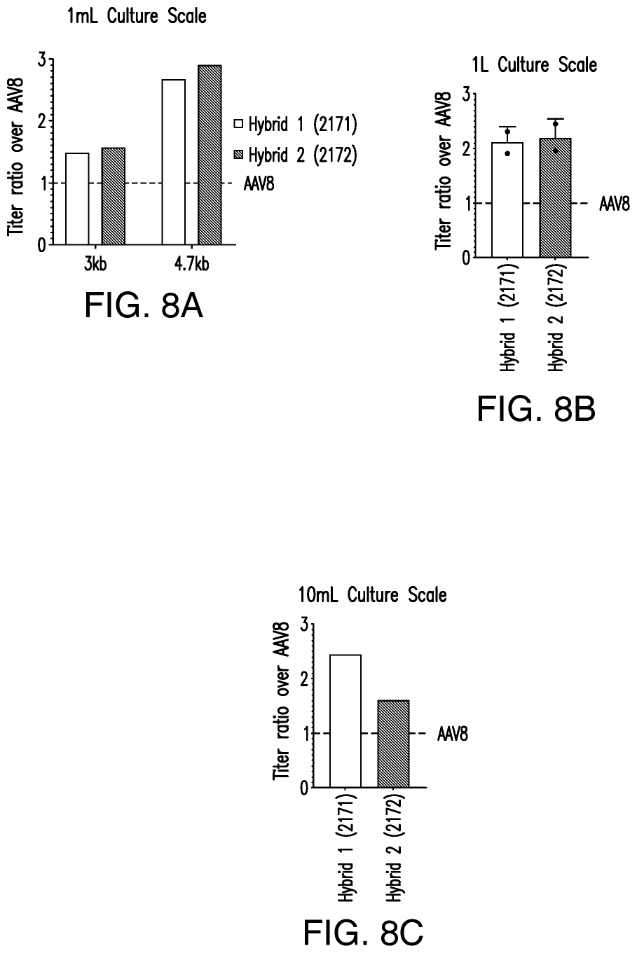

AAV5/AAV8 hybrid capsids transduce less efficiently (FIG. 7A) but are liver de-targeted on the transcriptional level (FIG. 7B) and express RNA more efficiently than wild type AAV8 in muscle, leading to an improved RNA/DNA ratio (2.5-3.5× increase over AAV8; FIG. 7C). Despite a decrease of transduction in skeletal muscle, transcription of genomes delivered by AAV5 hybrids was increased, leading to an improved RNA/DNA ratio. Without ascribing to any particular theory, AAV5 VP1u and VP1/2s may enable increased endosomal escape or nuclear trafficking abilities. Upon further evaluation of production capabilities, AAV5/AAV8 hybrids specifically produce 1.5-3× more vector than their parental capsid, AAV8, in standard triple transfection of HEK293 suspension culture (FIGS. 8A-8C), regardless of the packaged genome size (FIG. 8A; 1 mL culture) or culture scale (FIGS. 8B-8C; 10 mL and 1 L culture, respectively). In summary, AAV5 hybrid capsids may present a unique method of gene therapy liver de-targeting at the transcriptional level, and offer improved vector production.

### 6.3 Example 3: Evaluation of AAV Hybrid Capsids in a Non-Human Primate (NHP) Study

In this experiment, AAVhu32 was identified as a potentially superior clade F capsid candidate for muscle-specific disease indications. Such capsid was characterized in the context of a pool of 44 barcoded vectors administered intravenously into cynomolgus macaques (n=3) at 2×10¹³ genome copies per kg, and treated animal tissues were harvested three weeks post-injection and nucleic acids were extracted as detailed below.

Vector Production. Vector particles were produced by triple transfection of suspension-adapted HEK293 cells. Vector packaging included a TdTomato transgene with CAG promoter and 15 base pair. Uniquely barcoded AAV vectors were harvested via freeze-thaw and purified via an iodixanol gradient.

Vector Pooling. A total of 44 different iodixanol-purified, endotoxin-removed vector preparations were pooled in approximately equal particle numbers and quantified by ddPCR.

Dosing. Two female and one male cynomolgus macaques of approximately two to four years old were screened for AAV2, AAV8, and AAV9 neutralizing antibody status prior to injection, with any positive titers being below 1:10. Animals included in the study were injected intravenously at 2×10¹³ GC/kg total and subsequently sacrificed and tissues harvested 21 days after dosing.

NGS and Data Analysis. DNA extracted from NHP tissue was used as a template for amplification of the barcode region in each AAV transgene. Secondary amplification was performed to add Illumina Nextera adapters and indices to the amplicons, which were then pooled and purified. The resultant amplicon library was denatured and sequenced on a MiSeq instrument (Illumina) using the MiSeq v3 chemistry reagent kit. Barcode counts were quantified from forward reads using a custom Galaxy workflow and converted to relative abundance, adjusted for the input pool distribution, and normalized to 1. Extracted RNA was used for cDNA generation and subjected to a similar process as above to prepare for NGS analysis. Although the pool contained 44 different capsids, data analysis, as represented in FIGS. 10A and 10B, was focused on AAVhu32 DNA and RNA relative abundance compared to AAV9.

Compared to AAV9, AAVhu32 transcripts were significantly higher by double in gastrocnemius (P<0.0001) and bicep (P=0.03) tissue. The two capsids transduced quadricep and tibialis anterior similarly. In heart, AAVhu32 produced two-fold more transcript copies than AAV9, but it was not statistically significant (P=0.36), likely due to animal-to-animal variability. As hepatotoxicity is a concern in gene therapy, it is significant to note that AAVhu32 transduced the liver at half the level of AAV9 and produced one third of RNA transcript copies compared to AAV9 (P=0.03).

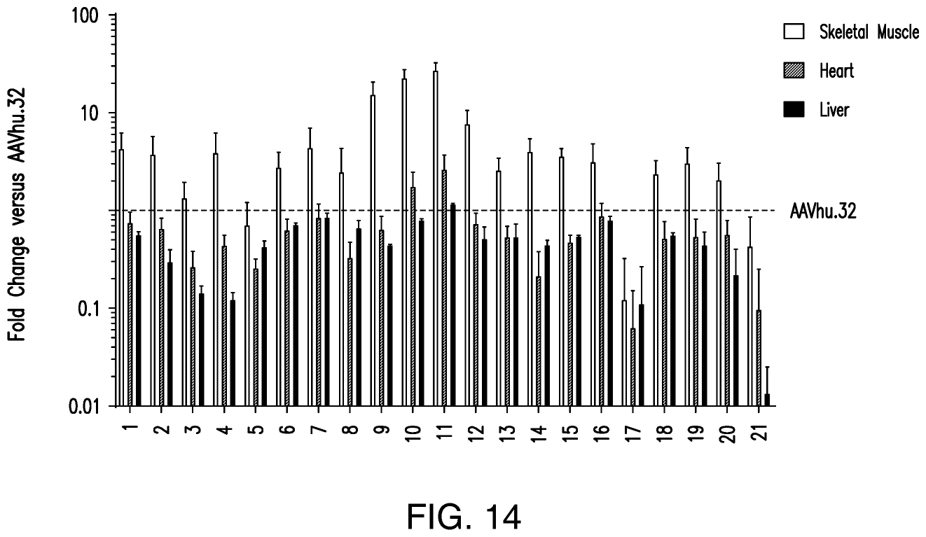

The AAVhu32 capsids (variants) represented in the capsid pool were analyzed for their transduction capability in muscle tissues versus liver. All skeletal muscle transcript data (bicep, gas, quad, TA, and diaphragm), heart transcript data (samples taken from aorta, apex, left and right atrium, left and right ventricle), and liver transcript data (left lobe sample) from 2 animals is represented in FIG. 14. Most variants expressed higher levels of transcript (relative abundance of mRNA transcript copies) compared to wild-type hu32.

### 6.4 Example 4: Evaluation of AAV Hybrid Capsids in a Single Vector Study in Mice

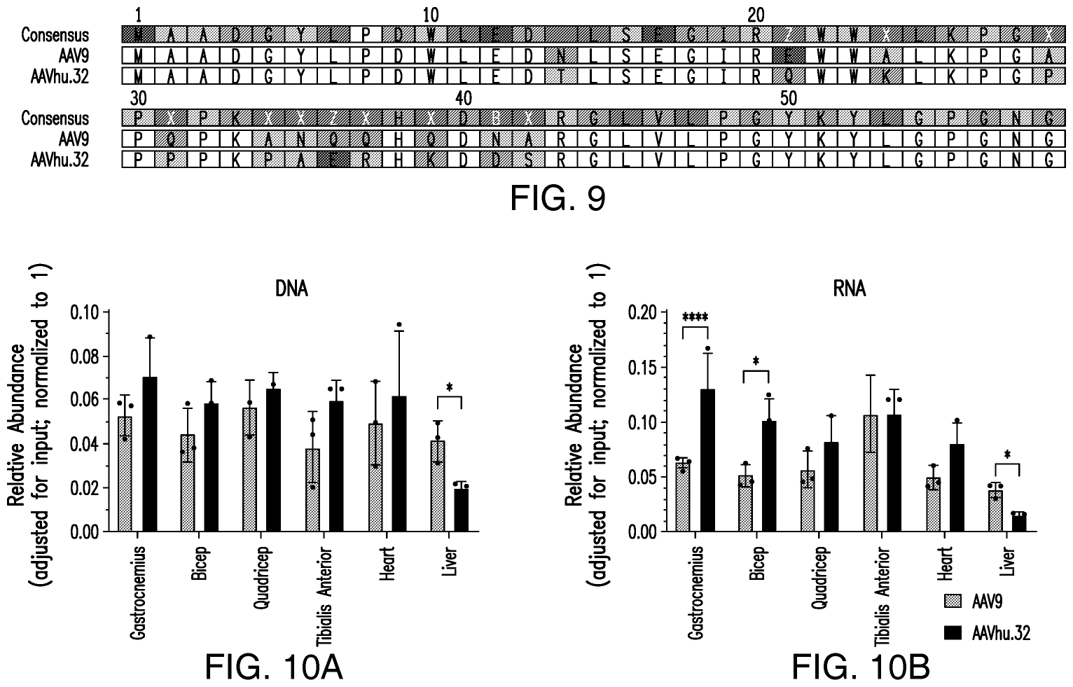

Additionally, AAVhu32 compared to AAV9 was evaluated in a single vector administration study in a mouse model of Duchenne muscular dystrophy (mdx, n=5) and recapitulated results seen in the pooled vector NHP study. Most of the amino acid differences between AAVhu32 and AAV9 lie in the VP1 unique (VP1u) region (FIG. 9), potentially adjacent to the PLA2 domain; however, the enzymatic (phospholipase) activity of recombinantly expressed AAVhu32 VP1u was found to be equivalent to that of AAV9 VP1u.

AAV9 and AAVhu32 vectors packaging a microdystrophin construct driven by a muscle-specific promoter (transgene) were produced. Five, 5 to 6-week-old mdx mice were dosed via tail vain injection with 5×10¹³ GC/kg. Tissues were harvested four weeks after vector dosing for DNA and RNA analysis using ddPCR.

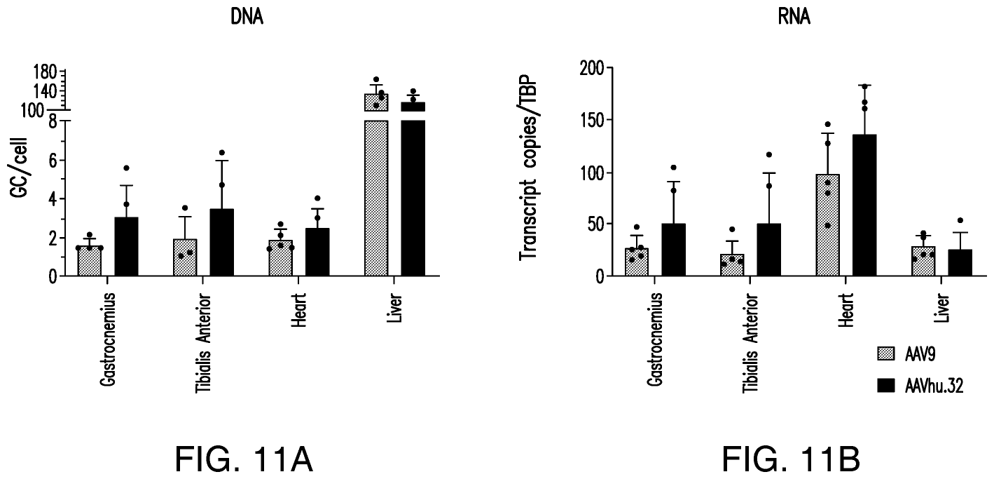

Biodistribution of AAVhu32 and AAV9 was assessed in mdx mouse tissues after intravenous administration (FIGS. 11A and 11B). Compared to AAV9, AAVhu32 had increased transduction of muscle tissues in terms of genome copy numbers (GC/cell) and RNA transcript copies by approximately double, similarly to NHP. Liver “de-targeting” of AAVhu32 was observed, but less reduced in the mdx mouse model compared to the differences seen between AAVhu32 and AAV9 in the NHP study.

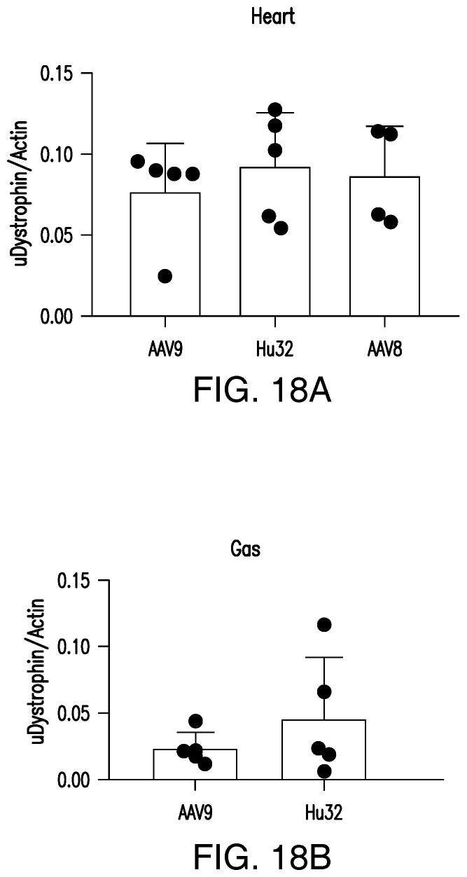

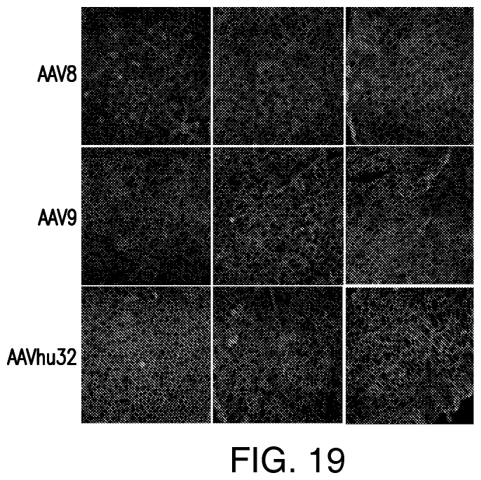

The biodistribution of AAVhu32 and AAV9 in mdx mouse tissues was also done by quantifying protein expression by Western Blot. After (systemic) intravenous administration, heart tissues from AAVhu32, AAV9, or AAV8-injected mice (FIG. 18A) or gastrocnemius (gas) tissues from AAVhu32 or AAV9-injected mice (FIG. 18B) were evaluated by protein quantification of the transgene (microdystrophin) and a representative muscle protein (actin) using standard protein blot transfer techniques. The protein data correlates well with the RNA transcript results for these tissues. Immunofluorescent staining of the gastrocnemius (gas) tissues from AAVhu32, AAV8 or AAV9-injected mice shows strong fluorescence detection of the microdystrophin transgene, especially in AAVhu32-injected mice (FIG. 19).

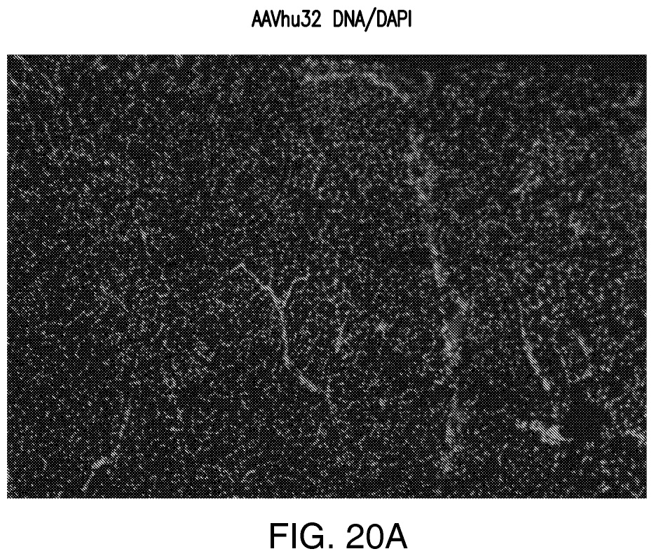

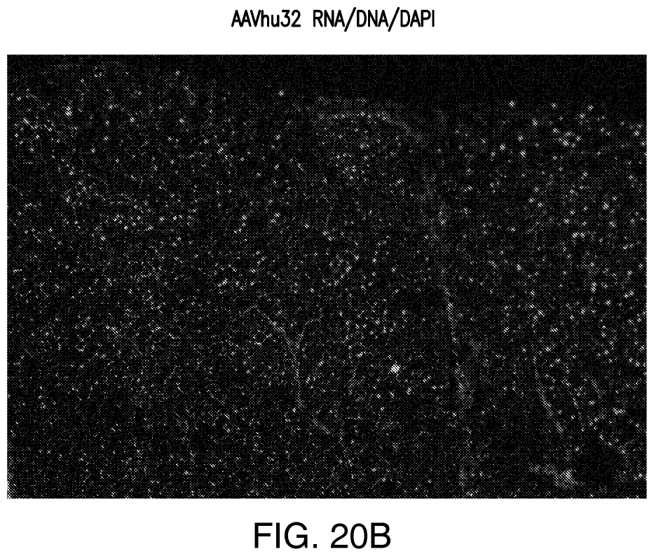

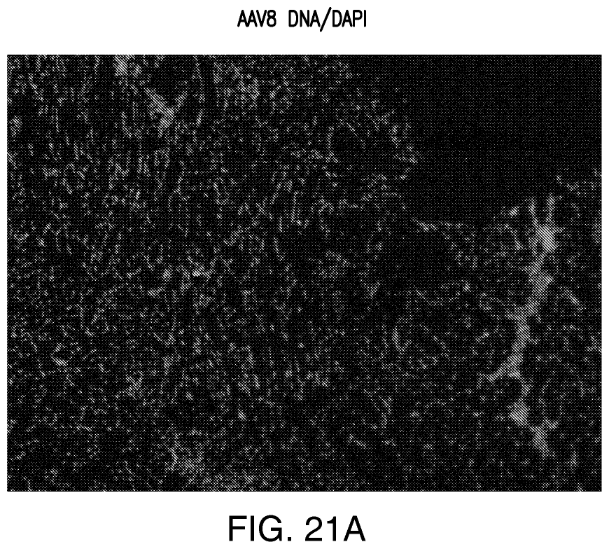

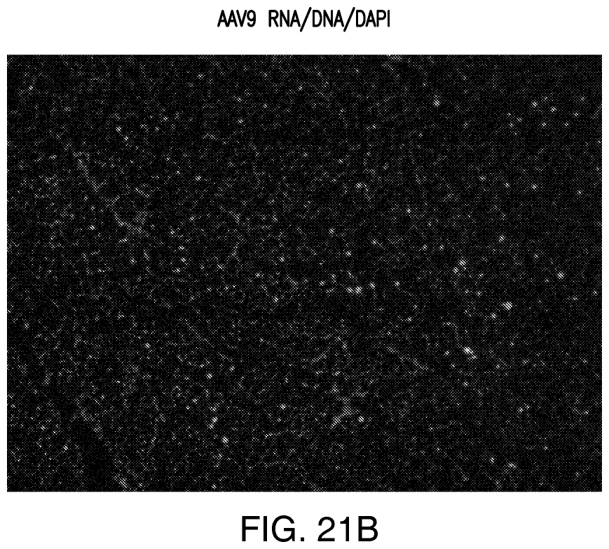

RNASCOPE experiments were performed on mdx mouse tissues following AAVhu32, AAV8 or AAV9-microdystrophin vector systemic administration. DNA and RNA probes were designed to detect vector or transgene in tissues, also markers for the specific tissue were used. RNASCOPE scoring was based on the number of green (AAV-microdystrophin DNA), red (AAV-microdystrophin RNA/DNA), or blue (DAPI) signals (dots) per cell, for semi-quantificiation of the transduction events. AAVhu32-microdystrophin DNA detection was observed at about 3.73 GC/diploid cell (FIG. 20A) and RNA detection at about 82.3 microidystrophin RNA copies/TBP (TBP=TATA-box binding protein control) (FIG. 20B). AAV8-microdystrophin DNA detection was observed at about 1 copy per cell (FIG. 21A), and AAV9-microdystrophin DNA detection was observed at about 1.4 copies per cell (FIG. 21B).

### 6.5 Example 5: Evaluation of AAV Hybrid Capsids in a Pooled, Engineered Vector Study in Mice

The ability of AAVhu32 and engineered variants of AAVhu32 to package a barcoded microdystrophin construct driven by a muscle-specific promoter (transgene) were produced by triple transfection in suspension HEK293 cells and purified by iodixanol gradient. Vector preparations were pooled in approximately equal particle numbers and quantified by ddPCR. 5 to 6-week-old, male C57BL10J and mdx mice were dosed via tail vain injection with 2×10¹³ GC/kg. Tissues were harvested three weeks post injection for nucleic acid extraction and NGS analysis as described above.

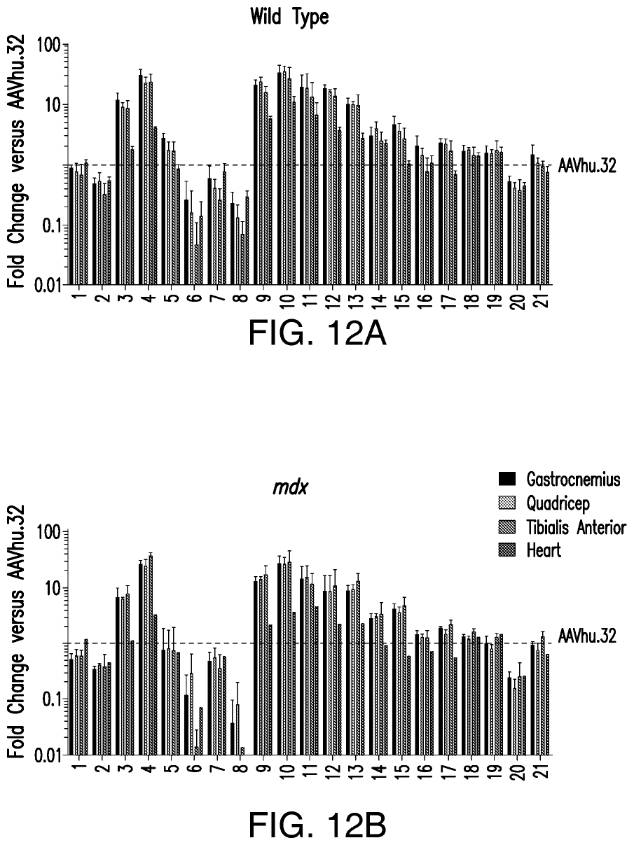

Engineered AAVhu32 capsids were capable of further enhancing transduction of skeletal muscle in wild type mice, as illustrated in FIGS. 12A and 12B. Some engineered AAVhu32-based capsids were capable of expression (relative abundance of mRNA transcript copies) up to 35× that of wild type AAVhu32 in several skeletal muscle tissues. Cardiac muscle expression was also improved, but to a lesser degree. Relative abundance of transcript copies was adjusted based on the vector input and presented as a fold change over AAVhu32. Similar trends were seen in the mdx mouse model.

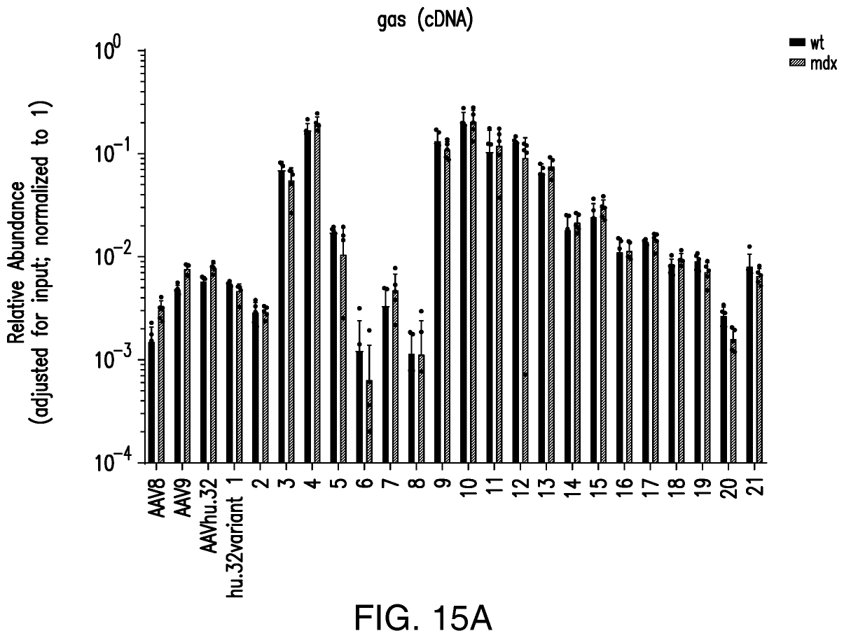

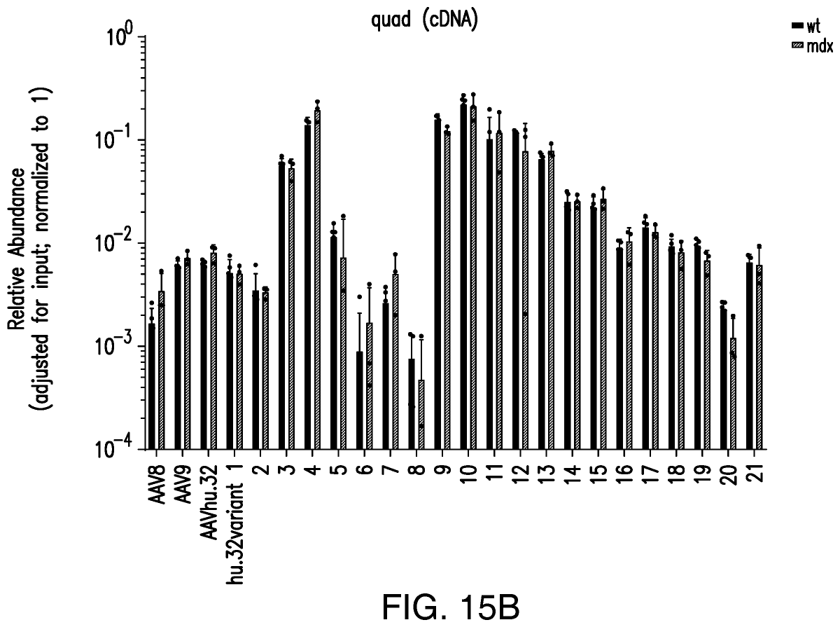

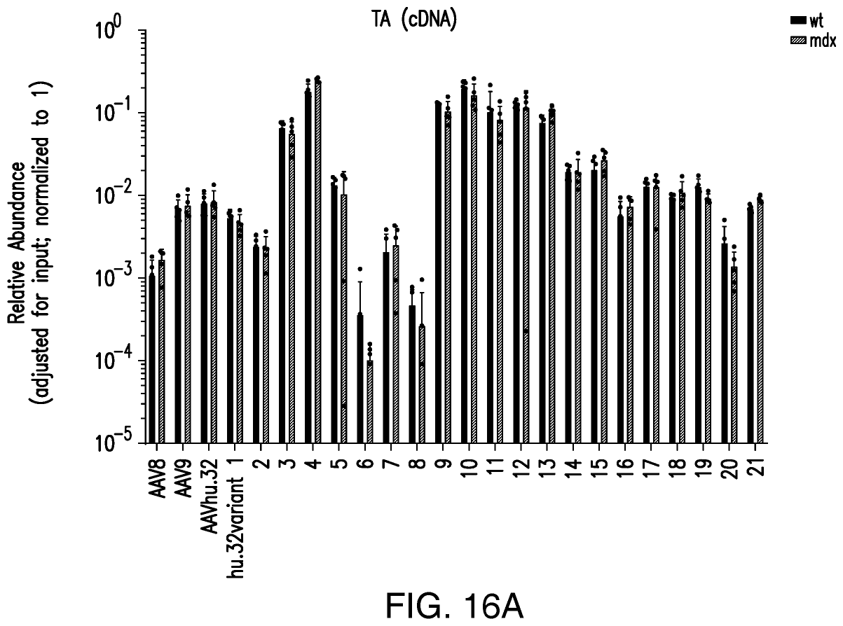

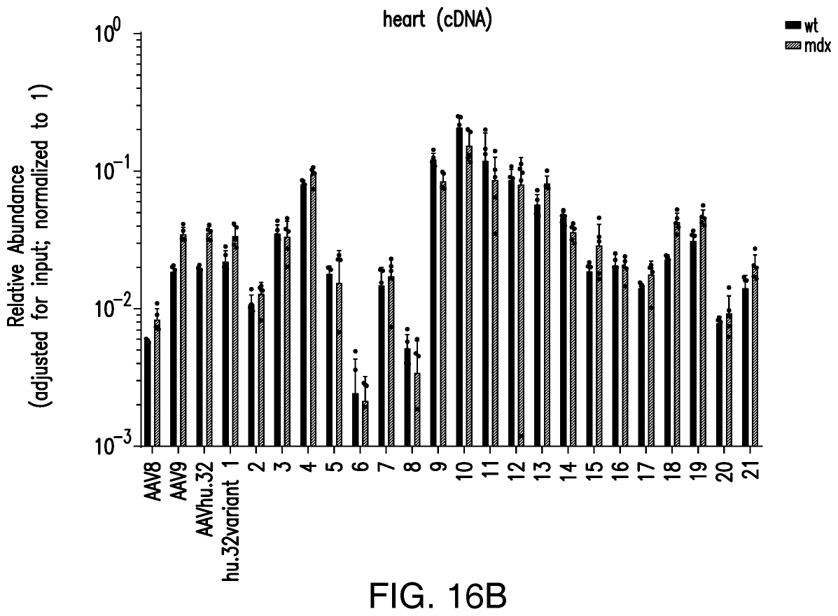

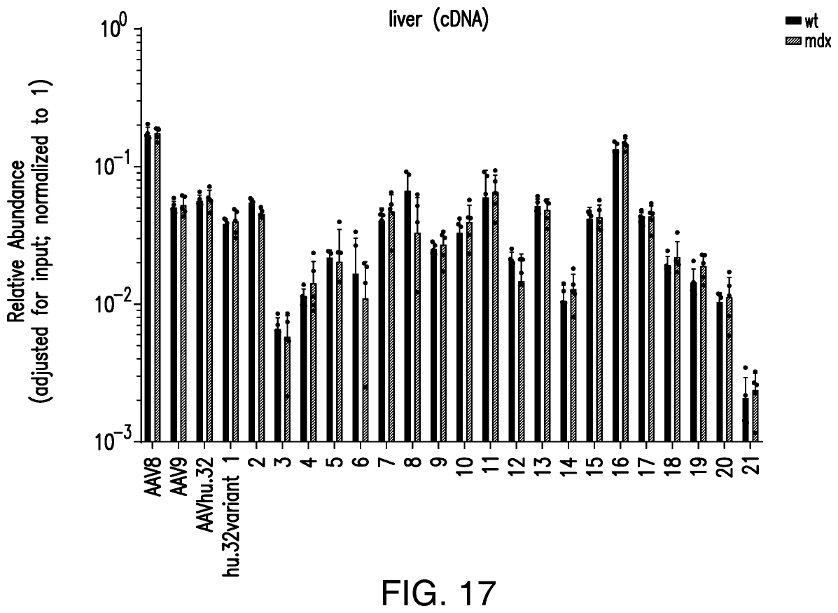

AAVhu32 capsid variants #1 to 21 were also analyzed compared to AAV8, AAV9, and wild-type AAVhu32 and the relative abundance of mRNA transcript copies (of transgene) were detected in each tissue, such as: gastrocnemius (gas) (FIG. 15A), quadriceps (quad) (FIG. 15B), tibialis anterior (TA) (FIG. 16A), heart (FIG. 16B), and liver (FIG. 17) tissues of wt and mdx mice.

As shown in the Examples hereinabove, Clade F vector AAVhu32 increased skeletal and cardiac muscle in NHP by approximately two fold compared to AAV9. AAVhu32 also showed improved transduction of muscle in mdx mice. In NHP, AAVhu32 showed decreased liver transduction compared to AAV9.

Although most similar to Clade F capsids by overall VP1 sequence alignment, AAVhu32 VP1u contains a proline-rich region (aa27-34) that is also present in Clade B (AAV2-like) vectors. AAVhu32 is considered an improved clade F capsid for muscle-specific disease indications, as compared to AAV9. Engineered AAVhu32 vectors may further increase transduction and/or transcription in wild type and mdx mouse muscle, and therefore AAVhu32-based vectors find therapeutic utility in muscle disease gene therapy treatments.

### 7. SEQUENCES

| SEQ ID | | |
|---|---|---|
| NO. | DESCRIPTION | SEQUENCE |
| 1 | AAV5 VP3 capsid | MSAGGGGPLGDNNQGADGVGNASGDWHCDSTWMGDRVVTK |
| | protein | STRTWVLPSYNNHQYREIKSGSVDGSNANAYFGYSTPWGY |
| | | FDFNRFHSHWSPRDWQRLINNYWGFRPRSLRVKIFNIQVK |
| | | EVTVQDSTTTIANNLTSTVQVFTDDDYQLPYVVGNGTEGC |
| | | LPAFPPQVFTLPQYGYATLNRDNTENPTERSSFFCLEYFP |
| | | SKMLRTGNNFEFTYNFEEVPFHSSFAPSQNLFKLANPLVD |
| | | QYLYRFVSTNNTGGVQFNKNLAGRYANTYKNWFPGPMGRT |
| | | QGWNLGSGVNRASVSAFATTNRMELEGASYQVPPQPNGMT |
| | | NNLQGSNTYALENTMIFNSQPANPGTTATYLEGNMLITSE |
| | | SETQPVNRVAYNVGGQMATNNQSSTTAPATGTYNLQEIVP |
| | | GSVWMERDVYLQGPIWAKIPETGAHFHPSPAMGGFGLKHP |
| | | PPMMLIKNTPVPGNITSFSDVPVSSFITQYSTGQVTVEME |
| | | WELKKENSKRWNPEIQYTNNYNDPQFVDFAPDSTGEYRTT |
| | | RPIGTRYLTRPL |
| 2 | AAV5 VP3 capsid | ATGTCTGCGGGAGGTGGCGGCCCATTGGGCGACAATAACC |
| | DNA | AAGGTGCCGATGGAGTGGGCAATGCCTCGGGAGATTGGCA |
| | | TTGCGATTCCACGTGGATGGGGGACAGAGTCGTCACCAAG |
| | | TCCACCCGAACCTGGGTGCTGCCCAGCTACAACAACCACC |
| | | AGTACCGAGAGATCAAAAGCGGCTCCGTCGACGGAAGCAA |
| | | CGCCAACGCCTACTTTGGATACAGCACCCCCTGGGGGTAC |
| | | TTTGACTTTAACCGCTTCCACAGCCACTGGAGCCCCCGAG |
| | | ACTGGCAAAGACTCATCAACAACTACTGGGGCTTCAGACC |
| | | CCGGTCCCTCAGAGTCAAAATCTTCAACATTCAAGTCAAA |
| | | GAGGTCACGGTGCAGGACTCCACCACCACCATCGCCAACA |
| | | ACCTCACCTCCACCGTCCAAGTGTTTACGGACGACGACTA |
| | | CCAGCTGCCCTACGTCGTCGGCAACGGGACCGAGGGATGC |
| | | CTGCCGGCCTTCCCTCCGCAGGTCTTTACGCTGCCGCAGT |
| | | ACGGTTACGCGACGCTGAACCGCGACAACACAGAAAATCC |
| | | CACCGAGAGGAGCAGCTTCTTCTGCCTAGAGTACTTTCCC |
| | | AGCAAGATGCTGAGAACGGGCAACAACTTTGAGTTTACCT |
| | | ACAACTTTGAGGAGGTGCCCTTCCACTCCAGCTTCGCTCC |
| | | CAGTCAGAACCTGTTCAAGCTGGCCAACCCGCTGGTGGAC |
| | | CAGTACTTGTACCGCTTCGTGAGCACAAATAACACTGGCG |
| | | GAGTCCAGTTCAACAAGAACCTGGCCGGGAGATACGCCAA |
| | | CACCTACAAAAACTGGTTCCCGGGGCCCATGGGCCGAACC |
| | | CAGGGCTGGAACCTGGGCTCCGGGGTCAACCGCGCCAGTG |
| | | TCAGCGCCTTCGCCACGACCAATAGGATGGAGCTCGAGGG |
| | | CGCGAGTTACCAGGTGCCCCCGCAGCCGAACGGCATGACC |
| | | AACAACCTCCAGGGCAGCAACACCTATGCCCTGGAGAACA |
| | | CTATGATCTTCAACAGCCAGCCGGCGAACCCGGGCACCAC |
| | | CGCCACGTACCTCGAGGGCAACATGCTCATCACCAGCGAG |
| | | AGCGAGACGCAGCCGGTGAACCGCGTGGCGTACAACGTCG |
| | | GCGGGCAGATGGCCACCAACAACCAGAGCTCCACCACTGC |
| | | CCCCGCGACCGGCACGTACAACCTCCAGGAAATCGTGCCC |
| | | GGCAGCGTGTGGATGGAGAGGGACGTGTACCTCCAAGGAC |
| | | CCATCTGGGCCAAGATCCCAGAGACGGGGGCGCACTTTCA |
| | | CCCCTCTCCGGCCATGGGCGGATTCGGACTCAAACACCCA |
| | | CCGCCCATGATGCTCATCAAGAACACGCCTGTGCCCGGAA |
| | | ATATCACCAGCTTCTCGGACGTGCCCGTCAGCAGCTTCAT |
| | | CACCCAGTACAGCACCGGGCAGGTCACCGTGGAGATGGAG |
| | | TGGGAGCTCAAGAAGGAAAACTCCAAGAGGTGGAACCCAG |
| | | AGATCCAGTACACAAACAACTACAACGACCCCCAGTTTGT |
| | | GGACTTTGCCCCGGACAGCACCGGGGAATACAGAACCACC |
| | | AGACCTATCGGAACCCGATACCTTACCCGACCCCTTTAA |
| 3 | AAV5 VP1/2s | TAPTGKRIDDHFPKRKKARTEEDSKPSTSSDAEAGPSGSQ |
| | common region | QLQIPAQPASSLGADT |
| 4 | AAV5 VP1/2s | ACGGCCCCTACCGGAAAGCGGATAGACGACCACTTTCCAA |
| | common region | AAAGAAAGAAGGCTCGGACCGAAGAGGACTCCAAGCCTTC |
| | | CACCTCGTCAGACGCCGAAGCTGGACCCAGCGGATCCCAG |
| | | CAGCTGCAAATCCCAGCCCAACCAGCCTCAAGTTTGGGAG |
| | | CTGATACA |
| 5 | AAV5 VP2 capsid | TAPTGKRIDDHFPKRKKARTEEDSKPSTSSDAEAGPSGSQ |
| | protein | QLQIPAQPASSLGADTMSAGGGGPLGDNNQGADGVGNASG |
| | | DWHCDSTWMGDRVVTKSTRTWVLPSYNNHQYREIKSGSVD |
| | | GSNANAYFGYSTPWGYFDFNRFHSHWSPRDWQRLINNYWG |
| | | FRPRSLRVKIFNIQVKEVTVQDSTTTIANNLTSTVQVFTD |
| | | DDYQLPYVVGNGTEGCLPAFPPQVFTLPQYGYATLNRDNT |
| | | ENPTERSSFFCLEYFPSKMLRTGNNFEFTYNFEEVPFHSS |
| | | FAPSQNLFKLANPLVDQYLYRFVSTNNTGGVQFNKNLAGR |
| | | YANTYKNWFPGPMGRTQGWNLGSGVNRASVSAFATTNRME |
| | | LEGASYQVPPQPNGMTNNLQGSNTYALENTMIFNSQPANP |
| | | GTTATYLEGNMLITSESETQPVNRVAYNVGGQMATNNQSS |
| | | TTAPATGTYNLQEIVPGSVWMERDVYLQGPIWAKIPETGA |
| | | HFHPSPAMGGFGLKHPPPMMLIKNTPVPGNITSFSDVPVS |
| | | SFITQYSTGQVTVEMEWELKKENSKRWNPEIQYTNNYNDP |
| | | QFVDFAPDSTGEYRTTRPIGTRYLTRPL |
| 6 | AAV5 VP2 capsid | ACGGCCCCTACCGGAAAGCGGATAGACGACCACTTTCCAA |
| | DNA | AAAGAAAGAAGGCTCGGACCGAAGAGGACTCCAAGCCTTC |
| | | CACCTCGTCAGACGCCGAAGCTGGACCCAGCGGATCCCAG |
| | | CAGCTGCAAATCCCAGCCCAACCAGCCTCAAGTTTGGGAG |
| | | CTGATACAATGTCTGCGGGAGGTGGCGGCCCATTGGGCGA |
| | | CAATAACCAAGGTGCCGATGGAGTGGGCAATGCCTCGGGA |
| | | GATTGGCATTGCGATTCCACGTGGATGGGGGACAGAGTCG |
| | | TCACCAAGTCCACCCGAACCTGGGTGCTGCCCAGCTACAA |
| | | CAACCACCAGTACCGAGAGATCAAAAGCGGCTCCGTCGAC |
| | | GGAAGCAACGCCAACGCCTACTTTGGATACAGCACCCCCT |
| | | GGGGGTACTTTGACTTTAACCGCTTCCACAGCCACTGGAG |
| | | CCCCCGAGACTGGCAAAGACTCATCAACAACTACTGGGGC |
| | | TTCAGACCCCGGTCCCTCAGAGTCAAAATCTTCAACATTC |
| | | AAGTCAAAGAGGTCACGGTGCAGGACTCCACCACCACCAT |
| | | CGCCAACAACCTCACCTCCACCGTCCAAGTGTTTACGGAC |
| | | GACGACTACCAGCTGCCCTACGTCGTCGGCAACGGGACCG |
| | | AGGGATGCCTGCCGGCCTTCCCTCCGCAGGTCTTTACGCT |
| | | GCCGCAGTACGGTTACGCGACGCTGAACCGCGACAACACA |
| | | GAAAATCCCACCGAGAGGAGCAGCTTCTTCTGCCTAGAGT |
| | | ACTTTCCCAGCAAGATGCTGAGAACGGGCAACAACTTTGA |
| | | GTTTACCTACAACTTTGAGGAGGTGCCCTTCCACTCCAGC |
| | | TTCGCTCCCAGTCAGAACCTGTTCAAGCTGGCCAACCCGC |
| | | TGGTGGACCAGTACTTGTACCGCTTCGTGAGCACAAATAA |
| | | CACTGGCGGAGTCCAGTTCAACAAGAACCTGGCCGGGAGA |
| | | TACGCCAACACCTACAAAAACTGGTTCCCGGGGCCCATGG |
| | | GCCGAACCCAGGGCTGGAACCTGGGCTCCGGGGTCAACCG |
| | | CGCCAGTGTCAGCGCCTTCGCCACGACCAATAGGATGGAG |
| | | CTCGAGGGCGCGAGTTACCAGGTGCCCCCGCAGCCGAACG |
| | | GCATGACCAACAACCTCCAGGGCAGCAACACCTATGCCCT |
| | | GGAGAACACTATGATCTTCAACAGCCAGCCGGCGAACCCG |
| | | GGCACCACCGCCACGTACCTCGAGGGCAACATGCTCATCA |
| | | CCAGCGAGAGCGAGACGCAGCCGGTGAACCGCGTGGCGTA |
| | | CAACGTCGGCGGGCAGATGGCCACCAACAACCAGAGCTCC |
| | | ACCACTGCCCCCGCGACCGGCACGTACAACCTCCAGGAAA |
| | | TCGTGCCCGGCAGCGTGTGGATGGAGAGGGACGTGTACCT |
| | | CCAAGGACCCATCTGGGCCAAGATCCCAGAGACGGGGGCG |
| | | CACTTTCACCCCTCTCCGGCCATGGGCGGATTCGGACTCA |
| | | AACACCCACCGCCCATGATGCTCATCAAGAACACGCCTGT |
| | | GCCCGGAAATATCACCAGCTTCTCGGACGTGCCCGTCAGC |
| | | AGCTTCATCACCCAGTACAGCACCGGGCAGGTCACCGTGG |
| | | AGATGGAGTGGGAGCTCAAGAAGGAAAACTCCAAGAGGTG |
| | | GAACCCAGAGATCCAGTACACAAACAACTACAACGACCCC |
| | | CAGTTTGTGGACTTTGCCCCGGACAGCACCGGGGAATACA |
| | | GAACCACCAGACCTATCGGAACCCGATACCTTACCCGACC |
| | | CCTTTAA |
| 7 | AAV5 VP1u region | MSFVDHPPDWLEEVGEGLREFLGLEAGPPKPKPNQQHQDQ |
| | (protein) | ARGLVLPGYNYLGPGNGLDRGEPVNRADEVAREHDISYNE |
| | | QLEAGDNPYLKYNHADAEFQEKLADDTSFGGNLGKAVFQA |
| | | KKRVLEPFGLVEEGAK |
| 8 | AAV5 VP1u region | ATGTCTTTTGTTGATCACCCTCCAGATTGGTTGGAAGAAG |
| | (DNA) | TTGGTGAAGGTCTTCGCGAGTTTTTGGGCCTTGAAGCGGG |
| | | CCCACCGAAACCAAAACCCAATCAGCAGCATCAAGATCAA |
| | | GCCCGTGGTCTTGTGCTGCCTGGTTATAACTATCTCGGAC |
| | | CCGGAAACGGTCTCGATCGAGGAGAGCCTGTCAACAGGGC |
| | | AGACGAGGTCGCGCGAGAGCACGACATCTCGTACAACGAG |
| | | CAGCTTGAGGCGGGAGACAACCCCTACCTCAAGTACAACC |
| | | ACGCGGACGCCGAGTTTCAGGAGAAGCTCGCCGACGACAC |
| | | ATCCTTCGGGGGAAACCTCGGAAAGGCAGTCTTTCAGGCC |
| | | AAGAAAAGGGTTCTCGAACCTTTTGGCCTGGTTGAAGAGG |
| | | GTGCTAAG |
| 9 | AAV5 VP1 capsid | MSFVDHPPDWLEEVGEGLREFLGLEAGPPKPKPNQQHQDQ |
| | protein | ARGLVLPGYNYLGPGNGLDRGEPVNRADEVAREHDISYNE |
| | | QLEAGDNPYLKYNHADAEFQEKLADDTSFGGNLGKAVFQA |
| | | KKRVLEPFGLVEEGAKTAPTGKRIDDHFPKRKKARTEEDS |
| | | KPSTSSDAEAGPSGSQQLQIPAQPASSLGADTMSAGGGGP |
| | | LGDNNQGADGVGNASGDWHCDSTWMGDRVVTKSTRTWVLP |
| | | SYNNHQYREIKSGSVDGSNANAYFGYSTPWGYFDFNRFHS |
| | | HWSPRDWQRLINNYWGFRPRSLRVKIFNIQVKEVTVQDST |
| | | TTIANNLTSTVQVFTDDDYQLPYVVGNGTEGCLPAFPPQV |
| | | FTLPQYGYATLNRDNTENPTERSSFFCLEYFPSKMLRTGN |
| | | NFEFTYNFEEVPFHSSFAPSQNLFKLANPLVDQYLYRFVS |
| | | TNNTGGVQFNKNLAGRYANTYKNWFPGPMGRTQGWNLGSG |
| | | VNRASVSAFATTNRMELEGASYQVPPQPNGMTNNLQGSNT |
| | | YALENTMIFNSQPANPGTTATYLEGNMLITSESETQPVNR |
| | | VAYNVGGQMATNNQSSTTAPATGTYNLQEIVPGSVWMERD |
| | | VYLQGPIWAKIPETGAHFHPSPAMGGFGLKHPPPMMLIKN |
| | | TPVPGNITSFSDVPVSSFITQYSTGQVTVEMEWELKKENS |
| | | KRWNPEIQYTNNYNDPQFVDFAPDSTGEYRTTRPIGTRYL |
| | | TRPL |
| 10 | AAV5 VP1 capsid | ATGTCTTTTGTTGATCACCCTCCAGATTGGTTGGAAGAAG |
| | DNA | TTGGTGAAGGTCTTCGCGAGTTTTTGGGCCTTGAAGCGGG |
| | | CCCACCGAAACCAAAACCCAATCAGCAGCATCAAGATCAA |
| | | GCCCGTGGTCTTGTGCTGCCTGGTTATAACTATCTCGGAC |
| | | CCGGAAACGGTCTCGATCGAGGAGAGCCTGTCAACAGGGC |
| | | AGACGAGGTCGCGCGAGAGCACGACATCTCGTACAACGAG |
| | | CAGCTTGAGGCGGGAGACAACCCCTACCTCAAGTACAACC |
| | | ACGCGGACGCCGAGTTTCAGGAGAAGCTCGCCGACGACAC |
| | | ATCCTTCGGGGGAAACCTCGGAAAGGCAGTCTTTCAGGCC |
| | | AAGAAAAGGGTTCTCGAACCTTTTGGCCTGGTTGAAGAGG |
| | | GTGCTAAGACGGCCCCTACCGGAAAGCGGATAGACGACCA |
| | | CTTTCCAAAAAGAAAGAAGGCTCGGACCGAAGAGGACTCC |
| | | AAGCCTTCCACCTCGTCAGACGCCGAAGCTGGACCCAGCG |
| | | GATCCCAGCAGCTGCAAATCCCAGCCCAACCAGCCTCAAG |
| | | TTTGGGAGCTGATACAATGTCTGCGGGAGGTGGCGGCCCA |
| | | TTGGGCGACAATAACCAAGGTGCCGATGGAGTGGGCAATG |
| | | CCTCGGGAGATTGGCATTGCGATTCCACGTGGATGGGGGA |
| | | CAGAGTCGTCACCAAGTCCACCCGAACCTGGGTGCTGCCC |
| | | AGCTACAACAACCACCAGTACCGAGAGATCAAAAGCGGCT |
| | | CCGTCGACGGAAGCAACGCCAACGCCTACTTTGGATACAG |
| | | CACCCCCTGGGGGTACTTTGACTTTAACCGCTTCCACAGC |
| | | CACTGGAGCCCCCGAGACTGGCAAAGACTCATCAACAACT |
| | | ACTGGGGCTTCAGACCCCGGTCCCTCAGAGTCAAAATCTT |
| | | CAACATTCAAGTCAAAGAGGTCACGGTGCAGGACTCCACC |
| | | ACCACCATCGCCAACAACCTCACCTCCACCGTCCAAGTGT |
| | | TTACGGACGACGACTACCAGCTGCCCTACGTCGTCGGCAA |
| | | CGGGACCGAGGGATGCCTGCCGGCCTTCCCTCCGCAGGTC |
| | | TTTACGCTGCCGCAGTACGGTTACGCGACGCTGAACCGCG |
| | | ACAACACAGAAAATCCCACCGAGAGGAGCAGCTTCTTCTG |
| | | CCTAGAGTACTTTCCCAGCAAGATGCTGAGAACGGGCAAC |
| | | AACTTTGAGTTTACCTACAACTTTGAGGAGGTGCCCTTCC |
| | | ACTCCAGCTTCGCTCCCAGTCAGAACCTGTTCAAGCTGGC |
| | | CAACCCGCTGGTGGACCAGTACTTGTACCGCTTCGTGAGC |
| | | ACAAATAACACTGGCGGAGTCCAGTTCAACAAGAACCTGG |
| | | CCGGGAGATACGCCAACACCTACAAAAACTGGTTCCCGGG |
| | | GCCCATGGGCCGAACCCAGGGCTGGAACCTGGGCTCCGGG |
| | | GTCAACCGCGCCAGTGTCAGCGCCTTCGCCACGACCAATA |
| | | GGATGGAGCTCGAGGGCGCGAGTTACCAGGTGCCCCCGCA |
| | | GCCGAACGGCATGACCAACAACCTCCAGGGCAGCAACACC |
| | | TATGCCCTGGAGAACACTATGATCTTCAACAGCCAGCCGG |
| | | CGAACCCGGGCACCACCGCCACGTACCTCGAGGGCAACAT |
| | | GCTCATCACCAGCGAGAGCGAGACGCAGCCGGTGAACCGC |
| | | GTGGCGTACAACGTCGGCGGGCAGATGGCCACCAACAACC |
| | | AGAGCTCCACCACTGCCCCCGCGACCGGCACGTACAACCT |
| | | CCAGGAAATCGTGCCCGGCAGCGTGTGGATGGAGAGGGAC |
| | | GTGTACCTCCAAGGACCCATCTGGGCCAAGATCCCAGAGA |
| | | CGGGGGCGCACTTTCACCCCTCTCCGGCCATGGGCGGATT |
| | | CGGACTCAAACACCCACCGCCCATGATGCTCATCAAGAAC |
| | | ACGCCTGTGCCCGGAAATATCACCAGCTTCTCGGACGTGC |
| | | CCGTCAGCAGCTTCATCACCCAGTACAGCACCGGGCAGGT |
| | | CACCGTGGAGATGGAGTGGGAGCTCAAGAAGGAAAACTCC |
| | | AAGAGGTGGAACCCAGAGATCCAGTACACAAACAACTACA |
| | | ACGACCCCCAGTTTGTGGACTTTGCCCCGGACAGCACCGG |
| | | GGAATACAGAACCACCAGACCTATCGGAACCCGATACCTT |
| | | ACCCGACCCCTTTAA |
| 11 | AAV8 VP3 capsid | MAAGGGAPMADNNEGADGVGSSSGNWHCDSTWLGDRVITT |
| | protein | STRTWALPTYNNHLYKQISNGTSGGATNDNTYFGYSTPWG |
| | | YFDFNRFHCHFSPRDWQRLINNNWGFRPKRLSFKLFNIQV |
| | | KEVTQNEGTKTIANNLTSTIQVFTDSEYQLPYVLGSAHQG |
| | | CLPPFPADVFMIPQYGYLTLNNGSQAVGRSSFYCLEYFPS |
| | | QMLRTGNNFQFTYTFEDVPFHSSYAHSQSLDRLMNPLIDQ |
| | | YLYYLSRTQTTGGTANTQTLGFSQGGPNTMANQAKNWLPG |
| | | PCYRQQRVSTTTGQNNNSNFAWTAGTKYHLNGRNSLANPG |
| | | IAMATHKDDEERFFPSNGILIFGKQNAARDNADYSDVMLT |
| | | SEEEIKTTNPVATEEYGIVADNLQQQNTAPQIGTVNSQGA |
| | | LPGMVWQNRDVYLQGPIWAKIPHTDGNFHPSPLMGGFGLK |
| | | HPPPQILIKNTPVPADPPTTFNQSKINSFITQYSTGQVSV |
| | | EIEWELQKENSKRWNPEIQYTSNYYKSTSVDFAVNTEGVY |
| | | SEPRPIGTRYLTRNL |
| 12 | AAV8 VP3 capsid | ATGGCTGCAGGCGGTGGCGCACCAATGGCAGACAATAACG |
| | DNA | AAGGCGCCGACGGAGTGGGTAGTTCCTCGGGAAATTGGCA |
| | | TTGCGATTCCACATGGCTGGGCGACAGAGTCATCACCACC |
| | | AGCACCCGAACCTGGGCCCTGCCCACCTACAACAACCACC |
| | | TCTACAAGCAAATCTCCAACGGGACATCGGGAGGAGCCAC |
| | | CAACGACAACACCTACTTCGGCTACAGCACCCCCTGGGGG |
| | | TATTTTGACTTTAACAGATTCCACTGCCACTTTTCACCAC |
| | | GTGACTGGCAGCGACTCATCAACAACAACTGGGGATTCCG |
| | | GCCCAAGAGACTCAGCTTCAAGCTCTTCAACATCCAGGTC |
| | | AAGGAGGTCACGCAGAATGAAGGCACCAAGACCATCGCCA |
| | | ATAACCTCACCAGCACCATCCAGGTGTTTACGGACTCGGA |
| | | GTACCAGCTGCCGTACGTTCTCGGCTCTGCCCACCAGGGC |
| | | TGCCTGCCTCCGTTCCCGGCGGACGTGTTCATGATTCCCC |
| | | AGTACGGCTACCTAACACTCAACAACGGTAGTCAGGCCGT |
| | | GGGACGCTCCTCCTTCTACTGCCTGGAATACTTTCCTTCG |
| | | CAGATGCTGAGAACCGGCAACAACTTCCAGTTTACTTACA |
| | | CCTTCGAGGACGTGCCTTTCCACAGCAGCTACGCCCACAG |
| | | CCAGAGCTTGGACCGGCTGATGAATCCTCTGATTGACCAG |
| | | TACCTGTACTACTTGTCTCGGACTCAAACAACAGGAGGCA |
| | | CGGCAAATACGCAGACTCTGGGCTTCAGCCAAGGTGGGCC |
| | | TAATACAATGGCCAATCAGGCAAAGAACTGGCTGCCAGGA |
| | | CCCTGTTACCGCCAACAACGCGTCTCAACGACAACCGGGC |
| | | AAAACAACAATAGCAACTTTGCCTGGACTGCTGGGACCAA |
| | | ATACCATCTGAATGGAAGAAATTCATTGGCTAATCCTGGC |
| | | ATCGCTATGGCAACACACAAAGACGACGAGGAGCGTTTTT |
| | | TTCCCAGTAACGGGATCCTGATTTTTGGCAAACAAAATGC |
| | | TGCCAGAGACAATGCGGATTACAGCGATGTCATGCTCACC |
| | | AGCGAGGAAGAAATCAAAACCACTAACCCTGTGGCTACAG |
| | | AGGAATACGGTATCGTGGCAGATAACTTGCAGCAGCAAAA |
| | | CACGGCTCCTCAAATTGGAACTGTCAACAGCCAGGGGGCC |
| | | TTACCCGGTATGGTCTGGCAGAACCGGGACGTGTACCTGC |
| | | AGGGTCCCATCTGGGCCAAGATTCCTCACACGGACGGCAA |
| | | CTTCCACCCGTCTCCGCTGATGGGCGGCTTTGGCCTGAAA |
| | | CATCCTCCGCCTCAGATCCTGATCAAGAACACGCCTGTAC |
| | | CTGCGGATCCTCCGACCACCTTCAACCAGTCAAAGCTGAA |
| | | CTCTTTCATCACGCAATACAGCACCGGACAGGTCAGCGTG |
| | | GAAATTGAATGGGAGCTGCAGAAGGAAAACAGCAAGCGCT |
| | | GGAACCCCGAGATCCAGTACACCTCCAACTACTACAAATC |
| | | TACAAGTGTGGACTTTGCTGTTAATACAGAAGGCGTGTAC |
| | | TCTGAACCCCGCCCCATTGGCACCCGTTACCTCACCCGTA |
| | | ATCTGTAA |
| 13 | AAV8 VP1/2s | TAPGKKRPVEPSPQRSPDSSTGIGKKGQQPARKRLNFGQT |
| | common region | GDSESVPDPQPLGEPPAAPSGVGPNT |
| | (protein) | |
| 14 | AAV8 VP1/2s | ACGGCTCCTGGAAAGAAGAGACCGGTAGAGCCATCACCCC |
| | common region | AGCGTTCTCCAGACTCCTCTACGGGCATCGGCAAGAAAGG |
| | (DNA) | CCAACAGCCCGCCAGAAAAAGACTCAATTTTGGTCAGACT |
| | | GGCGACTCAGAGTCAGTTCCAGACCCTCAACCTCTCGGAG |
| | | AACCTCCAGCAGCGCCCTCTGGTGTGGGACCTAATACA |
| 15 | AAV8 VP2 capsid | TAPGKKRPVEPSPQRSPDSSTGIGKKGQQPARKRLNFGQT |
| | protein | GDSESVPDPQPLGEPPAAPSGVGPNTMAAGGGAPMADNNE |
| | | GADGVGSSSGNWHCDSTWLGDRVITTSTRTWALPTYNNHL |
| | | YKQISNGTSGGATNDNTYFGYSTPWGYFDFNRFHCHFSPR |
| | | DWQRLINNNWGFRPKRLSFKLFNIQVKEVTQNEGTKTIAN |
| | | NLTSTIQVFTDSEYQLPYVLGSAHQGCLPPFPADVFMIPQ |
| | | YGYLTLNNGSQAVGRSSFYCLEYFPSQMLRTGNNFQFTYT |
| | | FEDVPFHSSYAHSQSLDRLMNPLIDQYLYYLSRTQTTGGT |
| | | ANTQTLGFSQGGPNTMANQAKNWLPGPCYRQQRVSTTTGQ |
| | | NNNSNFAWTAGTKYHLNGRNSLANPGIAMATHKDDEERFF |
| | | PSNGILIFGKQNAARDNADYSDVMLTSEEEIKTTNPVATE |
| | | EYGIVADNLQQQNTAPQIGTVNSQGALPGMVWQNRDVYLQ |
| | | GPIWAKIPHTDGNFHPSPLMGGFGLKHPPPQILIKNTPVP |
| | | ADPPTTFNQSKLNSFITQYSTGQVSVEIEWELQKENSKRW |
| | | NPEIQYTSNYYKSTSVDFAVNTEGVYSEPRPIGTRYLTRN |
| | | L |
| 16 | AAV8 VP2 capsid | ACGGCTCCTGGAAAGAAGAGACCGGTAGAGCCATCACCCC |
| | DNA | AGCGTTCTCCAGACTCCTCTACGGGCATCGGCAAGAAAGG |
| | | CCAACAGCCCGCCAGAAAAAGACTCAATTTTGGTCAGACT |
| | | GGCGACTCAGAGTCAGTTCCAGACCCTCAACCTCTCGGAG |
| | | AACCTCCAGCAGCGCCCTCTGGTGTGGGACCTAATACAAT |
| | | GGCTGCAGGCGGTGGCGCACCAATGGCAGACAATAACGAA |
| | | GGCGCCGACGGAGTGGGTAGTTCCTCGGGAAATTGGCATT |
| | | GCGATTCCACATGGCTGGGCGACAGAGTCATCACCACCAG |
| | | CACCCGAACCTGGGCCCTGCCCACCTACAACAACCACCTC |
| | | TACAAGCAAATCTCCAACGGGACATCGGGAGGAGCCACCA |
| | | ACGACAACACCTACTTCGGCTACAGCACCCCCTGGGGGTA |
| | | TTTTGACTTTAACAGATTCCACTGCCACTTTTCACCACGT |
| | | GACTGGCAGCGACTCATCAACAACAACTGGGGATTCCGGC |
| | | CCAAGAGACTCAGCTTCAAGCTCTTCAACATCCAGGTCAA |
| | | GGAGGTCACGCAGAATGAAGGCACCAAGACCATCGCCAAT |
| | | AACCTCACCAGCACCATCCAGGTGTTTACGGACTCGGAGT |
| | | ACCAGCTGCCGTACGTTCTCGGCTCTGCCCACCAGGGCTG |
| | | CCTGCCTCCGTTCCCGGCGGACGTGTTCATGATTCCCCAG |
| | | TACGGCTACCTAACACTCAACAACGGTAGTCAGGCCGTGG |
| | | GACGCTCCTCCTTCTACTGCCTGGAATACTTTCCTTCGCA |
| | | GATGCTGAGAACCGGCAACAACTTCCAGTTTACTTACACC |
| | | TTCGAGGACGTGCCTTTCCACAGCAGCTACGCCCACAGCC |
| | | AGAGCTTGGACCGGCTGATGAATCCTCTGATTGACCAGTA |
| | | CCTGTACTACTTGTCTCGGACTCAAACAACAGGAGGCACG |
| | | GCAAATACGCAGACTCTGGGCTTCAGCCAAGGTGGGCCTA |
| | | ATACAATGGCCAATCAGGCAAAGAACTGGCTGCCAGGACC |
| | | CTGTTACCGCCAACAACGCGTCTCAACGACAACCGGGCAA |
| | | AACAACAATAGCAACTTTGCCTGGACTGCTGGGACCAAAT |
| | | ACCATCTGAATGGAAGAAATTCATTGGCTAATCCTGGCAT |
| | | CGCTATGGCAACACACAAAGACGACGAGGAGCGTTTTTTT |
| | | CCCAGTAACGGGATCCTGATTTTTGGCAAACAAAATGCTG |
| | | CCAGAGACAATGCGGATTACAGCGATGTCATGCTCACCAG |
| | | CGAGGAAGAAATCAAAACCACTAACCCTGTGGCTACAGAG |
| | | GAATACGGTATCGTGGCAGATAACTTGCAGCAGCAAAACA |
| | | CGGCTCCTCAAATTGGAACTGTCAACAGCCAGGGGGCCTT |
| | | ACCCGGTATGGTCTGGCAGAACCGGGACGTGTACCTGCAG |
| | | GGTCCCATCTGGGCCAAGATTCCTCACACGGACGGCAACT |
| | | TCCACCCGTCTCCGCTGATGGGCGGCTTTGGCCTGAAACA |
| | | TCCTCCGCCTCAGATCCTGATCAAGAACACGCCTGTACCT |
| | | GCGGATCCTCCGACCACCTTCAACCAGTCAAAGCTGAACT |
| | | CTTTCATCACGCAATACAGCACCGGACAGGTCAGCGTGGA |
| | | AATTGAATGGGAGCTGCAGAAGGAAAACAGCAAGCGCTGG |
| | | AACCCCGAGATCCAGTACACCTCCAACTACTACAAATCTA |
| | | CAAGTGTGGACTTTGCTGTTAATACAGAAGGCGTGTACTC |
| | | TGAACCCCGCCCCATTGGCACCCGTTACCTCACCCGTAAT |
| | | CTGTAA |
| 17 | AAV8 VP1u region | MAADGYLPDWLEDNLSEGIREWWALKPGAPKPKANQQKQD |
| | (protein) | DGRGLVLPGYKYLGPFNGLDKGEPVNAADAAALEHDKAYD |
| | | QQLQAGDNPYLRYNHADAEFQERLQEDTSFGGNLGRAVFQ |
| | | AKKRVLEPLGLVEEGAK |
| 18 | AAV8 VP1u region | ATGGCTGCCGATGGTTATCTTCCAGATTGGCTCGAGGACA |
| | (DNA) | ACCTCTCTGAGGGCATTCGCGAGTGGTGGGCGCTGAAACC |
| | | TGGAGCCCCGAAGCCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGACGCAGCGGCCCTCGAGCACGACAAGGCCTACGAC |
| | | CAGCAGCTGCAGGCGGGTGACAATCCGTACCTGCGGTATA |
| | | ACCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGCGGGTTCTCGAACCTCTCGGTCTGGTTGAGG |
| | | AAGGCGCTAAG |
| 19 | AAV8 VP1 capsid | MAADGYLPDWLEDNLSEGIREWWALKPGAPKPKANQQKQD |
| | protein | DGRGLVLPGYKYLGPFNGLDKGEPVNAADAAALEHDKAYD |
| | | QQLQAGDNPYLRYNHADAEFQERLQEDTSFGGNLGRAVFQ |
| | | AKKRVLEPLGLVEEGAKTAPGKKRPVEPSPQRSPDSSTGI |
| | | GKKGQQPARKRLNFGQTGDSESVPDPQPLGEPPAAPSGVG |
| | | PNTMAAGGGAPMADNNEGADGVGSSSGNWHCDSTWLGDRV |
| | | ITTSTRTWALPTYNNHLYKQISNGTSGGATNDNTYFGYST |
| | | PWGYFDFNRFHCHFSPRDWQRLINNNWGFRPKRLSFKLFN |
| | | IQVKEVTQNEGTKTIANNLTSTIQVFTDSEYQLPYVLGSA |
| | | HQGCLPPFPADVFMIPQYGYLTLNNGSQAVGRSSFYCLEY |
| | | FPSQMLRTGNNFQFTYTFEDVPFHSSYAHSQSLDRLMNPL |
| | | IDQYLYYLSRTQTTGGTANTQTLGFSQGGPNTMANQAKNW |
| | | LPGPCYRQQRVSTTTGQNNNSNFAWTAGTKYHLNGRNSLA |
| | | NPGIAMATHKDDEERFFPSNGILIFGKQNAARDNADYSDV |
| | | MLTSEEEIKTTNPVATEEYGIVADNLQQQNTAPQIGTVNS |
| | | QGALPGMVWQNRDVYLQGPIWAKIPHTDGNFHPSPLMGGF |
| | | GLKHPPPQILIKNTPVPADPPTTFNQSKLNSFITQYSTGQ |
| | | VSVEIEWELQKENSKRWNPEIQYTSNYYKSTSVDFAVNTE |
| | | GVYSEPRPIGTRYLTRNL |
| 20 | AAV8 VP1 capsid | ATGGCTGCCGATGGTTATCTTCCAGATTGGCTCGAGGACA |
| | DNA | ACCTCTCTGAGGGCATTCGCGAGTGGTGGGCGCTGAAACC |
| | | TGGAGCCCCGAAGCCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGACGCAGCGGCCCTCGAGCACGACAAGGCCTACGAC |
| | | CAGCAGCTGCAGGCGGGTGACAATCCGTACCTGCGGTATA |
| | | ACCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGCGGGTTCTCGAACCTCTCGGTCTGGTTGAGG |
| | | AAGGCGCTAAGACGGCTCCTGGAAAGAAGAGACCGGTAGA |
| | | GCCATCACCCCAGCGTTCTCCAGACTCCTCTACGGGCATC |
| | | GGCAAGAAAGGCCAACAGCCCGCCAGAAAAAGACTCAATT |
| | | TTGGTCAGACTGGCGACTCAGAGTCAGTTCCAGACCCTCA |
| | | ACCTCTCGGAGAACCTCCAGCAGCGCCCTCTGGTGTGGGA |
| | | CCTAATACAATGGCTGCAGGCGGTGGCGCACCAATGGCAG |
| | | ACAATAACGAAGGCGCCGACGGAGTGGGTAGTTCCTCGGG |
| | | AAATTGGCATTGCGATTCCACATGGCTGGGCGACAGAGTC |
| | | ATCACCACCAGCACCCGAACCTGGGCCCTGCCCACCTACA |
| | | ACAACCACCTCTACAAGCAAATCTCCAACGGGACATCGGG |
| | | AGGAGCCACCAACGACAACACCTACTTCGGCTACAGCACC |
| | | CCCTGGGGGTATTTTGACTTTAACAGATTCCACTGCCACT |
| | | TTTCACCACGTGACTGGCAGCGACTCATCAACAACAACTG |
| | | GGGATTCCGGCCCAAGAGACTCAGCTTCAAGCTCTTCAAC |
| | | ATCCAGGTCAAGGAGGTCACGCAGAATGAAGGCACCAAGA |
| | | CCATCGCCAATAACCTCACCAGCACCATCCAGGTGTTTAC |
| | | GGACTCGGAGTACCAGCTGCCGTACGTTCTCGGCTCTGCC |
| | | CACCAGGGCTGCCTGCCTCCGTTCCCGGCGGACGTGTTCA |
| | | TGATTCCCCAGTACGGCTACCTAACACTCAACAACGGTAG |
| | | TCAGGCCGTGGGACGCTCCTCCTTCTACTGCCTGGAATAC |
| | | TTTCCTTCGCAGATGCTGAGAACCGGCAACAACTTCCAGT |
| | | TTACTTACACCTTCGAGGACGTGCCTTTCCACAGCAGCTA |
| | | CGCCCACAGCCAGAGCTTGGACCGGCTGATGAATCCTCTG |
| | | ATTGACCAGTACCTGTACTACTTGTCTCGGACTCAAACAA |
| | | CAGGAGGCACGGCAAATACGCAGACTCTGGGCTTCAGCCA |
| | | AGGTGGGCCTAATACAATGGCCAATCAGGCAAAGAACTGG |
| | | CTGCCAGGACCCTGTTACCGCCAACAACGCGTCTCAACGA |
| | | CAACCGGGCAAAACAACAATAGCAACTTTGCCTGGACTGC |
| | | TGGGACCAAATACCATCTGAATGGAAGAAATTCATTGGCT |
| | | AATCCTGGCATCGCTATGGCAACACACAAAGACGACGAGG |
| | | AGCGTTTTTTTCCCAGTAACGGGATCCTGATTTTTGGCAA |
| | | ACAAAATGCTGCCAGAGACAATGCGGATTACAGCGATGTC |
| | | ATGCTCACCAGCGAGGAAGAAATCAAAACCACTAACCCTG |
| | | TGGCTACAGAGGAATACGGTATCGTGGCAGATAACTTGCA |
| | | GCAGCAAAACACGGCTCCTCAAATTGGAACTGTCAACAGC |
| | | CAGGGGGCCTTACCCGGTATGGTCTGGCAGAACCGGGACG |
| | | TGTACCTGCAGGGTCCCATCTGGGCCAAGATTCCTCACAC |
| | | GGACGGCAACTTCCACCCGTCTCCGCTGATGGGCGGCTTT |
| | | GGCCTGAAACATCCTCCGCCTCAGATCCTGATCAAGAACA |
| | | CGCCTGTACCTGCGGATCCTCCGACCACCTTCAACCAGTC |
| | | AAAGCTGAACTCTTTCATCACGCAATACAGCACCGGACAG |
| | | GTCAGCGTGGAAATTGAATGGGAGCTGCAGAAGGAAAACA |
| | | GCAAGCGCTGGAACCCCGAGATCCAGTACACCTCCAACTA |
| | | CTACAAATCTACAAGTGTGGACTTTGCTGTTAATACAGAA |
| | | GGCGTGTACTCTGAACCCCGCCCCATTGGCACCCGTTACC |
| | | TCACCCGTAATCTGTAA |
| 21 | AAV5-AAV8 | TAPTGKRIDDHFPKRKKARTEEDSKPSTSSDAEAGPSGSQ |
| | hybrid VP2 capsid | QLQIPAQPASSLGADTMAAGGGAPMADNNEGADGVGSSSG |
| | protein | NWHCDSTWLGDRVITTSTRTWALPTYNNHLYKQISNGTSG |
| | | GATNDNTYFGYSTPWGYFDFNRFHCHFSPRDWQRLINNNW |
| | | GFRPKRLSFKLFNIQVKEVTQNEGTKTIANNLTSTIQVFT |
| | | DSEYQLPYVLGSAHQGCLPPFPADVFMIPQYGYLTLNNGS |
| | | QAVGRSSFYCLEYFPSQMLRTGNNFQFTYTFEDVPFHSSY |
| | | AHSQSLDRLMNPLIDQYLYYLSRTQTTGGTANTQTLGFSQ |
| | | GGPNTMANQAKNWLPGPCYRQQRVSTTTGQNNNSNFAWTA |
| | | GTKYHLNGRNSLANPGIAMATHKDDEERFFPSNGILIFGK |
| | | QNAARDNADYSDVMLTSEEEIKTTNPVATEEYGIVADNLQ |
| | | QQNTAPQIGTVNSQGALPGMVWQNRDVYLQGPIWAKIPHT |
| | | DGNFHPSPLMGGFGLKHPPPQILIKNTPVPADPPTTFNQS |
| | | KLNSFITQYSTGQVSVEIEWELQKENSKRWNPEIQYTSNY |
| | | YKSTSVDFAVNTEGVYSEPRPIGTRYLTRNL |
| 22 | AAV5-AAV8 | ACGGCCCCTACCGGAAAGCGGATAGACGACCACTTTCCAA |
| | hybrid VP2 capsid | AAAGAAAGAAGGCTCGGACCGAAGAGGACTCCAAGCCTTC |
| | DNA | CACCTCGTCAGACGCCGAAGCTGGACCCAGCGGATCCCAG |
| | | CAGCTGCAAATCCCAGCCCAACCAGCCTCAAGTTTGGGAG |
| | | CTGATACAATGGCTGCAGGCGGTGGCGCACCAATGGCAGA |
| | | CAATAACGAAGGCGCCGACGGAGTGGGTAGTTCCTCGGGA |
| | | AATTGGCATTGCGATTCCACATGGCTGGGCGACAGAGTCA |
| | | TCACCACCAGCACCCGAACCTGGGCCCTGCCCACCTACAA |
| | | CAACCACCTCTACAAGCAAATCTCCAACGGGACATCGGGA |
| | | GGAGCCACCAACGACAACACCTACTTCGGCTACAGCACCC |
| | | CCTGGGGGTATTTTGACTTTAACAGATTCCACTGCCACTT |
| | | TTCACCACGTGACTGGCAGCGACTCATCAACAACAACTGG |
| | | GGATTCCGGCCCAAGAGACTCAGCTTCAAGCTCTTCAACA |
| | | TCCAGGTCAAGGAGGTCACGCAGAATGAAGGCACCAAGAC |
| | | CATCGCCAATAACCTCACCAGCACCATCCAGGTGTTTACG |
| | | GACTCGGAGTACCAGCTGCCGTACGTTCTCGGCTCTGCCC |
| | | ACCAGGGCTGCCTGCCTCCGTTCCCGGCGGACGTGTTCAT |
| | | GATTCCCCAGTACGGCTACCTAACACTCAACAACGGTAGT |
| | | CAGGCCGTGGGACGCTCCTCCTTCTACTGCCTGGAATACT |
| | | TTCCTTCGCAGATGCTGAGAACCGGCAACAACTTCCAGTT |
| | | TACTTACACCTTCGAGGACGTGCCTTTCCACAGCAGCTAC |
| | | GCCCACAGCCAGAGCTTGGACCGGCTGATGAATCCTCTGA |
| | | TTGACCAGTACCTGTACTACTTGTCTCGGACTCAAACAAC |
| | | AGGAGGCACGGCAAATACGCAGACTCTGGGCTTCAGCCAA |
| | | GGTGGGCCTAATACAATGGCCAATCAGGCAAAGAACTGGC |
| | | TGCCAGGACCCTGTTACCGCCAACAACGCGTCTCAACGAC |
| | | AACCGGGCAAAACAACAATAGCAACTTTGCCTGGACTGCT |
| | | GGGACCAAATACCATCTGAATGGAAGAAATTCATTGGCTA |
| | | ATCCTGGCATCGCTATGGCAACACACAAAGACGACGAGGA |
| | | GCGTTTTTTTCCCAGTAACGGGATCCTGATTTTTGGCAAA |
| | | CAAAATGCTGCCAGAGACAATGCGGATTACAGCGATGTCA |
| | | TGCTCACCAGCGAGGAAGAAATCAAAACCACTAACCCTGT |
| | | GGCTACAGAGGAATACGGTATCGTGGCAGATAACTTGCAG |
| | | CAGCAAAACACGGCTCCTCAAATTGGAACTGTCAACAGCC |
| | | AGGGGGCCTTACCCGGTATGGTCTGGCAGAACCGGGACGT |
| | | GTACCTGCAGGGTCCCATCTGGGCCAAGATTCCTCACACG |
| | | GACGGCAACTTCCACCCGTCTCCGCTGATGGGCGGCTTTG |
| | | GCCTGAAACATCCTCCGCCTCAGATCCTGATCAAGAACAC |
| | | GCCTGTACCTGCGGATCCTCCGACCACCTTCAACCAGTCA |
| | | AAGCTGAACTCTTTCATCACGCAATACAGCACCGGACAGG |
| | | TCAGCGTGGAAATTGAATGGGAGCTGCAGAAGGAAAACAG |
| | | CAAGCGCTGGAACCCCGAGATCCAGTACACCTCCAACTAC |
| | | TACAAATCTACAAGTGTGGACTTTGCTGTTAATACAGAAG |
| | | GCGTGTACTCTGAACCCCGCCCCATTGGCACCCGTTACCT |
| | | CACCCGTAATCTGTAA |
| 23 | AAV5-AAV8 | MSFVDHPPDWLEEVGEGLREFLGLEAGPPKPKPNQQHQDQ |
| | hybrid VP1 capsid | ARGLVLPGYNYLGPGNGLDRGEPVNRADEVAREHDISYNE |
| | protein | QLEAGDNPYLKYNHADAEFQEKLADDTSFGGNLGKAVFQA |
| | | KKRVLEPFGLVEEGAKTAPTGKRIDDHFPKRKKARTEEDS |
| | | KPSTSSDAEAGPSGSQQLQIPAQPASSLGADTMAAGGGAP |
| | | MADNNEGADGVGSSSGNWHCDSTWLGDRVITTSTRTWALP |
| | | TYNNHLYKQISNGTSGGATNDNTYFGYSTPWGYFDFNRFH |
| | | CHFSPRDWQRLINNNWGFRPKRLSFKLFNIQVKEVTQNEG |
| | | TKTIANNLTSTIQVFTDSEYQLPYVLGSAHQGCLPPFPAD |
| | | VFMIPQYGYLTLNNGSQAVGRSSFYCLEYFPSQMLRTGNN |
| | | FQFTYTFEDVPFHSSYAHSQSLDRLMNPLIDQYLYYLSRT |
| | | QTTGGTANTQTLGFSQGGPNTMANQAKNWLPGPCYRQQRV |
| | | STTTGQNNNSNFAWTAGTKYHLNGRNSLANPGIAMATHKD |
| | | DEERFFPSNGILIFGKQNAARDNADYSDVMLTSEEEIKTT |
| | | NPVATEEYGIVADNLQQQNTAPQIGTVNSQGALPGMVWQN |
| | | RDVYLQGPIWAKIPHTDGNFHPSPLMGGFGLKHPPPQILI |
| | | KNTPVPADPPTTFNQSKLNSFITQYSTGQVSVEIEWELQK |
| | | ENSKRWNPEIQYTSNYYKSTSVDFAVNTEGVYSEPRPIGT |
| | | RYLTRNL |
| 24 | AAV5-AAV8 | ATGTCTTTTGTTGATCACCCTCCAGATTGGTTGGAAGAAG |
| | hybrid VP1 capsid | TTGGTGAAGGTCTTCGCGAGTTTTTGGGCCTTGAAGCGGG |
| | DNA | CCCACCGAAACCAAAACCCAATCAGCAGCATCAAGATCAA |
| | | GCCCGTGGTCTTGTGCTGCCTGGTTATAACTATCTCGGAC |
| | | CCGGAAACGGTCTCGATCGAGGAGAGCCTGTCAACAGGGC |
| | | AGACGAGGTCGCGCGAGAGCACGACATCTCGTACAACGAG |
| | | CAGCTTGAGGCGGGAGACAACCCCTACCTCAAGTACAACC |
| | | ACGCGGACGCCGAGTTTCAGGAGAAGCTCGCCGACGACAC |
| | | ATCCTTCGGGGGAAACCTCGGAAAGGCAGTCTTTCAGGCC |
| | | AAGAAAAGGGTTCTCGAACCTTTTGGCCTGGTTGAAGAGG |
| | | GTGCTAAGACGGCCCCTACCGGAAAGCGGATAGACGACCA |
| | | CTTTCCAAAAAGAAAGAAGGCTCGGACCGAAGAGGACTCC |
| | | AAGCCTTCCACCTCGTCAGACGCCGAAGCTGGACCCAGCG |
| | | GATCCCAGCAGCTGCAAATCCCAGCCCAACCAGCCTCAAG |
| | | TTTGGGAGCTGATACAATGGCTGCAGGCGGTGGCGCACCA |
| | | ATGGCAGACAATAACGAAGGCGCCGACGGAGTGGGTAGTT |
| | | CCTCGGGAAATTGGCATTGCGATTCCACATGGCTGGGCGA |
| | | CAGAGTCATCACCACCAGCACCCGAACCTGGGCCCTGCCC |
| | | ACCTACAACAACCACCTCTACAAGCAAATCTCCAACGGGA |
| | | CATCGGGAGGAGCCACCAACGACAACACCTACTTCGGCTA |
| | | CAGCACCCCCTGGGGGTATTTTGACTTTAACAGATTCCAC |
| | | TGCCACTTTTCACCACGTGACTGGCAGCGACTCATCAACA |
| | | ACAACTGGGGATTCCGGCCCAAGAGACTCAGCTTCAAGCT |
| | | CTTCAACATCCAGGTCAAGGAGGTCACGCAGAATGAAGGC |
| | | ACCAAGACCATCGCCAATAACCTCACCAGCACCATCCAGG |
| | | TGTTTACGGACTCGGAGTACCAGCTGCCGTACGTTCTCGG |
| | | CTCTGCCCACCAGGGCTGCCTGCCTCCGTTCCCGGCGGAC |
| | | GTGTTCATGATTCCCCAGTACGGCTACCTAACACTCAACA |
| | | ACGGTAGTCAGGCCGTGGGACGCTCCTCCTTCTACTGCCT |
| | | GGAATACTTTCCTTCGCAGATGCTGAGAACCGGCAACAAC |
| | | TTCCAGTTTACTTACACCTTCGAGGACGTGCCTTTCCACA |
| | | GCAGCTACGCCCACAGCCAGAGCTTGGACCGGCTGATGAA |
| | | TCCTCTGATTGACCAGTACCTGTACTACTTGTCTCGGACT |
| | | CAAACAACAGGAGGCACGGCAAATACGCAGACTCTGGGCT |
| | | TCAGCCAAGGTGGGCCTAATACAATGGCCAATCAGGCAAA |
| | | GAACTGGCTGCCAGGACCCTGTTACCGCCAACAACGCGTC |
| | | TCAACGACAACCGGGCAAAACAACAATAGCAACTTTGCCT |
| | | GGACTGCTGGGACCAAATACCATCTGAATGGAAGAAATTC |
| | | ATTGGCTAATCCTGGCATCGCTATGGCAACACACAAAGAC |
| | | GACGAGGAGCGTTTTTTTCCCAGTAACGGGATCCTGATTT |
| | | TTGGCAAACAAAATGCTGCCAGAGACAATGCGGATTACAG |
| | | CGATGTCATGCTCACCAGCGAGGAAGAAATCAAAACCACT |
| | | AACCCTGTGGCTACAGAGGAATACGGTATCGTGGCAGATA |
| | | ACTTGCAGCAGCAAAACACGGCTCCTCAAATTGGAACTGT |
| | | CAACAGCCAGGGGGCCTTACCCGGTATGGTCTGGCAGAAC |
| | | CGGGACGTGTACCTGCAGGGTCCCATCTGGGCCAAGATTC |
| | | CTCACACGGACGGCAACTTCCACCCGTCTCCGCTGATGGG |
| | | CGGCTTTGGCCTGAAACATCCTCCGCCTCAGATCCTGATC |
| | | AAGAACACGCCTGTACCTGCGGATCCTCCGACCACCTTCA |
| | | ACCAGTCAAAGCTGAACTCTTTCATCACGCAATACAGCAC |
| | | CGGACAGGTCAGCGTGGAAATTGAATGGGAGCTGCAGAAG |
| | | GAAAACAGCAAGCGCTGGAACCCCGAGATCCAGTACACCT |
| | | CCAACTACTACAAATCTACAAGTGTGGACTTTGCTGTTAA |
| | | TACAGAAGGCGTGTACTCTGAACCCCGCCCCATTGGCACC |
| | | CGTTACCTCACCCGTAATCTGTAA |
| 25 | AAV8/5-AAV8 | MAADGYLPDWLEDNLSEGIREWWALKPGAPKPKANQQKQD |
| | hybrid VP1 capsid | DGRGLVLPGYKYLGPFNGLDKGEPVNAADAAALEHDKAYD |
| | protein | QQLQAGDNPYLRYNHADAEFQERLQEDTSFGGNLGRAVFQ |
| | | AKKRVLEPLGLVEEGAKTAPTGKRIDDHFPKRKKARTEED |
| | | SKPSTSSDAEAGPSGSQQLQIPAQPASSLGADTMAAGGGA |
| | | PMADNNEGADGVGSSSGNWHCDSTWLGDRVITTSTRTWAL |
| | | PTYNNHLYKQISNGTSGGATNDNTYFGYSTPWGYFDFNRF |
| | | HCHFSPRDWQRLINNNWGFRPKRLSFKLFNIQVKEVTQNE |
| | | GTKTIANNLTSTIQVFTDSEYQLPYVLGSAHQGCLPPFPA |
| | | DVFMIPQYGYLTLNNGSQAVGRSSFYCLEYFPSQMLRTGN |
| | | NFQFTYTFEDVPFHSSYAHSQSLDRLMNPLIDQYLYYLSR |
| | | TQTTGGTANTQTLGFSQGGPNTMANQAKNWLPGPCYRQQR |
| | | VSTTTGQNNNSNFAWTAGTKYHLNGRNSLANPGIAMATHK |
| | | DDEERFFPSNGILIFGKQNAARDNADYSDVMLTSEEEIKT |
| | | TNPVATEEYGIVADNLQQQNTAPQIGTVNSQGALPGMVWQ |
| | | NRDVYLQGPIWAKIPHTDGNFHPSPLMGGFGLKHPPPQIL |
| | | IKNTPVPADPPTTFNQSKLNSFITQYSTGQVSVEIEWELQ |
| | | KENSKRWNPEIQYTSNYYKSTSVDFAVNTEGVYSEPRPIG |
| | | TRYLTRNL |
| 26 | AAV8/5-AAV8 | ATGGCTGCCGATGGTTATCTTCCAGATTGGCTCGAGGACA |
| | hybrid VP1 capsid | ACCTCTCTGAGGGCATTCGCGAGTGGTGGGCGCTGAAACC |
| | DNA | TGGAGCCCCGAAGCCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGACGCAGCGGCCCTCGAGCACGACAAGGCCTACGAC |
| | | CAGCAGCTGCAGGCGGGTGACAATCCGTACCTGCGGTATA |
| | | ACCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGCGGGTTCTCGAACCTCTCGGTCTGGTTGAGG |
| | | AAGGCGCTAAGACGGCCCCTACCGGAAAGCGGATAGACGA |
| | | CCACTTTCCAAAAAGAAAGAAGGCTCGGACCGAAGAGGAC |
| | | TCCAAGCCTTCCACCTCGTCAGACGCCGAAGCTGGACCCA |
| | | GCGGATCCCAGCAGCTGCAAATCCCAGCCCAACCAGCCTC |
| | | AAGTTTGGGAGCTGATACAATGGCTGCAGGCGGTGGCGCA |
| | | CCAATGGCAGACAATAACGAAGGCGCCGACGGAGTGGGTA |
| | | GTTCCTCGGGAAATTGGCATTGCGATTCCACATGGCTGGG |
| | | CGACAGAGTCATCACCACCAGCACCCGAACCTGGGCCCTG |
| | | CCCACCTACAACAACCACCTCTACAAGCAAATCTCCAACG |
| | | GGACATCGGGAGGAGCCACCAACGACAACACCTACTTCGG |
| | | CTACAGCACCCCCTGGGGGTATTTTGACTTTAACAGATTC |
| | | CACTGCCACTTTTCACCACGTGACTGGCAGCGACTCATCA |
| | | ACAACAACTGGGGATTCCGGCCCAAGAGACTCAGCTTCAA |
| | | GCTCTTCAACATCCAGGTCAAGGAGGTCACGCAGAATGAA |
| | | GGCACCAAGACCATCGCCAATAACCTCACCAGCACCATCC |
| | | AGGTGTTTACGGACTCGGAGTACCAGCTGCCGTACGTTCT |
| | | CGGCTCTGCCCACCAGGGCTGCCTGCCTCCGTTCCCGGCG |
| | | GACGTGTTCATGATTCCCCAGTACGGCTACCTAACACTCA |
| | | ACAACGGTAGTCAGGCCGTGGGACGCTCCTCCTTCTACTG |
| | | CCTGGAATACTTTCCTTCGCAGATGCTGAGAACCGGCAAC |
| | | AACTTCCAGTTTACTTACACCTTCGAGGACGTGCCTTTCC |
| | | ACAGCAGCTACGCCCACAGCCAGAGCTTGGACCGGCTGAT |
| | | GAATCCTCTGATTGACCAGTACCTGTACTACTTGTCTCGG |
| | | ACTCAAACAACAGGAGGCACGGCAAATACGCAGACTCTGG |
| | | GCTTCAGCCAAGGTGGGCCTAATACAATGGCCAATCAGGC |
| | | AAAGAACTGGCTGCCAGGACCCTGTTACCGCCAACAACGC |
| | | GTCTCAACGACAACCGGGCAAAACAACAATAGCAACTTTG |
| | | CCTGGACTGCTGGGACCAAATACCATCTGAATGGAAGAAA |
| | | TTCATTGGCTAATCCTGGCATCGCTATGGCAACACACAAA |
| | | GACGACGAGGAGCGTTTTTTTCCCAGTAACGGGATCCTGA |
| | | TTTTTGGCAAACAAAATGCTGCCAGAGACAATGCGGATTA |
| | | CAGCGATGTCATGCTCACCAGCGAGGAAGAAATCAAAACC |
| | | ACTAACCCTGTGGCTACAGAGGAATACGGTATCGTGGCAG |
| | | ATAACTTGCAGCAGCAAAACACGGCTCCTCAAATTGGAAC |
| | | TGTCAACAGCCAGGGGGCCTTACCCGGTATGGTCTGGCAG |
| | | AACCGGGACGTGTACCTGCAGGGTCCCATCTGGGCCAAGA |
| | | TTCCTCACACGGACGGCAACTTCCACCCGTCTCCGCTGAT |
| | | GGGCGGCTTTGGCCTGAAACATCCTCCGCCTCAGATCCTG |
| | | ATCAAGAACACGCCTGTACCTGCGGATCCTCCGACCACCT |
| | | TCAACCAGTCAAAGCTGAACTCTTTCATCACGCAATACAG |
| | | CACCGGACAGGTCAGCGTGGAAATTGAATGGGAGCTGCAG |
| | | AAGGAAAACAGCAAGCGCTGGAACCCCGAGATCCAGTACA |
| | | CCTCCAACTACTACAAATCTACAAGTGTGGACTTTGCTGT |
| | | TAATACAGAAGGCGTGTACTCTGAACCCCGCCCCATTGGC |
| | | ACCCGTTACCTCACCCGTAATCTGTAA |
| 27 | AAVrh13 VP3 | MAAGGGAPMADNNEGADGVGNASGNWHCDSTWLGDRVITT |
| | capsid protein | STRTWALPTYNNHLYKQISSQSGATNDNHFFGYSTPWGYF |
| | | DFNRFHCHFSPRDWQRLINNNWGFRPRKLRFKLFNIQVKE |
| | | VTTNDGVTTIANNLTSTIQVFSDSEYQLPYVLGSAHQGCL |
| | | PPFPADVFMIPQYGYLTLNNGSQSVGRSSFYCLEYFPSQM |
| | | LRTGNNFEFSYTFEEVPFHSSYAHSQSLDRLMNPLIDQYL |
| | | YYLARTQSTTGSTRELQFHQAGPNTMAEQSKNWLPGPCYR |
| | | QQRLSKNIDSNNNSNFAWTGATKYHLNGRNSLTNPGVAMA |
| | | TNKDDEDQFFPINGVLVFGETGAANKTTLENVLMTSEEEI |
| | | KTTNPVATEEYGVVSSNLQSSTAGPQTQTVNSQGALPGMV |
| | | WQNRDVYLQGPIWAKIPHTDGNFHPSPLMGGFGLKHPPPQ |
| | | ILIKNTPVPANPPEVFTPAKFASFITQYSTGQVSVEIEWE |
| | | LQKENSKRWNPEIQYTSNYAKSNNVEFAVNNEGVYTEPRP |
| | | IGTRYLTRNL |
| 28 | AAVrh13 VP3 | ATGGCTGCAGGCGGTGGCGCACCAATGGCAGACAATAACG |
| | capsid DNA | AAGGCGCCGACGGAGTGGGTAATGCCTCCGGAAATTGGCA |
| | | TTGCGATTCCACATGGCTGGGCGACAGAGTCATCACCACC |
| | | AGCACCCGCACCTGGGCCCTGCCCACCTACAACAACCACC |
| | | TCTACAAGCAGATATCAAGTCAGAGCGGGGCTACCAACGA |
| | | CAACCACTTCTTCGGCTACAGCACCCCCTGGGGCTATTTT |
| | | GACTTCAACAGATTCCACTGCCACTTCTCACCACGTGACT |
| | | GGCAGCGACTCATCAACAACAACTGGGGATTCCGGCCCAG |
| | | AAAGCTGCGGTTCAAGTTGTTCAACATCCAGGTCAAGGAG |
| | | GTCACGACGAACGACGGCGTTACGACCATCGCTAATAACC |
| | | TTACCAGCACGATTCAGGTCTTCTCGGACTCGGAGTACCA |
| | | ACTGCCGTACGTCCTCGGCTCTGCGCACCAGGGCTGCCTC |
| | | CCTCCGTTCCCTGCGGACGTGTTCATGATTCCTCAGTACG |
| | | GATATCTGACTCTAAACAACGGCAGTCAGTCTGTGGGACG |
| | | TTCCTCCTTCTACTGCCTGGAGTACTTTCCTTCTCAGATG |
| | | CTGAGAACGGGCAATAACTTTGAATTCAGCTACACCTTTG |
| | | AGGAAGTGCCTTTCCACAGCAGCTATGCGCACAGCCAGAG |
| | | CCTGGACCGGCTGATGAATCCCCTCATCGACCAGTACCTG |
| | | TACTACCTGGCCCGGACCCAGAGCACTACGGGGTCCACAA |
| | | GGGAGCTGCAGTTCCATCAGGCTGGGCCCAACACCATGGC |
| | | CGAGCAATCAAAGAACTGGCTGCCCGGACCCTGTTATCGG |
| | | CAGCAGAGACTGTCAAAAAACATAGACAGCAACAACAACA |
| | | GTAACTTTGCCTGGACCGGGGCCACTAAATACCATCTGAA |
| | | TGGTAGAAATTCATTAACCAACCCGGGCGTAGCCATGGCC |
| | | ACCAACAAGGACGACGAGGACCAGTTCTTTCCCATCAACG |
| | | GAGTGCTGGTTTTTGGCGAAACGGGGGCTGCCAACAAGAC |
| | | AACGCTGGAAAACGTGCTAATGACCAGCGAGGAGGAGATC |
| | | AAAACCACCAATCCCGTGGCTACAGAAGAATACGGTGTGG |
| | | TCTCCAGCAACCTGCAATCGTCTACGGCCGGACCCCAGAC |
| | | ACAGACTGTCAACAGCCAGGGGGCTCTGCCCGGCATGGTC |
| | | TGGCAGAACCGGGACGTGTACCTGCAGGGTCCCATCTGGG |
| | | CCAAAATTCCTCACACGGACGGCAACTTTCACCCGTCTCC |
| | | CCTGATGGGCGGATTTGGACTCAAACACCCGCCTCCTCAA |
| | | ATTCTCATCAAAAACACCCCGGTACCTGCTAATCCTCCAG |
| | | AGGTGTTTACTCCTGCCAAGTTTGCCTCATTTATCACGCA |
| | | GTACAGCACCGGCCAGGTCAGCGTGGAGATCGAGTGGGAA |
| | | CTGCAGAAAGAAAACAGCAAACGCTGGAATCCAGAGATTC |
| | | AGTACACCTCAAATTATGCCAAGTCTAATAATGTGGAATT |
| | | TGCTGTCAACAACGAAGGGGTTTATACTGAGCCTCGCCCC |
| | | ATTGGCACCCGTTACCTCACCCGTAACCTGTAA |
| 29 | AAVhu32- | (AAVhu32 VP1u + AAVhu32 VP1/2s + AAVrh13 |
| | AAVrh13 hybrid | VP3) |
| | VP1 capsid protein | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMAAGGGAPMADNNEGADGVGNASGNWHCDSTWLGDRVI |
| | | TTSTRTWALPTYNNHLYKQISSQSGATNDNHFFGYSTPWG |
| | | YFDFNRFHCHFSPRDWQRLINNNWGFRPRKLRFKLFNIQV |
| | | KEVTTNDGVTTIANNLTSTIQVFSDSEYQLPYVLGSAHQG |
| | | CLPPFPADVFMIPQYGYLTLNNGSQSVGRSSFYCLEYFPS |
| | | QMLRTGNNFEFSYTFEEVPFHSSYAHSQSLDRLMNPLIDQ |
| | | YLYYLARTQSTTGSTRELQFHQAGPNTMAEQSKNWLPGPC |
| | | YRQQRLSKNIDSNNNSNFAWTGATKYHLNGRNSLTNPGVA |
| | | MATNKDDEDQFFPINGVLVFGETGAANKTTLENVLMTSEE |
| | | EIKTTNPVATEEYGVVSSNLQSSTAGPQTQTVNSQGALPG |
| | | MVWQNRDVYLQGPIWAKIPHTDGNFHPSPLMGGFGLKHPP |
| | | PQILIKNTPVPANPPEVFTPAKFASFITQYSTGQVSVEIE |
| | | WELQKENSKRWNPEIQYTSNYAKSNNVEFAVNNEGVYTEP |
| | | RPIGTRYLTRNL |
| 30 | AAVhu32- | (AAVhu32 VP1u + AAVhu32 VP1/2s + AAVrh13 |
| | AAVrh13 hybrid | VP3) |
| | VP1 capsid DNA | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTGCAGGCGGTGGCGCACCAATGGCAGACA |
| | | ATAACGAAGGCGCCGACGGAGTGGGTAATGCCTCCGGAAA |
| | | TTGGCATTGCGATTCCACATGGCTGGGCGACAGAGTCATC |
| | | ACCACCAGCACCCGCACCTGGGCCCTGCCCACCTACAACA |
| | | ACCACCTCTACAAGCAGATATCAAGTCAGAGCGGGGCTAC |
| | | CAACGACAACCACTTCTTCGGCTACAGCACCCCCTGGGGC |
| | | TATTTTGACTTCAACAGATTCCACTGCCACTTCTCACCAC |
| | | GTGACTGGCAGCGACTCATCAACAACAACTGGGGATTCCG |
| | | GCCCAGAAAGCTGCGGTTCAAGTTGTTCAACATCCAGGTC |
| | | AAGGAGGTCACGACGAACGACGGCGTTACGACCATCGCTA |
| | | ATAACCTTACCAGCACGATTCAGGTCTTCTCGGACTCGGA |
| | | GTACCAACTGCCGTACGTCCTCGGCTCTGCGCACCAGGGC |
| | | TGCCTCCCTCCGTTCCCTGCGGACGTGTTCATGATTCCTC |
| | | AGTACGGATATCTGACTCTAAACAACGGCAGTCAGTCTGT |
| | | GGGACGTTCCTCCTTCTACTGCCTGGAGTACTTTCCTTCT |
| | | CAGATGCTGAGAACGGGCAATAACTTTGAATTCAGCTACA |
| | | CCTTTGAGGAAGTGCCTTTCCACAGCAGCTATGCGCACAG |
| | | CCAGAGCCTGGACCGGCTGATGAATCCCCTCATCGACCAG |
| | | TACCTGTACTACCTGGCCCGGACCCAGAGCACTACGGGGT |
| | | CCACAAGGGAGCTGCAGTTCCATCAGGCTGGGCCCAACAC |
| | | CATGGCCGAGCAATCAAAGAACTGGCTGCCCGGACCCTGT |
| | | TATCGGCAGCAGAGACTGTCAAAAAACATAGACAGCAACA |
| | | ACAACAGTAACTTTGCCTGGACCGGGGCCACTAAATACCA |
| | | TCTGAATGGTAGAAATTCATTAACCAACCCGGGCGTAGCC |
| | | ATGGCCACCAACAAGGACGACGAGGACCAGTTCTTTCCCA |
| | | TCAACGGAGTGCTGGTTTTTGGCGAAACGGGGGCTGCCAA |
| | | CAAGACAACGCTGGAAAACGTGCTAATGACCAGCGAGGAG |
| | | GAGATCAAAACCACCAATCCCGTGGCTACAGAAGAATACG |
| | | GTGTGGTCTCCAGCAACCTGCAATCGTCTACGGCCGGACC |
| | | CCAGACACAGACTGTCAACAGCCAGGGGGCTCTGCCCGGC |
| | | ATGGTCTGGCAGAACCGGGACGTGTACCTGCAGGGTCCCA |
| | | TCTGGGCCAAAATTCCTCACACGGACGGCAACTTTCACCC |
| | | GTCTCCCCTGATGGGCGGATTTGGACTCAAACACCCGCCT |
| | | CCTCAAATTCTCATCAAAAACACCCCGGTACCTGCTAATC |
| | | CTCCAGAGGTGTTTACTCCTGCCAAGTTTGCCTCATTTAT |
| | | CACGCAGTACAGCACCGGCCAGGTCAGCGTGGAGATCGAG |
| | | TGGGAACTGCAGAAAGAAAACAGCAAACGCTGGAATCCAG |
| | | AGATTCAGTACACCTCAAATTATGCCAAGTCTAATAATGT |
| | | GGAATTTGCTGTCAACAACGAAGGGGTTTATACTGAGCCT |
| | | CGCCCCATTGGCACCCGTTACCTCACCCGTAACCTGTAA |
| 31 | AAVhu32 VP1 | MAADGYLPDWLEDTLSEGIRQWWKLKPGPPPPKPAERHKD |
| | region protein | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQAQAQTGWVQNQG |
| | | ILPGMVWQDRDVYLQGPIWAKIPHTDGNFHPSPLMGGFGM |
| | | KHPPPQILIKNTPVPADPPTAFNKDKLNSFITQYSTGQVS |
| | | VEIEWELQKENSKRWNPEIQYTSNYYKSNNVEFAVNTEGV |
| | | YSEPRPIGTRYLTRNL |
| 32 | AAVhu32 VP1 | ATGGCTGCCGATGGTTATCTTCCAGATTGGCTCGAGGACA |
| | region DNA | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAGCACAGGCGCAGACCGGCTGGGTTCAAAACCAAGGA |
| | | ATACTTCCGGGTATGGTTTGGCAGGACAGAGATGTGTACC |
| | | TGCAAGGACCCATTTGGGCCAAAATTCCTCACACGGACGG |
| | | CAACTTTCACCCTTCTCCGCTAATGGGAGGGTTTGGAATG |
| | | AAGCACCCGCCTCCTCAGATCCTCATCAAAAACACACCTG |
| | | TACCTGCGGATCCTCCAACGGCTTTCAATAAGGACAAGCT |
| | | GAACTCTTTCATCACCCAGTATTCTACTGGCCAAGTCAGC |
| | | GTGGAGATTGAGTGGGAGCTGCAGAAGGAAAACAGCAAGC |
| | | GCTGGAACCCGGAGATCCAGTACACTTCCAACTATTACAA |
| | | GTCTAATAATGTTGAATTTGCTGTTAATACTGAAGGTGTA |
| | | TATAGTGAACCCCGCCCCATTGGCACCAGATACCTGACTC |
| | | GTAATCTGTAA |
| 33 | AAV6/AAV8 | MAADGYLPDWLEDNLSEGIREWWDLKPGAPKPKANQQKQD |
| | VP1 aa | DGRGLVLPGYKYLGPFNGLDKGEPVNAADAAALEHDKAYD |
| | | QQLKAGDNPYLRYNHADAEFQERLQEDTSFGGNLGRAVFQ |
| | | AKKRVLEPFGLVEEGAKTAPGKKRPVEQSPQEPDSSSGIG |
| | | KTGQQPAKKRLNFGQTGDSESVPDPQPLGEPPATPAAVGP |
| | | TTMAAGGGAPMADNNEGADGVGSSSGNWHCDSTWLGDRVI |
| | | TTSTRTWALPTYNNHLYKQISNGTSGGATNDNTYFGYSTP |
| | | WGYFDFNRFHCHFSPRDWQRLINNNWGFRPKRLSFKLFNI |
| | | QVKEVTQNEGTKTIANNLTSTIQVFTDSEYQLPYVLGSAH |
| | | QGCLPPFPADVFMIPQYGYLTLNNGSQAVGRSSFYCLEYF |
| | | PSQMLRTGNNFQFTYTFEDVPFHSSYAHSQSLDRLMNPLI |
| | | DQYLYYLSRTQTTGGTANTQTLGFSQGGPNTMANQAKNWL |
| | | PGPCYRQQRVSTTTGQNNNSNFAWTAGTKYHLNGRNSLAN |
| | | PGIAMATHKDDEERFFPSNGILIFGKQNAARDNADYSDVM |
| | | LTSEEEIKTTNPVATEEYGIVADNLQQQNTAPQIGTVNSQ |
| | | GALPGMVWQNRDVYLQGPIWAKIPHTDGNFHPSPLMGGFG |
| | | LKHPPPQILIKNTPVPADPPTTFNQSKLNSFITQYSTGQV |
| | | SVEIEWELQKENSKRWNPEIQYTSNYYKSTSVDFAVNTEG |
| | | VYSEPRPIGTRYLTRNL |
| 34 | AAV6/AAV8 | ATGGCTGCCGATGGTTATCTTCCAGATTGGCTCGAGGACA |
| | VP1 dna | ACCTCTCTGAGGGCATTCGCGAGTGGTGGGACTTGAAACC |
| | | TGGAGCCCCGAAACCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGATGCAGCGGCCCTCGAGCACGACAAGGCCTACGAC |
| | | CAGCAGCTCAAAGCGGGTGACAATCCGTACCTGCGGTATA |
| | | ACCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGAGGGTTCTCGAACCTTTTGGTCTGGTTGAGG |
| | | AAGGTGCTAAGACGGCTCCTGGAAAGAAACGTCCGGTAGA |
| | | GCAGTCGCCACAAGAGCCAGACTCCTCCTCGGGCATTGGC |
| | | AAGACAGGCCAGCAGCCCGCTAAAAAGAGACTCAATTTTG |
| | | GTCAGACTGGCGACTCAGAGTCAGTCCCCGACCCACAACC |
| | | TCTCGGAGAACCTCCAGCAACCCCCGCTGCTGTGGGACCT |
| | | ACTACAATGGCTGCAGGCGGTGGCGCACCAATGGCAGACA |
| | | ATAACGAAGGCGCCGACGGAGTGGGTAGTTCCTCGGGAAA |
| | | TTGGCATTGCGATTCCACATGGCTGGGCGACAGAGTCATC |
| | | ACCACCAGCACCCGAACCTGGGCCCTGCCCACCTACAACA |
| | | ACCACCTCTACAAGCAAATCTCCAACGGGACATCGGGAGG |
| | | AGCCACCAACGACAACACCTACTTCGGCTACAGCACCCCC |
| | | TGGGGGTATTTTGACTTTAACAGATTCCACTGCCACTTTT |
| | | CACCACGTGACTGGCAGCGACTCATCAACAACAACTGGGG |
| | | ATTCCGGCCCAAGAGACTCAGCTTCAAGCTCTTCAACATC |
| | | CAGGTCAAGGAGGTCACGCAGAATGAAGGCACCAAGACCA |
| | | TCGCCAATAACCTCACCAGCACCATCCAGGTGTTTACGGA |
| | | CTCGGAGTACCAGCTGCCGTACGTTCTCGGCTCTGCCCAC |
| | | CAGGGCTGCCTGCCTCCGTTCCCGGCGGACGTGTTCATGA |
| | | TTCCCCAGTACGGCTACCTAACACTCAACAACGGTAGTCA |
| | | GGCCGTGGGACGCTCCTCCTTCTACTGCCTGGAATACTTT |
| | | CCTTCGCAGATGCTGAGAACCGGCAACAACTTCCAGTTTA |
| | | CTTACACCTTCGAGGACGTGCCTTTCCACAGCAGCTACGC |
| | | CCACAGCCAGAGCTTGGACCGGCTGATGAATCCTCTGATT |
| | | GACCAGTACCTGTACTACTTGTCTCGGACTCAAACAACAG |
| | | GAGGCACGGCAAATACGCAGACTCTGGGCTTCAGCCAAGG |
| | | TGGGCCTAATACAATGGCCAATCAGGCAAAGAACTGGCTG |
| | | CCAGGACCCTGTTACCGCCAACAACGCGTCTCAACGACAA |
| | | CCGGGCAAAACAACAATAGCAACTTTGCCTGGACTGCTGG |
| | | GACCAAATACCATCTGAATGGAAGAAATTCATTGGCTAAT |
| | | CCTGGCATCGCTATGGCAACACACAAAGACGACGAGGAGC |
| | | GTTTTTTTCCCAGTAACGGGATCCTGATTTTTGGCAAACA |
| | | AAATGCTGCCAGAGACAATGCGGATTACAGCGATGTCATG |
| | | CTCACCAGCGAGGAAGAAATCAAAACCACTAACCCTGTGG |
| | | CTACAGAGGAATACGGTATCGTGGCAGATAACTTGCAGCA |
| | | GCAAAACACGGCTCCTCAAATTGGAACTGTCAACAGCCAG |
| | | GGGGCCTTACCCGGTATGGTCTGGCAGAACCGGGACGTGT |
| | | ACCTGCAGGGTCCCATCTGGGCCAAGATTCCTCACACGGA |
| | | CGGCAACTTCCACCCGTCTCCGCTGATGGGCGGCTTTGGC |
| | | CTGAAACATCCTCCGCCTCAGATCCTGATCAAGAACACGC |
| | | CTGTACCTGCGGATCCTCCGACCACCTTCAACCAGTCAAA |
| | | GCTGAACTCTTTCATCACGCAATACAGCACCGGACAGGTC |
| | | AGCGTGGAAATTGAATGGGAGCTGCAGAAGGAAAACAGCA |
| | | AGCGCTGGAACCCCGAGATCCAGTACACCTCCAACTACTA |
| | | CAAATCTACAAGTGTGGACTTTGCTGTTAATACAGAAGGC |
| | | GTGTACTCTGAACCCCGCCCCATTGGCACCCGTTACCTCA |
| | | CCCGTAATCTGTAA |
| 35 | AAV6/AAVrh.21 | MAADGYLPDWLEDNLSEGIREWWDLKPGAPKPKANQQKQD |
| | VP1 aa | DGRGLVLPGYKYLGPFNGLDKGEPVNAADAAALEHDKAYD |
| | | QQLKAGDNPYLRYNHADAEFQERLQEDTSFGGNLGRAVFQ |
| | | AKKRVLEPLGLVEEGAKTAPGKKRPVEPSPQRSPDSSTGI |
| | | GKKGQQPARKRLNFGQTGDSESVPDPQPLGEPPAAPSSVG |
| | | SGTMAAGGGAPMADNNEGADGVGSSSGNWHCDSTWLGDRV |
| | | ITTSTRTWALPTYNNHLYKQISNGTSGGATNDNTYFGYST |
| | | PWGYFDFNRFHCHFSPRDWQRLINNNWGFRPKRLSFKLFN |
| | | IQVKEVTQNEGTKTIANNLTSTIQVFTDSEYQLPYVLGSA |
| | | HQGCLPPFPADVFMIPQYGYLTLNNGSQAVGRSSFYCLEY |
| | | FPSQMLRTGNNFQFTYTFEDVPFHSSYAHSQSLDRLMNPL |
| | | IDQYLYYLSRTQTTGGTANTQTLGFSQGGPNTMANQAKNW |
| | | LPGPCYRQQRVSTTTGQNNNSNFAWTAGTKYHLNGRNSLA |
| | | NPGIAMATHKDDEERFFPSNGILIFGKQNAARDNADYSDV |
| | | MLTSEEEIKTTNPVATEEYGIVADNLQQQNTAPQIGTVNS |
| | | QGALPGMVWQNRDVYLQGPIWAKIPHTDGNFHPSPLMGGF |
| | | GLKHPPPQILIKNTPVPADPPTTFNQSKLNSFITQYSTGQ |
| | | VSVEIEWELQKENSKRWNPEIQYTSNYYKSTSVDFAVNTE |
| | | GVYSEPRPIGTRYLTRNL |
| 36 | AAV6/AAVrh.21 | ATGGCTGCCGATGGTTATCTTCCAGATTGGCTCGAGGACA |
| | VP1 dna | ACCTCTCTGAGGGCATTCGCGAGTGGTGGGACCTGAAACC |
| | | TGGAGCCCCGAAACCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGACGCAGCGGCCCTCGAGCACGACAAGGCCTACGAC |
| | | CAGCAGCTCAAAGCGGGTGACAATCCGTACCTGCGGTATA |
| | | ATCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGCGGGTTCTCGAACCTCTCGGTCTGGTTGAGG |
| | | AAGGCGCTAAGACGGCTCCTGGAAAGAAGAGACCGGTAGA |
| | | GCCGTCACCACAGCGTTCCCCCGACTCCTCCACGGGCATC |
| | | GGCAAGAAAGGCCAGCAGCCCGCCAGAAAGAGACTCAATT |
| | | TCGGTCAGACTGGCGACTCAGAGTCAGTCCCCGACCCTCA |
| | | ACCTCTGGGAGAACCTCCAGCAGCGCCCTCTAGTGTGGGA |
| | | TCTGGTACAATGGCTGCAGGCGGTGGCGCACCAATGGCAG |
| | | ACAATAACGAAGGCGCCGACGGAGTGGGTAGTTCCTCGGG |
| | | AAATTGGCATTGCGATTCCACATGGCTGGGCGACAGAGTC |
| | | ATCACCACCAGCACCCGAACCTGGGCCCTGCCCACCTACA |
| | | ACAACCACCTCTACAAGCAAATCTCCAACGGGACATCGGG |
| | | AGGAGCCACCAACGACAACACCTACTTCGGCTACAGCACC |
| | | CCCTGGGGGTATTTTGACTTTAACAGATTCCACTGCCACT |
| | | TTTCACCACGTGACTGGCAGCGACTCATCAACAACAACTG |
| | | GGGATTCCGGCCCAAGAGACTCAGCTTCAAGCTCTTCAAC |
| | | ATCCAGGTCAAGGAGGTCACGCAGAATGAAGGCACCAAGA |
| | | CCATCGCCAATAACCTCACCAGCACCATCCAGGTGTTTAC |
| | | GGACTCGGAGTACCAGCTGCCGTACGTTCTCGGCTCTGCC |
| | | CACCAGGGCTGCCTGCCTCCGTTCCCGGCGGACGTGTTCA |
| | | TGATTCCCCAGTACGGCTACCTAACACTCAACAACGGTAG |
| | | TCAGGCCGTGGGACGCTCCTCCTTCTACTGCCTGGAATAC |
| | | TTTCCTTCGCAGATGCTGAGAACCGGCAACAACTTCCAGT |
| | | TTACTTACACCTTCGAGGACGTGCCTTTCCACAGCAGCTA |
| | | CGCCCACAGCCAGAGCTTGGACCGGCTGATGAATCCTCTG |
| | | ATTGACCAGTACCTGTACTACTTGTCTCGGACTCAAACAA |
| | | CAGGAGGCACGGCAAATACGCAGACTCTGGGCTTCAGCCA |
| | | AGGTGGGCCTAATACAATGGCCAATCAGGCAAAGAACTGG |
| | | CTGCCAGGACCCTGTTACCGCCAACAACGCGTCTCAACGA |
| | | CAACCGGGCAAAACAACAATAGCAACTTTGCCTGGACTGC |
| | | TGGGACCAAATACCATCTGAATGGAAGAAATTCATTGGCT |
| | | AATCCTGGCATCGCTATGGCAACACACAAAGACGACGAGG |
| | | AGCGTTTTTTTCCCAGTAACGGGATCCTGATTTTTGGCAA |
| | | ACAAAATGCTGCCAGAGACAATGCGGATTACAGCGATGTC |
| | | ATGCTCACCAGCGAGGAAGAAATCAAAACCACTAACCCTG |
| | | TGGCTACAGAGGAATACGGTATCGTGGCAGATAACTTGCA |
| | | GCAGCAAAACACGGCTCCTCAAATTGGAACTGTCAACAGC |
| | | CAGGGGGCCTTACCCGGTATGGTCTGGCAGAACCGGGACG |
| | | TGTACCTGCAGGGTCCCATCTGGGCCAAGATTCCTCACAC |
| | | GGACGGCAACTTCCACCCGTCTCCGCTGATGGGCGGCTTT |
| | | GGCCTGAAACATCCTCCGCCTCAGATCCTGATCAAGAACA |
| | | CGCCTGTACCTGCGGATCCTCCGACCACCTTCAACCAGTC |
| | | AAAGCTGAACTCTTTCATCACGCAATACAGCACCGGACAG |
| | | GTCAGCGTGGAAATTGAATGGGAGCTGCAGAAGGAAAACA |
| | | GCAAGCGCTGGAACCCCGAGATCCAGTACACCTCCAACTA |
| | | CTACAAATCTACAAGTGTGGACTTTGCTGTTAATACAGAA |
| | | GGCGTGTACTCTGAACCCCGCCCCATTGGCACCCGTTACC |
| | | TCACCCGTAATCTGTAA |
| 37 | AAV6/AAVrh.73 | MAADGYLPDWLEDNLSEGIREWWDLKPGAPKPKANQQKQD |
| | VP1 aa | DGRGLVLPGYKYLGPFNGLDKGEPVNAADAAALEHDKAYD |
| | | QQLKAGDNPYLRYNHADAEFQERLQEDTSFGGNLGRAVFQ |
| | | AKKRVLEPFGLVEEGAKTAPGKKRPVEQSPQEPDSSSGIG |
| | | KTGQQPAKKRLNFGQTGDSESVPDPQPLGEPPATPAAVGP |
| | | TTMAAGGGAPMADNNEGADGVGSSSGNWHCDSTWLGDRVI |
| | | TTSTRTWALPTYNNHLYKQISNGTSGGSTNDNTYFGYSTP |
| | | WGYFDFNRFHCHFSPRDWQRLINNNWGFRPKRLSFKLFNI |
| | | QVKEVTQNEGTKTIANNLTSTIQVFTDSEYQLPYVLGSAH |
| | | QGCLPPFPADVFMIPQYGYLTLNNGSQAVGRSSFYCLEYF |
| | | PSQMLRTGNNFSFSYTFEDVPFHSSYAHSQSLDRLMNPLI |
| | | DQYLYYLSRTQSTGGTAGTQQLLFSQAGPSNMSAQARNWL |
| | | PGPCYRQQRVSTTLSQNNNSNFAWTGATKYHLNGRDSLVN |
| | | PGVAMATNKDDEDRFFPSSGILMFGKQGAGKDNVDYSNVM |
| | | LTSEEEIKTTNPVATEQYGVVADNLQRQNTAPIVGAVNSQ |
| | | GALPGMVWQNRDVYLQGPIWAKIPHTDGNFHPSPLMGGFG |
| | | LKHPPPQILIKNTPVPADPPTAFNQAKLNSFITQYSTGQV |
| | | SVEIEWELQKENSKRWNPEIQYTSNYYKSTNVDFAVNTEG |
| | | VYSEPRPIGTRYLTRNL |
| 38 | AAV6/AAVrh.73 | ATGGCTGCCGATGGTTATCTTCCAGATTGGCTCGAGGACA |
| | VP1 dna | ACCTCTCTGAGGGCATTCGCGAGTGGTGGGACTTGAAACC |
| | | TGGAGCCCCGAAACCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGATGCAGCGGCCCTCGAGCACGACAAGGCCTACGAC |
| | | CAGCAGCTCAAAGCGGGTGACAATCCGTACCTGCGGTATA |
| | | ACCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGAGGGTTCTCGAACCTTTTGGTCTGGTTGAGG |
| | | AAGGTGCTAAGACGGCTCCTGGAAAGAAACGTCCGGTAGA |
| | | GCAGTCGCCACAAGAGCCAGACTCCTCCTCGGGCATTGGC |
| | | AAGACAGGCCAGCAGCCCGCTAAAAAGAGACTCAATTTTG |
| | | GTCAGACTGGCGACTCAGAGTCAGTCCCCGACCCACAACC |
| | | TCTCGGAGAACCTCCAGCAACCCCCGCTGCTGTGGGACCT |
| | | ACTACAATGGCTGCAGGCGGTGGCGCACCAATGGCAGACA |
| | | ATAACGAAGGTGCCGACGGAGTGGGTAGTTCCTCGGGAAA |
| | | TTGGCATTGCGATTCCACATGGCTGGGCGACAGAGTCATC |
| | | ACCACCAGCACCCGAACCTGGGCCCTGCCCACCTACAACA |
| | | ACCACCTCTACAAGCAAATCTCCAACGGGACCTCGGGAGG |
| | | CAGCACCAACGACAACACCTACTTTGGCTACAGCACCCCC |
| | | TGGGGGTATTTTGACTTTAACAGATTCCACTGCCACTTCT |
| | | CACCACGTGACTGGCAGCGACTCATCAACAACAACTGGGG |
| | | ATTCCGGCCCAAGAGACTCAGCTTCAAGCTCTTCAACATC |
| | | CAGGTCAAAGAGGTCACGCAGAATGAAGGCACCAAGACCA |
| | | TCGCCAATAACCTCACCAGCACCATCCAGGTGTTTACGGA |
| | | CTCGGAATACCAGCTGCCGTACGTCCTCGGCTCTGCCCAC |
| | | CAGGGCTGCCTGCCTCCGTTCCCGGCGGACGTCTTCATGA |
| | | TTCCTCAGTACGGCTACCTGACTCTCAACAACGGTAGTCA |
| | | GGCCGTGGGACGTTCCTCCTTCTACTGCCTGGAGTACTTC |
| | | CCCTCTCAGATGCTGAGAACGGGCAACAACTTTTCCTTCA |
| | | GCTACACTTTCGAGGACGTGCCTTTCCACAGCAGCTACGC |
| | | GCACAGCCAGAGTTTGGACAGGCTGATGAATCCTCTCATC |
| | | GACCAGTACCTGTACTACCTGTCAAGAACCCAGTCTACGG |
| | | GAGGCACAGCGGGAACCCAGCAGTTGCTGTTTTCTCAGGC |
| | | CGGGCCTAGCAACATGTCGGCTCAGGCCAGAAACTGGCTG |
| | | CCTGGACCCTGCTACAGACAGCAGCGCGTCTCCACGACAC |
| | | TGTCGCAAAACAACAACAGCAACTTTGCCTGGACTGGTGC |
| | | CACCAAGTATCATCTGAACGGCAGAGACTCTCTGGTGAAT |
| | | CCGGGCGTCGCCATGGCAACCAACAAGGACGACGAGGACC |
| | | GCTTCTTCCCATCCAGCGGCATCCTCATGTTTGGCAAGCA |
| | | GGGAGCTGGAAAAGACAACGTGGACTATAGCAACGTGATG |
| | | CTAACCAGCGAGGAAGAAATCAAGACCACCAACCCCGTGG |
| | | CCACAGAACAGTATGGCGTGGTGGCTGATAACCTACAGAG |
| | | ACAAAACACCGCTCCTATTGTGGGGGCCGTCAACAGCCAG |
| | | GGAGCCTTACCTGGCATGGTCTGGCAGAACCGGGACGTGT |
| | | ACCTGCAGGGTCCTATTTGGGCCAAGATTCCTCACACAGA |
| | | TGGCAACTTTCACCCGTCTCCTTTAATGGGCGGCTTTGGA |
| | | CTTAAACATCCGCCTCCTCAGATCCTCATCAAAAACACTC |
| | | CTGTTCCTGCGGATCCTCCAACAGCGTTCAACCAGGCCAA |
| | | GCTGAATTCTTTCATCACGCAGTACAGCACCGGACAAGTC |
| | | AGCGTGGAGATCGAGTGGGAGCTGCAGAAGGAGAACAGCA |
| | | AGCGCTGGAACCCAGAGATTCAGTATACTTCCAACTACTA |
| | | CAAATCTACAAATGTGGACTTTGCTGTTAATACTGAGGGT |
| | | GTTTACTCTGAGCCTCGCCCCATTGGCACTCGTTACCTCA |
| | | CCCGTAATCTGTAA |
| 39 | AAV8/AAV6 | MAADGYLPDWLEDNLSEGIREWWALKPGAPKPKANQQKQD |
| | VP1 aa | DGRGLVLPGYKYLGPFNGLDKGEPVNAADAAALEHDKAYD |
| | | QQLQAGDNPYLRYNHADAEFQERLQEDTSFGGNLGRAVFQ |
| | | AKKRVLEPLGLVEEGAKTAPGKKRPVEPSPQRSPDSSTGI |
| | | GKKGQQPARKRLNFGQTGDSESVPDPQPLGEPPAAPSGVG |
| | | PNTMASGGGAPMADNNEGADGVGNASGNWHCDSTWLGDRV |
| | | ITTSTRTWALPTYNNHLYKQISSASTGASNDNHYFGYSTP |
| | | WGYFDFNRFHCHFSPRDWQRLINNNWGFRPKRLNFKLFNI |
| | | QVKEVTTNDGVTTIANNLTSTVQVFSDSEYQLPYVLGSAH |
| | | QGCLPPFPADVFMIPQYGYLTLNNGSQAVGRSSFYCLEYF |
| | | PSQMLRTGNNFTFSYTFEDVPFHSSYAHSQSLDRLMNPLI |
| | | DQYLYYLNRTQNQSGSAQNKDLLFSRGSPAGMSVQPKNWL |
| | | PGPCYRQQRVSKTKTDNNNSNFTWTGASKYNLNGRESIIN |
| | | PGTAMASHKDDKDKFFPMSGVMIFGKESAGASNTALDNVM |
| | | ITDEEEIKATNPVATERFGTVAVNLQSSSTDPATGDVHVM |
| | | GALPGMVWQDRDVYLQGPIWAKIPHTDGHFHPSPLMGGFG |
| | | LKHPPPQILIKNTPVPANPPAEFSATKFASFITQYSTGQV |
| | | SVEIEWELQKENSKRWNPEVQYTSNYAKSANVDFTVDNNG |
| | | LYTEPRPIGTRYLTRPL |
| 40 | AAV8/AAV6 | ATGGCTGCCGATGGTTATCTTCCAGATTGGCTCGAGGACA |
| | VP1 dna | ACCTCTCTGAGGGCATTCGCGAGTGGTGGGCGCTGAAACC |
| | | TGGAGCCCCGAAGCCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGACGCAGCGGCCCTCGAGCACGACAAGGCCTACGAC |
| | | CAGCAGCTGCAGGCGGGTGACAATCCGTACCTGCGGTATA |
| | | ACCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGCGGGTTCTCGAACCTCTCGGTCTGGTTGAGG |
| | | AAGGCGCTAAGACGGCTCCTGGAAAGAAGAGACCGGTAGA |
| | | GCCATCACCCCAGCGTTCTCCAGACTCCTCTACGGGCATC |
| | | GGCAAGAAAGGCCAACAGCCCGCCAGAAAAAGACTCAATT |
| | | TTGGTCAGACTGGCGACTCAGAGTCAGTTCCAGACCCTCA |
| | | ACCTCTCGGAGAACCTCCAGCAGCGCCCTCTGGTGTGGGA |
| | | CCTAATACAATGGCTTCAGGCGGTGGCGCACCAATGGCAG |
| | | ACAATAACGAAGGCGCCGACGGAGTGGGTAATGCCTCAGG |
| | | AAATTGGCATTGCGATTCCACATGGCTGGGCGACAGAGTC |
| | | ATCACCACCAGCACCCGAACATGGGCCTTGCCCACCTATA |
| | | ACAACCACCTCTACAAGCAAATCTCCAGTGCTTCAACGGG |
| | | GGCCAGCAACGACAACCACTACTTCGGCTACAGCACCCCC |
| | | TGGGGGTATTTTGATTTCAACAGATTCCACTGCCATTTCT |
| | | CACCACGTGACTGGCAGCGACTCATCAACAACAATTGGGG |
| | | ATTCCGGCCCAAGAGACTCAACTTCAAGCTCTTCAACATC |
| | | CAAGTCAAGGAGGTCACGACGAATGATGGCGTCACGACCA |
| | | TCGCTAATAACCTTACCAGCACGGTTCAAGTCTTCTCGGA |
| | | CTCGGAGTACCAGTTGCCGTACGTCCTCGGCTCTGCGCAC |
| | | CAGGGCTGCCTCCCTCCGTTCCCGGCGGACGTGTTCATGA |
| | | TTCCGCAGTACGGCTACCTAACGCTCAACAATGGCAGCCA |
| | | GGCAGTGGGACGGTCATCCTTTTACTGCCTGGAATATTTC |
| | | CCATCGCAGATGCTGAGAACGGGCAATAACTTTACCTTCA |
| | | GCTACACCTTCGAGGACGTGCCTTTCCACAGCAGCTACGC |
| | | GCACAGCCAGAGCCTGGACCGGCTGATGAATCCTCTCATC |
| | | GACCAGTACCTGTATTACCTGAACAGAACTCAGAATCAGT |
| | | CCGGAAGTGCCCAAAACAAGGACTTGCTGTTTAGCCGGGG |
| | | GTCTCCAGCTGGCATGTCTGTTCAGCCCAAAAACTGGCTA |
| | | CCTGGACCCTGTTACCGGCAGCAGCGCGTTTCTAAAACAA |
| | | AAACAGACAACAACAACAGCAACTTTACCTGGACTGGTGC |
| | | TTCAAAATATAACCTTAATGGGCGTGAATCTATAATCAAC |
| | | CCTGGCACTGCTATGGCCTCACACAAAGACGACAAAGACA |
| | | AGTTCTTTCCCATGAGCGGTGTCATGATTTTTGGAAAGGA |
| | | GAGCGCCGGAGCTTCAAACACTGCATTGGACAATGTCATG |
| | | ATCACAGACGAAGAGGAAATCAAAGCCACTAACCCCGTGG |
| | | CCACCGAAAGATTTGGGACTGTGGCAGTCAATCTCCAGAG |
| | | CAGCAGCACAGACCCTGCGACCGGAGATGTGCATGTTATG |
| | | GGAGCCTTACCTGGAATGGTGTGGCAAGACAGAGACGTAT |
| | | ACCTGCAGGGTCCTATTTGGGCCAAAATTCCTCACACGGA |
| | | TGGACACTTTCACCCGTCTCCTCTCATGGGCGGCTTTGGA |
| | | CTTAAGCACCCGCCTCCTCAGATCCTCATCAAAAACACGC |
| | | CTGTTCCTGCGAATCCTCCGGCAGAGTTTTCGGCTACAAA |
| | | GTTTGCTTCATTCATCACCCAGTATTCCACAGGACAAGTG |
| | | AGCGTGGAGATTGAATGGGAGCTGCAGAAAGAAAACAGCA |
| | | AACGCTGGAATCCCGAAGTGCAGTATACATCTAACTATGC |
| | | AAAATCTGCCAACGTTGATTTCACTGTGGACAACAATGGA |
| | | CTTTATACTGAGCCTCGCCCCATTGGCACCCGTTACCTCA |
| | | CCCGTCCCCTGTAA |
| 41 | AAV8/AAVrh.21 | MAADGYLPDWLEDNLSEGIREWWALKPGAPKPKANQQKQD |
| | VP1 aa | DGRGLVLPGYKYLGPFNGLDKGEPVNAADAAALEHDKAYD |
| | | QQLQAGDNPYLRYNHADAEFQERLQEDTSFGGNLGRAVFQ |
| | | AKKRVLEPLGLVEEGAKTAPGKKRPVEPSPQRSPDSSTGI |
| | | GKKGQQPARKRLNFGQTGDSESVPDPQPLGEPPAAPSGVG |
| | | PNTMAAGGGAPMADNNEGADGVGNASGNWHCDSTWLGDRV |
| | | ITTSTRTWALPTYNNHLYKQISSQSGATNDNHFFGYSTPW |
| | | GYFDFNRFHCHFSPRDWQRLINNNWGFRPRKLRFKLFNIQ |
| | | VKEVTTNDGVTTIANNLTSTIQVFSDSEYQLPYVLGSAHQ |
| | | GCLPPFPADVFMIPQYGYLTLNNGSQSVGRSSFYCLEYFP |
| | | SQMLRTGNNFEFSYTFEEVPFHSSYAHSQSLDRLMNPLID |
| | | QYLYYLARTQSTTGSTRELQFHQAGPNTMAEQSKNWLPGP |
| | | CYRQQRLSKNIDSNNNSNFAWTGATKYHLNGRNSLTNPGV |
| | | AMATNKDDEDQFFPINGVLVFGKTGAANKTTLENVLMTSE |
| | | EEIKTTNPVATEEYGVVSSNLQSSTAGPQTQTVNSQGALP |
| | | GMVWQNRDVYLQGPIWAKIPHTDGNFHPSPLMGGFGLKHP |
| | | PPQILIKNTPVPANPPEVFTPAKFASFITQYSTGQVSVEI |
| | | EWELQKENSKRWNPEIQYTSNYAKSNNVEFAVNNEGVYTE |
| | | PRPIGTRYLTRNL |
| 42 | AAV8/AAVrh.21 | ATGGCTGCCGATGGTTATCTTCCAGATTGGCTCGAGGACA |
| | VP1 dna | ACCTCTCTGAGGGCATTCGCGAGTGGTGGGCGCTGAAACC |
| | | TGGAGCCCCGAAGCCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGACGCAGCGGCCCTCGAGCACGACAAGGCCTACGAC |
| | | CAGCAGCTGCAGGCGGGTGACAATCCGTACCTGCGGTATA |
| | | ACCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGCGGGTTCTCGAACCTCTCGGTCTGGTTGAGG |
| | | AAGGCGCTAAGACGGCTCCTGGAAAGAAGAGACCGGTAGA |
| | | GCCATCACCCCAGCGTTCTCCAGACTCCTCTACGGGCATC |
| | | GGCAAGAAAGGCCAACAGCCCGCCAGAAAAAGACTCAATT |
| | | TTGGTCAGACTGGCGACTCAGAGTCAGTTCCAGACCCTCA |
| | | ACCTCTCGGAGAACCTCCAGCAGCGCCCTCTGGTGTGGGA |
| | | CCTAATACAATGGCTGCAGGCGGTGGCGCTCCAATGGCAG |
| | | ACAATAACGAAGGCGCCGACGGAGTGGGTAATGCCTCCGG |
| | | AAATTGGCATTGCGATTCCACATGGCTGGGCGACAGAGTC |
| | | ATCACCACCAGCACCCGCACCTGGGCCCTGCCCACCTACA |
| | | ACAACCACCTCTACAAGCAGATATCAAGTCAGAGCGGGGC |
| | | TACCAACGACAACCACTTCTTCGGCTACAGCACCCCCTGG |
| | | GGCTATTTTGACTTCAACAGATTCCACTGCCACTTCTCAC |
| | | CACGTGACTGGCAGCGACTCATCAACAACAACTGGGGATT |
| | | CCGGCCCAGAAAGCTGCGGTTCAAGTTGTTCAACATCCAG |
| | | GTCAAGGAGGTCACGACGAACGACGGCGTTACGACCATCG |
| | | CCAATAACCTTACCAGCACGATTCAGGTCTTCTCGGACTC |
| | | GGAGTACCAACTGCCGTACGTCCTCGGCTCTGCGCACCAG |
| | | GGCTGCCTCCCTCCGTTCCCTGCGGACGTGTTCATGATTC |
| | | CTCAGTACGGATATCTGACTCTAAACAACGGCAGTCAGTC |
| | | TGTGGGACGTTCCTCCTTCTACTGCCTGGAGTACTTTCCT |
| | | TCTCAGATGCTGAGAACGGGCAATAACTTTGAATTCAGCT |
| | | ACACCTTTGAGGAAGTGCCTTTCCACAGCAGCTATGCGCA |
| | | CAGCCAGAGCCTGGACCGGCTGATGAATCCCCTCATCGAC |
| | | CAGTACCTGTACTACCTGGCCCGGACCCAGAGCACTACGG |
| | | GGTCCACAAGGGAGCTGCAGTTCCATCAGGCTGGGCCCAA |
| | | CACCATGGCCGAGCAATCAAAGAACTGGCTGCCCGGACCC |
| | | TGTTATCGGCAGCAGAGACTGTCAAAAAACATAGACAGCA |
| | | ACAACAACAGTAACTTTGCCTGGACCGGGGCCACTAAATA |
| | | CCATCTGAATGGTAGAAATTCATTAACCAACCCGGGCGTA |
| | | GCCATGGCCACCAACAAGGACGACGAGGACCAGTTCTTTC |
| | | CCATCAACGGAGTGCTGGTTTTTGGCAAAACGGGGGCTGC |
| | | CAACAAGACAACGCTGGAAAACGTGCTAATGACCAGCGAG |
| | | GAGGAGATCAAAACCACCAATCCCGTGGCTACAGAAGAAT |
| | | ACGGTGTGGTCTCCAGCAACCTGCAATCGTCTACGGCCGG |
| | | ACCCCAGACACAGACTGTCAACAGCCAGGGGGCTCTGCCC |
| | | GGCATGGTCTGGCAGAACCGGGACGTGTACCTGCAGGGTC |
| | | CCATCTGGGCCAAAATTCCTCACACGGACGGCAACTTTCA |
| | | CCCGTCTCCCCTGATGGGCGGATTTGGACTCAAACACCCG |
| | | CCTCCTCAAATTCTCATCAAAAACACCCCGGTACCTGCTA |
| | | ATCCTCCAGAGGTGTTTACTCCTGCCAAGTTTGCCTCATT |
| | | TATCACGCAGTACAGCACCGGCCAGGTCAGCGTGGAGATC |
| | | GAGTGGGAACTGCAGAAAGAAAACAGCAAACGCTGGAATC |
| | | CAGAGATTCAGTACACCTCAAATTATGCCAAGTCTAATAA |
| | | TGTGGAATTTGCTGTCAACAACGAAGGGGTTTATACTGAG |
| | | CCTCGCCCCATTGGCACCCGTTACCTCACCCGTAACCTGT |
| | | AA |
| 43 | AAV8/AAVrh.73 | MAADGYLPDWLEDNLSEGIREWWALKPGAPKPKANQQKQD |
| | VP1 aa | DGRGLVLPGYKYLGPFNGLDKGEPVNAADAAALEHDKAYD |
| | | QQLQAGDNPYLRYNHADAEFQERLQEDTSFGGNLGRAVFQ |
| | | AKKRVLEPLGLVEEGAKTAPGKKRPVEPSPQRSPDSSTGI |
| | | GKKGQQPARKRLNFGQTGDSESVPDPQPLGEPPAAPSGVG |
| | | PNTMAAGGGAPMADNNEGADGVGSSSGNWHCDSTWLGDRV |
| | | ITTSTRTWALPTYNNHLYKQISNGTSGGSTNDNTYFGYST |
| | | PWGYFDFNRFHCHFSPRDWQRLINNNWGFRPKRLSFKLFN |
| | | IQVKEVTQNEGTKTIANNLTSTIQVFTDSEYQLPYVLGSA |
| | | HQGCLPPFPADVFMIPQYGYLTLNNGSQAVGRSSFYCLEY |
| | | FPSQMLRTGNNFSFSYTFEDVPFHSSYAHSQSLDRLMNPL |
| | | IDQYLYYLSRTQSTGGTAGTQQLLFSQAGPSNMSAQARNW |
| | | LPGPCYRQQRVSTTLSQNNNSNFAWTGATKYHLNGRDSLV |
| | | NPGVAMATNKDDEDRFFPSSGILMFGKQGAGKDNVDYSNV |
| | | MLTSEEEIKTTNPVATEQYGVVADNLQRQNTAPIVGAVNS |
| | | QGALPGMVWQNRDVYLQGPIWAKIPHTDGNFHPSPLMGGF |
| | | GLKHPPPQILIKNTPVPADPPTAFNQAKLNSFITQYSTGQ |
| | | VSVEIEWELQKENSKRWNPEIQYTSNYYKSTNVDFAVNTE |
| | | GVYSEPRPIGTRYLTRNL |
| 44 | AAV8/AAVrh.73 | ATGGCTGCCGATGGTTATCTTCCAGATTGGCTCGAGGACA |
| | VP1 dna | ACCTCTCTGAGGGCATTCGCGAGTGGTGGGCGCTGAAACC |
| | | TGGAGCCCCGAAGCCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGACGCAGCGGCCCTCGAGCACGACAAGGCCTACGAC |
| | | CAGCAGCTGCAGGCGGGTGACAATCCGTACCTGCGGTATA |
| | | ACCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGCGGGTTCTCGAACCTCTCGGTCTGGTTGAGG |
| | | AAGGCGCTAAGACGGCTCCTGGAAAGAAGAGACCGGTAGA |
| | | GCCATCACCCCAGCGTTCTCCAGACTCCTCTACGGGCATC |
| | | GGCAAGAAAGGCCAACAGCCCGCCAGAAAAAGACTCAATT |
| | | TTGGTCAGACTGGCGACTCAGAGTCAGTTCCAGACCCTCA |
| | | ACCTCTCGGAGAACCTCCAGCAGCGCCCTCTGGTGTGGGA |
| | | CCTAATACAATGGCTGCAGGCGGTGGCGCACCAATGGCAG |
| | | ACAATAACGAAGGTGCCGACGGAGTGGGTAGTTCCTCGGG |
| | | AAATTGGCATTGCGATTCCACATGGCTGGGCGACAGAGTC |
| | | ATCACCACCAGCACCCGAACCTGGGCCCTGCCCACCTACA |
| | | ACAACCACCTCTACAAGCAAATCTCCAACGGGACCTCGGG |
| | | AGGCAGCACCAACGACAACACCTACTTTGGCTACAGCACC |
| | | CCCTGGGGGTATTTTGACTTTAACAGATTCCACTGCCACT |
| | | TCTCACCACGTGACTGGCAGCGACTCATCAACAACAACTG |
| | | GGGATTCCGGCCCAAGAGACTCAGCTTCAAGCTCTTCAAC |
| | | ATCCAGGTCAAAGAGGTCACGCAGAATGAAGGCACCAAGA |
| | | CCATCGCCAATAACCTCACCAGCACCATCCAGGTGTTTAC |
| | | GGACTCGGAATACCAGCTGCCGTACGTCCTCGGCTCTGCC |
| | | CACCAGGGCTGCCTGCCTCCGTTCCCGGCGGACGTCTTCA |
| | | TGATTCCTCAGTACGGCTACCTGACTCTCAACAACGGTAG |
| | | TCAGGCCGTGGGACGTTCCTCCTTCTACTGCCTGGAGTAC |
| | | TTCCCCTCTCAGATGCTGAGAACGGGCAACAACTTTTCCT |
| | | TCAGCTACACTTTCGAGGACGTGCCTTTCCACAGCAGCTA |
| | | CGCGCACAGCCAGAGTTTGGACAGGCTGATGAATCCTCTC |
| | | ATCGACCAGTACCTGTACTACCTGTCAAGAACCCAGTCTA |
| | | CGGGAGGCACAGCGGGAACCCAGCAGTTGCTGTTTTCTCA |
| | | GGCCGGGCCTAGCAACATGTCGGCTCAGGCCAGAAACTGG |
| | | CTGCCTGGACCCTGCTACAGACAGCAGCGCGTCTCCACGA |
| | | CACTGTCGCAAAACAACAACAGCAACTTTGCCTGGACTGG |
| | | TGCCACCAAGTATCATCTGAACGGCAGAGACTCTCTGGTG |
| | | AATCCGGGCGTCGCCATGGCAACCAACAAGGACGACGAGG |
| | | ACCGCTTCTTCCCATCCAGCGGCATCCTCATGTTTGGCAA |
| | | GCAGGGAGCTGGAAAAGACAACGTGGACTATAGCAACGTG |
| | | ATGCTAACCAGCGAGGAAGAAATCAAGACCACCAACCCCG |
| | | TGGCCACAGAACAGTATGGCGTGGTGGCTGATAACCTACA |
| | | GAGACAAAACACCGCTCCTATTGTGGGGGCCGTCAACAGC |
| | | CAGGGAGCCTTACCTGGCATGGTCTGGCAGAACCGGGACG |
| | | TGTACCTGCAGGGTCCTATTTGGGCCAAGATTCCTCACAC |
| | | AGATGGCAACTTTCACCCGTCTCCTTTAATGGGCGGCTTT |
| | | GGACTTAAACATCCGCCTCCTCAGATCCTCATCAAAAACA |
| | | CTCCTGTTCCTGCGGATCCTCCAACAGCGTTCAACCAGGC |
| | | CAAGCTGAATTCTTTCATCACGCAGTACAGCACCGGACAA |
| | | GTCAGCGTGGAGATCGAGTGGGAGCTGCAGAAGGAGAACA |
| | | GCAAGCGCTGGAACCCAGAGATTCAGTATACTTCCAACTA |
| | | CTACAAATCTACAAATGTGGACTTTGCTGTTAATACTGAG |
| | | GGTGTTTACTCTGAGCCTCGCCCCATTGGCACTCGTTACC |
| | | TCACCCGTAATCTGTAA |
| 45 | AAV9/AAV4 | MAADGYLPDWLEDNLSEGIREWWALKPGAPQPKANQQHQD |
| | VP1 aa | NARGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGAQPAKKRLNFGQTGDTESVPDPQPIGEPPAAPSGVGS |
| | | LTMRAAAGGAAVEGGQGADGVGNASGDWHCDSTWSEGHVT |
| | | TTSTRTWVLPTYNNHLYKRLGESLQSNTYNGFSTPWGYFD |
| | | FNRFHCHFSPRDWQRLINNNWGMRPKAMRVKIFNIQVKEV |
| | | TTSNGETTVANNLTSTVQIFADSSYELPYVMDAGQEGSLP |
| | | PFPNDVFMVPQYGYCGLVTGNTSQQQTDRNAFYCLEYFPS |
| | | QMLRTGNNFEITYSFEKVPFHSMYAHSQSLDRLMNPLIDQ |
| | | YLWGLQSTTTGTTLNAGTATTNFTKLRPTNFSNFKKNWLP |
| | | GPSIKQQGFSKTANQNYKIPATGSDSLIKYETHSTLDGRW |
| | | SALTPGPPMATAGPADSKFSNSQLIFAGPKQNGNTATVPG |
| | | TLIFTSEEELAATNATDTDMWGNLPGGDQSNSNLPTVDRL |
| | | TALGAVPGMVWQNRDIYYQGPIWAKIPHTDGHFHPSPLIG |
| | | GFGLKHPPPQIFIKNTPVPANPATTFSSTPVNSFITQYST |
| | | GQVSVQIDWEIQKERSKRWNPEVQFTSNYGQQNSLLWAPD |
| | | AAGKYTEPRAIGTRYLTHHL |
| 46 | AAV9/AAV4 | ATGGCTGCCGATGGTTATCTTCCAGATTGGCTCGAGGACA |
| | VP1 dna | ACCTTAGTGAAGGAATTCGCGAGTGGTGGGCTTTGAAACC |
| | | TGGAGCCCCTCAACCCAAGGCAAATCAACAACATCAAGAC |
| | | AACGCTCGAGGTCTTGTGCTTCCGGGTTACAAATACCTTG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTGCACAGCCCGCTAAAAAGAGACTCAATTTCG |
| | | GTCAGACTGGCGACACAGAGTCAGTCCCAGACCCTCAACC |
| | | AATCGGAGAACCTCCCGCAGCCCCCTCAGGTGTGGGATCT |
| | | CTTACAATGCGTGCAGCAGCTGGCGGAGCTGCAGTCGAGG |
| | | GCGGACAAGGTGCCGATGGAGTGGGTAATGCCTCGGGTGA |
| | | TTGGCATTGCGATTCCACCTGGTCTGAGGGCCACGTCACG |
| | | ACCACCAGCACCAGAACCTGGGTCTTGCCCACCTACAACA |
| | | ACCACCTCTACAAGCGACTCGGAGAGAGCCTGCAGTCCAA |
| | | CACCTACAACGGATTCTCCACCCCCTGGGGATACTTTGAC |
| | | TTCAACCGCTTCCACTGCCACTTCTCACCACGTGACTGGC |
| | | AGCGACTCATCAACAACAACTGGGGCATGCGACCCAAAGC |
| | | CATGCGGGTCAAAATCTTCAACATCCAGGTCAAGGAGGTC |
| | | ACGACGTCGAACGGCGAGACAACGGTGGCTAATAACCTTA |
| | | CCAGCACGGTTCAGATCTTTGCGGACTCGTCGTACGAACT |
| | | GCCGTACGTGATGGATGCGGGTCAAGAGGGCAGCCTGCCT |
| | | CCTTTTCCCAACGACGTCTTTATGGTGCCCCAGTACGGCT |
| | | ACTGTGGACTGGTGACCGGCAACACTTCGCAGCAACAGAC |
| | | TGACAGAAATGCCTTCTACTGCCTGGAGTACTTTCCTTCG |
| | | CAGATGCTGCGGACTGGCAACAACTTTGAAATTACGTACA |
| | | GTTTTGAGAAGGTGCCTTTCCACTCGATGTACGCGCACAG |
| | | CCAGAGCCTGGACCGGCTGATGAACCCTCTCATCGACCAG |
| | | TACCTGTGGGGACTGCAATCGACCACCACCGGAACCACCC |
| | | TGAATGCCGGGACTGCCACCACCAACTTTACCAAGCTGCG |
| | | GCCTACCAACTTTTCCAACTTTAAAAAGAACTGGCTGCCC |
| | | GGGCCTTCAATCAAGCAGCAGGGCTTCTCAAAGACTGCCA |
| | | ATCAAAACTACAAGATCCCTGCCACCGGGTCAGACAGTCT |
| | | CATCAAATACGAGACGCACAGCACTCTGGACGGAAGATGG |
| | | AGTGCCCTGACCCCCGGACCTCCAATGGCCACGGCTGGAC |
| | | CTGCGGACAGCAAGTTCAGCAACAGCCAGCTCATCTTTGC |
| | | GGGGCCTAAACAGAACGGCAACACGGCCACCGTACCCGGG |
| | | ACTCTGATCTTCACCTCTGAGGAGGAGCTGGCAGCCACCA |
| | | ACGCCACCGATACGGACATGTGGGGCAACCTACCTGGCGG |
| | | TGACCAGAGCAACAGCAACCTGCCGACCGTGGACAGACTG |
| | | ACAGCCTTGGGAGCCGTGCCTGGAATGGTCTGGCAAAACA |
| | | GAGACATTTACTACCAGGGTCCCATTTGGGCCAAGATTCC |
| | | TCATACCGATGGACACTTTCACCCCTCACCGCTGATTGGT |
| | | GGGTTTGGGCTGAAACACCCGCCTCCTCAAATTTTTATCA |
| | | AGAACACCCCGGTACCTGCGAATCCTGCAACGACCTTCAG |
| | | CTCTACTCCGGTAAACTCCTTCATTACTCAGTACAGCACT |
| | | GGCCAGGTGTCGGTGCAGATTGACTGGGAGATCCAGAAGG |
| | | AGCGGTCCAAACGCTGGAACCCCGAGGTCCAGTTTACCTC |
| | | CAACTACGGACAGCAAAACTCTCTGTTGTGGGCTCCCGAT |
| | | GCGGCTGGGAAATACACTGAGCCTAGGGCTATCGGTACCC |
| | | GCTACCTCACCCACCACCTGTAA |
| 47 | AAV9/AAV8 | MAADGYLPDWLEDNLSEGIREWWALKPGAPQPKANQQHQD |
| | VP1 aa | NARGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGAQPAKKRLNFGQTGDTESVPDPQPIGEPPAAPSGVGS |
| | | LTMAAGGGAPMADNNEGADGVGSSSGNWHCDSTWLGDRVI |
| | | TTSTRTWALPTYNNHLYKQISNGTSGGATNDNTYFGYSTP |
| | | WGYFDFNRFHCHFSPRDWQRLINNNWGFRPKRLSFKLFNI |
| | | QVKEVTQNEGTKTIANNLTSTIQVFTDSEYQLPYVLGSAH |
| | | QGCLPPFPADVFMIPQYGYLTLNNGSQAVGRSSFYCLEYF |
| | | PSQMLRTGNNFQFTYTFEDVPFHSSYAHSQSLDRLMNPLI |
| | | DQYLYYLSRTQTTGGTANTQTLGFSQGGPNTMANQAKNWL |
| | | PGPCYRQQRVSTTTGQNNNSNFAWTAGTKYHLNGRNSLAN |
| | | PGIAMATHKDDEERFFPSNGILIFGKQNAARDNADYSDVM |
| | | LTSEEEIKTTNPVATEEYGIVADNLQQQNTAPQIGTVNSQ |
| | | GALPGMVWQNRDVYLQGPIWAKIPHTDGNFHPSPLMGGFG |
| | | LKHPPPQILIKNTPVPADPPTTFNQSKLNSFITQYSTGQV |
| | | SVEIEWELQKENSKRWNPEIQYTSNYYKSTSVDFAVNTEG |
| | | VYSEPRPIGTRYLTRNL |
| 48 | AAV9/AAV8 | ATGGCTGCCGATGGTTATCTTCCAGATTGGCTCGAGGACA |
| | VP1 dna | ACCTTAGTGAAGGAATTCGCGAGTGGTGGGCTTTGAAACC |
| | | TGGAGCCCCTCAACCCAAGGCAAATCAACAACATCAAGAC |
| | | AACGCTCGAGGTCTTGTGCTTCCGGGTTACAAATACCTTG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTGCACAGCCCGCTAAAAAGAGACTCAATTTCG |
| | | GTCAGACTGGCGACACAGAGTCAGTCCCAGACCCTCAACC |
| | | AATCGGAGAACCTCCCGCAGCCCCCTCAGGTGTGGGATCT |
| | | CTTACAATGGCTGCAGGCGGTGGCGCACCAATGGCAGACA |
| | | ATAACGAAGGCGCCGACGGAGTGGGTAGTTCCTCGGGAAA |
| | | TTGGCATTGCGATTCCACATGGCTGGGCGACAGAGTCATC |
| | | ACCACCAGCACCCGAACCTGGGCCCTGCCCACCTACAACA |
| | | ACCACCTCTACAAGCAAATCTCCAACGGGACATCGGGAGG |
| | | AGCCACCAACGACAACACCTACTTCGGCTACAGCACCCCC |
| | | TGGGGGTATTTTGACTTTAACAGATTCCACTGCCACTTTT |
| | | CACCACGTGACTGGCAGCGACTCATCAACAACAACTGGGG |
| | | ATTCCGGCCCAAGAGACTCAGCTTCAAGCTCTTCAACATC |
| | | CAGGTCAAGGAGGTCACGCAGAATGAAGGCACCAAGACCA |
| | | TCGCCAATAACCTCACCAGCACCATCCAGGTGTTTACGGA |
| | | CTCGGAGTACCAGCTGCCGTACGTTCTCGGCTCTGCCCAC |
| | | CAGGGCTGCCTGCCTCCGTTCCCGGCGGACGTGTTCATGA |
| | | TTCCCCAGTACGGCTACCTAACACTCAACAACGGTAGTCA |
| | | GGCCGTGGGACGCTCCTCCTTCTACTGCCTGGAATACTTT |
| | | CCTTCGCAGATGCTGAGAACCGGCAACAACTTCCAGTTTA |
| | | CTTACACCTTCGAGGACGTGCCTTTCCACAGCAGCTACGC |
| | | CCACAGCCAGAGCTTGGACCGGCTGATGAATCCTCTGATT |
| | | GACCAGTACCTGTACTACTTGTCTCGGACTCAAACAACAG |
| | | GAGGCACGGCAAATACGCAGACTCTGGGCTTCAGCCAAGG |
| | | TGGGCCTAATACAATGGCCAATCAGGCAAAGAACTGGCTG |
| | | CCAGGACCCTGTTACCGCCAACAACGCGTCTCAACGACAA |
| | | CCGGGCAAAACAACAATAGCAACTTTGCCTGGACTGCTGG |
| | | GACCAAATACCATCTGAATGGAAGAAATTCATTGGCTAAT |
| | | CCTGGCATCGCTATGGCAACACACAAAGACGACGAGGAGC |
| | | GTTTTTTTCCCAGTAACGGGATCCTGATTTTTGGCAAACA |
| | | AAATGCTGCCAGAGACAATGCGGATTACAGCGATGTCATG |
| | | CTCACCAGCGAGGAAGAAATCAAAACCACTAACCCTGTGG |
| | | CTACAGAGGAATACGGTATCGTGGCAGATAACTTGCAGCA |
| | | GCAAAACACGGCTCCTCAAATTGGAACTGTCAACAGCCAG |
| | | GGGGCCTTACCCGGTATGGTCTGGCAGAACCGGGACGTGT |
| | | ACCTGCAGGGTCCCATCTGGGCCAAGATTCCTCACACGGA |
| | | CGGCAACTTCCACCCGTCTCCGCTGATGGGCGGCTTTGGC |
| | | CTGAAACATCCTCCGCCTCAGATCCTGATCAAGAACACGC |
| | | CTGTACCTGCGGATCCTCCGACCACCTTCAACCAGTCAAA |
| | | GCTGAACTCTTTCATCACGCAATACAGCACCGGACAGGTC |
| | | AGCGTGGAAATTGAATGGGAGCTGCAGAAGGAAAACAGCA |
| | | AGCGCTGGAACCCCGAGATCCAGTACACCTCCAACTACTA |
| | | CAAATCTACAAGTGTGGACTTTGCTGTTAATACAGAAGGC |
| | | GTGTACTCTGAACCCCGCCCCATTGGCACCCGTTACCTCA |
| | | CCCGTAATCTGTAA |
| 49 | AAV9/AAVrh18 | MAADGYLPDWLEDNLSEGIREWWALKPGAPQPKANQQHQD |
| | VP1 aa | NARGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGAQPAKKRLNFGQTGDTESVPDPQPIGEPPAAPSGVGS |
| | | LTMAAGGGAPMADNNEGADGVGSSSGNWHCDSTWLGDRVI |
| | | TTSTRTWALPTYNNHLYKQISNGTSGGSTNDNTYFGYSTP |
| | | WGYFDFNRFHCHFSPRDWQRLINNNWGFRPKRLNFKLFNI |
| | | QVKEVTQNEGTKTIANNLTSTIQVFTDSEYQLPYVLGSAH |
| | | QGCLPPFPADVFMIPQYGYLTLNNGSQAVGRSSFYCLEYF |
| | | PSQMLRTGNNFEFSYQFEDVPFHSSYAHSQSLDRLMNPLI |
| | | DQYLYYLSRTQSTGGTAGTQQLLFSQAGPNNMSAQAKNWL |
| | | PGPCYRQQRVSTTLSQNNNSNFAWTGATKYHLNGRDSLVN |
| | | PGVAMATHKDDEERFFPSSGVLMFGKQGAGKDNVDYSSVM |
| | | LTSEEEIKTTNPVATEQYGVVADNLQQQNAAPIVGAVNSQ |
| | | GALPGMVWQNRDVYLQGPIWAKIPHTDGNFHPSPLMGGFG |
| | | LKHPPPQILIKNTPVPADPPTTFSQAKLASFITQYSTGQV |
| | | SVEIEWELQKENSKRWNPEIQYTSNYYKSTNVDFAVNTEG |
| | | TYSEPRPIGTRYLTRNL |
| 50 | AAV9/AAVrh18 | ATGGCTGCCGATGGTTATCTTCCAGATTGGCTCGAGGACA |
| | VP1 dna | ACCTTAGTGAAGGAATTCGCGAGTGGTGGGCTTTGAAACC |
| | | TGGAGCCCCTCAACCCAAGGCAAATCAACAACATCAAGAC |
| | | AACGCTCGAGGTCTTGTGCTTCCGGGTTACAAATACCTTG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTGCACAGCCCGCTAAAAAGAGACTCAATTTCG |
| | | GTCAGACTGGCGACACAGAGTCAGTCCCAGACCCTCAACC |
| | | AATCGGAGAACCTCCCGCAGCCCCCTCAGGTGTGGGATCT |
| | | CTTACAATGGCTGCAGGCGGTGGCGCTCCAATGGCAGACA |
| | | ATAACGAAGGCGCCGACGGAGTGGGTAGTTCCTCAGGAAA |
| | | TTGGCATTGCGATTCCACATGGCTGGGCGACAGAGTCATC |
| | | ACCACCAGCACCCGAACCTGGGCCCTCCCCACCTACAACA |
| | | ACCACCTCTACAAGCAAATCTCCAACGGGACATCGGGAGG |
| | | AAGCACCAACGACAACACCTACTTCGGCTACAGCACCCCC |
| | | TGGGGGTATTTTGACTTTAACAGATTCCACTGCCACTTCT |
| | | CACCACGTGACTGGCAGCGACTCATCAACAACAACTGGGG |
| | | ATTCCGGCCCAAGAGACTCAACTTCAAGCTCTTCAACATC |
| | | CAGGTCAAGGAGGTCACGCAGAATGAAGGCACCAAGACCA |
| | | TCGCCAATAACCTTACCAGCACGATTCAGGTCTTTACGGA |
| | | CTCGGAATACCAGCTCCCGTACGTCCTCGGCTCTGCGCAC |
| | | CAGGGCTGCCTGCCTCCGTTCCCGGCGGACGTCTTCATGA |
| | | TTCCTCAGTACGGGTACCTGACTCTGAACAACGGCAGTCA |
| | | GGCCGTGGGCCGTTCCTCCTTCTACTGCCTGGAGTACTTT |
| | | CCTTCTCAAATGCTGAGAACGGGCAACAACTTTGAGTTCA |
| | | GCTACCAGTTTGAGGACGTGCCTTTTCACAGCAGCTACGC |
| | | GCACAGCCAAAGCCTGGACCGGCTGATGAACCCCCTCATC |
| | | GACCAGTACCTGTACTACCTGTCTCGGACTCAGTCCACGG |
| | | GAGGTACCGCAGGAACTCAGCAGTTGCTATTTTCTCAGGC |
| | | CGGGCCTAATAACATGTCGGCTCAGGCCAAAAACTGGCTA |
| | | CCCGGGCCCTGCTACCGGCAGCAACGCGTCTCCACGACAC |
| | | TGTCGCAAAATAACAACAGCAACTTTGCTTGGACCGGTGC |
| | | CACCAAGTATCATCTGAATGGCAGAGACTCTCTGGTAAAT |
| | | CCCGGTGTCGCTATGGCAACGCACAAGGACGACGAAGAGC |
| | | GATTTTTTCCATCCAGCGGAGTCTTGATGTTTGGGAAACA |
| | | GGGAGCTGGAAAAGACAACGTGGACTATAGCAGCGTTATG |
| | | CTAACCAGTGAGGAAGAAATCAAAACCACCAACCCAGTGG |
| | | CCACAGAACAGTACGGCGTGGTGGCCGATAACCTGCAACA |
| | | GCAAAACGCCGCTCCTATTGTAGGGGCCGTCAACAGTCAA |
| | | GGAGCCTTACCTGGCATGGTCTGGCAGAACCGGGACGTGT |
| | | ACCTGCAGGGTCCTATCTGGGCCAAGATTCCTCACACGGA |
| | | CGGCAACTTTCATCCTTCGCCGCTGATGGGAGGCTTTGGA |
| | | CTGAAACACCCGCCTCCTCAGATCCTGATTAAGAATACAC |
| | | CTGTTCCCGCGGATCCTCCAACTACCTTCAGTCAAGCCAA |
| | | GCTGGCGTCGTTCATCACGCAGTACAGCACCGGACAGGTC |
| | | AGCGTGGAAATTGAATGGGAGCTGCAGAAAGAGAACAGCA |
| | | AGCGCTGGAACCCAGAGATTCAGTATACTTCCAACTACTA |
| | | CAAATCTACAAATGTGGACTTTGCTGTCAATACTGAGGGT |
| | | ACTTATTCAGAGCCTCGCCCCATTGGCACCCGTTACCTCA |
| | | CCCGTAACCTGTAA |
| 51 | AAVrh21/AAV8 | MAADGYLPDWLEDNLSEGIREWWDLKPGAPKPKANQQKQD |
| | VP1 aa | DGRGLVLPGYKYLGPFNGLDKGEPVNEADAAALEHDKAYD |
| | | KQLEQGDNPYLKYNHADAEFQERLQEDTSFGGNLGRAVFQ |
| | | AKKRVLEPLGLVEEGAKTAPGKKRPIESPDSSTGIGRKGQ |
| | | QPAKKKLNFGQTGDSESVPDPQPIGEPPAGPSGLGSGTMA |
| | | AGGGAPMADNNEGADGVGSSSGNWHCDSTWLGDRVITTST |
| | | RTWALPTYNNHLYKQISNGTSGGATNDNTYFGYSTPWGYF |
| | | DFNRFHCHFSPRDWQRLINNNWGFRPKRLSFKLFNIQVKE |
| | | VTQNEGTKTIANNLTSTIQVFTDSEYQLPYVLGSAHQGCL |
| | | PPFPADVFMIPQYGYLTLNNGSQAVGRSSFYCLEYFPSQM |
| | | LRTGNNFQFTYTFEDVPFHSSYAHSQSLDRLMNPLIDQYL |
| | | YYLSRTQTTGGTANTQTLGFSQGGPNTMANQAKNWLPGPC |
| | | YRQQRVSTTTGQNNNSNFAWTAGTKYHLNGRNSLANPGIA |
| | | MATHKDDEERFFPSNGILIFGKQNAARDNADYSDVMLTSE |
| | | EEIKTTNPVATEEYGIVADNLQQQNTAPQIGTVNSQGALP |
| | | GMVWQNRDVYLQGPIWAKIPHTDGNFHPSPLMGGFGLKHP |
| | | PPQILIKNTPVPADPPTTFNQSKLNSFITQYSTGQVSVEI |
| | | EWELQKENSKRWNPEIQYTSNYYKSTSVDFAVNTEGVYSE |
| | | PRPIGTRYLTRNL |
| 52 | AAVrh21/AAV8 | ATGGCTGCCGATGGTTATCTTCCAGATTGGCTCGAGGACA |
| | VP1 dna | ACCTCTCTGAGGGCATTCGCGAGTGGTGGGACTTGAAACC |
| | | TGGAGCCCCGAAACCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGAGAGCCGGTCAACGA |
| | | GGCAGACGCCGCGGCCCTCGAGCACGACAAGGCCTACGAC |
| | | AAGCAGCTCGAGCAGGGGGACAACCCGTACCTCAAGTACA |
| | | ACCACGCCGACGCCGAGTTTCAGGAGCGTCTTCAAGAAGA |
| | | TACGTCTTTTGGGGGCAACCTCGGGCGAGCAGTCTTCCAG |
| | | GCCAAGAAGCGGGTTCTCGAACCTCTCGGTCTGGTTGAGG |
| | | AAGGCGCTAAGACGGCTCCTGGAAAGAAGAGACCCATAGA |
| | | ATCCCCCGACTCCTCCACGGGCATCGGCAGGAAAGGCCAG |
| | | CAGCCCGCTAAAAAGAAGCTCAACTTTGGGCAGACTGGCG |
| | | ACTCAGAGTCAGTGCCCGACCCTCAACCAATCGGAGAACC |
| | | CCCCGCAGGCCCCTCTGGTCTGGGATCTGGTACAATGGCT |
| | | GCAGGCGGTGGCGCACCAATGGCAGACAATAACGAAGGCG |
| | | CCGACGGAGTGGGTAGTTCCTCGGGAAATTGGCATTGCGA |
| | | TTCCACATGGCTGGGCGACAGAGTCATCACCACCAGCACC |
| | | CGAACCTGGGCCCTGCCCACCTACAACAACCACCTCTACA |
| | | AGCAAATCTCCAACGGGACATCGGGAGGAGCCACCAACGA |
| | | CAACACCTACTTCGGCTACAGCACCCCCTGGGGGTATTTT |
| | | GACTTTAACAGATTCCACTGCCACTTTTCACCACGTGACT |
| | | GGCAGCGACTCATCAACAACAACTGGGGATTCCGGCCCAA |
| | | GAGACTCAGCTTCAAGCTCTTCAACATCCAGGTCAAGGAG |
| | | GTCACGCAGAATGAAGGCACCAAGACCATCGCCAATAACC |
| | | TCACCAGCACCATCCAGGTGTTTACGGACTCGGAGTACCA |
| | | GCTGCCGTACGTTCTCGGCTCTGCCCACCAGGGCTGCCTG |
| | | CCTCCGTTCCCGGCGGACGTGTTCATGATTCCCCAGTACG |
| | | GCTACCTAACACTCAACAACGGTAGTCAGGCCGTGGGACG |
| | | CTCCTCCTTCTACTGCCTGGAATACTTTCCTTCGCAGATG |
| | | CTGAGAACCGGCAACAACTTCCAGTTTACTTACACCTTCG |
| | | AGGACGTGCCTTTCCACAGCAGCTACGCCCACAGCCAGAG |
| | | CTTGGACCGGCTGATGAATCCTCTGATTGACCAGTACCTG |
| | | TACTACTTGTCTCGGACTCAAACAACAGGAGGCACGGCAA |
| | | ATACGCAGACTCTGGGCTTCAGCCAAGGTGGGCCTAATAC |
| | | AATGGCCAATCAGGCAAAGAACTGGCTGCCAGGACCCTGT |
| | | TACCGCCAACAACGCGTCTCAACGACAACCGGGCAAAACA |
| | | ACAATAGCAACTTTGCCTGGACTGCTGGGACCAAATACCA |
| | | TCTGAATGGAAGAAATTCATTGGCTAATCCTGGCATCGCT |
| | | ATGGCAACACACAAAGACGACGAGGAGCGTTTTTTTCCCA |
| | | GTAACGGGATCCTGATTTTTGGCAAACAAAATGCTGCCAG |
| | | AGACAATGCGGATTACAGCGATGTCATGCTCACCAGCGAG |
| | | GAAGAAATCAAAACCACTAACCCTGTGGCTACAGAGGAAT |
| | | ACGGTATCGTGGCAGATAACTTGCAGCAGCAAAACACGGC |
| | | TCCTCAAATTGGAACTGTCAACAGCCAGGGGGCCTTACCC |
| | | GGTATGGTCTGGCAGAACCGGGACGTGTACCTGCAGGGTC |
| | | CCATCTGGGCCAAGATTCCTCACACGGACGGCAACTTCCA |
| | | CCCGTCTCCGCTGATGGGCGGCTTTGGCCTGAAACATCCT |
| | | CCGCCTCAGATCCTGATCAAGAACACGCCTGTACCTGCGG |
| | | ATCCTCCGACCACCTTCAACCAGTCAAAGCTGAACTCTTT |
| | | CATCACGCAATACAGCACCGGACAGGTCAGCGTGGAAATT |
| | | GAATGGGAGCTGCAGAAGGAAAACAGCAAGCGCTGGAACC |
| | | CCGAGATCCAGTACACCTCCAACTACTACAAATCTACAAG |
| | | TGTGGACTTTGCTGTTAATACAGAAGGCGTGTACTCTGAA |
| | | CCCCGCCCCATTGGCACCCGTTACCTCACCCGTAATCTGT |
| | | AA |
| 53 | AAVrh73/AAV8 | MAADGYLPDWLEDNLSEGIREWWDLKPGAPKPKANQQKQD |
| | VP1 aa | DGRGLVLPGYKYLGPFNGLDKGEPVNAADAAALEHDKAYD |
| | | QQLKAGDNPYLRYNHADAEFQERLQEDTSFGGNLGRAVFQ |
| | | AKKRVLEPLGLVEEGAKTAPGKKRPVEPSPQRSPDSSTGI |
| | | GKKGQQPARKRLNFGQTGDSESVPDPQPLGEPPAAPSSVG |
| | | SGTMAAGGGAPMADNNEGADGVGSSSGNWHCDSTWLGDRV |
| | | ITTSTRTWALPTYNNHLYKQISNGTSGGATNDNTYFGYST |
| | | PWGYFDFNRFHCHFSPRDWQRLINNNWGFRPKRLSFKLFN |
| | | IQVKEVTQNEGTKTIANNLTSTIQVFTDSEYQLPYVLGSA |
| | | HQGCLPPFPADVFMIPQYGYLTLNNGSQAVGRSSFYCLEY |
| | | FPSQMLRTGNNFQFTYTFEDVPFHSSYAHSQSLDRLMNPL |
| | | IDQYLYYLSRTQTTGGTANTQTLGFSQGGPNTMANQAKNW |
| | | LPGPCYRQQRVSTTTGQNNNSNFAWTAGTKYHLNGRNSLA |
| | | NPGIAMATHKDDEERFFPSNGILIFGKQNAARDNADYSDV |
| | | MLTSEEEIKTTNPVATEEYGIVADNLQQQNTAPQIGTVNS |
| | | QGALPGMVWQNRDVYLQGPIWAKIPHTDGNFHPSPLMGGF |
| | | GLKHPPPQILIKNTPVPADPPTTFNQSKLNSFITQYSTGQ |
| | | VSVEIEWELQKENSKRWNPEIQYTSNYYKSTSVDFAVNTE |
| | | GVYSEPRPIGTRYLTRNL |
| 54 | AAVrh73/AAV8 | ATGGCTGCCGATGGTTATCTTCCAGATTGGCTCGAGGACA |
| | VP1 dna | ACCTCTCTGAGGGCATTCGCGAGTGGTGGGACCTGAAACC |
| | | TGGAGCCCCGAAACCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGACGCAGCGGCCCTCGAGCACGACAAGGCCTACGAC |
| | | CAGCAGCTCAAAGCGGGTGACAATCCGTACCTGCGGTATA |
| | | ATCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGCGGGTTCTCGAACCTCTCGGTCTGGTTGAGG |
| | | AAGGCGCTAAGACGGCTCCTGGAAAGAAGAGACCGGTAGA |
| | | GCCGTCACCACAGCGTTCCCCCGACTCCTCCACGGGCATC |
| | | GGCAAGAAAGGCCAGCAGCCCGCCAGAAAGAGACTCAATT |
| | | TCGGTCAGACTGGCGACTCAGAGTCAGTCCCCGACCCTCA |
| | | ACCTCTGGGAGAACCTCCAGCAGCGCCCTCTAGTGTGGGA |
| | | TCTGGTACAATGGCTGCAGGCGGTGGCGCACCAATGGCAG |
| | | ACAATAACGAAGGCGCCGACGGAGTGGGTAGTTCCTCGGG |
| | | AAATTGGCATTGCGATTCCACATGGCTGGGCGACAGAGTC |
| | | ATCACCACCAGCACCCGAACCTGGGCCCTGCCCACCTACA |
| | | ACAACCACCTCTACAAGCAAATCTCCAACGGGACATCGGG |
| | | AGGAGCCACCAACGACAACACCTACTTCGGCTACAGCACC |
| | | CCCTGGGGGTATTTTGACTTTAACAGATTCCACTGCCACT |
| | | TTTCACCACGTGACTGGCAGCGACTCATCAACAACAACTG |
| | | GGGATTCCGGCCCAAGAGACTCAGCTTCAAGCTCTTCAAC |
| | | ATCCAGGTCAAGGAGGTCACGCAGAATGAAGGCACCAAGA |
| | | CCATCGCCAATAACCTCACCAGCACCATCCAGGTGTTTAC |
| | | GGACTCGGAGTACCAGCTGCCGTACGTTCTCGGCTCTGCC |
| | | CACCAGGGCTGCCTGCCTCCGTTCCCGGCGGACGTGTTCA |
| | | TGATTCCCCAGTACGGCTACCTAACACTCAACAACGGTAG |
| | | TCAGGCCGTGGGACGCTCCTCCTTCTACTGCCTGGAATAC |
| | | TTTCCTTCGCAGATGCTGAGAACCGGCAACAACTTCCAGT |
| | | TTACTTACACCTTCGAGGACGTGCCTTTCCACAGCAGCTA |
| | | CGCCCACAGCCAGAGCTTGGACCGGCTGATGAATCCTCTG |
| | | ATTGACCAGTACCTGTACTACTTGTCTCGGACTCAAACAA |
| | | CAGGAGGCACGGCAAATACGCAGACTCTGGGCTTCAGCCA |
| | | AGGTGGGCCTAATACAATGGCCAATCAGGCAAAGAACTGG |
| | | CTGCCAGGACCCTGTTACCGCCAACAACGCGTCTCAACGA |
| | | CAACCGGGCAAAACAACAATAGCAACTTTGCCTGGACTGC |
| | | TGGGACCAAATACCATCTGAATGGAAGAAATTCATTGGCT |
| | | AATCCTGGCATCGCTATGGCAACACACAAAGACGACGAGG |
| | | AGCGTTTTTTTCCCAGTAACGGGATCCTGATTTTTGGCAA |
| | | ACAAAATGCTGCCAGAGACAATGCGGATTACAGCGATGTC |
| | | ATGCTCACCAGCGAGGAAGAAATCAAAACCACTAACCCTG |
| | | TGGCTACAGAGGAATACGGTATCGTGGCAGATAACTTGCA |
| | | GCAGCAAAACACGGCTCCTCAAATTGGAACTGTCAACAGC |
| | | CAGGGGGCCTTACCCGGTATGGTCTGGCAGAACCGGGACG |
| | | TGTACCTGCAGGGTCCCATCTGGGCCAAGATTCCTCACAC |
| | | GGACGGCAACTTCCACCCGTCTCCGCTGATGGGCGGCTTT |
| | | GGCCTGAAACATCCTCCGCCTCAGATCCTGATCAAGAACA |
| | | CGCCTGTACCTGCGGATCCTCCGACCACCTTCAACCAGTC |
| | | AAAGCTGAACTCTTTCATCACGCAATACAGCACCGGACAG |
| | | GTCAGCGTGGAAATTGAATGGGAGCTGCAGAAGGAAAACA |
| | | GCAAGCGCTGGAACCCCGAGATCCAGTACACCTCCAACTA |
| | | CTACAAATCTACAAGTGTGGACTTTGCTGTTAATACAGAA |
| | | GGCGTGTACTCTGAACCCCGCCCCATTGGCACCCGTTACC |
| | | TCACCCGTAATCTGTAA |
| 55 | AAV3B/AAV8 | MAAGGGAPMADNNEGADGVGSSSGNWHCDSTWLGDRVITT |
| | VP1 aa | STRTWALPTYNNHLYKQISNGTSGGATNDNTYFGYSTPWG |
| | | YFDFNRFHCHFSPRDWQRLINNNWGFRPKRLSFKLFNIQV |
| | | KEVTQNEGTKTIANNLTSTIQVFTDSEYQLPYVLGSAHQG |
| | | CLPPFPADVFMIPQYGYLTLNNGSQAVGRSSFYCLEYFPS |
| | | QMLRTGNNFQFTYTFEDVPFHSSYAHSQSLDRLMNPLIDQ |
| | | YLYYLSRTQTTGGTANTQTLGFSQGGPNTMANQAKNWLPG |
| | | PCYRQQRVSTTTGQNNNSNFAWTAGTKYHLNGRNSLANPG |
| | | IAMATHKDDEERFFPSNGILIFGKQNAARDNADYSDVMLT |
| | | SEEEIKTTNPVATEEYGIVADNLQQQNTAPQIGTVNSQGA |
| | | LPGMVWQNRDVYLQGPIWAKIPHTDGNFHPSPLMGGFGLK |
| | | HPPPQILIKNTPVPADPPTTFNQSKLNSFITQYSTGQVSV |
| | | EIEWELQKENSKRWNPEIQYTSNYYKSTSVDFAVNTEGVY |
| | | SEPRPIGTRYLTRNL |
| 56 | AAV3B/AAV8 | ATGGCTGCAGGCGGTGGCGCACCAATGGCAGACAATAACG |
| | VP1 dna | AAGGCGCCGACGGAGTGGGTAGTTCCTCGGGAAATTGGCA |
| | | TTGCGATTCCACATGGCTGGGCGACAGAGTCATCACCACC |
| | | AGCACCCGAACCTGGGCCCTGCCCACCTACAACAACCACC |
| | | TCTACAAGCAAATCTCCAACGGGACATCGGGAGGAGCCAC |
| | | CAACGACAACACCTACTTCGGCTACAGCACCCCCTGGGGG |
| | | TATTTTGACTTTAACAGATTCCACTGCCACTTTTCACCAC |
| | | GTGACTGGCAGCGACTCATCAACAACAACTGGGGATTCCG |
| | | GCCCAAGAGACTCAGCTTCAAGCTCTTCAACATCCAGGTC |
| | | AAGGAGGTCACGCAGAATGAAGGCACCAAGACCATCGCCA |
| | | ATAACCTCACCAGCACCATCCAGGTGTTTACGGACTCGGA |
| | | GTACCAGCTGCCGTACGTTCTCGGCTCTGCCCACCAGGGC |
| | | TGCCTGCCTCCGTTCCCGGCGGACGTGTTCATGATTCCCC |
| | | AGTACGGCTACCTAACACTCAACAACGGTAGTCAGGCCGT |
| | | GGGACGCTCCTCCTTCTACTGCCTGGAATACTTTCCTTCG |
| | | CAGATGCTGAGAACCGGCAACAACTTCCAGTTTACTTACA |
| | | CCTTCGAGGACGTGCCTTTCCACAGCAGCTACGCCCACAG |
| | | CCAGAGCTTGGACCGGCTGATGAATCCTCTGATTGACCAG |
| | | TACCTGTACTACTTGTCTCGGACTCAAACAACAGGAGGCA |
| | | CGGCAAATACGCAGACTCTGGGCTTCAGCCAAGGTGGGCC |
| | | TAATACAATGGCCAATCAGGCAAAGAACTGGCTGCCAGGA |
| | | CCCTGTTACCGCCAACAACGCGTCTCAACGACAACCGGGC |
| | | AAAACAACAATAGCAACTTTGCCTGGACTGCTGGGACCAA |
| | | ATACCATCTGAATGGAAGAAATTCATTGGCTAATCCTGGC |
| | | ATCGCTATGGCAACACACAAAGACGACGAGGAGCGTTTTT |
| | | TTCCCAGTAACGGGATCCTGATTTTTGGCAAACAAAATGC |
| | | TGCCAGAGACAATGCGGATTACAGCGATGTCATGCTCACC |
| | | AGCGAGGAAGAAATCAAAACCACTAACCCTGTGGCTACAG |
| | | AGGAATACGGTATCGTGGCAGATAACTTGCAGCAGCAAAA |
| | | CACGGCTCCTCAAATTGGAACTGTCAACAGCCAGGGGGCC |
| | | TTACCCGGTATGGTCTGGCAGAACCGGGACGTGTACCTGC |
| | | AGGGTCCCATCTGGGCCAAGATTCCTCACACGGACGGCAA |
| | | CTTCCACCCGTCTCCGCTGATGGGCGGCTTTGGCCTGAAA |
| | | CATCCTCCGCCTCAGATCCTGATCAAGAACACGCCTGTAC |
| | | CTGCGGATCCTCCGACCACCTTCAACCAGTCAAAGCTGAA |
| | | CTCTTTCATCACGCAATACAGCACCGGACAGGTCAGCGTG |
| | | GAAATTGAATGGGAGCTGCAGAAGGAAAACAGCAAGCGCT |
| | | GGAACCCCGAGATCCAGTACACCTCCAACTACTACAAATC |
| | | TACAAGTGTGGACTTTGCTGTTAATACAGAAGGCGTGTAC |
| | | TCTGAACCCCGCCCCATTGGCACCCGTTACCTCACCCGTA |
| | | ATCTGTAA |
| 57 | AAVrh15/ | MAADGYLPDWLEDNLSEGIREWWDLKPGAPKPKANQQKQD |
| | AAVhu32 | DGRGLVLPGYKYLGPFNGLDKGEPVNEADAAALEHDKAYD |
| | VP1 aa | KQLEQGDNPYLKYNHADAEFQERLQEDTSFGGNLGRAVFQ |
| | | AKKRVLEPLGLVEEGAKTAPGKKRPIESPDSSTGIGKKGQ |
| | | QPAKKKLNFGQTGDSESVPDPQPIGEPPAGPSGLGSGTMA |
| | | SGGGAPVADNNEGADGVGSSSGNWHCDSQWLGDRVITTST |
| | | RTWALPTYNNHLYKQISNSTSGGSSNDNAYFGYSTPWGYF |
| | | DFNRFHCHFSPRDWQRLINNNWGFRPKRLNFKLFNIQVKE |
| | | VTDNNGVKTIANNLTSTVQVFTDSDYQLPYVLGSAHEGCL |
| | | PPFPADVFMIPQYGYLTLNDGSQAVGRSSFYCLEYFPSQM |
| | | LRTGNNFQFSYEFENVPFHSSYAHSQSLDRLMNPLIDQYL |
| | | YYLSKTINGSGQNQQTLKFSVAGPSNMAVQGRNYIPGPSY |
| | | RQQRVSTTVTQNNNSEFAWPGASSWALNGRNSLMNPGPAM |
| | | ASHKEGEDRFFPLSGSLIFGKQGTGRDNVDADKVMITNEE |
| | | EIKTTNPVATESYGQVATNHQSAQAQAQTGWVQNQGILPG |
| | | MVWQDRDVYLQGPIWAKIPHTDGNFHPSPLMGGFGMKHPP |
| | | PQILIKNTPVPADPPTAFNKDKLNSFITQYSTGQVSVEIE |
| | | WELQKENSKRWNPEIQYTSNYYKSNNVEFAVNTEGVYSEP |
| | | RPIGTRYLTRNL |
| 58 | AAVrh15/ | ATGGCTGCCGATGGTTATCTTCCAGATTGGCTCGAGGACA |
| | AAVhu32 | ACCTCTCTGAGGGCATTCGCGAGTGGTGGGACTTGAAACC |
| | VP1 dna | TGGAGCCCCGAAACCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGAGAGCCGGTCAACGA |
| | | GGCAGACGCCGCGGCCCTCGAGCACGACAAGGCCTACGAC |
| | | AAGCAGCTCGAGCAGGGGGACAACCCGTACCTCAAGTACA |
| | | ACCACGCCGACGCCGAGTTTCAGGAGCGTCTTCAAGAAGA |
| | | TACGTCTTTTGGGGGCAACCTCGGGCGAGCAGTCTTCCAG |
| | | GCCAAGAAGCGGGTTCTCGAACCTCTCGGTCTGGTTGAGG |
| | | AAGGCGCTAAGACGGCTCCTGGAAAGAAGAGACCCATAGA |
| | | ATCCCCCGACTCCTCCACGGGCATCGGCAAGAAAGGCCAG |
| | | CAGCCCGCTAAAAAGAAGCTCAACTTTGGGCAGACTGGCG |
| | | ACTCAGAGTCAGTGCCCGACCCTCAACCAATCGGAGAACC |
| | | CCCCGCAGGCCCCTCTGGTCTGGGATCTGGTACAATGGCT |
| | | TCAGGTGGTGGCGCACCAGTGGCAGACAATAACGAAGGTG |
| | | CCGATGGAGTGGGTAGTTCCTCGGGAAATTGGCATTGCGA |
| | | TTCCCAATGGCTGGGGGACAGAGTCATCACCACCAGCACC |
| | | CGAACCTGGGCCCTGCCCACCTACAACAATCACCTCTACA |
| | | AGCAAATCTCCAACAGCACATCTGGAGGATCTTCAAATGA |
| | | CAACGCCTACTTCGGCTACAGCACCCCCTGGGGGTATTTT |
| | | GACTTCAACAGATTCCACTGCCACTTCTCACCACGTGACT |
| | | GGCAGCGACTCATCAACAACAACTGGGGATTCCGGCCTAA |
| | | GCGACTCAACTTCAAGCTCTTCAACATTCAGGTCAAAGAG |
| | | GTTACGGACAACAATGGAGTCAAGACCATCGCCAATAACC |
| | | TTACCAGCACGGTCCAGGTCTTCACGGACTCAGACTATCA |
| | | GCTCCCGTACGTGCTCGGGTCGGCTCACGAGGGCTGCCTC |
| | | CCGCCGTTCCCAGCGGACGTTTTCATGATTCCTCAGTACG |
| | | GGTATCTGACGCTTAATGATGGGAGCCAGGCCGTGGGTCG |
| | | TTCGTCCTTTTACTGCCTGGAATATTTCCCGTCGCAAATG |
| | | CTAAGAACGGGTAACAACTTCCAGTTCAGCTACGAGTTTG |
| | | AGAACGTACCTTTCCATAGCAGCTACGCTCACAGCCAAAG |
| | | CCTGGACCGACTAATGAATCCACTCATCGACCAATACTTG |
| | | TACTATCTCTCAAAGACTATTAACGGTTCTGGACAGAATC |
| | | AACAAACGCTAAAATTCAGCGTGGCCGGACCCAGCAACAT |
| | | GGCTGTCCAGGGAAGAAACTACATACCTGGACCCAGCTAC |
| | | CGACAACAACGTGTCTCAACCACTGTGACTCAAAACAACA |
| | | ACAGCGAATTTGCTTGGCCTGGAGCTTCTTCTTGGGCTCT |
| | | CAATGGACGTAATAGCTTGATGAATCCTGGACCTGCTATG |
| | | GCCAGCCACAAAGAAGGAGAGGACCGTTTCTTTCCTTTGT |
| | | CTGGATCTTTAATTTTTGGCAAACAAGGAACTGGAAGAGA |
| | | CAACGTGGATGCGGACAAAGTCATGATAACCAACGAAGAA |
| | | GAAATTAAAACTACTAACCCGGTAGCAACGGAGTCCTATG |
| | | GACAAGTGGCCACAAACCACCAGAGTGCCCAAGCACAGGC |
| | | GCAGACCGGCTGGGTTCAAAACCAAGGAATACTTCCGGGT |
| | | ATGGTTTGGCAGGACAGAGATGTGTACCTGCAAGGACCCA |
| | | TTTGGGCCAAAATTCCTCACACGGACGGCAACTTTCACCC |
| | | TTCTCCGCTAATGGGAGGGTTTGGAATGAAGCACCCGCCT |
| | | CCTCAGATCCTCATCAAAAACACACCTGTACCTGCGGATC |
| | | CTCCAACGGCTTTCAATAAGGACAAGCTGAACTCTTTCAT |
| | | CACCCAGTATTCTACTGGCCAAGTCAGCGTGGAGATTGAG |
| | | TGGGAGCTGCAGAAGGAAAACAGCAAGCGCTGGAACCCGG |
| | | AGATCCAGTACACTTCCAACTATTACAAGTCTAATAATGT |
| | | TGAATTTGCTGTTAATACTGAAGGTGTATATAGTGAACCC |
| | | CGCCCCATTGGCACCAGATACCTGACTCGTAATCTGTAA |
| 59 | AAVrh13/ | MAADGYLPDWLEDNLSEGIREWWDLKPGAPKPKANQQKQD |
| | AAVhu32 | DGRGLVLPGYKYLGPFNGLDKGEPVNEADAAALEHDKAYD |
| | VP1 aa | KQLEQGDNPYLKYNHADAEFQERLQEDTSFGGNLGRAVFQ |
| | | AKKRVLEPLGLVEEGAKTAPGKKRPIESPDSSTGIGKKGQ |
| | | QPAKKKLNFGQTGDSESVPDPQPLGEPPAAPSGLGSGTMA |
| | | SGGGAPVADNNEGADGVGSSSGNWHCDSQWLGDRVITTST |
| | | RTWALPTYNNHLYKQISNSTSGGSSNDNAYFGYSTPWGYF |
| | | DFNRFHCHFSPRDWQRLINNNWGFRPKRLNFKLFNIQVKE |
| | | VTDNNGVKTIANNLTSTVQVFTDSDYQLPYVLGSAHEGCL |
| | | PPFPADVFMIPQYGYLTLNDGSQAVGRSSFYCLEYFPSQM |
| | | LRTGNNFQFSYEFENVPFHSSYAHSQSLDRLMNPLIDQYL |
| | | YYLSKTINGSGQNQQTLKFSVAGPSNMAVQGRNYIPGPSY |
| | | RQQRVSTTVTQNNNSEFAWPGASSWALNGRNSLMNPGPAM |
| | | ASHKEGEDRFFPLSGSLIFGKQGTGRDNVDADKVMITNEE |
| | | EIKTTNPVATESYGQVATNHQSAQAQAQTGWVQNQGILPG |
| | | MVWQDRDVYLQGPIWAKIPHTDGNFHPSPLMGGFGMKHPP |
| | | PQILIKNTPVPADPPTAFNKDKLNSFITQYSTGQVSVEIE |
| | | WELQKENSKRWNPEIQYTSNYYKSNNVEFAVNTEGVYSEP |
| | | RPIGTRYLTRNL |
| 60 | AAVrh13/ | ATGGCTGCCGATGGTTATCTTCCAGATTGGCTCGAGGACA |
| | AAVhu32 | ACCTCTCTGAGGGCATTCGCGAGTGGTGGGACTTGAAACC |
| | VP1 dna | TGGAGCCCCGAAACCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGAGAGCCGGTCAACGA |
| | | GGCAGACGCCGCGGCCCTCGAGCACGACAAGGCCTACGAC |
| | | AAGCAGCTCGAGCAGGGGGACAACCCGTACCTCAAGTACA |
| | | ACCACGCCGACGCCGAGTTTCAGGAGCGTCTTCAAGAAGA |
| | | TACGTCTTTTGGGGGCAACCTCGGGCGAGCAGTCTTCCAG |
| | | GCCAAGAAGCGGGTTCTCGAACCTCTCGGTCTGGTTGAGG |
| | | AAGGCGCTAAGACGGCTCCTGGAAAGAAGAGACCCATAGA |
| | | ATCCCCCGACTCCTCCACGGGCATCGGCAAGAAAGGCCAG |
| | | CAGCCCGCTAAAAAGAAGCTCAACTTTGGGCAGACTGGCG |
| | | ACTCAGAGTCAGTGCCCGACCCCCAACCTCTCGGAGAACC |
| | | TCCCGCCGCGCCCTCAGGTCTGGGATCTGGTACAATGGCT |
| | | TCAGGTGGTGGCGCACCAGTGGCAGACAATAACGAAGGTG |
| | | CCGATGGAGTGGGTAGTTCCTCGGGAAATTGGCATTGCGA |
| | | TTCCCAATGGCTGGGGGACAGAGTCATCACCACCAGCACC |
| | | CGAACCTGGGCCCTGCCCACCTACAACAATCACCTCTACA |
| | | AGCAAATCTCCAACAGCACATCTGGAGGATCTTCAAATGA |
| | | CAACGCCTACTTCGGCTACAGCACCCCCTGGGGGTATTTT |
| | | GACTTCAACAGATTCCACTGCCACTTCTCACCACGTGACT |
| | | GGCAGCGACTCATCAACAACAACTGGGGATTCCGGCCTAA |
| | | GCGACTCAACTTCAAGCTCTTCAACATTCAGGTCAAAGAG |
| | | GTTACGGACAACAATGGAGTCAAGACCATCGCCAATAACC |
| | | TTACCAGCACGGTCCAGGTCTTCACGGACTCAGACTATCA |
| | | GCTCCCGTACGTGCTCGGGTCGGCTCACGAGGGCTGCCTC |
| | | CCGCCGTTCCCAGCGGACGTTTTCATGATTCCTCAGTACG |
| | | GGTATCTGACGCTTAATGATGGGAGCCAGGCCGTGGGTCG |
| | | TTCGTCCTTTTACTGCCTGGAATATTTCCCGTCGCAAATG |
| | | CTAAGAACGGGTAACAACTTCCAGTTCAGCTACGAGTTTG |
| | | AGAACGTACCTTTCCATAGCAGCTACGCTCACAGCCAAAG |
| | | CCTGGACCGACTAATGAATCCACTCATCGACCAATACTTG |
| | | TACTATCTCTCAAAGACTATTAACGGTTCTGGACAGAATC |
| | | AACAAACGCTAAAATTCAGCGTGGCCGGACCCAGCAACAT |
| | | GGCTGTCCAGGGAAGAAACTACATACCTGGACCCAGCTAC |
| | | CGACAACAACGTGTCTCAACCACTGTGACTCAAAACAACA |
| | | ACAGCGAATTTGCTTGGCCTGGAGCTTCTTCTTGGGCTCT |
| | | CAATGGACGTAATAGCTTGATGAATCCTGGACCTGCTATG |
| | | GCCAGCCACAAAGAAGGAGAGGACCGTTTCTTTCCTTTGT |
| | | CTGGATCTTTAATTTTTGGCAAACAAGGAACTGGAAGAGA |
| | | CAACGTGGATGCGGACAAAGTCATGATAACCAACGAAGAA |
| | | GAAATTAAAACTACTAACCCGGTAGCAACGGAGTCCTATG |
| | | GACAAGTGGCCACAAACCACCAGAGTGCCCAAGCACAGGC |
| | | GCAGACCGGCTGGGTTCAAAACCAAGGAATACTTCCGGGT |
| | | ATGGTTTGGCAGGACAGAGATGTGTACCTGCAAGGACCCA |
| | | TTTGGGCCAAAATTCCTCACACGGACGGCAACTTTCACCC |
| | | TTCTCCGCTAATGGGAGGGTTTGGAATGAAGCACCCGCCT |
| | | CCTCAGATCCTCATCAAAAACACACCTGTACCTGCGGATC |
| | | CTCCAACGGCTTTCAATAAGGACAAGCTGAACTCTTTCAT |
| | | CACCCAGTATTCTACTGGCCAAGTCAGCGTGGAGATTGAG |
| | | TGGGAGCTGCAGAAGGAAAACAGCAAGCGCTGGAACCCGG |
| | | AGATCCAGTACACTTCCAACTATTACAAGTCTAATAATGT |
| | | TGAATTTGCTGTTAATACTGAAGGTGTATATAGTGAACCC |
| | | CGCCCCATTGGCACCAGATACCTGACTCGTAATCTGTAA |
| 61 | AAV6/AAVhu32 | MAADGYLPDWLEDNLSEGIREWWDLKPGAPKPKANQQKQD |
| | VP1 aa | DGRGLVLPGYKYLGPFNGLDKGEPVNAADAAALEHDKAYD |
| | | QQLKAGDNPYLRYNHADAEFQERLQEDTSFGGNLGRAVFQ |
| | | AKKRVLEPFGLVEEGAKTAPGKKRPVEQSPQEPDSSSGIG |
| | | KTGQQPAKKRLNFGQTGDSESVPDPQPLGEPPATPAAVGP |
| | | TTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQAQAQTGWVQNQG |
| | | ILPGMVWQDRDVYLQGPIWAKIPHTDGNFHPSPLMGGFGM |
| | | KHPPPQILIKNTPVPADPPTAFNKDKLNSFITQYSTGQVS |
| | | VEIEWELQKENSKRWNPEIQYTSNYYKSNNVEFAVNTEGV |
| | | YSEPRPIGTRYLTRNL |
| 62 | AAV6/AAVhu32 | ATGGCTGCCGATGGTTATCTTCCAGATTGGCTCGAGGACA |
| | VP1 dna | ACCTCTCTGAGGGCATTCGCGAGTGGTGGGACTTGAAACC |
| | | TGGAGCCCCGAAACCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGATGCAGCGGCCCTCGAGCACGACAAGGCCTACGAC |
| | | CAGCAGCTCAAAGCGGGTGACAATCCGTACCTGCGGTATA |
| | | ACCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGAGGGTTCTCGAACCTTTTGGTCTGGTTGAGG |
| | | AAGGTGCTAAGACGGCTCCTGGAAAGAAACGTCCGGTAGA |
| | | GCAGTCGCCACAAGAGCCAGACTCCTCCTCGGGCATTGGC |
| | | AAGACAGGCCAGCAGCCCGCTAAAAAGAGACTCAATTTTG |
| | | GTCAGACTGGCGACTCAGAGTCAGTCCCCGACCCACAACC |
| | | TCTCGGAGAACCTCCAGCAACCCCCGCTGCTGTGGGACCT |
| | | ACTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAGCACAGGCGCAGACCGGCTGGGTTCAAAACCAAGGA |
| | | ATACTTCCGGGTATGGTTTGGCAGGACAGAGATGTGTACC |
| | | TGCAAGGACCCATTTGGGCCAAAATTCCTCACACGGACGG |
| | | CAACTTTCACCCTTCTCCGCTAATGGGAGGGTTTGGAATG |
| | | AAGCACCCGCCTCCTCAGATCCTCATCAAAAACACACCTG |
| | | TACCTGCGGATCCTCCAACGGCTTTCAATAAGGACAAGCT |
| | | GAACTCTTTCATCACCCAGTATTCTACTGGCCAAGTCAGC |
| | | GTGGAGATTGAGTGGGAGCTGCAGAAGGAAAACAGCAAGC |
| | | GCTGGAACCCGGAGATCCAGTACACTTCCAACTATTACAA |
| | | GTCTAATAATGTTGAATTTGCTGTTAATACTGAAGGTGTA |
| | | TATAGTGAACCCCGCCCCATTGGCACCAGATACCTGACTC |
| | | GTAATCTGTAA |
| 63 | AAV5/AAVhu32 | MSFVDHPPDWLEEVGEGLREFLGLEAGPPKPKPNQQHQDQ |
| | VP1 aa | ARGLVLPGYNYLGPGNGLDRGEPVNRADEVAREHDISYNE |
| | | QLEAGDNPYLKYNHADAEFQEKLADDTSFGGNLGKAVFQA |
| | | KKRVLEPFGLVEEGAKTAPTGKRIDDHFPKRKKARTEEDS |
| | | KPSTSSDAEAGPSGSQQLQIPAQPASSLGADTMASGGGAP |
| | | VADNNEGADGVGSSSGNWHCDSQWLGDRVITTSTRTWALP |
| | | TYNNHLYKQISNSTSGGSSNDNAYFGYSTPWGYFDFNRFH |
| | | CHFSPRDWQRLINNNWGFRPKRLNFKLFNIQVKEVTDNNG |
| | | VKTIANNLTSTVQVFTDSDYQLPYVLGSAHEGCLPPFPAD |
| | | VFMIPQYGYLTLNDGSQAVGRSSFYCLEYFPSQMLRTGNN |
| | | FQFSYEFENVPFHSSYAHSQSLDRLMNPLIDQYLYYLSKT |
| | | INGSGQNQQTLKFSVAGPSNMAVQGRNYIPGPSYRQQRVS |
| | | TTVTQNNNSEFAWPGASSWALNGRNSLMNPGPAMASHKEG |
| | | EDRFFPLSGSLIFGKQGTGRDNVDADKVMITNEEEIKTTN |
| | | PVATESYGQVATNHQSAQAQAQTGWVQNQGILPGMVWQDR |
| | | DVYLQGPIWAKIPHTDGNFHPSPLMGGFGMKHPPPQILIK |
| | | NTPVPADPPTAFNKDKLNSFITQYSTGQVSVEIEWELQKE |
| | | NSKRWNPEIQYTSNYYKSNNVEFAVNTEGVYSEPRPIGTR |
| | | YLTRNL |
| 64 | AAV5/AAVhu32 | ATGTCTTTTGTTGATCACCCTCCAGATTGGTTGGAAGAAG |
| | VP1 dna | TTGGTGAAGGTCTTCGCGAGTTTTTGGGCCTTGAAGCGGG |
| | | CCCACCGAAACCAAAACCCAATCAGCAGCATCAAGATCAA |
| | | GCCCGTGGTCTTGTGCTGCCTGGTTATAACTATCTCGGAC |
| | | CCGGAAACGGTCTCGATCGAGGAGAGCCTGTCAACAGGGC |
| | | AGACGAGGTCGCGCGAGAGCACGACATCTCGTACAACGAG |
| | | CAGCTTGAGGCGGGAGACAACCCCTACCTCAAGTACAACC |
| | | ACGCGGACGCCGAGTTTCAGGAGAAGCTCGCCGACGACAC |
| | | ATCCTTCGGGGGAAACCTCGGAAAGGCAGTCTTTCAGGCC |
| | | AAGAAAAGGGTTCTCGAACCTTTTGGCCTGGTTGAAGAGG |
| | | GTGCTAAGACGGCCCCTACCGGAAAGCGGATAGACGACCA |
| | | CTTTCCAAAAAGAAAGAAGGCTCGGACCGAAGAGGACTCC |
| | | AAGCCTTCCACCTCGTCAGACGCCGAAGCTGGACCCAGCG |
| | | GATCCCAGCAGCTGCAAATCCCAGCCCAACCAGCCTCAAG |
| | | TTTGGGAGCTGATACAATGGCTTCAGGTGGTGGCGCACCA |
| | | GTGGCAGACAATAACGAAGGTGCCGATGGAGTGGGTAGTT |
| | | CCTCGGGAAATTGGCATTGCGATTCCCAATGGCTGGGGGA |
| | | CAGAGTCATCACCACCAGCACCCGAACCTGGGCCCTGCCC |
| | | ACCTACAACAATCACCTCTACAAGCAAATCTCCAACAGCA |
| | | CATCTGGAGGATCTTCAAATGACAACGCCTACTTCGGCTA |
| | | CAGCACCCCCTGGGGGTATTTTGACTTCAACAGATTCCAC |
| | | TGCCACTTCTCACCACGTGACTGGCAGCGACTCATCAACA |
| | | ACAACTGGGGATTCCGGCCTAAGCGACTCAACTTCAAGCT |
| | | CTTCAACATTCAGGTCAAAGAGGTTACGGACAACAATGGA |
| | | GTCAAGACCATCGCCAATAACCTTACCAGCACGGTCCAGG |
| | | TCTTCACGGACTCAGACTATCAGCTCCCGTACGTGCTCGG |
| | | GTCGGCTCACGAGGGCTGCCTCCCGCCGTTCCCAGCGGAC |
| | | GTTTTCATGATTCCTCAGTACGGGTATCTGACGCTTAATG |
| | | ATGGGAGCCAGGCCGTGGGTCGTTCGTCCTTTTACTGCCT |
| | | GGAATATTTCCCGTCGCAAATGCTAAGAACGGGTAACAAC |
| | | TTCCAGTTCAGCTACGAGTTTGAGAACGTACCTTTCCATA |
| | | GCAGCTACGCTCACAGCCAAAGCCTGGACCGACTAATGAA |
| | | TCCACTCATCGACCAATACTTGTACTATCTCTCAAAGACT |
| | | ATTAACGGTTCTGGACAGAATCAACAAACGCTAAAATTCA |
| | | GCGTGGCCGGACCCAGCAACATGGCTGTCCAGGGAAGAAA |
| | | CTACATACCTGGACCCAGCTACCGACAACAACGTGTCTCA |
| | | ACCACTGTGACTCAAAACAACAACAGCGAATTTGCTTGGC |
| | | CTGGAGCTTCTTCTTGGGCTCTCAATGGACGTAATAGCTT |
| | | GATGAATCCTGGACCTGCTATGGCCAGCCACAAAGAAGGA |
| | | GAGGACCGTTTCTTTCCTTTGTCTGGATCTTTAATTTTTG |
| | | GCAAACAAGGAACTGGAAGAGACAACGTGGATGCGGACAA |
| | | AGTCATGATAACCAACGAAGAAGAAATTAAAACTACTAAC |
| | | CCGGTAGCAACGGAGTCCTATGGACAAGTGGCCACAAACC |
| | | ACCAGAGTGCCCAAGCACAGGCGCAGACCGGCTGGGTTCA |
| | | AAACCAAGGAATACTTCCGGGTATGGTTTGGCAGGACAGA |
| | | GATGTGTACCTGCAAGGACCCATTTGGGCCAAAATTCCTC |
| | | ACACGGACGGCAACTTTCACCCTTCTCCGCTAATGGGAGG |
| | | GTTTGGAATGAAGCACCCGCCTCCTCAGATCCTCATCAAA |
| | | AACACACCTGTACCTGCGGATCCTCCAACGGCTTTCAATA |
| | | AGGACAAGCTGAACTCTTTCATCACCCAGTATTCTACTGG |
| | | CCAAGTCAGCGTGGAGATTGAGTGGGAGCTGCAGAAGGAA |
| | | AACAGCAAGCGCTGGAACCCGGAGATCCAGTACACTTCCA |
| | | ACTATTACAAGTCTAATAATGTTGAATTTGCTGTTAATAC |
| | | TGAAGGTGTATATAGTGAACCCCGCCCCATTGGCACCAGA |
| | | TACCTGACTCGTAATCTGTAA |
| 65 | AAVhu32/ | MAADGYLPDWLEDTLSEGIRQWWKLKPGPPPPKPAERHKD |
| | AAVrh15 | DSRGLVLPGYKYLGPGNGLDKGEPVNAADAAALEHDKAYD |
| | VP1 aa | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMAAGGGAPMADNNEGADGVGNASGNWHCDSTWLGDRVI |
| | | TTSTRTWALPTYNNHLYKQISSQSGATNDNHFFGYSTPWG |
| | | YFDFNRFHCHFSPRDWQRLINNNWGFRPRKLRFKLFNIQV |
| | | KEVTTNDGVTTIANNLTSTIQVFSDSEYQLPYVLGSAHQG |
| | | CLPPFPADVFMIPQYGYLTLNNGSQSVGRSSFYCLEYFPS |
| | | QMLRTGNNFEFSYTFEEVPFHSSYAHSQSLDRLMNPLIDQ |
| | | YLYYLARTQSTTGSTRELQFHQAGPNTMAEQSKNWLPGPC |
| | | YRQQRLSKNIDSNNTSNFAWTGATKYHLNGRNSLTNPGVA |
| | | MATNKDDEDQFFPINGVLVFGKTGAANKTTLENVLMTSEE |
| | | EIKTTNPVATEQYGVVSSNLQSSTAGPQTQTVNSQGALPG |
| | | MVWQNRDVYLQGPIWAKIPHTDGNFHPSPLMGGFGLKHPP |
| | | PQILIKNTPVPANPPEVFTPAKFASFITQYSTGQVSVEIE |
| | | WELQKENSKRWNPEIQYTSNYAKSNNVEFAVNNEGVYTEP |
| | | RPIGTRYLTRNL |
| 66 | AAVhu32/ | ATGGCTGCCGATGGTTATCTTCCAGATTGGCTCGAGGACA |
| | AAVrh15 | CTCTCTCTGAAGGAATAAGACAGTGGTGGAAGCTCAAACC |
| | VP1 dna | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTGCAGGCGGTGGCGCTCCAATGGCAGACA |
| | | ATAACGAAGGCGCCGACGGAGTGGGTAATGCCTCCGGAAA |
| | | TTGGCATTGCGATTCCACATGGCTGGGCGACAGAGTCATC |
| | | ACCACCAGCACCCGCACCTGGGCCCTGCCCACCTACAACA |
| | | ACCACCTCTACAAGCAGATATCAAGTCAGAGCGGGGCTAC |
| | | CAACGACAACCACTTCTTCGGCTACAGCACCCCCTGGGGC |
| | | TATTTTGACTTCAACAGATTCCACTGCCACTTCTCACCAC |
| | | GTGACTGGCAGCGACTCATCAACAACAACTGGGGATTCCG |
| | | GCCCAGAAAGCTGCGGTTCAAGTTGTTCAACATCCAGGTC |
| | | AAGGAGGTCACGACGAACGACGGCGTTACGACCATCGCTA |
| | | ATAACCTTACCAGCACGATTCAGGTCTTCTCGGACTCGGA |
| | | GTACCAACTGCCGTACGTCCTCGGCTCTGCGCACCAGGGC |
| | | TGCCTCCCTCCGTTCCCTGCGGACGTGTTCATGATTCCTC |
| | | AGTACGGATATCTGACTCTAAACAACGGCAGTCAGTCTGT |
| | | GGGACGTTCCTCCTTCTACTGCCTGGAGTACTTTCCTTCT |
| | | CAGATGCTGAGAACGGGCAATAACTTTGAATTCAGCTACA |
| | | CCTTTGAGGAAGTGCCTTTCCACAGCAGCTATGCGCACAG |
| | | CCAGAGCCTGGACCGGCTGATGAATCCCCTCATCGACCAG |
| | | TACCTGTACTACCTGGCCCGGACCCAGAGCACTACGGGGT |
| | | CCACAAGGGAGCTGCAGTTCCATCAGGCTGGGCCCAACAC |
| | | CATGGCCGAGCAATCAAAGAACTGGCTGCCCGGACCCTGT |
| | | TATCGGCAGCAGAGACTGTCAAAAAACATAGACAGCAACA |
| | | ACACCAGTAACTTTGCCTGGACCGGGGCCACTAAATACCA |
| | | TCTGAATGGTAGAAATTCATTAACCAACCCGGGCGTAGCC |
| | | ATGGCCACCAACAAGGACGACGAGGACCAGTTCTTTCCCA |
| | | TCAACGGAGTGCTGGTTTTTGGCAAAACGGGGGCTGCCAA |
| | | CAAGACAACGCTGGAAAACGTGCTAATGACCAGCGAGGAG |
| | | GAGATCAAAACCACCAATCCCGTGGCTACAGAACAGTACG |
| | | GTGTGGTCTCCAGCAACCTGCAATCGTCTACGGCCGGACC |
| | | CCAGACACAGACTGTCAACAGCCAGGGGGCTCTGCCCGGC |
| | | ATGGTCTGGCAGAACCGGGACGTGTACCTGCAGGGTCCCA |
| | | TCTGGGCCAAAATTCCTCACACGGACGGCAACTTTCACCC |
| | | GTCTCCCCTGATGGGCGGATTTGGACTCAAACACCCGCCT |
| | | CCTCAAATTCTCATCAAAAACACCCCGGTACCTGCTAATC |
| | | CTCCAGAGGTGTTTACTCCTGCCAAGTTTGCCTCATTTAT |
| | | CACGCAGTACAGCACCGGCCAGGTCAGCGTGGAGATCGAG |
| | | TGGGAACTGCAGAAAGAAAACAGCAAACGCTGGAATCCAG |
| | | AGATTCAGTACACCTCAAATTATGCCAAGTCTAATAATGT |
| | | GGAATTTGCTGTCAACAACGAAGGGGTTTATACTGAGCCT |
| | | CGCCCCATTGGCACCCGTTACCTCACCCGTAACCTGTAA |
| 67 | AAV6/AAVrh15 | MAADGYLPDWLEDNLSEGIREWWDLKPGAPKPKANQQKQD |
| | VP1 aa | DGRGLVLPGYKYLGPFNGLDKGEPVNAADAAALEHDKAYD |
| | | QQLKAGDNPYLRYNHADAEFQERLQEDTSFGGNLGRAVFQ |
| | | AKKRVLEPFGLVEEGAKTAPGKKRPVEQSPQEPDSSSGIG |
| | | KTGQQPAKKRLNFGQTGDSESVPDPQPLGEPPATPAAVGP |
| | | TTMAAGGGAPMADNNEGADGVGNASGNWHCDSTWLGDRVI |
| | | TTSTRTWALPTYNNHLYKQISSQSGATNDNHFFGYSTPWG |
| | | YFDFNRFHCHFSPRDWQRLINNNWGFRPRKLRFKLFNIQV |
| | | KEVTTNDGVTTIANNLTSTIQVFSDSEYQLPYVLGSAHQG |
| | | CLPPFPADVFMIPQYGYLTLNNGSQSVGRSSFYCLEYFPS |
| | | QMLRTGNNFEFSYTFEEVPFHSSYAHSQSLDRLMNPLIDQ |
| | | YLYYLARTQSTTGSTRELQFHQAGPNTMAEQSKNWLPGPC |
| | | YRQQRLSKNIDSNNTSNFAWTGATKYHLNGRNSLTNPGVA |
| | | MATNKDDEDQFFPINGVLVFGKTGAANKTTLENVLMTSEE |
| | | EIKTTNPVATEQYGVVSSNLQSSTAGPQTQTVNSQGALPG |
| | | MVWQNRDVYLQGPIWAKIPHTDGNFHPSPLMGGFGLKHPP |
| | | PQILIKNTPVPANPPEVFTPAKFASFITQYSTGQVSVEIE |
| | | WELQKENSKRWNPEIQYTSNYAKSNNVEFAVNNEGVYTEP |
| | | RPIGTRYLTRNL |
| 68 | AAV6/AAVrh15 | ATGGCTGCCGATGGTTATCTTCCAGATTGGCTCGAGGACA |
| | VP1 dna | ACCTCTCTGAGGGCATTCGCGAGTGGTGGGACTTGAAACC |
| | | TGGAGCCCCGAAACCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGATGCAGCGGCCCTCGAGCACGACAAGGCCTACGAC |
| | | CAGCAGCTCAAAGCGGGTGACAATCCGTACCTGCGGTATA |
| | | ACCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGAGGGTTCTCGAACCTTTTGGTCTGGTTGAGG |
| | | AAGGTGCTAAGACGGCTCCTGGAAAGAAACGTCCGGTAGA |
| | | GCAGTCGCCACAAGAGCCAGACTCCTCCTCGGGCATTGGC |
| | | AAGACAGGCCAGCAGCCCGCTAAAAAGAGACTCAATTTTG |
| | | GTCAGACTGGCGACTCAGAGTCAGTCCCCGACCCACAACC |
| | | TCTCGGAGAACCTCCAGCAACCCCCGCTGCTGTGGGACCT |
| | | ACTACAATGGCTGCAGGCGGTGGCGCTCCAATGGCAGACA |
| | | ATAACGAAGGCGCCGACGGAGTGGGTAATGCCTCCGGAAA |
| | | TTGGCATTGCGATTCCACATGGCTGGGCGACAGAGTCATC |
| | | ACCACCAGCACCCGCACCTGGGCCCTGCCCACCTACAACA |
| | | ACCACCTCTACAAGCAGATATCAAGTCAGAGCGGGGCTAC |
| | | CAACGACAACCACTTCTTCGGCTACAGCACCCCCTGGGGC |
| | | TATTTTGACTTCAACAGATTCCACTGCCACTTCTCACCAC |
| | | GTGACTGGCAGCGACTCATCAACAACAACTGGGGATTCCG |
| | | GCCCAGAAAGCTGCGGTTCAAGTTGTTCAACATCCAGGTC |
| | | AAGGAGGTCACGACGAACGACGGCGTTACGACCATCGCTA |
| | | ATAACCTTACCAGCACGATTCAGGTCTTCTCGGACTCGGA |
| | | GTACCAACTGCCGTACGTCCTCGGCTCTGCGCACCAGGGC |
| | | TGCCTCCCTCCGTTCCCTGCGGACGTGTTCATGATTCCTC |
| | | AGTACGGATATCTGACTCTAAACAACGGCAGTCAGTCTGT |
| | | GGGACGTTCCTCCTTCTACTGCCTGGAGTACTTTCCTTCT |
| | | CAGATGCTGAGAACGGGCAATAACTTTGAATTCAGCTACA |
| | | CCTTTGAGGAAGTGCCTTTCCACAGCAGCTATGCGCACAG |
| | | CCAGAGCCTGGACCGGCTGATGAATCCCCTCATCGACCAG |
| | | TACCTGTACTACCTGGCCCGGACCCAGAGCACTACGGGGT |
| | | CCACAAGGGAGCTGCAGTTCCATCAGGCTGGGCCCAACAC |
| | | CATGGCCGAGCAATCAAAGAACTGGCTGCCCGGACCCTGT |
| | | TATCGGCAGCAGAGACTGTCAAAAAACATAGACAGCAACA |
| | | ACACCAGTAACTTTGCCTGGACCGGGGCCACTAAATACCA |
| | | TCTGAATGGTAGAAATTCATTAACCAACCCGGGCGTAGCC |
| | | ATGGCCACCAACAAGGACGACGAGGACCAGTTCTTTCCCA |
| | | TCAACGGAGTGCTGGTTTTTGGCAAAACGGGGGCTGCCAA |
| | | CAAGACAACGCTGGAAAACGTGCTAATGACCAGCGAGGAG |
| | | GAGATCAAAACCACCAATCCCGTGGCTACAGAACAGTACG |
| | | GTGTGGTCTCCAGCAACCTGCAATCGTCTACGGCCGGACC |
| | | CCAGACACAGACTGTCAACAGCCAGGGGGCTCTGCCCGGC |
| | | ATGGTCTGGCAGAACCGGGACGTGTACCTGCAGGGTCCCA |
| | | TCTGGGCCAAAATTCCTCACACGGACGGCAACTTTCACCC |
| | | GTCTCCCCTGATGGGCGGATTTGGACTCAAACACCCGCCT |
| | | CCTCAAATTCTCATCAAAAACACCCCGGTACCTGCTAATC |
| | | CTCCAGAGGTGTTTACTCCTGCCAAGTTTGCCTCATTTAT |
| | | CACGCAGTACAGCACCGGCCAGGTCAGCGTGGAGATCGAG |
| | | TGGGAACTGCAGAAAGAAAACAGCAAACGCTGGAATCCAG |
| | | AGATTCAGTACACCTCAAATTATGCCAAGTCTAATAATGT |
| | | GGAATTTGCTGTCAACAACGAAGGGGTTTATACTGAGCCT |
| | | CGCCCCATTGGCACCCGTTACCTCACCCGTAACCTGTAA |
| 69 | AAV5/AAVrh15 | MSFVDHPPDWLEEVGEGLREFLGLEAGPPKPKPNQQHQDQ |
| | VP1 aa | ARGLVLPGYNYLGPGNGLDRGEPVNRADEVAREHDISYNE |
| | | QLEAGDNPYLKYNHADAEFQEKLADDTSFGGNLGKAVFQA |
| | | KKRVLEPFGLVEEGAKTAPTGKRIDDHFPKRKKARTEEDS |
| | | KPSTSSDAEAGPSGSQQLQIPAQPASSLGADTMAAGGGAP |
| | | MADNNEGADGVGNASGNWHCDSTWLGDRVITTSTRTWALP |
| | | TYNNHLYKQISSQSGATNDNHFFGYSTPWGYFDFNRFHCH |
| | | FSPRDWQRLINNNWGFRPRKLRFKLFNIQVKEVTTNDGVT |
| | | TIANNLTSTIQVFSDSEYQLPYVLGSAHQGCLPPFPADVF |
| | | MIPQYGYLTLNNGSQSVGRSSFYCLEYFPSQMLRTGNNFE |
| | | FSYTFEEVPFHSSYAHSQSLDRLMNPLIDQYLYYLARTQS |
| | | TTGSTRELQFHQAGPNTMAEQSKNWLPGPCYRQQRLSKNI |
| | | DSNNTSNFAWTGATKYHLNGRNSLTNPGVAMATNKDDEDQ |
| | | FFPINGVLVFGKTGAANKTTLENVLMTSEEEIKTTNPVAT |
| | | EQYGVVSSNLQSSTAGPQTQTVNSQGALPGMVWQNRDVYL |
| | | QGPIWAKIPHTDGNFHPSPLMGGFGLKHPPPQILIKNTPV |
| | | PANPPEVFTPAKFASFITQYSTGQVSVEIEWELQKENSKR |
| | | WNPEIQYTSNYAKSNNVEFAVNNEGVYTEPRPIGTRYLTR |
| | | NL |
| 70 | AAV5/AAVrh15 | ATGTCTTTTGTTGATCACCCTCCAGATTGGTTGGAAGAAG |
| | VP1 dna | TTGGTGAAGGTCTTCGCGAGTTTTTGGGCCTTGAAGCGGG |
| | | CCCACCGAAACCAAAACCCAATCAGCAGCATCAAGATCAA |
| | | GCCCGTGGTCTTGTGCTGCCTGGTTATAACTATCTCGGAC |
| | | CCGGAAACGGTCTCGATCGAGGAGAGCCTGTCAACAGGGC |
| | | AGACGAGGTCGCGCGAGAGCACGACATCTCGTACAACGAG |
| | | CAGCTTGAGGCGGGAGACAACCCCTACCTCAAGTACAACC |
| | | ACGCGGACGCCGAGTTTCAGGAGAAGCTCGCCGACGACAC |
| | | ATCCTTCGGGGGAAACCTCGGAAAGGCAGTCTTTCAGGCC |
| | | AAGAAAAGGGTTCTCGAACCTTTTGGCCTGGTTGAAGAGG |
| | | GTGCTAAGACGGCCCCTACCGGAAAGCGGATAGACGACCA |
| | | CTTTCCAAAAAGAAAGAAGGCTCGGACCGAAGAGGACTCC |
| | | AAGCCTTCCACCTCGTCAGACGCCGAAGCTGGACCCAGCG |
| | | GATCCCAGCAGCTGCAAATCCCAGCCCAACCAGCCTCAAG |
| | | TTTGGGAGCTGATACAATGGCTGCAGGCGGTGGCGCTCCA |
| | | ATGGCAGACAATAACGAAGGCGCCGACGGAGTGGGTAATG |
| | | CCTCCGGAAATTGGCATTGCGATTCCACATGGCTGGGCGA |
| | | CAGAGTCATCACCACCAGCACCCGCACCTGGGCCCTGCCC |
| | | ACCTACAACAACCACCTCTACAAGCAGATATCAAGTCAGA |
| | | GCGGGGCTACCAACGACAACCACTTCTTCGGCTACAGCAC |
| | | CCCCTGGGGCTATTTTGACTTCAACAGATTCCACTGCCAC |
| | | TTCTCACCACGTGACTGGCAGCGACTCATCAACAACAACT |
| | | GGGGATTCCGGCCCAGAAAGCTGCGGTTCAAGTTGTTCAA |
| | | CATCCAGGTCAAGGAGGTCACGACGAACGACGGCGTTACG |
| | | ACCATCGCTAATAACCTTACCAGCACGATTCAGGTCTTCT |
| | | CGGACTCGGAGTACCAACTGCCGTACGTCCTCGGCTCTGC |
| | | GCACCAGGGCTGCCTCCCTCCGTTCCCTGCGGACGTGTTC |
| | | ATGATTCCTCAGTACGGATATCTGACTCTAAACAACGGCA |
| | | GTCAGTCTGTGGGACGTTCCTCCTTCTACTGCCTGGAGTA |
| | | CTTTCCTTCTCAGATGCTGAGAACGGGCAATAACTTTGAA |
| | | TTCAGCTACACCTTTGAGGAAGTGCCTTTCCACAGCAGCT |
| | | ATGCGCACAGCCAGAGCCTGGACCGGCTGATGAATCCCCT |
| | | CATCGACCAGTACCTGTACTACCTGGCCCGGACCCAGAGC |
| | | ACTACGGGGTCCACAAGGGAGCTGCAGTTCCATCAGGCTG |
| | | GGCCCAACACCATGGCCGAGCAATCAAAGAACTGGCTGCC |
| | | CGGACCCTGTTATCGGCAGCAGAGACTGTCAAAAAACATA |
| | | GACAGCAACAACACCAGTAACTTTGCCTGGACCGGGGCCA |
| | | CTAAATACCATCTGAATGGTAGAAATTCATTAACCAACCC |
| | | GGGCGTAGCCATGGCCACCAACAAGGACGACGAGGACCAG |
| | | TTCTTTCCCATCAACGGAGTGCTGGTTTTTGGCAAAACGG |
| | | GGGCTGCCAACAAGACAACGCTGGAAAACGTGCTAATGAC |
| | | CAGCGAGGAGGAGATCAAAACCACCAATCCCGTGGCTACA |
| | | GAACAGTACGGTGTGGTCTCCAGCAACCTGCAATCGTCTA |
| | | CGGCCGGACCCCAGACACAGACTGTCAACAGCCAGGGGGC |
| | | TCTGCCCGGCATGGTCTGGCAGAACCGGGACGTGTACCTG |
| | | CAGGGTCCCATCTGGGCCAAAATTCCTCACACGGACGGCA |
| | | ACTTTCACCCGTCTCCCCTGATGGGCGGATTTGGACTCAA |
| | | ACACCCGCCTCCTCAAATTCTCATCAAAAACACCCCGGTA |
| | | CCTGCTAATCCTCCAGAGGTGTTTACTCCTGCCAAGTTTG |
| | | CCTCATTTATCACGCAGTACAGCACCGGCCAGGTCAGCGT |
| | | GGAGATCGAGTGGGAACTGCAGAAAGAAAACAGCAAACGC |
| | | TGGAATCCAGAGATTCAGTACACCTCAAATTATGCCAAGT |
| | | CTAATAATGTGGAATTTGCTGTCAACAACGAAGGGGTTTA |
| | | TACTGAGCCTCGCCCCATTGGCACCCGTTACCTCACCCGT |
| | | AACCTGTAA |
| 71 | AAV6/AAVrh13 | MAADGYLPDWLEDNLSEGIREWWDLKPGAPKPKANQQKQD |
| | VP1 aa | DGRGLVLPGYKYLGPFNGLDKGEPVNAADAAALEHDKAYD |
| | | QQLKAGDNPYLRYNHADAEFQERLQEDTSFGGNLGRAVFQ |
| | | AKKRVLEPFGLVEEGAKTAPGKKRPVEQSPQEPDSSSGIG |
| | | KTGQQPAKKRLNFGQTGDSESVPDPQPLGEPPATPAAVGP |
| | | TTMAAGGGAPMADNNEGADGVGNASGNWHCDSTWLGDRVI |
| | | TTSTRTWALPTYNNHLYKQISSQSGATNDNHFFGYSTPWG |
| | | YFDFNRFHCHFSPRDWQRLINNNWGFRPRKLRFKLFNIQV |
| | | KEVTTNDGVTTIANNLTSTIQVFSDSEYQLPYVLGSAHQG |
| | | CLPPFPADVFMIPQYGYLTLNNGSQSVGRSSFYCLEYFPS |
| | | QMLRTGNNFEFSYTFEEVPFHSSYAHSQSLDRLMNPLIDQ |
| | | YLYYLARTQSTTGSTRELQFHQAGPNTMAEQSKNWLPGPC |
| | | YRQQRLSKNIDSNNNSNFAWTGATKYHLNGRNSLTNPGVA |
| | | MATNKDDEDQFFPINGVLVFGETGAANKTTLENVLMTSEE |
| | | EIKTTNPVATEEYGVVSSNLQSSTAGPQTQTVNSQGALPG |
| | | MVWQNRDVYLQGPIWAKIPHTDGNFHPSPLMGGFGLKHPP |
| | | PQILIKNTPVPANPPEVFTPAKFASFITQYSTGQVSVEIE |
| | | WELQKENSKRWNPEIQYTSNYAKSNNVEFAVNNEGVYTEP |
| | | RPIGTRYLTRNL |
| 72 | AAV6/AAVrh13 | ATGGCTGCCGATGGTTATCTTCCAGATTGGCTCGAGGACA |
| | VP1 dna | ACCTCTCTGAGGGCATTCGCGAGTGGTGGGACTTGAAACC |
| | | TGGAGCCCCGAAACCCAAAGCCAACCAGCAAAAGCAGGAC |
| | | GACGGCCGGGGTCTGGTGCTTCCTGGCTACAAGTACCTCG |
| | | GACCCTTCAACGGACTCGACAAGGGGGAGCCCGTCAACGC |
| | | GGCGGATGCAGCGGCCCTCGAGCACGACAAGGCCTACGAC |
| | | CAGCAGCTCAAAGCGGGTGACAATCCGTACCTGCGGTATA |
| | | ACCACGCCGACGCCGAGTTTCAGGAGCGTCTGCAAGAAGA |
| | | TACGTCTTTTGGGGGCAACCTCGGGCGAGCAGTCTTCCAG |
| | | GCCAAGAAGAGGGTTCTCGAACCTTTTGGTCTGGTTGAGG |
| | | AAGGTGCTAAGACGGCTCCTGGAAAGAAACGTCCGGTAGA |
| | | GCAGTCGCCACAAGAGCCAGACTCCTCCTCGGGCATTGGC |
| | | AAGACAGGCCAGCAGCCCGCTAAAAAGAGACTCAATTTTG |
| | | GTCAGACTGGCGACTCAGAGTCAGTCCCCGACCCACAACC |
| | | TCTCGGAGAACCTCCAGCAACCCCCGCTGCTGTGGGACCT |
| | | ACTACAATGGCTGCAGGCGGTGGCGCACCAATGGCAGACA |
| | | ATAACGAAGGCGCCGACGGAGTGGGTAATGCCTCCGGAAA |
| | | TTGGCATTGCGATTCCACATGGCTGGGCGACAGAGTCATC |
| | | ACCACCAGCACCCGCACCTGGGCCCTGCCCACCTACAACA |
| | | ACCACCTCTACAAGCAGATATCAAGTCAGAGCGGGGCTAC |
| | | CAACGACAACCACTTCTTCGGCTACAGCACCCCCTGGGGC |
| | | TATTTTGACTTCAACAGATTCCACTGCCACTTCTCACCAC |
| | | GTGACTGGCAGCGACTCATCAACAACAACTGGGGATTCCG |
| | | GCCCAGAAAGCTGCGGTTCAAGTTGTTCAACATCCAGGTC |
| | | AAGGAGGTCACGACGAACGACGGCGTTACGACCATCGCTA |
| | | ATAACCTTACCAGCACGATTCAGGTCTTCTCGGACTCGGA |
| | | GTACCAACTGCCGTACGTCCTCGGCTCTGCGCACCAGGGC |
| | | TGCCTCCCTCCGTTCCCTGCGGACGTGTTCATGATTCCTC |
| | | AGTACGGATATCTGACTCTAAACAACGGCAGTCAGTCTGT |
| | | GGGACGTTCCTCCTTCTACTGCCTGGAGTACTTTCCTTCT |
| | | CAGATGCTGAGAACGGGCAATAACTTTGAATTCAGCTACA |
| | | CCTTTGAGGAAGTGCCTTTCCACAGCAGCTATGCGCACAG |
| | | CCAGAGCCTGGACCGGCTGATGAATCCCCTCATCGACCAG |
| | | TACCTGTACTACCTGGCCCGGACCCAGAGCACTACGGGGT |
| | | CCACAAGGGAGCTGCAGTTCCATCAGGCTGGGCCCAACAC |
| | | CATGGCCGAGCAATCAAAGAACTGGCTGCCCGGACCCTGT |
| | | TATCGGCAGCAGAGACTGTCAAAAAACATAGACAGCAACA |
| | | ACAACAGTAACTTTGCCTGGACCGGGGCCACTAAATACCA |
| | | TCTGAATGGTAGAAATTCATTAACCAACCCGGGCGTAGCC |
| | | ATGGCCACCAACAAGGACGACGAGGACCAGTTCTTTCCCA |
| | | TCAACGGAGTGCTGGTTTTTGGCGAAACGGGGGCTGCCAA |
| | | CAAGACAACGCTGGAAAACGTGCTAATGACCAGCGAGGAG |
| | | GAGATCAAAACCACCAATCCCGTGGCTACAGAAGAATACG |
| | | GTGTGGTCTCCAGCAACCTGCAATCGTCTACGGCCGGACC |
| | | CCAGACACAGACTGTCAACAGCCAGGGGGCTCTGCCCGGC |
| | | ATGGTCTGGCAGAACCGGGACGTGTACCTGCAGGGTCCCA |
| | | TCTGGGCCAAAATTCCTCACACGGACGGCAACTTTCACCC |
| | | GTCTCCCCTGATGGGCGGATTTGGACTCAAACACCCGCCT |
| | | CCTCAAATTCTCATCAAAAACACCCCGGTACCTGCTAATC |
| | | CTCCAGAGGTGTTTACTCCTGCCAAGTTTGCCTCATTTAT |
| | | CACGCAGTACAGCACCGGCCAGGTCAGCGTGGAGATCGAG |
| | | TGGGAACTGCAGAAAGAAAACAGCAAACGCTGGAATCCAG |
| | | AGATTCAGTACACCTCAAATTATGCCAAGTCTAATAATGT |
| | | GGAATTTGCTGTCAACAACGAAGGGGTTTATACTGAGCCT |
| | | CGCCCCATTGGCACCCGTTACCTCACCCGTAACCTGTAA |
| 81 | Hu32 Variant 1 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQSTTLYSPAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 82 | Hu32 Variant 1 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAATCAACGACGCTCTATTCTCCAGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 83 | Hu32 Variant 2 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQFVVGQSYAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 84 | Hu32 Variant 2 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAATTCGTTGTGGGACAGTCCTATGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 85 | Hu32 Variant 3 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQSGRGDLGLGQNQQTLKFSVAGPSN |
| | | MAVQGRNYIPGPSYRQQRVSTTVTQNNNSEFAWPGASSWA |
| | | LNGRNSLMNPGPAMASHKEGEDRFFPLSGSLIFGKQGTGR |
| | | DNVDADKVMITNEEEIKTTNPVATESYGQVATNHQSAQAQ |
| | | AQTGWVQNQGILPGMVWQDRDVYLQGPIWAKIPHTDGNFH |
| | | PSPLMGGFGMKHPPPQILIKNTPVPADPPTAFNKDKLNSF |
| | | ITQYSTGQVSVEIEWELQKENSKRWNPEIQYTSNYYKSNN |
| | | VEFAVNTEGVYSEPRPIGTRYLTRNL |
| 86 | Hu32 Variant 3 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGCCAGAGTGGCCGCGGCGATCTGGGCCTGGGACAGAA |
| | | TCAACAAACGCTAAAATTCAGCGTGGCCGGACCCAGCAAC |
| | | ATGGCTGTCCAGGGAAGAAACTACATACCTGGACCCAGCT |
| | | ACCGACAACAACGTGTCTCAACCACTGTGACTCAAAACAA |
| | | CAACAGCGAATTTGCTTGGCCTGGAGCTTCTTCTTGGGCT |
| | | CTCAATGGACGTAATAGCTTGATGAATCCTGGACCTGCTA |
| | | TGGCCAGCCACAAAGAAGGAGAGGACCGTTTCTTTCCTTT |
| | | GTCTGGATCTTTAATTTTTGGCAAACAAGGAACTGGAAGA |
| | | GACAACGTGGATGCGGACAAAGTCATGATAACCAACGAAG |
| | | AAGAAATTAAAACTACTAACCCGGTAGCAACGGAGTCCTA |
| | | TGGACAAGTGGCCACAAACCACCAGAGTGCCCAAGCACAG |
| | | GCGCAGACCGGCTGGGTTCAAAACCAAGGAATACTTCCGG |
| | | GTATGGTTTGGCAGGACAGAGATGTGTACCTGCAAGGACC |
| | | CATTTGGGCCAAAATTCCTCACACGGACGGCAACTTTCAC |
| | | CCTTCTCCGCTAATGGGAGGGTTTGGAATGAAGCACCCGC |
| | | CTCCTCAGATCCTCATCAAAAACACACCTGTACCTGCGGA |
| | | TCCTCCAACGGCTTTCAATAAGGACAAGCTGAACTCTTTC |
| | | ATCACCCAGTATTCTACTGGCCAAGTCAGCGTGGAGATTG |
| | | AGTGGGAGCTGCAGAAGGAAAACAGCAAGCGCTGGAACCC |
| | | GGAGATCCAGTACACTTCCAACTATTACAAGTCTAATAAT |
| | | GTTGAATTTGCTGTTAATACTGAAGGTGTATATAGTGAAC |
| | | CCCGCCCCATTGGCACCAGATACCTGACTCGTAATCTGTA |
| | | A |
| 87 | Hu32 variant 4 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQGQSGRGDLGLSAQAA |
| | | QTGWVQNQGILPGMVWQDRDVYLQGPIWAKIPHTDGNFHP |
| | | SPLMGGFGMKHPPPQILIKNTPVPADPPTAFNKDKLNSFI |
| | | TQYSTGQVSVEIEWELQKENSKRWNPEIQYTSNYYKSNNV |
| | | EFAVNTEGVYSEPRPIGTRYLTRNL |
| 88 | Hu32 variant 4 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGGGCCA |
| | | GAGTGGCCGCGGCGATCTGGGCCTGAGCGCCCAGGCGGCC |
| | | CAGACCGGCTGGGTTCAAAACCAAGGAATACTTCCGGGTA |
| | | TGGTTTGGCAGGACAGAGATGTGTACCTGCAAGGACCCAT |
| | | TTGGGCCAAAATTCCTCACACGGACGGCAACTTTCACCCT |
| | | TCTCCGCTAATGGGAGGGTTTGGAATGAAGCACCCGCCTC |
| | | CTCAGATCCTCATCAAAAACACACCTGTACCTGCGGATCC |
| | | TCCAACGGCTTTCAATAAGGACAAGCTGAACTCTTTCATC |
| | | ACCCAGTATTCTACTGGCCAAGTCAGCGTGGAGATTGAGT |
| | | GGGAGCTGCAGAAGGAAAACAGCAAGCGCTGGAACCCGGA |
| | | GATCCAGTACACTTCCAACTATTACAAGTCTAATAATGTT |
| | | GAATTTGCTGTTAATACTGAAGGTGTATATAGTGAACCCC |
| | | GCCCCATTGGCACCAGATACCTGACTCGTAATCTGTAA |
| 89 | Hu32 variant 5 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQGRGDLGLAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 90 | Hu32 variant 5 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAGGCCGCGGCGATCTGGGCCTGGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 91 | Hu32 variant 6 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQQRGDYNSAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 92 | Hu32 variant 6 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAACAGAGAGGCGACTACAACTCTGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 93 | Hu32 variant 7 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQNSRGDYNAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 94 | Hu32 variant 7 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAAATTCTAGAGGCGACTACAACGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 95 | Hu32 variant 8 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQENRRGDFAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 96 | Hu32 variant 8 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAGAAAACCGGAGAGGCGACTTCGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 97 | Hu32 variant 9 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQRGDYVGLAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 98 | Hu32 variant 9 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAAGAGGCGACTACGTGGGCCTGGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 99 | Hu32 variant 10 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSNSRGDYNSLAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 100 | Hu32 variant 10 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGCAA |
| | | CTCTAGAGGCGACTACAACAGCCTGGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 101 | Hu32 variant 11 | MAADGYLPDWLEDTLSEGIRQWWKLKPGPPPPKPAERHK |
| | | DDSRGLVLPGYKYLGPGNGLDKGEPVNAADAAALEHDKAY |
| | | DQQLKAGDNPYLKYNHADAEFQERLKEDTSFGGNLGRAVF |
| | | QAKKRLLEPLGLVEEAAKTAPGKKRPVEQSPQEPDSSAGI |
| | | GKSGSQPAKKKLNFGQTGDTESVPDPQPIGEPPAAPSGVG |
| | | SLTMASGGGAPVADNNEGADGVGSSSGNWHCDSQWLGDRV |
| | | ITTSTRTWALPTYNNHLYKQISNSTSGGSSNDNAYFGYST |
| | | PWGYFDFNRFHCHFSPRDWQRLINNNWGFRPKRLNFKLFN |
| | | IQVKEVTDNNGVKTIANNLTSTVQVFTDSDYQLPYVLGSA |
| | | HEGCLPPFPADVFMIPQYGYLTLNDGSQAVGRSSFYCLEY |
| | | FPSQMLRTGNNFQFSYEFENVPFHSSYAHSQSLDRLMNPL |
| | | IDQYLYYLSKTINGSGQNQQTLKFSVAGPSNMAVQGRNYI |
| | | PGPSYRQQRVSTTVTQNNNSEFAWPGASSWALNGRNSLMN |
| | | PGPAMASHKEGEDRFFPLSGSLIFGKQGTGRDNVDADKVM |
| | | ITNEEEIKTTNPVATESYGQVATNHQENRRGDFNNTAQAQ |
| | | TGWVQNQGILPGMVWQDRDVYLQGPIWAKIPHTDGNFHPS |
| | | PLMGGFGMKHPPPQILIKNTPVPADPPTAFNKDKLNSFIT |
| | | QYSTGQVSVEIEWELQKENSKRWNPEIQYTSNYYKSNNVE |
| | | FAVNTEGVYSEPRPIGTRYLTRNL |
| 102 | Hu32 variant 11 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGGAGAA |
| | | TAGACGGGGCGACTTCAACAACACCGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 103 | Hu32 variant 12 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQRGDYVGVAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 104 | Hu32 variant 12 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAAGAGGCGACTACGTGGGCGTGGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 105 | Hu32 variant 13 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQRGDVLGSAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 106 | Hu32 variant 13 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAAGAGGCGACGTGCTGGGCTCTGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 107 | Hu32 variant 14 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQRGDFVGLAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 108 | Hu32 variant 14 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAAGAGGCGACTTCGTGGGCCTGGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 109 | Hu32 variant 15 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQRGDYTLSAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 110 | Hu32 variant 15 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAAGAGGCGACTACACCCTGTCTGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 111 | Hu32 variant 16 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQSRGDFNLAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 112 | Hu32 variant 16 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAATCTAGAGGCGACTTCAACCTGGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 113 | Hu32 variant 17 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQRGDYVAQAQTGW |
| | | VQNQGILPGMVWQDRDVYLQGPIWAKIPHTDGNFHPSPLM |
| | | GGFGMKHPPPQILIKNTPVPADPPTAFNKDKLNSFITQYS |
| | | TGQVSVEIEWELQKENSKRWNPEIQYTSNYYKSNNVEFAV |
| | | NTEGVYSEPRPIGTRYLTRNL |
| 114 | Hu32 variant 17 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTTGGCCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAAGAGGCGACTACGTGGCACAGGCGCAGACCGGCTGG |
| | | GTTCAAAACCAAGGAATACTTCCGGGTATGGTTTGGCAGG |
| | | ACAGAGATGTGTACCTGCAAGGACCCATTTGGGCCAAAAT |
| | | TCCTCACACGGACGGCAACTTTCACCCTTCTCCGCTAATG |
| | | GGAGGGTTTGGAATGAAGCACCCGCCTCCTCAGATCCTCA |
| | | TCAAAAACACACCTGTACCTGCGGATCCTCCAACGGCTTT |
| | | CAATAAGGACAAGCTGAACTCTTTCATCACCCAGTATTCT |
| | | ACTGGCCAAGTCAGCGTGGAGATTGAGTGGGAGCTGCAGA |
| | | AGGAAAACAGCAAGCGCTGGAACCCGGAGATCCAGTACAC |
| | | TTCCAACTATTACAAGTCTAATAATGTTGAATTTGCTGTT |
| | | AATACTGAAGGTGTATATAGTGAACCCCGCCCCATTGGCA |
| | | CCAGATACCTGACTCGTAATCTGTAA |
| 115 | Hu32 variant 18 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFARPGASSWALNGRNSLMNP |
| | | GPAMASHKEGEDRFFPLSGSLIFGKQGTGRDNVDADKVMI |
| | | TNEEEIKTTNPVATESYGQVATNHQSAQAQAQTGWVQNQG |
| | | ILPGMVWQDRDVYLQGPIWAKIPHTDGNFHPSPLMGGFGM |
| | | KHPPPQILIKNTPVPADPPTAFNKDKLNSFITQYSTGQVS |
| | | VEIEWELQKENSKRWNPEIQYTSNYYKSNNVEFAVNTEGV |
| | | YSEPRPIGTRYLTRNL |
| 116 | Hu32 variant 18 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTCGACCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAAGCACAGGCGCAGACCGGCTGGGTTCAAAACCAAGGA |
| | | ATACTTCCGGGTATGGTTTGGCAGGACAGAGATGTGTACC |
| | | TGCAAGGACCCATTTGGGCCAAAATTCCTCACACGGACGG |
| | | CAACTTTCACCCTTCTCCGCTAATGGGAGGGTTTGGAATG |
| | | AAGCACCCGCCTCCTCAGATCCTCATCAAAAACACACCTG |
| | | TACCTGCGGATCCTCCAACGGCTTTCAATAAGGACAAGCT |
| | | GAACTCTTTCATCACCCAGTATTCTACTGGCCAAGTCAGC |
| | | GTGGAGATTGAGTGGGAGCTGCAGAAGGAAAACAGCAAGC |
| | | GCTGGAACCCGGAGATCCAGTACACTTCCAACTATTACAA |
| | | GTCTAATAATGTTGAATTTGCTGTTAATACTGAAGGTGTA |
| | | TATAGTGAACCCCGCCCCATTGGCACCAGATACCTGACTC |
| | | GTAATCTGTAA |
| 117 | Hu32 variant 19 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFARPGASSWALNGRNSLMNP |
| | | GPAMASHKEGEDRFFPLSGSLIFGKQGTGRDNVDADKVMI |
| | | TNEEEIKTTNPVATESYGQVATNHQSAQSTTLYSPAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 118 | Hu32 variant 19 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTCGACCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAATCAACGACGCTCTATTCTCCAGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 119 | Hu32 variant 20 | MAADGYLPDWLEDTLSEGIRQWWKLKPGPPPPKPAERHKD |
| | | DSRGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGSQPAKKKLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFARPGASSWALNGRNSLMNP |
| | | GPAMASHKEGEDRFFPLSGSLIFGKQGTGRDNVDADKVMI |
| | | TNEEEIKTTNPVATESYGQVATNHQSAQFVVGQSYAQAQT |
| | | GWVQNQGILPGMVWQDRDVYLQGPIWAKIPHTDGNFHPSP |
| | | LMGGFGMKHPPPQILIKNTPVPADPPTAFNKDKLNSFITQ |
| | | YSTGQVSVEIEWELQKENSKRWNPEIQYTSNYYKSNNVEF |
| | | AVNTEGVYSEPRPIGTRYLTRNL |
| 120 | Hu32 variant 20 | ATGGCTGCCGATGGTTATCTTCCAGATTGGCTCGAGGACA |
| | | CTCTCTCTGAAGGAATAAGACAGTGGTGGAAGCTCAAACC |
| | | TGGCCCACCACCACCAAAGCCCGCAGAGCGGCATAAGGAC |
| | | GACAGCAGGGGTCTTGTGCTTCCTGGGTACAAGTACCTCG |
| | | GACCCGGCAACGGACTCGACAAGGGGGAGCCGGTCAACGC |
| | | AGCAGACGCGGCGGCCCTCGAGCACGACAAGGCCTACGAC |
| | | CAGCAGCTCAAGGCCGGAGACAACCCGTACCTCAAGTACA |
| | | ACCACGCCGACGCCGAGTTCCAGGAGCGGCTCAAAGAAGA |
| | | TACGTCTTTTGGGGGCAACCTCGGGCGAGCAGTCTTCCAG |
| | | GCCAAAAAGAGGCTTCTTGAACCTCTTGGTCTGGTTGAGG |
| | | AAGCGGCTAAGACGGCTCCTGGAAAGAAGAGGCCTGTAGA |
| | | GCAGTCTCCTCAGGAACCGGACTCCTCCGCGGGTATTGGC |
| | | AAATCGGGTTCACAGCCCGCTAAAAAGAAACTCAATTTCG |
| | | GTCAGACTGGCGACACAGAGTCAGTCCCCGACCCTCAACC |
| | | AATCGGAGAACCTCCCGCAGCCCCCTCAGGTGTGGGATCT |
| | | CTTACAATGGCTTCAGGTGGTGGCGCACCAGTGGCAGACA |
| | | ATAACGAAGGTGCCGATGGAGTGGGTAGTTCCTCGGGAAA |
| | | TTGGCATTGCGATTCCCAATGGCTGGGGGACAGAGTCATC |
| | | ACCACCAGCACCCGAACCTGGGCCCTGCCCACCTACAACA |
| | | ATCACCTCTACAAGCAAATCTCCAACAGCACATCTGGAGG |
| | | ATCTTCAAATGACAACGCCTACTTCGGCTACAGCACCCCC |
| | | TGGGGGTATTTTGACTTCAACAGATTCCACTGCCACTTCT |
| | | CACCACGTGACTGGCAGCGACTCATCAACAACAACTGGGG |
| | | ATTCCGGCCTAAGCGACTCAACTTCAAGCTCTTCAACATT |
| | | CAGGTCAAAGAGGTTACGGACAACAATGGAGTCAAGACCA |
| | | TCGCCAATAACCTTACCAGCACGGTCCAGGTCTTCACGGA |
| | | CTCAGACTATCAGCTCCCGTACGTGCTCGGGTCGGCTCAC |
| | | GAGGGCTGCCTCCCGCCGTTCCCAGCGGACGTTTTCATGA |
| | | TTCCTCAGTACGGGTATCTGACGCTTAATGATGGGAGCCA |
| | | GGCCGTGGGTCGTTCGTCCTTTTACTGCCTGGAATATTTC |
| | | CCGTCGCAAATGCTAAGAACGGGTAACAACTTCCAGTTCA |
| | | GCTACGAGTTTGAGAACGTACCTTTCCATAGCAGCTACGC |
| | | TCACAGCCAAAGCCTGGACCGACTAATGAATCCACTCATC |
| | | GACCAATACTTGTACTATCTCTCAAAGACTATTAACGGTT |
| | | CTGGACAGAATCAACAAACGCTAAAATTCAGCGTGGCCGG |
| | | ACCCAGCAACATGGCTGTCCAGGGAAGAAACTACATACCT |
| | | GGACCCAGCTACCGACAACAACGTGTCTCAACCACTGTGA |
| | | CTCAAAACAACAACAGCGAATTTGCTCGACCTGGAGCTTC |
| | | TTCTTGGGCTCTCAATGGACGTAATAGCTTGATGAATCCT |
| | | GGACCTGCTATGGCCAGCCACAAAGAAGGAGAGGACCGTT |
| | | TCTTTCCTTTGTCTGGATCTTTAATTTTTGGCAAACAAGG |
| | | AACTGGAAGAGACAACGTGGATGCGGACAAAGTCATGATA |
| | | ACCAACGAAGAAGAAATTAAAACTACTAACCCGGTAGCAA |
| | | CGGAGTCCTATGGACAAGTGGCCACAAACCACCAGAGTGC |
| | | CCAATTCGTTGTGGGACAGTCCTATGCACAGGCGCAGACC |
| | | GGCTGGGTTCAAAACCAAGGAATACTTCCGGGTATGGTTT |
| | | GGCAGGACAGAGATGTGTACCTGCAAGGACCCATTTGGGC |
| | | CAAAATTCCTCACACGGACGGCAACTTTCACCCTTCTCCG |
| | | CTAATGGGAGGGTTTGGAATGAAGCACCCGCCTCCTCAGA |
| | | TCCTCATCAAAAACACACCTGTACCTGCGGATCCTCCAAC |
| | | GGCTTTCAATAAGGACAAGCTGAACTCTTTCATCACCCAG |
| | | TATTCTACTGGCCAAGTCAGCGTGGAGATTGAGTGGGAGC |
| | | TGCAGAAGGAAAACAGCAAGCGCTGGAACCCGGAGATCCA |
| | | GTACACTTCCAACTATTACAAGTCTAATAATGTTGAATTT |
| | | GCTGTTAATACTGAAGGTGTATATAGTGAACCCCGCCCCA |
| | | TTGGCACCAGATACCTGACTCGTAATCTGTAA |
| 121 | AAV9/hu32 | MAADGYLPDWLEDXLSEGIRXWWXLKPGXPXPKXXXXHXD |
| | consensus N- | XXRGLVLPGYKYLGPGNG |
| | terminal sequence | [14X = N or T; 21X = E or Q; 24X = A or K; |
| | | 29X = A or P; 31X = Q or P; 33X = A or P; |
| | | 34X = A or P; 35X = N or A; 36X = Q or E; |
| | | 37X = Q or R; 39X = Q or K; 41X = N or D; |
| | | and 42X = A or S] |
| 122 | AAV9 N-terminal | MAADGYLPDWLEDNLSEGIREWWALKPGAPQPKANQQHQD |
| | sequence | NARGLVLPGYKYLGPGNG |
| 123 | Hu32 N-terminal | MAADGYLPDWLEDTLSEGIRQWWKLKPGPPPPKPAERHKD |
| | sequence | DSRGLVLPGYKYLGPGNG |
| 124 | AAV9 | MAADGYLPDWLEDNLSEGIREWWALKPGAPQPKANQQHQD |
| | | NARGLVLPGYKYLGPGNGLDKGEPVNAADAAALEHDKAYD |
| | | QQLKAGDNPYLKYNHADAEFQERLKEDTSFGGNLGRAVFQ |
| | | AKKRLLEPLGLVEEAAKTAPGKKRPVEQSPQEPDSSAGIG |
| | | KSGAQPAKKRLNFGQTGDTESVPDPQPIGEPPAAPSGVGS |
| | | LTMASGGGAPVADNNEGADGVGSSSGNWHCDSQWLGDRVI |
| | | TTSTRTWALPTYNNHLYKQISNSTSGGSSNDNAYFGYSTP |
| | | WGYFDFNRFHCHFSPRDWQRLINNNWGFRPKRLNFKLFNI |
| | | QVKEVTDNNGVKTIANNLTSTVQVFTDSDYQLPYVLGSAH |
| | | EGCLPPFPADVFMIPQYGYLTLNDGSQAVGRSSFYCLEYF |
| | | PSQMLRTGNNFQFSYEFENVPFHSSYAHSQSLDRLMNPLI |
| | | DQYLYYLSKTINGSGQNQQTLKFSVAGPSNMAVQGRNYIP |
| | | GPSYRQQRVSTTVTQNNNSEFAWPGASSWALNGRNSLMNP |
| | | GPAMASHKEGEDRFFPLSGSLIFGKQGTGRDNVDADKVMI |
| | | TNEEEIKTTNPVATESYGQVATNHQSAQAQAQTGWVQNOG |
| | | ILPGMVWQDRDVYLQGPIWAKIPHTDGNFHPSPLMGGFGM |
| | | KHPPPQILIKNTPVPADPPTAFNKDKLNSFITQYSTGQVS |
| | | VEIEWELQKENSKRWNPEIQYTSNYYKSNNVEFAVNTEGV |
| | | YSEPRPIGTRYLTRNL |
| 125 | AAV9 | atggctgccg atggttatct tccagattgg |
| | | ctcgaggaca accttagtga aggaattcgc |
| | | gagtggtggg ctttgaaacc tggagcccct |
| | | caacccaagg caaatcaaca acatcaagac |
| | | aacgctcgag gtcttgtgct tccgggttac |
| | | aaataccttg gacccggcaa cggactcgac |
| | | aagggggagc cggtcaacgc agcagacgcg |
| | | gcggccctcg agcacgacaa ggcctacgac |
| | | cagcagctca aggccggaga caacccgtac |
| | | ctcaagtaca accacgccga cgccgagttc |
| | | caggagcggc tcaaagaaga tacgtctttt |
| | | gggggcaacc tcgggcgagc agtcttccag |
| | | gccaaaaaga ggcttcttga acctcttggt |
| | | ctggttgagg aagcggctaa gacggctcct |
| | | ggaaagaaga ggcctgtaga gcagtctcct |
| | | caggaaccgg actcctccgc gggtattggc |
| | | aaatcgggtg cacagcccgc taaaaagaga |
| | | ctcaatttcg gtcagactgg cgacacagag |
| | | tcagtcccag accctcaacc aatcggagaa |
| | | cctcccgcag ccccctcagg tgtgggatct |
| | | cttacaatgg cttcaggtgg tggcgcacca |
| | | gtggcagaca ataacgaagg tgccgatgga |
| | | gtgggtagtt cctcgggaaa ttggcattgc |
| | | gattcccaat ggctggggga cagagtcatc |
| | | accaccagca cccgaacctg ggccctgccc |
| | | acctacaaca atcacctcta caagcaaatc |
| | | tccaacagca catctggagg atottcaaat |
| | | gacaacgcct acttcggcta cagcaccccc |
| | | tgggggtatt ttgacttcaa cagattccac |
| | | tgccacttct caccacgtga ctggcagcga |
| | | ctcatcaaca acaactgggg attccggcct |
| | | aagcgactca acttcaagct cttcaacatt |
| | | caggtcaaag aggttacgga caacaatgga |
| | | gtcaagacca tcgccaataa ccttaccagc |
| | | acggtccagg tottcacgga ctcagactat |
| | | cagctcccgt acgtgctcgg gtcggctcac |
| | | gagggctgcc tcccgccgtt cccagcggac |
| | | gttttcatga ttcctcagta cgggtatctg |
| | | acgcttaatg atggaagcca ggccgtgggt |
| | | cgttcgtcct tttactgcct ggaatatttc |
| | | ccgtcgcaaa tgctaagaac gggtaacaac |
| | | ttccagttca gctacgagtt tgagaacgta |
| | | cctttccata gcagctacgc tcacagccaa |
| | | agcctggacc gactaatgaa tccactcatc |
| | | gaccaatact tgtactatct ctcaaagact |
| | | attaacggtt ctggacagaa tcaacaaacg |
| | | ctaaaattca gtgtggccgg acccagcaac |
| | | atggctgtcc agggaagaaa ctacatacct |
| | | ggacccagct accgacaaca acgtgtctca |
| | | accactgtga ctcaaaacaa caacagcgaa |
| | | tttgcttggc ctggagcttc ttcttgggct |
| | | ctcaatggac gtaatagctt gatgaatcct |
| | | ggacctgcta tggccagcca caaagaagga |
| | | gaggaccgtt totttccttt gtctggatct |
| | | ttaatttttg gcaaacaagg aactggaaga |
| | | gacaacgtgg atgcggacaa agtcatgata |
| | | accaacgaag aagaaattaa aactactaac |
| | | ccggtagcaa cggagtccta tggacaagtg |
| | | gccacaaacc accagagtgc ccaagcacag |
| | | gcgcagaccg gctgggttca aaaccaagga |
| | | atacttccgg gtatggtttg gcaggacaga |
| | | gatgtgtacc tgcaaggacc catttgggcc |
| | | aaaattcctc acacggacgg caactttcac |
| | | ccttctccgc tgatgggagg gtttggaatg |
| | | aagcacccgc ctcctcagat cctcatcaaa |
| | | aacacacctg tacctgcgga tectccaacg |
| | | gccttcaaca aggacaagct gaactctttc |
| | | atcacccagt attctactgg ccaagtcagc |
| | | gtggagatcg agtgggagct gcagaaggaa |
| | | aacagcaagc gctggaaccc ggagatccag |
| | | tacacttcca actattacaa gtctaataat |
| | | gttgaatttg ctgttaatac tgaaggtgta |
| | | tatagtgaac cccgccccat tggcaccaga |
| | | tacctgactc gtaatctgta a |
| 126 | AAV1 | MAADGYLPDWLEDNLSEGIREWWDLKPGAPKPKANQQK |
| | | QDDGRGLVLPGYKYLGPFNGLDKGEPVNAADAAALEHD |
| | | KAYDQQLKAGDNPYLRYNHADAEFQERLQEDTSFGGNL |
| | | GRAVFQAKKRVLEPLGLVEEGAKTAPGKKRPVEQSPQE |
| | | PDSSSGIGKTGQQPAKKRLNFGQTGDSESVPDPQPLGE |
| | | PPATPAAVGPTTMASGGGAPMADNNEGADGVGNASGNW |
| | | HCDSTWLGDRVITTSTRTWALPTYNNHLYKQISSASTG |
| | | ASNDNHYFGYSTPWGYFDFNRFHCHFSPRDWQRLINNN |
| | | WGFRPKRLNFKLFNIQVKEVTTNDGVTTIANNLTSTVQ |
| | | VFSDSEYQLPYVLGSAHQGCLPPFPADVFMIPQYGYLT |
| | | LNNGSQAVGRSSFYCLEYFPSQMLRTGNNFTFSYTFEE |
| | | VPFHSSYAHSQSLDRLMNPLIDQYLYYLNRTQNQSGSA |
| | | QNKDLLFSRGSPAGMSVQPKNWLPGPCYRQQRVSKTKT |
| | | DNNNSNFTWTGASKYNLNGRESIINPGTAMASHKDDED |
| | | KFFPMSGVMIFGKESAGASNTALDNVMITDEEEIKATN |
| | | PVATERFGTVAVNFQSSSTDPATGDVHAMGALPGMVWQ |
| | | DRDVYLQGPIWAKIPHTDGHFHPSPLMGGFGLKNPPPQ |
| | | ILIKNTPVPANPPAEFSATKFASFITQYSTGQVSVEIE |
| | | WELQKENSKRWNPEVQYTSNYAKSANVDFTVDNNGLYT |
| | | EPRPIGTRYLTRPL |
| 127 | AAV2 | MAADGYLPDWLEDTLSEGIRQWWKLKPGPPPPKPAERH |
| | | KDDSRGLVLPGYKYLGPFNGLDKGEPVNEADAAALEHD |
| | | KAYDRQLDSGDNPYLKYNHADAEFQERLKEDTSFGGNL |
| | | GRAVFQAKKRVLEPLGLVEEPVKTAPGKKRPVEHSPVE |
| | | PDSSSGTGKAGQQPARKRLNFGQTGDADSVPDPQPLGQ |
| | | PPAAPSGLGTNTMATGSGAPMADNNEGADGVGNSSGNW |
| | | HCDSTWMGDRVITTSTRTWALPTYNNHLYKQISSQSGA |
| | | SNDNHYFGYSTPWGYFDFNRFHCHFSPRDWQRLINNNW |
| | | GFRPKRLNFKLFNIQVKEVTQNDGTTTIANNLTSTVQV |
| | | FTDSEYQLPYVLGSAHQGCLPPFPADVFMVPQYGYLTL |
| | | NNGSQAVGRSSFYCLEYFPSQMLRTGNNFTFSYTFEDV |
| | | PFHSSYAHSQSLDRLMNPLIDQYLYYLSRTNTPSGTTT |
| | | QSRLQFSQAGASDIRDQSRNWLPGPCYRQQRVSKTSAD |
| | | NNNSEYSWTGATKYHLNGRDSLVNPGPAMASHKDDEEK |
| | | FFPQSGVLIFGKQGSEKTNVDIEKVMITDEEEIRTTNP |
| | | VATEQYGSVSTNLQRGNRQAATADVNTQGVLPGMVWQD |
| | | RDVYLQGPIWAKIPHTDGHFHPSPLMGGFGLKHPPPQI |
| | | LIKNTPVPANPSTTFSAAKFASFITQYSTGQVSVEIEW |
| | | ELQKENSKRWNPEIQYTSNYNKSVNVDFTVDINGVYSE |
| | | PRPIGTRYLTRNL |
| 128 | AAV3 | MAADGYLPDWLEDNLSEGIREWWALKPGVPQPKANQQH |
| | | QDNRRGLVLPGYKYLGPGNGLDKGEPVNEADAAALEHD |
| | | KAYDQQLKAGDNPYLKYNHADAEFQERLQEDTSFGGNL |
| | | GRAVFQAKKRILEPLGLVEEAAKTAPGKKGAVDQSPQE |
| | | PDSSSGVGKSGKQPARKRLNFGQTGDSESVPDPQPLGE |
| | | PPAAPTSLGSNTMASGGGAPMADNNEGADGVGNSSGNW |
| | | HCDSQWLGDRVITTSTRTWALPTYNNHLYKQISSQSGA |
| | | SNDNHYFGYSTPWGYFDFNRFHCHFSPRDWQRLINNNW |
| | | GFRPKKLSFKLFNIQVRGVTQNDGTTTIANNLTSTVQV |
| | | FTDSEYQLPYVLGSAHQGCLPPFPADVFMVPQYGYLTL |
| | | NNGSQAVGRSSFYCLEYFPSQMLRTGNNFQFSYTFEDV |
| | | PFHSSYAHSQSLDRLMNPLIDQYLYYLNRTQGTTSGTT |
| | | NQSRLLFSQAGPQSMSLQARNWLPGPCYRQQRLSKTAN |
| | | DNNNSNFPWTAASKYHLNGRDSLVNPGPAMASHKDDEE |
| | | KFFPMHGNLIFGKEGTTASNAELDNVMITDEEEIRTTN |
| | | PVATEQYGTVANNLQSSNTAPTTGTVNHQGALPGMVWQ |
| | | DRDVYLQGPIWAKIPHTDGHFHPSPLMGGFGLKHPPPQ |
| | | IMIKNTPVPANPPTTFSPAKFASFITQYSTGQVSVEIE |
| | | WELQKENSKRWNPEIQYTSNYNKSVNVDFTVDINGVYS |
| | | EPRPIGTRYLTRNL |
| 129 | AAV4 | MTDGYLPDWLEDNLSEGVREWWALQPGAPKPKANQQHQ |
| | | DNARGLVLPGYKYLGPGNGLDKGEPVNAADAAALEHDK |
| | | AYDQQLKAGDNPYLKYNHADAEFQQRLQGDTSFGGNLG |
| | | RAVFQAKKRVLEPLGLVEQAGETAPGKKRPLIESPQQP |
| | | DSSTGIGKKGKQPAKKKLVFEDETGAGDGPPEGSTSGA |
| | | MSDDSEMRAAAGGAAVEGGQGADGVGNASGDWHCDSTW |
| | | SEGHVTTTSTRTWVLPTYNNHLYKRLGESLQSNTYNGF |
| | | STPWGYFDFNRFHCHFSPRDWQRLINNNWGMRPKAMRV |
| | | KIFNIQVKEVTTSNGETTVANNLTSTVQIFADSSYELP |
| | | YVMDAGQEGSLPPFPNDVFMVPQYGYCGLVTGNTSQQQ |
| | | TDRNAFYCLEYFPSQMLRTGNNFEITYSFEKVPFHSMY |
| | | AHSQSLDRLMNPLIDQYLWGLQSTTTGTTLNAGTATTN |
| | | FTKLRPTNFSNFKKNWLPGPSIKQQGFSKTANQNYKIP |
| | | ATGSDSLIKYETHSTLDGRWSALTPGPPMATAGPADSK |
| | | FSNSQLIFAGPKQNGNTATVPGTLIFTSEEELAATNAT |
| | | DTDMWGNLPGGDQSNSNLPTVDRLTALGAVPGMVWQNR |
| | | DIYYQGPIWAKIPHTDGHFHPSPLIGGFGLKHPPPQIF |
| | | IKNTPVPANPATTFSSTPVNSFITQYSTGQVSVQIDWE |
| | | IQKERSKRWNPEVQFTSNYGQQNSLLWAPDAAGKYTEP |
| | | RAIGTRYLTHHL |
| 130 | AAV6 | MAADGYLPDWLEDNLSEGIREWWDLKPGAPKPKANQQK |
| | | QDDGRGLVLPGYKYLGPFNGLD |
| | | KGEPVNAADAAALEHDKAYDQQLKAGDNPYLRYNHADA |
| | | EFQERLQEDTSFGGNLGRAVFQAKKRVLEPFGLVEEGA |
| | | KTAPGKKRPVEQSPQEPDSSSGIGKTGQQPAKKRLNFG |
| | | QTGDSESVPDPQPLGEPPATPAAVGPTTMASGGGAPMA |
| | | DNNEGADGVGNASGNWHCDSTWLGDRVITTSTRTWALP |
| | | TYNNHLYKQISSASTGASNDNHYFGYSTPWGYFDFNRF |
| | | HCHFSPRDWQRLINNNWGFRPKRLNFKLFNIQVKEVTT |
| | | NDGVTTIANNLTSTVQVFSDSEYQLPYVLGSAHQGCLP |
| | | PFPADVFMIPQYGYLTLNNGSQAVGRSSFYCLEYFPSQ |
| | | MLRTGNNFTFSYTFEDVPFHSSYAHSQSLDRLMNPLID |
| | | QYLYYLNRTQNQSGSAQNKDLLFSRGSPAGMSVQPKNW |
| | | LPGPCYRQQRVSKTKTDNNNSNFTWTGASKYNLNGRES |
| | | IINPGTAMASHKDDKDKFFPMSGVMIFGKESAGASNTA |
| | | LDNVMITDEEEIKATNPVATERFGTVAVNLQSSSTDPA |
| | | TGDVHVMGALPGMVWQDRDVYLQGPIWAKIPHTDGHFH |
| | | PSPLMGGFGLKHPPPQILIKNTPVPANPPAEFSATKFA |
| | | SFITQYSTGQVSVEIEWELQKENSKRWNPEVQYTSNY |
| | | AKSANVDFTVDNNGLYTEPRPIGTRYLTRPL |
| 131 | AAV7 | MAADGYLPDWLEDNLSEGIREWWDLKPGAPKPKANQQK |
| | | QDNGRGLVLPGYKYLGPFNGLDKGEPVNAADAAALEHD |
| | | KAYDQQLKAGDNPYLRYNHADAEFQERLQEDTSFGGNL |
| | | GRAVFQAKKRVLEPLGLVEEGAKTAPAKKRPVEPSPQR |
| | | SPDSSTGIGKKGQQPARKRLNFGQTGDSESVPDPQPLG |
| | | EPPAAPSSVGSGTVAAGGGAPMADNNEGADGVGNASGN |
| | | WHCDSTWLGDRVITTSTRTWALPTYNNHLYKQISSETA |
| | | GSTNDNTYFGYSTPWGYFDFNRFHCHFSPRDWQRLINN |
| | | NWGFRPKKLRFKLFNIQVKEVTTNDGVTTIANNLTSTI |
| | | QVFSDSEYQLPYVLGSAHQGCLPPFPADVFMIPQYGYL |
| | | TLNNGSQSVGRSSFYCLEYFPSQMLRTGNNFEFSYSFE |
| | | DVPFHSSYAHSQSLDRLMNPLIDQYLYYLARTQSNPGG |
| | | TAGNRELQFYQGGPSTMAEQAKNWLPGPCFRQQRVSKT |
| | | LDQNNNSNFAWTGATKYHLNGRNSLVNPGVAMATHKDD |
| | | EDRFFPSSGVLIFGKTGATNKTTLENVLMTNEEEIRPT |
| | | NPVATEEYGIVSSNLQAANTAAQTQVVNNQGALPGMVW |
| | | QNRDVYLQGPIWAKIPHTDGNFHPSPLMGGFGLKHPPP |
| | | QILIKNTPVPANPPEVFTPAKFASFITQYSTGQVSVEI |
| | | EWELQKENSKRWNPEIQYTSNFEKQTGVDFAVDSQGVY |
| | | SEPRPIGTRYLTRNL |
| 132 | AAVhu31 | MAADGYLPDWLEDTLSEGIRQWWKLKPGPPPPKPAERH |
| | | KDDSRGLVLPGYKYLGPGNGLDKGEPVNAADAAALEHD |
| | | KAYDQQLKAGDNPYLKYNHADAEFQERLKEDTSFGGNL |
| | | GRAVFQAKKRLLEPLGLVEEAAKTAPGKKRPVEQSPQE |
| | | PDSSAGIGKSGSQPAKKKLNFGQTGDTESVPDPQPIGE |
| | | PPAAPSGVGSLTMASGGGAPVADNNEGADGVGSSSGNW |
| | | HCDSQWLGDRVITTSTRTWALPTYNNHLYKQISNSTSG |
| | | GSSNDNAYFGYSTPWGYFDFNRFHCHFSPRDWQRLINN |
| | | NWGFRPKRLNFKLFNIQVKEVTDNNGVKTIANNLTSTV |
| | | QVFTDSDYQLPYVLGSAHEGCLPPFPADVFMIPQYGYL |
| | | TLNDGGQAVGRSSFYCLEYFPSQMLRTGNNFQFSYEFE |
| | | NVPFHSSYAHSQSLDRLMNPLIDQYLYYLSKTINGSGQ |
| | | NQQTLKFSVAGPSNMAVQGRNYIPGPSYRQQRVSTTVT |
| | | QNNNSEFAWPGASSWALNGRNSLMNPGPAMASHKEGED |
| | | RFFPLSGSLIFGKQGTGRDNVDADKVMITNEEEIKTTN |
| | | PVATESYGQVATNHQSAQAQAQTGWVQNQGILPGMVWQ |
| | | DRDVYLQGPIWAKIPHTDGNFHPSPLMGGFGMKHPPPQ |
| | | ILIKNTPVPADPPTAFNKDKLNSFITQYSTGQVSVEIE |
| | | WELQKENSKRWNPEIQYTSNYYKSNNVEFAVSTEGVYS |
| | | EPRPIGTRYLTRNL |
| 133 | AAVrh10 | MAADGYLPDWLEDNLSEGIREWWDLKPGAPKPKANQQK |
| | | QDDGRGLVLPGYKYLGPFNGLDKGEPVNAADAAALEHD |
| | | KAYDQQLKAGDNPYLRYNHADAEFQERLQEDTSFGGNL |
| | | GRAVFQAKKRVLEPLGLVEEGAKTAPGKKRPVEPSPQR |
| | | SPDSSTGIGKKGQQPAKKRLNFGQTGDSESVPDPQPIG |
| | | EPPAGPSGLGSGTMAAGGGAPMADNNEGADGVGSSSGN |
| | | WHCDSTWLGDRVITTSTRTWALPTYNNHLYKQISNGTS |
| | | GGSTNDNTYFGYSTPWGYFDFNRFHCHFSPRDWQRLIN |
| | | NNWGFRPKRLNFKLFNIQVKEVTQNEGTKTIANNLTST |
| | | IQVFTDSEYQLPYVLGSAHQGCLPPFPADVFMIPQYGY |
| | | LTLNNGSQAVGRSSFYCLEYFPSQMLRTGNNFEFSYQF |
| | | EDVPFHSSYAHSQSLDRLMNPLIDQYLYYLSRTQSTGG |
| | | TAGTQQLLFSQAGPNNMSAQAKNWLPGPCYRQQRVSTT |
| | | LSQNNNSNFAWTGATKYHLNGRDSLVNPGVAMATHKDD |
| | | EERFFPSSGVLMFGKQGAGKDNVDYSSVMLTSEEEIKT |
| | | TNPVATEQYGVVADNLQQQNAAPIVGAVNSQGALPGMV |
| | | WQNRDVYLQGPIWAKIPHTDGNFHPSPLMGGFGLKHPP |
| | | PQILIKNTPVPADPPTTFSQAKLASFITQYSTGQVSVE |
| | | IEWELQKENSKRWNPEIQYTSNYYKSTNVDFAVNTDGT |
| | | YSEPRPIGTRYLTRNL |
| 146 | AAVhu32 VP1u | MAADGYLPDWLEDTLSEGIRQWWKLKPGPPPPKPAERH |
| | (amino acids 1- | KDDSRGLVLPGYKYLGPGNGLDKGEPVNAADAAALEHD |
| | 137) | KAYDQQLKAGDNPYLKYNHADAEFQERLKEDTSFGGNL |
| | | GRAVFQAKKRLLEPLGLVEEAAK |
| 147 | AAVhu32 VP1u | ATGGCTGCCGATGGTTATCTTCCAGATTGGCTCGAGGA |
| | | CACTCTCTCTGAAGGAATAAGACAGTGGTGGAAGCTCA |
| | | AACCTGGCCCACCACCACCAAAGCCCGCAGAGCGGCAT |
| | | AAGGACGACAGCAGGGGTCTTGTGCTTCCTGGGTACAA |
| | | GTACCTCGGACCCGGCAACGGACTCGACAAGGGGGAGC |
| | | CGGTCAACGCAGCAGACGCGGCGGCCCTCGAGCACGAC |
| | | AAGGCCTACGACCAGCAGCTCAAGGCCGGAGACAACCC |
| | | GTACCTCAAGTACAACCACGCCGACGCCGAGTTCCAGG |
| | | AGCGGCTCAAAGAAGATACGTCTTTTGGGGGCAACCTC |
| | | GGGCGAGCAGTCTTCCAGGCCAAAAAGAGGCTTCTTGA |
| | | ACCTCTTGGTCTGGTTGAGGAAGCGGCTAAG |
| 148 | AAVhu32 VP1/2s | TAPGKKRPVEQSPQEPDSSAGIGKSGSQPAKKKLNFGQ |
| | (amino acids 138- | TGDTESVPDPQPIGEPPAAPSGVGSLT |
| | 202) | |
| 149 | AAVhu32 VP1/2s | ACGGCTCCTGGAAAGAAGAGGCCTGTAGAGCAGTCTCC |
| | | TCAGGAACCGGACTCCTCCGCGGGTATTGGCAAATCGG |
| | | GTTCACAGCCCGCTAAAAAGAAACTCAATTTCGGTCAG |
| | | ACTGGCGACACAGAGTCAGTCCCCGACCCTCAACCAAT |
| | | CGGAGAACCTCCCGCAGCCCCCTCAGGTGTGGGATCTC |
| | | TTACA |

### 8. EQUIVALENTS AND INCORPORATIONS BY REFERENCE

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A polypeptide comprising:
a. the VP1/2s region of AAV5; and
b. the VP3 region of AAV8.

2. The polypeptide of claim 1, further comprising the VP1u region of AAV5 or AAV8.

3. The polypeptide of claim 2, wherein the regions are present in the following order from amino- to carboxy-terminus of the polypeptide:
a. the VP1u region of AAV5 or AAV8;
b. the VP1/2s region of AAV5; and
C. the VP3 region of AAV8.

4. A polypeptide comprising:
a. the VP1u region of AAV Hu32;
b. the VP1/2s region of AAV Hu32; and
c. the VP3 region of AAV Rh13.

5. The polypeptide of any one of claims 1-3, wherein the VP1/2s region of AAV5 comprises SEQ ID NO: 3.

6. The polypeptide of any one of claims 2, 3, and 5, wherein the VP1u region of AAV5 or AAV8 comprises SEQ ID NO: 7 or 17, respectively.

7. The polypeptide of any one of claims 1-3 and 5-6, wherein the VP3 region of AAV8 comprises SEQ ID NO: 11.

8. The polypeptide of any one of claims 1-3 and 6-7, wherein the VP1/2s region of AAV5 comprises an amino acid sequence consisting of SEQ ID NO: 3.

9. The polypeptide of any one of claims 2, 3, 5, and 7-8, wherein the VP1u region of AAV5 or AAV8 comprises an amino acid sequence consisting of SEQ ID NO: 7 or 17, respectively.

10. The polypeptide of any one of claims 1-3, 5-6, and 8-9, wherein the VP3 region of AAV8 comprises an amino acid sequence consisting of SEQ ID NO: 11.

11. The polypeptide of any one of claims 1-3, 6-7, and 8-9, wherein the VP1/2s region of AAV5 consists of SEQ ID NO: 3.

12. The polypeptide of any one of claims 2, 3, 5, 7, 8, 10, and 11, wherein the VP1u region of AAV5 or AAV8 consists of SEQ ID NO: 7 or 17, respectively.

13. The polypeptide of any one of claims 1-3, 5, 6, 8, 9, 11, and 12, wherein the VP3 region of AAV8 consists of SEQ ID NO: 11.

14. The polypeptide of any one of claims 1-3, wherein the polypeptide comprises SEQ ID NO: 23 or 25.

15. The polypeptide of any one of claims 1-3, wherein the polypeptide consists of SEQ ID NO: 23 or 25.

16. The polypeptide of any one of claims 1-3, wherein the polypeptide comprises an amino acid sequence consisting of SEQ ID NO: 23 or 25.

17. The polypeptide of claim 4, wherein the VP1u region of AAVhu32 comprises the VP1u portion of SEQ ID NO: 29, or SEQ ID NO: 146.

18. The polypeptide of claim 4, wherein the VP1/2s region of AAVhu32 comprises the VP1/2s portion of SEQ ID NO: 29, or SEQ ID NO: 148.

19. The polypeptide of claim 4, wherein the VP3 region of AAVrh13 comprises SEQ ID NO: 27.

20. The polypeptide of any one of claims 4 and 18-19, wherein the VP1u region of AAVhu32 comprises the VP1u portion of an amino acid sequence consisting of SEQ ID NO: 29, or SEQ ID NO: 146.

21. The polypeptide of any one of claims 4, 17, and 19-20, wherein the VP1/2s region of AAVhu32 comprises the VP1/2s portion of an amino acid sequence consisting of SEQ ID NO: 29, or SEQ ID NO: 148.

22. The polypeptide of any one of claims 4, 17, 18, and 20-21, wherein the VP3 region of AAVrh13 comprises an amino acid sequence consisting of SEQ ID NO: 27.

23. The polypeptide of any one of claims 4, 18-19, and 21-22, wherein the VP1u region of AAVhu32 consists of the VP1u portion of SEQ ID NO: 29, or SEQ ID NO: 146.

24. The polypeptide of any one of claims 4, 17, 19-20, and 22-23, wherein the VP1/2s region of AAVhu32 consists of the VP1/2s portion of SEQ ID NO: 29, or SEQ ID NO: 148.

25. The polypeptide of any one of claims 4, 17, 18, 20-21, and 23-24, wherein the VP3 region of AAVrh13 consists of SEQ ID NO: 27.

26. The polypeptide of claim 4, wherein the polypeptide comprises an amino acid sequence of SEQ ID NO: 29.

27. The polypeptide of claim 4, wherein the polypeptide consists of an amino acid sequence of SEQ ID NO: 29.

28. The polypeptide of claim 4, wherein the polypeptide comprises an amino acid sequence consisting of SEQ ID NO: 29.

29. A nucleotide sequence encoding the amino acid sequence of the polypeptide of any one of claims 1-28.

30. An expression vector comprising the nucleotide sequence of claim 29.

31. A host cell comprising the nucleotide sequence of claim 29 or the expression vector of claim 30.

32. A recombinant AAV comprising the polypeptide of any one of claims 1-28.

33. A recombinant adeno-associated virus (AAV) comprising a capsid protein, wherein the capsid protein comprises: (i) an amino acid sequence that is at least about 95% sequence identical to VP1/2s region of AAV5, and (ii) an amino acid sequence that is at least about 95% sequence identical to VP3 region of AAV8, and wherein the rAAV comprises an expression cassette.

34. The rAAV of claim 33, wherein the expression cassette comprises an open reading frame (ORF) encoding a transgene.

35. The rAAV of claim 33 or 34, wherein the expression cassette further comprises a promoter.

36. The rAAV of any one of claims 33-35, wherein the capsid protein further comprises an amino acid sequence that is at least about 95% sequence identical to VP1u region of AAV5 or AAV8.

37. The rAAV of any one of claims 33-35, wherein the capsid protein further comprises an amino acid sequence that is at least about 98% sequence identical to VP1u region of AAV5 or AAV8.

38. The rAAV of any one of claims 33-35, wherein the capsid protein further comprises an amino acid sequence of the VP1u region of AAV5 or AAV8.

39. The rAAV of any one of claims 33-38, wherein the capsid protein comprises: (i) an amino acid sequence that is at least about 98% sequence identical to VP1/2s region of AAV5, and (ii) an amino acid sequence that is at least about 98% sequence identical to VP3 region of AAV8.

40. The rAAV of any one of claims 33-38, wherein the capsid protein comprises: (i) an amino acid sequence of the VP1/2s region of AAV5, and (ii) an amino acid sequence of the VP3 region of AAV8.

41. The rAAV of any one of claims 33-40, wherein the VP1/2s region of AAV5 comprises SEQ ID NO: 3.

42. The rAAV of any one of claims 36-41, wherein the VP1u region of AAV5 or AAV8 comprises SEQ ID NO: 7 or 17, respectively.

43. The rAAV of any one of claims 33-42, wherein the VP3 region of AAV8 comprises SEQ ID NO: 11.

44. The rAAV of any one of claims 33-40 and 42-43, wherein the VP1/2s region of AAV5 comprises an amino acid sequence consisting of SEQ ID NO: 3.

45. The rAAV of any one of claims 36-41 and 43-44, wherein the VP1u region of AAV5 or AAV8 comprises an amino acid sequence consisting of SEQ ID NO: 7 or 17, respectively.

46. The rAAV of any one of claims 33-42 and 44-45, wherein the VP3 region of AAV8 comprises an amino acid sequence consisting of SEQ ID NO: 11.

47. The rAAV of any one of claims 33-40, 42-43, and 45-46, wherein the VP1/2s region of AAV5 consists of SEQ ID NO: 3.

48. The rAAV of any one of claims 36-41, 43-44, and 46-47, wherein the VP1u region of AAV5 or AAV8 consists of SEQ ID NO: 7 or 17, respectively.

49. The rAAV of any one of claims 33-42, 44-45, and 47-48, wherein the VP3 region of AAV8 consists of SEQ ID NO: 11.

50. A recombinant adeno-associated virus (AAV) comprising a capsid protein, wherein the capsid protein comprises: (i) an amino acid sequence that is at least about 95% sequence identical to VP1u region of AAVhu32, (ii) an amino acid sequence that is at least about 95% sequence identical to VP1/2s region of AAVhu32, and (iii) an amino acid sequence that is at least about 95% sequence identical to VP3 region of AAVrh13, and an expression cassette.

51. The rAAV of claim 50, wherein the capsid protein comprises: (i) an amino acid sequence that is at least about 98% sequence identical to VP1u region of AAVhu32, (ii) an amino acid sequence that is at least about 98% sequence identical to VP1/2s region of AAVhu32, and (iii) an amino acid sequence that is at least about 98% sequence identical to VP3 region of AAVrh13.

52. The rAAV of any one of claims 50-51, wherein the capsid protein comprises: (i) an amino acid sequence of the VP1u region of AAVhu32, (ii) an amino acid sequence of the VP1/2s region of AAVhu32, and (iii) an amino acid sequence of the VP3 region of AAVrh13.

53. The rAAV of any one of claims 50-52, wherein the expression cassette comprises an open reading frame (ORF) encoding a transgene.

54. The rAAV of any one of claims 50-53, wherein the expression cassette further comprises a promoter.

55. The rAAV of any one of claims 50-54, wherein the VP1u region of AAVhu32 comprises the VP1u portion of SEQ ID NO: 29, or SEQ ID NO: 146.

56. The rAAV of any one of claims 50-54, wherein the VP1u region of AAVhu32 consists of the VP1u portion of SEQ ID NO: 29, or SEQ ID NO: 146.

57. The rAAV of any one of claims 50-54, wherein the VP1u region of AAVhu32 comprises the VP1u portion of an amino acid sequence consisting of SEQ ID NO: 29, or SEQ ID NO: 146.

58. The rAAV of any one of claims 50-57, wherein the VP1/2s region of AAVhu32 comprises the VP1/2s portion of SEQ ID NO: 29, or SEQ ID NO: 148.

59. The rAAV of any one of claims 50-57, wherein the VP1/2s region of AAVhu32 consists the VP1/2s portion of SEQ ID NO: 29, or SEQ ID NO: 148.

60. The rAAV of any one of claims 50-57, wherein the VP1/2s region of AAVhu32 comprises an amino acid sequence consisting of the VP1/2s portion of SEQ ID NO: 29, or SEQ ID NO: 148.

61. The rAAV of any one of claims 50-60, wherein the VP3 region of AAVrh13 comprises an amino acid sequence of SEQ ID NO: 27.

62. The rAAV of any one of claims 50-60, wherein the VP3 region of AAVrh13 consists of an amino acid sequence of SEQ ID NO: 27.

63. The rAAV of any one of claims 50-60, wherein the VP3 region of AAVrh13 comprises an amino acid sequence consisting of SEQ ID NO: 27.

64. The rAAV of any one of claims 33-49, wherein the capsid protein comprises an amino acid sequence of SEQ ID NO: 21 or 23.

65. The rAAV of any one of claims 33-49, wherein the capsid protein consists of an amino acid sequence of SEQ ID NO: 21 or 23.

66. The rAAV of any one of claims 33-49, wherein the capsid protein comprises an amino acid sequence consisting of SEQ ID NO: 21 or 23.

67. The rAAV of any one of claims 50-63, wherein the capsid protein comprises an amino acid sequence of SEQ ID NO: 29.

68. The rAAV of any one of claims 50-63, wherein the capsid protein consists of an amino acid sequence of SEQ ID NO: 29.

69. The rAAV of any one of claims 50-63, wherein the capsid protein comprises an amino acid sequence consisting of SEQ ID NO: 29.

70. The rAAV of any one of claims 33-69, further comprising an AAV inverted terminal repeat.

71. A composition comprising the rAAV of any one of claims 33-70, and a physiologically acceptable carrier.

72. The rAAV of claim 32, wherein the rAAV further comprises a transgene.

73. A method of delivering the transgene to a cell, comprising contacting the cell with the rAAV of any one of claims 33-70 and 72.

74. The method of claim 73, wherein the cell is a muscle cell.

75. The rAAV of any one of claims 33-69 and 72, or the method of claim 73 or 74, wherein the transgene comprises a heterologous gene associated with a muscle related disease or disorder.

76. The rAAV or the method of claim 75, wherein the heterologous gene is operably linked to a regulatory sequence that controls expression of the heterologous gene in a host cell.

77. The host cell of claim 31, or the rAAV of claim 76, or the method of claim 76, wherein the host cell is a muscle cell.

78. The rAAV of any one of claims 33-69 and 72, or the method of claim 73 or 74, wherein the transgene encodes a therapeutic protein.

79. The rAAV or the method of claim 78, wherein the therapeutic protein is associated with a muscle related disease or disorder.

80. The rAAV or the method of claim 79, wherein the muscle related disease or disorder is at least one of Duchenne muscular dystrophy, Becker muscular dystrophy, Bethlem congenital muscular dystrophy (CMD), Fukuyama CMD, muscle-eye-brain disease, rigid spine syndrome, Ullrich CMD, walker-warburg syndrome, Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), amyotrophic lateral sclerosis (ALS), Spinal-bulbar muscular atrophy (SBMA), Spinal muscular atrophy (SMA), Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia, Friedreich's ataxia (FA), Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO), Congenital myasthenic syndromes (CMS), Lambert-Eaton myasthenic syndrome (LEMS), Myasthenia gravis (MG), Charcot-Marie-Tooth disease (CMT), Giant axonal neuropathy (GAN), Myofibrillar myopathy (MFM), Scapuloperoneal myopathy, Metabolic myopathy, inflammatory myopathy, endocrine myopathy, distal myopathy, and congenital myopathy.

81. The rAAV or the method of claim 80, wherein the muscle related disease or disorder is Duchenne muscular dystrophy.

82. The rAAV or the method of any one of claims 78-81, wherein the transgene encodes a microdystrophin protein.

83. A vector comprising a nucleic acid sequence encoding the capsid protein as defined in any one of claims 33-70.

84. The vector of claim 83, wherein the nucleic acid sequence is operably linked to a heterologous regulatory element that controls expression of the capsid protein in a host cell.

85. An in vitro cell comprising the vector of claim 83 or 84.

86. A method of producing a recombinant adeno-associated virus (rAAV), the method comprising the steps of:
i. culturing a cell in a cell culture to produce the rAAV, the cell comprising a nucleotide sequence encoding a capsid protein, and a nucleotide sequence comprising a transgene, and wherein the capsid protein comprises: (i) an amino acid sequence that is at least about 95% sequence identical to VP1/2s region of AAV5, and (ii) an amino acid sequence that is at least about 95% sequence identical to VP3 region of AAV8; and
ii. collecting the rAAV from the cell culture.

87. The method of claim 86, wherein the capsid protein further comprises an amino acid sequence that is at least about 95% sequence identical to VP1u region of AAV5 or AAV8.

88. The method of claim 86, wherein the capsid protein further comprises an amino acid sequence that is at least about 98% sequence identical to VP1u region of AAV5 or AAV8.

89. The method of claim 86, wherein the capsid protein further comprises an amino acid sequence of VP1u region of AAV5 or AAV8.

90. The method of any one of claims 86-89, wherein the capsid protein comprises: (i) an amino acid sequence that is at least about 98% sequence identical to VP1/2s region of AAV5, and (ii) an amino acid sequence that is at least about 98% sequence identical to VP3 region of AAV8.

91. The method of any one of claims 86-89, wherein the capsid protein comprises: (i) an amino acid sequence of VP1/2s region of AAV5, and (ii) an amino acid sequence of VP3 region of AAV8.

92. A method of producing a recombinant adeno-associated virus (rAAV), the method comprising the steps of:
i. culturing a cell in a cell culture to produce the rAAV, the cell comprising a nucleotide sequence encoding a capsid protein, and a nucleotide sequence comprising a transgene, and wherein the capsid protein comprises: (i) an amino acid sequence that is at least about 95% sequence identical to VP1u region of AAVhu32, (ii) an amino acid sequence that is at least about 95% sequence identical to VP1/2s region of AAVhu32, and (iii) an amino acid sequence that is at least about 95% sequence identical to VP3 region of AAVrh13; and
ii. collecting the rAAV from the cell culture.

93. The method of claim 92, wherein the capsid protein comprises: (i) an amino acid sequence that is at least about 98% sequence identical to VP1u region of AAVhu32, (ii) an amino acid sequence that is at least about 98% sequence identical to VP1/2s region of AAVhu32, and (iii) an amino acid sequence that is at least about 98% sequence identical to VP3 region of AAVrh13.

94. The method of claim 92 or 93, wherein the capsid protein comprises: (i) an amino acid sequence of VP1u region of AAVhu32, (ii) an amino acid sequence of VP1/2s region of AAVhu32, and (iii) an amino acid sequence of VP3 region of AAVrh13.

95. The method of any one of claims 86-94, wherein the cell comprises a nucleotide sequence encoding an AAV rep protein.

96. The method of any one of claims 86-95, wherein the transgene is flanked by AAV inverted terminal repeats.

97. The method of any one of claims 86-96, wherein the transgene comprises a heterologous gene associated with a muscle related disease or disorder.

98. The method of claim 97, wherein the heterologous gene is operably linked to a regulatory sequence that controls expression of the heterologous gene in a host cell.

99. The method of any one of claims 86-96, wherein the transgene encodes a therapeutic protein.

100. The method of claim 99, wherein the therapeutic protein is associated with a muscle related disease or disorder.

101. The method of claim 99 or 100, wherein the therapeutic protein encodes microdystrophin protein.

102. The method of any one of claims 86-96, wherein the transgene encodes a functional gene product.

103. The method of any one of claims of any one of claims 86-102, wherein the cell is selected from an invertebrate cell, an insect cell, or a mammalian cell.

104. The method of claim 103, wherein the mammalian cell is selected from HEK293, HeLa, CHO, NS0, SP2/0, PER.C6, Vero, RD, BHK, HT-1080, A549, Cos-7, ARPE-19, MRC-5, or any combination thereof.

105. The method of claim 103, wherein the insect cell is selected from High Five, Sf9, Se301, SeIZD2109, SeUCR1, Sf900+, Sf21, Bti-tn-5b1-4, MG-1, Tn368, HzAm1, BM-N, Ha2302, Hz2E5, Ao38, or any combination thereof.

106. A method of treating a muscle related disease or disorder in a subject in need thereof, the method comprising administering the rAAV of any one of claims 33-70, 72, and 78-82, or the composition of claim 71, to the subject.

107. The method of claim 106, wherein the muscle related disease or disorder is at least one of Duchenne muscular dystrophy, Becker muscular dystrophy, Bethlem congenital muscular dystrophy (CMD), Fukuyama CMD, muscle-eye-brain disease, rigid spine syndrome, Ullrich CMD, walker-warburg syndrome, Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), amyotrophic lateral sclerosis (ALS), Spinal-bulbar muscular atrophy (SBMA), Spinal muscular atrophy (SMA), Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia, Friedreich's ataxia (FA), Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO), Congenital myasthenic syndromes (CMS), Lambert-Eaton myasthenic syndrome (LEMS), Myasthenia gravis (MG), Charcot-Marie-Tooth disease (CMT), Giant axonal neuropathy (GAN), Myofibrillar myopathy (MFM), Scapuloperoneal myopathy, Metabolic myopathy, inflammatory myopathy, endocrine myopathy, distal myopathy, and congenital myopathy.

108. The method of claim 106, wherein the muscle related disease or disorder is muscular dystrophy.

109. The method of claim 108, wherein the muscular dystrophy is Duchenne muscular dystrophy.

110. The rAAV of any one of claims 33-70, 72, and 78-82, wherein the rAAV provides at least one improvement of packaging efficiency, yield, titer, infectivity, transduction efficiency, and transfection efficiency, as compared to a reference AAV.

111. The rAAV of claim 110, wherein the reference AAV is AAV8.

112. The rAAV of claim 110, wherein the reference AAV comprises VP1u of AAV8.

113. The rAAV of claim 110, wherein the reference AAV is AAVrh13.

114. The rAAV of any one of claims 110-113, wherein the improvement is by about or at least about 1-fold, 1.5-fold, 2-fold, 2.5-fold, 3-fold, 3.5-fold, 4-fold, 4.5-fold, 5 fold, 5.5-fold, 6-fold, 6.5-fold, 7-fold, 7.5-fold, 8-fold, 8.5-fold, 9-fold, 9.5-fold, 10-fold, or higher than 10-fold.

115. The rAAV of claim 114, wherein the improvement is by about or at least about 2.5-fold.

116. The rAAV of any one of claims 110-115, wherein the at least one improvement is an improvement in packaging efficiency.

117. The rAAV of any one of claims 110-115, wherein the at least one improvement is an improvement in titer.

118. The rAAV of any one of claims 33-70, 72, and 78-82, wherein the rAAV transduces liver tissues at lower levels as compared to a reference AAV.

119. The rAAV of any one of claims 33-70, 72, and 78-82, wherein the rAAV results in lower RNA expression in liver as compared to a reference AAV.

120. The rAAV of claim 119, wherein the RNA expression is lower by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100% as compared to the RNA expression from a reference AAV.

121. The rAAV of any one of claims 33-70, 72, and 78-82, wherein the rAAV results in higher RNA/DNA ratio in muscle tissue as compared to a reference AAV.

122. The rAAV of claim 121, wherein the RNA/DNA ratio is higher by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100%.

123. The rAAV of any one of claims 33-70, 72, and 78-82, wherein the rAAV results in lower DNA levels in muscle tissue as compared to a reference AAV.

124. The rAAV of claim 123, wherein the DNA levels is lower by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100% as compared to the DNA levels from a reference AAV.

125. The rAAV of any one of claims 33-70, 72, and 78-82, wherein the rAAV results in a lower level of liver toxicity as compared to the level of liver toxicity from a reference AAV.

126. The rAAV of claim 125, wherein the level of liver toxicity is lower by about or at least about 5%, 10%, 15%, 20%, 25%, 30%, 35%, 40%, 45%, 50%, 55%, 60%, 65%, 70%, 75%, 80%, 85%, 90%, 95%, 100%, or more than 100% as compared to the level of liver toxicity from a reference AAV.

127. The rAAV of any one of claims 33-70, 72, and 78-82, wherein the rAAV results in transcription-specific liver de-targeting as compared to a reference AAV.

128. The method of claim 106, wherein the administering comprises administering a lower amount of vector genome of the rAAV as compared to the amount of vector genome of a reference AAV necessary to obtain the same therapeutic effect after the administering.

129. The rAAV of any one of claims 118-127 or the method of claim 128, wherein the reference AAV is AAV8.

130. The rAAV of any one of claims 118-127 or the method of claim 128, wherein the reference AAV comprises VP1u of AAV8.

131. The rAAV of any one of claims 118-127 or the method of claim 128, wherein the reference AAV is AAVrh13.

132. A polypeptide comprising one or more of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71.

133. A polypeptide consisting of one or more of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71.

134. A polypeptide comprising one or more of any one of SEQ ID NOs: 81, 83, 85, 87, 89, 91, 93, 95, 97, 99, 101, 103, 105, 107, 109, 111, 113, 115, 117, and 119.

135. A polypeptide consisting of one or more of any one of SEQ ID NOs: 81, 83, 85, 87, 89, 91, 93, 95, 97, 99, 101, 103, 105, 107, 109, 111, 113, 115, 117, and 119.

136. A polynucleotide encoding an AAV capsid protein comprising the amino acid sequence of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71.

137. A polynucleotide encoding an AAV capsid protein consisting of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, and 71.

138. A polynucleotide comprising any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72, wherein the polynucleotide encodes an AAV capsid protein.

139. A polynucleotide encoding an AAV capsid protein comprising the amino acid sequence of any one of SEQ ID NOs: 81, 83, 85, 87, 89, 91, 93, 95, 97, 99, 101, 103, 105, 107, 109, 111, 113, 115, 117, and 119.

140. A polynucleotide encoding an AAV capsid protein consisting of any one of SEQ ID NOs: 81, 83, 85, 87, 89, 91, 93, 95, 97, 99, 101, 103, 105, 107, 109, 111, 113, 115, 117, and 119.

141. A polynucleotide comprising any one of SEQ ID NOs: 82, 84, 86, 88, 90, 92, 94, 96, 98, 100, 102, 104, 106, 108, 110, 112, 114, 116, 118, and 120, wherein the polynucleotide encodes an AAV capsid protein.

142. A polynucleotide consisting of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, and 72, wherein the polynucleotide encodes an AAV capsid protein.

143. A polynucleotide consisting of any one of SEQ ID NOs: 82, 84, 86, 88, 90, 92, 94, 96, 98, 100, 102, 104, 106, 108, 110, 112, 114, 116, 118, and 120, wherein the polynucleotide encodes an AAV capsid protein.

144. A method of treating a muscle related disease or disorder in a subject in need thereof, the method comprising administering an rAAV comprising a polypeptide comprising an amino acid sequence of SEQ ID NO:31 or an rAAV encoded by SEQ ID NO:32.

145. The method of claim 144, wherein the muscle related disease or disorder is at least one of Duchenne muscular dystrophy, Becker muscular dystrophy, Bethlem congenital muscular dystrophy (CMD), Fukuyama CMD, muscle-eye-brain disease, rigid spine syndrome, Ullrich CMD, walker-warburg syndrome, Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), amyotrophic lateral sclerosis (ALS), Spinal-bulbar muscular atrophy (SBMA), Spinal muscular atrophy (SMA), Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia, Friedreich's ataxia (FA), Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO), Congenital myasthenic syndromes (CMS), Lambert-Eaton myasthenic syndrome (LEMS), Myasthenia gravis (MG), Charcot-Marie-Tooth disease (CMT), Giant axonal neuropathy (GAN), Myofibrillar myopathy (MFM), Scapuloperoneal myopathy, Metabolic myopathy, inflammatory myopathy, endocrine myopathy, distal myopathy, and congenital myopathy.

146. The method of claim 144, wherein the muscle related disease or disorder is muscular dystrophy.

147. The method of claim 146, wherein the muscular dystrophy is Duchenne muscular dystrophy.

148. The method of any one of claims 146-147, wherein the rAAV further comprises a transgene encoding a functional dystrophin, a minidystrophin, a microdystrophin, or a dystrophin exon-skipping snRNA.

149. The method of claim 148, wherein the transgene comprises a polynucleotide sequence encoding any one of SEQ ID NOs: 73, 74, 75, 76, 77, 78, 79, and 80.

150. The method of any one of claims 144-149, wherein the rAAV provides at least one improvement of packaging efficiency, yield, titer, infectivity, transduction efficiency, and transfection efficiency, as compared to a reference AAV, wherein the reference AAV is AAV9.

151. The method of any one of claims 106-109, 128-131, and 144-150, wherein the rAAV provides a higher vector yield after the rAAV is administered to the subject, as compared to a reference AAV.

152. The method of claim 151, wherein the vector yield is higher by about or at least about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10 times, or more than 10 times.

153. The method of claim 151, wherein the vector yield is higher by between about 1.5 to about 3 times.

154. The method of any one of claims 106-109, 128-131, and 144-153, wherein the RNA level is increased after the rAAV is administered to the subject, as compared to a reference AAV.

155. The method of claim 154, wherein the RNA level is increased by about or at least about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10 times, or more than 10 times.

156. The method of claim 154, wherein the RNA level is increased by between about 2.5 to about 3.5 times.

157. The method of any one of claims 106-109, 128-131, and 144-156, wherein the ratio of RNA to DNA is increased after the rAAV is administered to the subject.

158. The method of claim 157, wherein the ratio of RNA to DNA is increased by about or at least about 0.5, 1, 1.5, 2, 2.5, 3, 3.5, 4, 4.5, 5, 5.5, 6, 6.5, 7, 7.5, 8, 8.5, 9, 9.5, 10 times, or more than 10 times.

159. The method of claim 157, wherein the ratio of RNA to DNA is increased by between about 2.5 to about 3.5 times.

160. The method of any one of claims 106-109, 128-131, and 144-159, wherein the reference AAV is AAV5.

161. The method of any one of claims 106-109, 128-131, and 144-159, wherein the reference AAV is AAV8.

162. The method of any one of claims 106-109, 128-131, and 144-161, where the reference AAV comprises the expression cassette.

163. A recombinant adeno-associated virus (rAAV) hu32 capsid protein comprising SEQ ID NO: 31, or an rAAV capsid protein comprising an amino acid sequence that is about or at least about 90% identical to SEQ ID NO: 31, comprising a peptide insertion of at least 4 and up to 12 contiguous amino acids from a heterologous protein that is not an AAV protein, wherein the peptide insertion is inserted immediately after an amino acid residue corresponding to one of any one of amino acids 451 to 461 of AAVhu32 capsid protein of SEQ ID NO: 31.

164. The method of claim 163, wherein the peptide insertion is between amino acid 454 and amino acid 455 of SEQ ID NO: 31.

165. A recombinant adeno-associated virus (rAAV) hu32 capsid protein comprising SEQ ID NO: 31, or an rAAV capsid protein comprising an amino acid sequence that is about or at least about 90% identical to SEQ ID NO: 31, comprising a peptide insertion of at least 4 and up to 12 contiguous amino acids from a heterologous protein that is not an AAV protein, wherein the peptide insertion is inserted immediately after an amino acid residue corresponding to one of amino acids 570 to 595 of AAVhu32 capsid protein of SEQ ID NO: 31.

166. The method of claim 165, wherein the peptide insertion is between amino acid 589 and amino acid 590 of SEQ ID NO: 31.

167. The method of any one of claims 163 to 166, wherein the peptide insertion is a muscle-homing peptide.

168. The method of claim 167, wherein the muscle-homing peptide comprises an integrin receptor-binding domain or an integrin-binding domain.

169. A recombinant adeno-associated virus (rAAV) capsid protein comprising an amino acid sequence that is about or at least about 90% identical to any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119.

170. The rAAV capsid protein of claim 169, wherein the rAAV capsid protein comprises an amino acid sequence that is about or at least about 98% identical to any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119.

171. The rAAV capsid protein of claim 169 or 170, wherein the rAAV capsid protein comprises an amino acid sequence of any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119.

172. A recombinant adeno-associated virus (rAAV) vector comprising a nucleic acid sequence encoding the rAAV capsid protein of any one of claims 169 to 171.

173. A recombinant adeno-associated virus (rAAV) vector comprising a nucleic acid sequence of any one of SEQ ID NOs: 22, 24, 26, 30, 34, 36, 38, 40, 42, 44, 46, 48, 50, 52, 54, 56, 58, 60, 62, 64, 66, 68, 70, 72, 82, 84, 86, 88, 90, 92, 94, 96, 98, 100, 102, 104, 106, 108, 110, 112, 114, 116, 118, and 120.

174. A cell comprising the rAAV vector of claim 172 or 173.

175. A cell expressing the rAAV capsid protein of any one of claims 169 to 171.

176. A recombinant AAV viral particle comprising the rAAV capsid protein of any one of claims 169 to 171.

177. The rAAV capsid protein of any one of claims 169 to 171, wherein a first AAV viral particle comprising the rAAV capsid protein results in increased transduction of muscle cells or heart cells relative to a second AAV viral particle comprising a capsid protein that comprises any one of SEQ ID NOs: 19, 31, and 124.

178. The rAAV capsid protein of claim 177, wherein the first AAV viral particle has at least about 1.5 fold increase in transduction of muscle cells or heart cells relative to the second AAV viral particle.

179. The rAAV capsid protein of any one of claims 169 to 171, wherein a first AAV viral particle comprising the rAAV capsid protein has reduced transduction of liver cells relative to a second AAV viral particle comprising a capsid protein that comprises any one of SEQ ID NOs: 11, 15, 19, 31, and 124.

180. The rAAV capsid protein of claim 179, wherein the first AAV viral particle has about or at least about 1 fold decrease in transduction of liver cells relative to the second AAV viral particle.

181. A pharmaceutical composition comprising a recombinant adeno-associated virus (rAAV) vector comprising a recombinant AAV capsid protein, wherein the recombinant AAV capsid protein comprises an amino acid sequence that is at least 90% identical to any one of SEQ ID NOs: 21, 23, 25, 29, 33, 35, 37, 39, 41, 43, 45, 47, 49, 51, 53, 55, 57, 59, 61, 63, 65, 67, 69, 71, 81, 83, 85, 87, 89, 91, 93, 95, 97, 99, 101, 103, 105, 107, 109, 111, 113, 115, 117, and 119.

182. A method of treating a muscle related disease or disorder in a subject in need thereof, the method comprising administering the rAAV viral particle of claim 176, the cell of claim 174 or 175, the rAAV vector of claim 172 or 173, or the pharmaceutical composition of claim 181 to the subject.

183. The method of claim 182, wherein the muscle related disease or disorder is at least one of Duchenne muscular dystrophy, Becker muscular dystrophy, Bethlem congenital muscular dystrophy (CMD), Fukuyama CMD, muscle-eye-brain disease, rigid spine syndrome, Ullrich CMD, walker-warburg syndrome, Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), amyotrophic lateral sclerosis (ALS), Spinal-bulbar muscular atrophy (SBMA), Spinal muscular atrophy (SMA), Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia, Friedreich's ataxia (FA), Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO), Congenital myasthenic syndromes (CMS), Lambert-Eaton myasthenic syndrome (LEMS), Myasthenia gravis (MG), Charcot-Marie-Tooth disease (CMT), Giant axonal neuropathy (GAN), Myofibrillar myopathy (MFM), Scapuloperoneal myopathy, Metabolic myopathy, inflammatory myopathy, endocrine myopathy, distal myopathy, and congenital myopathy.

183. The method of claim 182 or 183, wherein the muscle related disease or disorder is muscular dystrophy.

184. The method of claim 183, wherein the muscular dystrophy is Duchenne muscular dystrophy.

185. The method of any one of claims 182 to 184, wherein the rAAV further comprises a transgene encoding a functional dystrophin, a minidystrophin, a microdystrophin, or a dystrophin exon-skipping snRNA.

186. An isolated nucleic acid molecule comprising a nucleic acid sequence encoding the rAAV capsid protein of any one of claims 169 to 171.

187. An isolated nucleic acid molecule comprising the nucleic acid sequence of any one of SEQ ID NOs: 22, 24, 26, 30, 34, 36, 38, 40, 42, 44, 46, 48, 50, 52, 54, 56, 58, 60, 62, 64, 66, 68, 70, 72, 82, 84, 86, 88, 90, 92, 94, 96, 98, 100, 102, 104, 106, 108, 110, 112, 114, 116, 118, and 120.

188. A cultured cell comprising the nucleic acid molecule of claim 186 or 187.

189. The rAAV capsid protein of any one of claims 177 to 180, wherein the second AAV viral particle comprises an AAVhu32, AAV8, and/or AAV9 capsid protein.


SEQUENCE LISTING (151 sequences)

SEQ ID NO: 1 - protein, 532 aa, Adeno-associated virus
  source 1..532: note = AAV5 VP3 capsid protein
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 2 - DNA, 1599 bp, Adeno-associated virus
  source 1..1599: mol_type = genomic DNA; note = AAV5 VP3 capsid DNA
atgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 3 - protein, 56 aa, Adeno-associated virus
  source 1..56: note = AAV5 VP1/2s common region
TAPTGKRIDDHFPKRKKARTEEDSKPSTSSDAEAGPSGSQQLQIPAQPASSLGADT

SEQ ID NO: 4 - DNA, 168 bp, Adeno-associated virus
  source 1..168: mol_type = genomic DNA; note = AAV5 VP1/2s common region
acggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgataca

SEQ ID NO: 5 - protein, 588 aa, Adeno-associated virus
  source 1..588: note = AAV5 VP2 capsid protein
TAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - DNA, 1767 bp, Adeno-associated virus
  source 1..1767: mol_type = genomic DNA; note = AAV5 VP2 capsid DNA
acggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 7 - protein, 136 aa, Adeno-associated virus
  source 1..136: note = AAV5 VP1u region (protein)
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAK

SEQ ID NO: 8 - DNA, 408 bp, Adeno-associated virus
  source 1..408: mol_type = genomic DNA; note = AAV5 VP1u region (DNA)
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaag

SEQ ID NO: 9 - protein, 724 aa, Adeno-associated virus
  source 1..724: note = AAV5 VP1 capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 10 - DNA, 2175 bp, Adeno-associated virus
  source 1..2175: mol_type = genomic DNA; note = AAV5 VP1 capsid DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 11 - protein, 535 aa, Adeno-associated virus
  source 1..535: note = AAV8 VP3 capsid protein
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 12 - DNA, 1608 bp, Adeno-associated virus
  source 1..1608: mol_type = genomic DNA; note = AAV8 VP3 capsid DNA
atggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 13 - protein, 66 aa, Adeno-associated virus
  source 1..66: note = AAV8 VP1/2s common region (protein)
TAPGKKRPVEPSPQRSPDSSTGIGKKGQQPARKRLNFGQTGDSESVPDPQPLGEPPAAPSGVGPNT

SEQ ID NO: 14 - DNA, 198 bp, Adeno-associated virus
  source 1..198: mol_type = genomic DNA; note = AAV8 VP1/2s common region (DNA)
acggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaataca

SEQ ID NO: 15 - protein, 601 aa, Adeno-associated virus
  source 1..601: note = AAV8 VP2 capsid protein
TAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 16 - DNA, 1806 bp, Adeno-associated virus
  source 1..1806: mol_type = genomic DNA; note = AAV8 VP2 capsid DNA
acggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 17 - protein, 137 aa, Adeno-associated virus
  source 1..137: note = AAV8 VP1u region (protein)
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAK

SEQ ID NO: 18 - DNA, 411 bp, Adeno-associated virus
  source 1..411: mol_type = genomic DNA; note = AAV8 VP1u region (DNA)
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaag

SEQ ID NO: 19 - protein, 738 aa, Adeno-associated virus
  source 1..738: note = AAV8 VP1 capsid protein
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 20 - DNA, 2217 bp, Adeno-associated virus
  source 1..2217: mol_type = genomic DNA; note = AAV8 VP1 capsid DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 21 - protein, 591 aa, Synthetic construct
  source 1..591: note = AAV5-AAV8 hybrid VP2 capsid protein
TAPTGKRIDDHFPKRKKARTEEDSKPSTSSDAEAGPSGSQQLQIPAQPASSLGADTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 22 - DNA, 1776 bp, Synthetic construct
  source 1..1776: mol_type = other DNA; note = AAV5-AAV8 hybrid VP2 capsid DNA
acggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 23 - protein, 727 aa, Synthetic construct
  source 1..727: note = AAV5-AAV8 hybrid VP1 capsid protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 24 - DNA, 2184 bp, Synthetic construct
  source 1..2184: mol_type = other DNA; note = AAV5-AAV8 hybrid VP1 capsid DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 25 - protein, 728 aa, Synthetic construct
  source 1..728: note = AAV8/5-AAV8 hybrid VP1 capsid protein
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPTGKRIDDHFPKRKKARTEEDSKPSTSSDAEAGPSGSQQLQIPAQPASSLGADTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 26 - DNA, 2187 bp, Synthetic construct
  source 1..2187: mol_type = other DNA; note = AAV8/5-AAV8 hybrid VP1 capsid DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 27 - protein, 530 aa, Adeno-associated virus
  source 1..530: note = AAVrh13 VP3 capsid protein
MAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPRKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYTFEEVPFHSSYAHSQSLDRLMNPLIDQYLYYLARTQSTTGSTRELQFHQAGPNTMAEQSKNWLPGPCYRQQRLSKNIDSNNNSNFAWTGATKYHLNGRNSLTNPGVAMATNKDDEDQFFPINGVLVFGETGAANKTTLENVLMTSEEEIKTTNPVATEEYGVVSSNLQSSTAGPQTQTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYAKSNNVEFAVNNEGVYTEPRPIGTRYLTRNL

SEQ ID NO: 28 - DNA, 1593 bp, Adeno-associated virus
  source 1..1593: mol_type = genomic DNA; note = AAVrh13 VP3 capsid DNA
atggctgcaggcggtggcgcaccaatggcagacaataacgaaggcgccgacggagtgggtaatgcctccggaaattggcattgcgattccacatggctgggcgacagagtcatcaccaccagcacccgcacctgggccctgcccacctacaacaaccacctctacaagcagatatcaagtcagagcggggctaccaacgacaaccacttcttcggctacagcaccccctggggctattttgacttcaacagattccactgccacttctcaccacgtgactggcagcgactcatcaacaacaactggggattccggcccagaaagctgcggttcaagttgttcaacatccaggtcaaggaggtcacgacgaacgacggcgttacgaccatcgctaataaccttaccagcacgattcaggtcttctcggactcggagtaccaactgccgtacgtcctcggctctgcgcaccagggctgcctccctccgttccctgcggacgtgttcatgattcctcagtacggatatctgactctaaacaacggcagtcagtctgtgggacgttcctccttctactgcctggagtactttccttctcagatgctgagaacgggcaataactttgaattcagctacacctttgaggaagtgcctttccacagcagctatgcgcacagccagagcctggaccggctgatgaatcccctcatcgaccagtacctgtactacctggcccggacccagagcactacggggtccacaagggagctgcagttccatcaggctgggcccaacaccatggccgagcaatcaaagaactggctgcccggaccctgttatcggcagcagagactgtcaaaaaacatagacagcaacaacaacagtaactttgcctggaccggggccactaaataccatctgaatggtagaaattcattaaccaacccgggcgtagccatggccaccaacaaggacgacgaggaccagttctttcccatcaacggagtgctggtttttggcgaaacgggggctgccaacaagacaacgctggaaaacgtgctaatgaccagcgaggaggagatcaaaaccaccaatcccgtggctacagaagaatacggtgtggtctccagcaacctgcaatcgtctacggccggaccccagacacagactgtcaacagccagggggctctgcccggcatggtctggcagaaccgggacgtgtacctgcagggtcccatctgggccaaaattcctcacacggacggcaactttcacccgtctcccctgatgggcggatttggactcaaacacccgcctcctcaaattctcatcaaaaacaccccggtacctgctaatcctccagaggtgtttactcctgccaagtttgcctcatttatcacgcagtacagcaccggccaggtcagcgtggagatcgagtgggaactgcagaaagaaaacagcaaacgctggaatccagagattcagtacacctcaaattatgccaagtctaataatgtggaatttgctgtcaacaacgaaggggtttatactgagcctcgccccattggcacccgttacctcacccgtaacctgtaa

SEQ ID NO: 29 - protein, 732 aa, Synthetic construct
  source 1..732: note = AAVhu32 / AAVrh13 hybrid VP1 capsid protein (AAVhu32 VP1u + AAVhu32 VP1/2s + AAVrh13 VP3)
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPRKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYTFEEVPFHSSYAHSQSLDRLMNPLIDQYLYYLARTQSTTGSTRELQFHQAGPNTMAEQSKNWLPGPCYRQQRLSKNIDSNNNSNFAWTGATKYHLNGRNSLTNPGVAMATNKDDEDQFFPINGVLVFGETGAANKTTLENVLMTSEEEIKTTNPVATEEYGVVSSNLQSSTAGPQTQTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYAKSNNVEFAVNNEGVYTEPRPIGTRYLTRNL

SEQ ID NO: 30 - DNA, 2199 bp, Synthetic construct
  source 1..2199: mol_type = other DNA; note = AAVhu32 / AAVrh13 hybrid VP1 capsid DNA (AAVhu32 VP1u + AAVhu32 VP1/2s + AAVrh13 VP3)
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggctgcaggcggtggcgcaccaatggcagacaataacgaaggcgccgacggagtgggtaatgcctccggaaattggcattgcgattccacatggctgggcgacagagtcatcaccaccagcacccgcacctgggccctgcccacctacaacaaccacctctacaagcagatatcaagtcagagcggggctaccaacgacaaccacttcttcggctacagcaccccctggggctattttgacttcaacagattccactgccacttctcaccacgtgactggcagcgactcatcaacaacaactggggattccggcccagaaagctgcggttcaagttgttcaacatccaggtcaaggaggtcacgacgaacgacggcgttacgaccatcgctaataaccttaccagcacgattcaggtcttctcggactcggagtaccaactgccgtacgtcctcggctctgcgcaccagggctgcctccctccgttccctgcggacgtgttcatgattcctcagtacggatatctgactctaaacaacggcagtcagtctgtgggacgttcctccttctactgcctggagtactttccttctcagatgctgagaacgggcaataactttgaattcagctacacctttgaggaagtgcctttccacagcagctatgcgcacagccagagcctggaccggctgatgaatcccctcatcgaccagtacctgtactacctggcccggacccagagcactacggggtccacaagggagctgcagttccatcaggctgggcccaacaccatggccgagcaatcaaagaactggctgcccggaccctgttatcggcagcagagactgtcaaaaaacatagacagcaacaacaacagtaactttgcctggaccggggccactaaataccatctgaatggtagaaattcattaaccaacccgggcgtagccatggccaccaacaaggacgacgaggaccagttctttcccatcaacggagtgctggtttttggcgaaacgggggctgccaacaagacaacgctggaaaacgtgctaatgaccagcgaggaggagatcaaaaccaccaatcccgtggctacagaagaatacggtgtggtctccagcaacctgcaatcgtctacggccggaccccagacacagactgtcaacagccagggggctctgcccggcatggtctggcagaaccgggacgtgtacctgcagggtcccatctgggccaaaattcctcacacggacggcaactttcacccgtctcccctgatgggcggatttggactcaaacacccgcctcctcaaattctcatcaaaaacaccccggtacctgctaatcctccagaggtgtttactcctgccaagtttgcctcatttatcacgcagtacagcaccggccaggtcagcgtggagatcgagtgggaactgcagaaagaaaacagcaaacgctggaatccagagattcagtacacctcaaattatgccaagtctaataatgtggaatttgctgtcaacaacgaaggggtttatactgagcctcgccccattggcacccgttacctcacccgtaacctgtaa

SEQ ID NO: 31 - protein, 736 aa, Adeno-associated virus
  source 1..736: note = AAVhu32 VP1 region protein
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - DNA, 2211 bp, Adeno-associated virus
  source 1..2211: mol_type = genomic DNA; note = AAVhu32 VP1 region DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 33 - protein, 737 aa, Synthetic construct
  source 1..737: note = AAV6/AAV8 VP1 aa
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 34 - DNA, 2214 bp, Synthetic construct
  source 1..2214: mol_type = other DNA; note = AAV6/AAV8 VP1 dna
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 35 - protein, 738 aa, Synthetic construct
  source 1..738: note = AAV6/AAVrh.21 VP1 aa
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 36 - DNA, 2217 bp, Synthetic construct
  source 1..2217: mol_type = other DNA; note = AAV6/AAVrh.21 VP1 dna
atggctgccgatggttatcttccagattggctcgaggacaacctctctgagggcattcgcgagtggtgggacctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataatcacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccgtcaccacagcgttcccccgactcctccacgggcatcggcaagaaaggccagcagcccgccagaaagagactcaatttcggtcagactggcgactcagagtcagtccccgaccctcaacctctgggagaacctccagcagcgccctctagtgtgggatctggtacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 37 - protein, 737 aa, Synthetic construct
  source 1..737: note = AAV6/AAVrh.73 VP1 aa
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQRQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 38 - DNA, 2214 bp, Synthetic construct
  source 1..2214: mol_type = other DNA; note = AAV6/AAVrh.73 VP1 dna
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctactttggctacagcaccccctgggggtattttgactttaacagattccactgccacttctcaccacgtgactggcagcgactcatcaacaacaactggggattccggcccaagagactcagcttcaagctcttcaacatccaggtcaaagaggtcacgcagaatgaaggcaccaagaccatcgccaataacctcaccagcaccatccaggtgtttacggactcggaataccagctgccgtacgtcctcggctctgcccaccagggctgcctgcctccgttcccggcggacgtcttcatgattcctcagtacggctacctgactctcaacaacggtagtcaggccgtgggacgttcctccttctactgcctggagtacttcccctctcagatgctgagaacgggcaacaacttttccttcagctacactttcgaggacgtgcctttccacagcagctacgcgcacagccagagtttggacaggctgatgaatcctctcatcgaccagtacctgtactacctgtcaagaacccagtctacgggaggcacagcgggaacccagcagttgctgttttctcaggccgggcctagcaacatgtcggctcaggccagaaactggctgcctggaccctgctacagacagcagcgcgtctccacgacactgtcgcaaaacaacaacagcaactttgcctggactggtgccaccaagtatcatctgaacggcagagactctctggtgaatccgggcgtcgccatggcaaccaacaaggacgacgaggaccgcttcttcccatccagcggcatcctcatgtttggcaagcagggagctggaaaagacaacgtggactatagcaacgtgatgctaaccagcgaggaagaaatcaagaccaccaaccccgtggccacagaacagtatggcgtggtggctgataacctacagagacaaaacaccgctcctattgtgggggccgtcaacagccagggagccttacctggcatggtctggcagaaccgggacgtgtacctgcagggtcctatttgggccaagattcctcacacagatggcaactttcacccgtctcctttaatgggcggctttggacttaaacatccgcctcctcagatcctcatcaaaaacactcctgttcctgcggatcctccaacagcgttcaaccaggccaagctgaattctttcatcacgcagtacagcaccggacaagtcagcgtggagatcgagtgggagctgcagaaggagaacagcaagcgctggaacccagagattcagtatacttccaactactacaaatctacaaatgtggactttgctgttaatactgagggtgtttactctgagcctcgccccattggcactcgttacctcacccgtaatctgtaa

SEQ ID NO: 39 - protein, 737 aa, Synthetic construct
  source 1..737: note = AAV8/AAV6 VP1 aa
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 40 - DNA, 2214 bp, Synthetic construct
  source 1..2214: mol_type = other DNA; note = AAV8/AAV6 VP1 dna
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 41 - protein, 733 aa, Synthetic construct
  source 1..733: note = AAV8/AAVrh.21 VP1 aa
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPRKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYTFEEVPFHSSYAHSQSLDRLMNPLIDQYLYYLARTQSTTGSTRELQFHQAGPNTMAEQSKNWLPGPCYRQQRLSKNIDSNNNSNFAWTGATKYHLNGRNSLTNPGVAMATNKDDEDQFFPINGVLVFGKTGAANKTTLENVLMTSEEEIKTTNPVATEEYGVVSSNLQSSTAGPQTQTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYAKSNNVEFAVNNEGVYTEPRPIGTRYLTRNL

SEQ ID NO: 42 - DNA, 2202 bp, Synthetic construct
  source 1..2202: mol_type = other DNA; note = AAV8/AAVrh.21 VP1 dna
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgctccaatggcagacaataacgaaggcgccgacggagtgggtaatgcctccggaaattggcattgcgattccacatggctgggcgacagagtcatcaccaccagcacccgcacctgggccctgcccacctacaacaaccacctctacaagcagatatcaagtcagagcggggctaccaacgacaaccacttcttcggctacagcaccccctggggctattttgacttcaacagattccactgccacttctcaccacgtgactggcagcgactcatcaacaacaactggggattccggcccagaaagctgcggttcaagttgttcaacatccaggtcaaggaggtcacgacgaacgacggcgttacgaccatcgccaataaccttaccagcacgattcaggtcttctcggactcggagtaccaactgccgtacgtcctcggctctgcgcaccagggctgcctccctccgttccctgcggacgtgttcatgattcctcagtacggatatctgactctaaacaacggcagtcagtctgtgggacgttcctccttctactgcctggagtactttccttctcagatgctgagaacgggcaataactttgaattcagctacacctttgaggaagtgcctttccacagcagctatgcgcacagccagagcctggaccggctgatgaatcccctcatcgaccagtacctgtactacctggcccggacccagagcactacggggtccacaagggagctgcagttccatcaggctgggcccaacaccatggccgagcaatcaaagaactggctgcccggaccctgttatcggcagcagagactgtcaaaaaacatagacagcaacaacaacagtaactttgcctggaccggggccactaaataccatctgaatggtagaaattcattaaccaacccgggcgtagccatggccaccaacaaggacgacgaggaccagttctttcccatcaacggagtgctggtttttggcaaaacgggggctgccaacaagacaacgctggaaaacgtgctaatgaccagcgaggaggagatcaaaaccaccaatcccgtggctacagaagaatacggtgtggtctccagcaacctgcaatcgtctacggccggaccccagacacagactgtcaacagccagggggctctgcccggcatggtctggcagaaccgggacgtgtacctgcagggtcccatctgggccaaaattcctcacacggacggcaactttcacccgtctcccctgatgggcggatttggactcaaacacccgcctcctcaaattctcatcaaaaacaccccggtacctgctaatcctccagaggtgtttactcctgccaagtttgcctcatttatcacgcagtacagcaccggccaggtcagcgtggagatcgagtgggaactgcagaaagaaaacagcaaacgctggaatccagagattcagtacacctcaaattatgccaagtctaataatgtggaatttgctgtcaacaacgaaggggtttatactgagcctcgccccattggcacccgttacctcacccgtaacctgtaa

SEQ ID NO: 43 - protein, 738 aa, Synthetic construct
  source 1..738: note = AAV8/AAVrh.73 VP1 aa
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQRQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 44 - DNA, 2217 bp, Synthetic construct
  source 1..2217: mol_type = other DNA; note = AAV8/AAVrh.73 VP1 dna
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctactttggctacagcaccccctgggggtattttgactttaacagattccactgccacttctcaccacgtgactggcagcgactcatcaacaacaactggggattccggcccaagagactcagcttcaagctcttcaacatccaggtcaaagaggtcacgcagaatgaaggcaccaagaccatcgccaataacctcaccagcaccatccaggtgtttacggactcggaataccagctgccgtacgtcctcggctctgcccaccagggctgcctgcctccgttcccggcggacgtcttcatgattcctcagtacggctacctgactctcaacaacggtagtcaggccgtgggacgttcctccttctactgcctggagtacttcccctctcagatgctgagaacgggcaacaacttttccttcagctacactttcgaggacgtgcctttccacagcagctacgcgcacagccagagtttggacaggctgatgaatcctctcatcgaccagtacctgtactacctgtcaagaacccagtctacgggaggcacagcgggaacccagcagttgctgttttctcaggccgggcctagcaacatgtcggctcaggccagaaactggctgcctggaccctgctacagacagcagcgcgtctccacgacactgtcgcaaaacaacaacagcaactttgcctggactggtgccaccaagtatcatctgaacggcagagactctctggtgaatccgggcgtcgccatggcaaccaacaaggacgacgaggaccgcttcttcccatccagcggcatcctcatgtttggcaagcagggagctggaaaagacaacgtggactatagcaacgtgatgctaaccagcgaggaagaaatcaagaccaccaaccccgtggccacagaacagtatggcgtggtggctgataacctacagagacaaaacaccgctcctattgtgggggccgtcaacagccagggagccttacctggcatggtctggcagaaccgggacgtgtacctgcagggtcctatttgggccaagattcctcacacagatggcaactttcacccgtctcctttaatgggcggctttggacttaaacatccgcctcctcagatcctcatcaaaaacactcctgttcctgcggatcctccaacagcgttcaaccaggccaagctgaattctttcatcacgcagtacagcaccggacaagtcagcgtggagatcgagtgggagctgcagaaggagaacagcaagcgctggaacccagagattcagtatacttccaactactacaaatctacaaatgtggactttgctgttaatactgagggtgtttactctgagcctcgccccattggcactcgttacctcacccgtaatctgtaa

SEQ ID NO: 45 - protein, 740 aa, Synthetic construct
  source 1..740: note = AAV9/AAV4 VP1 aa
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 46 - DNA, 2223 bp, Synthetic construct
  source 1..2223: mol_type = other DNA; note = AAV9/AAV4 VP1 dna
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaa

SEQ ID NO: 47 - protein, 737 aa, Synthetic construct
  source 1..737: note = AAV9/AAV8 VP1 aa
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 48 - DNA, 2214 bp, Synthetic construct
  source 1..2214: mol_type = other DNA; note = AAV9/AAV8 VP1 dna
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 49 - protein, 737 aa, Synthetic construct
  source 1..737: note = AAV9/AAVrh18 VP1 aa
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 50 - DNA, 2214 bp, Synthetic construct
  source 1..2214: mol_type = other DNA; note = AAV9/AAVrh18 VP1 dna
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacatcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaacggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcttggaccggtgccaccaagtatcatctgaatggcagagactctctggtaaatcccggtgtcgctatggcaacgcacaaggacgacgaagagcgattttttccatccagcggagtcttgatgtttgggaaacagggagctggaaaagacaacgtggactatagcagcgttatgctaaccagtgaggaagaaatcaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggcaactttcatccttcgccgctgatgggaggctttggactgaaacacccgcctcctcagatcctgattaagaatacacctgttcccgcggatcctccaactaccttcagtcaagccaagctggcgtcgttcatcacgcagtacagcaccggacaggtcagcgtggaaattgaatgggagctgcagaaagagaacagcaagcgctggaacccagagattcagtatacttccaactactacaaatctacaaatgtggactttgctgtcaatactgagggtacttattcagagcctcgccccattggcacccgttacctcacccgtaacctgtaa

SEQ ID NO: 51 - protein, 733 aa, Synthetic construct
  source 1..733: note = AAVrh21/AAV8 VP1 aa
MAADGYLPDWLEDNLSEGIREWWDLKPGAPKPKANQQKQDDGRGLVLPGYKYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPIESPDSSTGIGRKGQQPAKKKLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 52 - DNA, 2202 bp, Synthetic construct
  source 1..2202: mol_type = other DNA; note = AAVrh21/AAV8 VP1 dna
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaacctcgggcgagcagtcttccaggccaagaagcgggttctcgaacctctcggtctggttgaggaaggcgctaagacggctcctggaaagaagagacccatagaatcccccgactcctccacgggcatcggcaggaaaggccagcagcccgctaaaaagaagctcaactttgggcagactggcgactcagagtcagtgcccgaccctcaaccaatcggagaaccccccgcaggcccctctggtctgggatctggtacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 53 - protein, 738 aa, Synthetic construct; identical to SEQ ID NO: 35
  source 1..738: note = AAVrh73/AAV8 VP1 aa

SEQ ID NO: 54 - DNA, 2217 bp, Synthetic construct; identical to SEQ ID NO: 36
  source 1..2217: mol_type = other DNA; note = AAVrh73/AAV8 VP1 dna

SEQ ID NO: 55 - protein, 535 aa, Synthetic construct; identical to SEQ ID NO: 11
  source 1..535: note = AAV3B/AAV8 VP1 aa

SEQ ID NO: 56 - DNA, 1608 bp, Synthetic construct; identical to SEQ ID NO: 12
  source 1..1608: mol_type = other DNA; note = AAV3B/AAV8 VP1 dna

SEQ ID NO: 57 - protein, 732 aa, Synthetic construct
  source 1..732: note = AAVrh15/AAVhu32 VP1 aa
MAADGYLPDWLEDNLSEGIREWWDLKPGAPKPKANQQKQDDGRGLVLPGYKYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPIESPDSSTGIGKKGQQPAKKKLNFGQTGDSESVPDPQPIGEPPAGPSGLGSGTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 58 - DNA, 2199 bp, Synthetic construct
  source 1..2199: mol_type = other DNA; note = AAVrh15/AAVhu32 VP1 dna
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaacctcgggcgagcagtcttccaggccaagaagcgggttctcgaacctctcggtctggttgaggaaggcgctaagacggctcctggaaagaagagacccatagaatcccccgactcctccacgggcatcggcaagaaaggccagcagcccgctaaaaagaagctcaactttgggcagactggcgactcagagtcagtgcccgaccctcaaccaatcggagaaccccccgcaggcccctctggtctgggatctggtacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 59 - protein, 732 aa, Synthetic construct
  source 1..732: note = AAVrh13/AAVhu32 VP1 aa
MAADGYLPDWLEDNLSEGIREWWDLKPGAPKPKANQQKQDDGRGLVLPGYKYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPIESPDSSTGIGKKGQQPAKKKLNFGQTGDSESVPDPQPLGEPPAAPSGLGSGTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 60 - DNA, 2199 bp, Synthetic construct
  source 1..2199: mol_type = other DNA; note = AAVrh13/AAVhu32 VP1 dna
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggagagccggtcaacgaggcagacgccgcggccctcgagcacgacaaggcctacgacaagcagctcgagcagggggacaacccgtacctcaagtacaaccacgccgacgccgagtttcaggagcgtcttcaagaagatacgtcttttgggggcaacctcgggcgagcagtcttccaggccaagaagcgggttctcgaacctctcggtctggttgaggaaggcgctaagacggctcctggaaagaagagacccatagaatcccccgactcctccacgggcatcggcaagaaaggccagcagcccgctaaaaagaagctcaactttgggcagactggcgactcagagtcagtgcccgacccccaacctctcggagaacctcccgccgcgccctcaggtctgggatctggtacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 61 - protein, 736 aa, Synthetic construct
  source 1..736: note = AAV6/AAVhu32 VP1 aa
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 62 - DNA, 2211 bp, Synthetic construct
  source 1..2211: mol_type = other DNA; note = AAV6/AAVhu32 VP1 dna
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 63 - protein, 726 aa, Synthetic construct
  source 1..726: note = AAV5/AAVhu32 VP1 aa
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 64 - DNA, 2181 bp, Synthetic construct
  source 1..2181: mol_type = other DNA; note = AAV5/AAVhu32 VP1 dna
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 65 - protein, 732 aa, Synthetic construct
  source 1..732: note = AAVhu32/AAVrh15 VP1 aa
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPRKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYTFEEVPFHSSYAHSQSLDRLMNPLIDQYLYYLARTQSTTGSTRELQFHQAGPNTMAEQSKNWLPGPCYRQQRLSKNIDSNNTSNFAWTGATKYHLNGRNSLTNPGVAMATNKDDEDQFFPINGVLVFGKTGAANKTTLENVLMTSEEEIKTTNPVATEQYGVVSSNLQSSTAGPQTQTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYAKSNNVEFAVNNEGVYTEPRPIGTRYLTRNL

SEQ ID NO: 66 - DNA, 2199 bp, Synthetic construct
  source 1..2199: mol_type = other DNA; note = AAVhu32/AAVrh15 VP1 dna
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggctgcaggcggtggcgctccaatggcagacaataacgaaggcgccgacggagtgggtaatgcctccggaaattggcattgcgattccacatggctgggcgacagagtcatcaccaccagcacccgcacctgggccctgcccacctacaacaaccacctctacaagcagatatcaagtcagagcggggctaccaacgacaaccacttcttcggctacagcaccccctggggctattttgacttcaacagattccactgccacttctcaccacgtgactggcagcgactcatcaacaacaactggggattccggcccagaaagctgcggttcaagttgttcaacatccaggtcaaggaggtcacgacgaacgacggcgttacgaccatcgctaataaccttaccagcacgattcaggtcttctcggactcggagtaccaactgccgtacgtcctcggctctgcgcaccagggctgcctccctccgttccctgcggacgtgttcatgattcctcagtacggatatctgactctaaacaacggcagtcagtctgtgggacgttcctccttctactgcctggagtactttccttctcagatgctgagaacgggcaataactttgaattcagctacacctttgaggaagtgcctttccacagcagctatgcgcacagccagagcctggaccggctgatgaatcccctcatcgaccagtacctgtactacctggcccggacccagagcactacggggtccacaagggagctgcagttccatcaggctgggcccaacaccatggccgagcaatcaaagaactggctgcccggaccctgttatcggcagcagagactgtcaaaaaacatagacagcaacaacaccagtaactttgcctggaccggggccactaaataccatctgaatggtagaaattcattaaccaacccgggcgtagccatggccaccaacaaggacgacgaggaccagttctttcccatcaacggagtgctggtttttggcaaaacgggggctgccaacaagacaacgctggaaaacgtgctaatgaccagcgaggaggagatcaaaaccaccaatcccgtggctacagaacagtacggtgtggtctccagcaacctgcaatcgtctacggccggaccccagacacagactgtcaacagccagggggctctgcccggcatggtctggcagaaccgggacgtgtacctgcagggtcccatctgggccaaaattcctcacacggacggcaactttcacccgtctcccctgatgggcggatttggactcaaacacccgcctcctcaaattctcatcaaaaacaccccggtacctgctaatcctccagaggtgtttactcctgccaagtttgcctcatttatcacgcagtacagcaccggccaggtcagcgtggagatcgagtgggaactgcagaaagaaaacagcaaacgctggaatccagagattcagtacacctcaaattatgccaagtctaataatgtggaatttgctgtcaacaacgaaggggtttatactgagcctcgccccattggcacccgttacctcacccgtaacctgtaa

SEQ ID NO: 67 - protein, 732 aa, Synthetic construct
  source 1..732: note = AAV6/AAVrh15 VP1 aa
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPRKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYTFEEVPFHSSYAHSQSLDRLMNPLIDQYLYYLARTQSTTGSTRELQFHQAGPNTMAEQSKNWLPGPCYRQQRLSKNIDSNNTSNFAWTGATKYHLNGRNSLTNPGVAMATNKDDEDQFFPINGVLVFGKTGAANKTTLENVLMTSEEEIKTTNPVATEQYGVVSSNLQSSTAGPQTQTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYAKSNNVEFAVNNEGVYTEPRPIGTRYLTRNL

SEQ ID NO: 68 - DNA, 2199 bp, Synthetic construct
  source 1..2199: mol_type = other DNA; note = AAV6/AAVrh15 VP1 dna
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggctgcaggcggtggcgctccaatggcagacaataacgaaggcgccgacggagtgggtaatgcctccggaaattggcattgcgattccacatggctgggcgacagagtcatcaccaccagcacccgcacctgggccctgcccacctacaacaaccacctctacaagcagatatcaagtcagagcggggctaccaacgacaaccacttcttcggctacagcaccccctggggctattttgacttcaacagattccactgccacttctcaccacgtgactggcagcgactcatcaacaacaactggggattccggcccagaaagctgcggttcaagttgttcaacatccaggtcaaggaggtcacgacgaacgacggcgttacgaccatcgctaataaccttaccagcacgattcaggtcttctcggactcggagtaccaactgccgtacgtcctcggctctgcgcaccagggctgcctccctccgttccctgcggacgtgttcatgattcctcagtacggatatctgactctaaacaacggcagtcagtctgtgggacgttcctccttctactgcctggagtactttccttctcagatgctgagaacgggcaataactttgaattcagctacacctttgaggaagtgcctttccacagcagctatgcgcacagccagagcctggaccggctgatgaatcccctcatcgaccagtacctgtactacctggcccggacccagagcactacggggtccacaagggagctgcagttccatcaggctgggcccaacaccatggccgagcaatcaaagaactggctgcccggaccctgttatcggcagcagagactgtcaaaaaacatagacagcaacaacaccagtaactttgcctggaccggggccactaaataccatctgaatggtagaaattcattaaccaacccgggcgtagccatggccaccaacaaggacgacgaggaccagttctttcccatcaacggagtgctggtttttggcaaaacgggggctgccaacaagacaacgctggaaaacgtgctaatgaccagcgaggaggagatcaaaaccaccaatcccgtggctacagaacagtacggtgtggtctccagcaacctgcaatcgtctacggccggaccccagacacagactgtcaacagccagggggctctgcccggcatggtctggcagaaccgggacgtgtacctgcagggtcccatctgggccaaaattcctcacacggacggcaactttcacccgtctcccctgatgggcggatttggactcaaacacccgcctcctcaaattctcatcaaaaacaccccggtacctgctaatcctccagaggtgtttactcctgccaagtttgcctcatttatcacgcagtacagcaccggccaggtcagcgtggagatcgagtgggaactgcagaaagaaaacagcaaacgctggaatccagagattcagtacacctcaaattatgccaagtctaataatgtggaatttgctgtcaacaacgaaggggtttatactgagcctcgccccattggcacccgttacctcacccgtaacctgtaa

SEQ ID NO: 69 - protein, 722 aa, Synthetic construct
  source 1..722: note = AAV5/AAVrh15 VP1 aa
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPRKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYTFEEVPFHSSYAHSQSLDRLMNPLIDQYLYYLARTQSTTGSTRELQFHQAGPNTMAEQSKNWLPGPCYRQQRLSKNIDSNNTSNFAWTGATKYHLNGRNSLTNPGVAMATNKDDEDQFFPINGVLVFGKTGAANKTTLENVLMTSEEEIKTTNPVATEQYGVVSSNLQSSTAGPQTQTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYAKSNNVEFAVNNEGVYTEPRPIGTRYLTRNL

SEQ ID NO: 70 - DNA, 2169 bp, Synthetic construct
  source 1..2169: mol_type = other DNA; note = AAV5/AAVrh15 VP1 dna
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatggctgcaggcggtggcgctccaatggcagacaataacgaaggcgccgacggagtgggtaatgcctccggaaattggcattgcgattccacatggctgggcgacagagtcatcaccaccagcacccgcacctgggccctgcccacctacaacaaccacctctacaagcagatatcaagtcagagcggggctaccaacgacaaccacttcttcggctacagcaccccctggggctattttgacttcaacagattccactgccacttctcaccacgtgactggcagcgactcatcaacaacaactggggattccggcccagaaagctgcggttcaagttgttcaacatccaggtcaaggaggtcacgacgaacgacggcgttacgaccatcgctaataaccttaccagcacgattcaggtcttctcggactcggagtaccaactgccgtacgtcctcggctctgcgcaccagggctgcctccctccgttccctgcggacgtgttcatgattcctcagtacggatatctgactctaaacaacggcagtcagtctgtgggacgttcctccttctactgcctggagtactttccttctcagatgctgagaacgggcaataactttgaattcagctacacctttgaggaagtgcctttccacagcagctatgcgcacagccagagcctggaccggctgatgaatcccctcatcgaccagtacctgtactacctggcccggacccagagcactacggggtccacaagggagctgcagttccatcaggctgggcccaacaccatggccgagcaatcaaagaactggctgcccggaccctgttatcggcagcagagactgtcaaaaaacatagacagcaacaacaccagtaactttgcctggaccggggccactaaataccatctgaatggtagaaattcattaaccaacccgggcgtagccatggccaccaacaaggacgacgaggaccagttctttcccatcaacggagtgctggtttttggcaaaacgggggctgccaacaagacaacgctggaaaacgtgctaatgaccagcgaggaggagatcaaaaccaccaatcccgtggctacagaacagtacggtgtggtctccagcaacctgcaatcgtctacggccggaccccagacacagactgtcaacagccagggggctctgcccggcatggtctggcagaaccgggacgtgtacctgcagggtcccatctgggccaaaattcctcacacggacggcaactttcacccgtctcccctgatgggcggatttggactcaaacacccgcctcctcaaattctcatcaaaaacaccccggtacctgctaatcctccagaggtgtttactcctgccaagtttgcctcatttatcacgcagtacagcaccggccaggtcagcgtggagatcgagtgggaactgcagaaagaaaacagcaaacgctggaatccagagattcagtacacctcaaattatgccaagtctaataatgtggaatttgctgtcaacaacgaaggggtttatactgagcctcgccccattggcacccgttacctcacccgtaacctgtaa

SEQ ID NO: 71 - protein, 732 aa, Synthetic construct
  source 1..732: note = AAV6/AAVrh13 VP1 aa
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPRKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYTFEEVPFHSSYAHSQSLDRLMNPLIDQYLYYLARTQSTTGSTRELQFHQAGPNTMAEQSKNWLPGPCYRQQRLSKNIDSNNNSNFAWTGATKYHLNGRNSLTNPGVAMATNKDDEDQFFPINGVLVFGETGAANKTTLENVLMTSEEEIKTTNPVATEEYGVVSSNLQSSTAGPQTQTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYAKSNNVEFAVNNEGVYTEPRPIGTRYLTRNL

SEQ ID NO: 72 - DNA, 2199 bp, Synthetic construct
  source 1..2199: mol_type = other DNA; note = AAV6/AAVrh13 VP1 dna
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggctgcaggcggtggcgcaccaatggcagacaataacgaaggcgccgacggagtgggtaatgcctccggaaattggcattgcgattccacatggctgggcgacagagtcatcaccaccagcacccgcacctgggccctgcccacctacaacaaccacctctacaagcagatatcaagtcagagcggggctaccaacgacaaccacttcttcggctacagcaccccctggggctattttgacttcaacagattccactgccacttctcaccacgtgactggcagcgactcatcaacaacaactggggattccggcccagaaagctgcggttcaagttgttcaacatccaggtcaaggaggtcacgacgaacgacggcgttacgaccatcgctaataaccttaccagcacgattcaggtcttctcggactcggagtaccaactgccgtacgtcctcggctctgcgcaccagggctgcctccctccgttccctgcggacgtgttcatgattcctcagtacggatatctgactctaaacaacggcagtcagtctgtgggacgttcctccttctactgcctggagtactttccttctcagatgctgagaacgggcaataactttgaattcagctacacctttgaggaagtgcctttccacagcagctatgcgcacagccagagcctggaccggctgatgaatcccctcatcgaccagtacctgtactacctggcccggacccagagcactacggggtccacaagggagctgcagttccatcaggctgggcccaacaccatggccgagcaatcaaagaactggctgcccggaccctgttatcggcagcagagactgtcaaaaaacatagacagcaacaacaacagtaactttgcctggaccggggccactaaataccatctgaatggtagaaattcattaaccaacccgggcgtagccatggccaccaacaaggacgacgaggaccagttctttcccatcaacggagtgctggtttttggcgaaacgggggctgccaacaagacaacgctggaaaacgtgctaatgaccagcgaggaggagatcaaaaccaccaatcccgtggctacagaagaatacggtgtggtctccagcaacctgcaatcgtctacggccggaccccagacacagactgtcaacagccagggggctctgcccggcatggtctggcagaaccgggacgtgtacctgcagggtcccatctgggccaaaattcctcacacggacggcaactttcacccgtctcccctgatgggcggatttggactcaaacacccgcctcctcaaattctcatcaaaaacaccccggtacctgctaatcctccagaggtgtttactcctgccaagtttgcctcatttatcacgcagtacagcaccggccaggtcagcgtggagatcgagtgggaactgcagaaagaaaacagcaaacgctggaatccagagattcagtacacctcaaattatgccaagtctaataatgtggaatttgctgtcaacaacgaaggggtttatactgagcctcgccccattggcacccgttacctcacccgtaacctgtaa

SEQ ID NO: 73 - protein, 1337 aa, Synthetic construct
  source 1..1337: note = DYS1
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPR

SEQ ID NO: 74 - protein, 1191 aa, Synthetic construct
  source 1..1191: note = DYS3
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 75 - protein, 1283 aa, Synthetic construct
  source 1..1283: note = DYS5
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLES

SEQ ID NO: 76 - protein, 1197 aa, Synthetic construct
  source 1..1197: note = human MD1 (R4-R23/deltaCT)
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 77 - protein, 1192 aa, Synthetic construct
  source 1..1192: note = Human microdystrophin
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 78 - protein, 1325 aa, Synthetic construct
  source 1..1325: note = Dys3978
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDQPDLAPGLTTIGASPTQTVTLVTQPVVTKETAISKLEMPSSLMLEVPTHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 79 - protein, 1392 aa, Synthetic construct
  source 1..1392: note = Human MD3
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEDTM

SEQ ID NO: 80 - protein, 1471 aa, Synthetic construct
  source 1..1471: note = Human MD4
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 81 - protein, 743 aa, Synthetic construct
  source 1..743: note = Hu32 Variant 1
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSTTLYSPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 82 - DNA, 2232 bp, Synthetic construct
  source 1..2232: mol_type = other DNA; note = Hu32 Variant 1
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatcaacgacgctctattctccagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 83 - protein, 743 aa, Synthetic construct
  source 1..743: note = Hu32 Variant 2
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQFVVGQSYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 84 - DNA, 2232 bp, Synthetic construct
  source 1..2232: mol_type = other DNA; note = Hu32 Variant 2
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaattcgttgtgggacagtcctatgcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 85 - protein, 746 aa, Synthetic construct
  source 1..746: note = Hu32 Variant 3
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQSGRGDLGLGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 86 - DNA, 2241 bp, Synthetic construct
  source 1..2241: mol_type = other DNA; note = Hu32 Variant 3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggccagagtggccgcggcgatctgggcctgggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 87 - protein, 745 aa, Synthetic construct
  source 1..745: note = Hu32 variant 4
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSGRGDLGLSAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 88 - DNA, 2238 bp, Synthetic construct
  source 1..2238: mol_type = other DNA; note = Hu32 variant 4
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagggccagagtggccgcggcgatctgggcctgagcgcccaggcggcccagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 89 - protein, 743 aa, Synthetic construct
  source 1..743: note = Hu32 variant 5
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGRGDLGLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 90 - DNA, 2232 bp, Synthetic construct
  source 1..2232: mol_type = other DNA; note = Hu32 variant 5
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaggccgcggcgatctgggcctggcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 91 - protein, 743 aa, Synthetic construct
  source 1..743: note = Hu32 variant 6
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQRGDYNSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 92 - DNA, 2232 bp, Synthetic construct
  source 1..2232: mol_type = other DNA; note = Hu32 variant 6
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaacagagaggcgactacaactctgcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 93 - protein, 743 aa, Synthetic construct
  source 1..743: note = Hu32 variant 7
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQNSRGDYNAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 94 - DNA, 2232 bp, Synthetic construct
  source 1..2232: mol_type = other DNA; note = Hu32 variant 7
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaaattctagaggcgactacaacgcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 95 - protein, 743 aa, Synthetic construct
  source 1..743: note = Hu32 variant 8
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQENRRGDFAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 96 - DNA, 2232 bp, Synthetic construct
  source 1..2232: mol_type = other DNA; note = Hu32 variant 8
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagaaaaccggagaggcgacttcgcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 97 - protein, 743 aa, Synthetic construct
  source 1..743: note = Hu32 variant 9
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDYVGLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 98 - DNA, 2232 bp, Synthetic construct
  source 1..2232: mol_type = other DNA; note = Hu32 variant 9
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaagaggcgactacgtgggcctggcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 99 - protein, 743 aa, Synthetic construct
  source 1..743: note = Hu32 variant 10
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNSRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 100 - DNA, 2232 bp, Synthetic construct
  source 1..2232: mol_type = other DNA; note = Hu32 variant 10
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagcaactctagaggcgactacaacagcctggcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 101 - protein, 743 aa, Synthetic construct
  source 1..743: note = Hu32 variant 11
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDFNNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 102 - DNA, 2232 bp, Synthetic construct
  source 1..2232: mol_type = other DNA; note = Hu32 variant 11
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccaggagaatagacggggcgacttcaacaacaccgcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 103 - protein, 743 aa, Synthetic construct
  source 1..743: note = Hu32 variant 12
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDYVGVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 104 - DNA, 2232 bp, Synthetic construct
  source 1..2232: mol_type = other DNA; note = Hu32 variant 12
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaagaggcgactacgtgggcgtggcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 105 - protein, 743 aa, Synthetic construct
  source 1..743: note = Hu32 variant 13
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDVLGSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 106 - DNA, 2232 bp, Synthetic construct
  source 1..2232: mol_type = other DNA; note = Hu32 variant 13
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaagaggcgacgtgctgggctctgcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 107 - protein, 743 aa, Synthetic construct
  source 1..743: note = Hu32 variant 14
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDFVGLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 108 - DNA, 2232 bp, Synthetic construct
  source 1..2232: mol_type = other DNA; note = Hu32 variant 14
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaagaggcgacttcgtgggcctggcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 109 - protein, 743 aa, Synthetic construct
  source 1..743: note = Hu32 variant 15
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDYTLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 110 - DNA, 2232 bp, Synthetic construct
  source 1..2232: mol_type = other DNA; note = Hu32 variant 15
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaagaggcgactacaccctgtctgcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 111 - protein, 743 aa, Synthetic construct
  source 1..743: note = Hu32 variant 16
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSRGDFNLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 112 - DNA, 2232 bp, Synthetic construct
  source 1..2232: mol_type = other DNA; note = Hu32 variant 16
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatctagaggcgacttcaacctggcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 113 - protein, 741 aa, Synthetic construct
  source 1..741: note = Hu32 variant 17
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDYVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 114 - DNA, 2226 bp, Synthetic construct
  source 1..2226: mol_type = other DNA; note = Hu32 variant 17
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaaagaggcgactacgtggcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 115 - protein, 736 aa, Synthetic construct
  source 1..736: note = Hu32 variant 18
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFARPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 116 - DNA, 2211 bp, Synthetic construct
  source 1..2211: mol_type = other DNA; note = Hu32 variant 18
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgctcgacctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 117 - protein, 743 aa, Synthetic construct
  source 1..743: note = Hu32 variant 19
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFARPGASSWALNGRNSLMNPGPAMASHKEGEDRFFPLSGSLIFGKQGTGRDNVDADKVMITNEEEIKTTNPVATESYGQVATNHQSAQSTTLYSPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 118 - DNA, 2232 bp, Synthetic construct
  source 1..2232: mol_type = other DNA; note = Hu32 variant 19
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgctcgacctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatcaacgacgctctattctccagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 119 - protein, 743 aa, Synthetic construct
  source 1..743: note = Hu32 variant 20
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFARPGASSWALNGRNSLMNPGPAMASHKEGEDRFFPLSGSLIFGKQGTGRDNVDADKVMITNEEEIKTTNPVATESYGQVATNHQSAQFVVGQSYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 120 - DNA, 2232 bp, Synthetic construct
  source 1..2232: mol_type = other DNA; note = Hu32 variant 20
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgctcgacctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaattcgttgtgggacagtcctatgcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 121 - protein, 58 aa, Synthetic construct
  source 1..58: note = AAV9/hu32 consensus N-terminal sequence
  VARIANT 14: note = X = N or T
  VARIANT 21: note = X = E or Q
  VARIANT 24: note = X = A or K
  VARIANT 29: note = X = A or P
  VARIANT 31: note = X = Q or P
  VARIANT 33: note = X = A or P
  VARIANT 35: note = X = N or A
  VARIANT 36: note = X = Q or E
  VARIANT 37: note = X = Q or R
  VARIANT 39: note = X = Q or K
  VARIANT 41: note = X = N or D
  VARIANT 42: note = X = A or S
  VARIANT 34: note = X = A or P
MAADGYLPDWLEDXLSEGIRXWWXLKPGXPXPKXXXXHXDXXRGLVLPGYKYLGPGNG

SEQ ID NO: 122 - protein, 58 aa, Synthetic construct
  source 1..58: note = AAV9 N-terminal seqeunce
MAADGYLPDWLEDNLSEGIREWWALKPGAPQPKANQQHQDNARGLVLPGYKYLGPGNG

SEQ ID NO: 123 - protein, 58 aa, Synthetic construct
  source 1..58: note = Hu32 N-terminal seqeunce
MAADGYLPDWLEDTLSEGIRQWWKLKPGPPPPKPAERHKDDSRGLVLPGYKYLGPGNG

SEQ ID NO: 124 - protein, 736 aa, Adeno-associated virus
  source 1..736: note = AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 125 - DNA, 2211 bp, Adeno-associated virus
  source 1..2211: mol_type = genomic DNA; note = AAV9
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 126 - protein, 736 aa, Adeno-associated virus
  source 1..736: note = AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 127 - protein, 735 aa, Adeno-associated virus [sequence omitted]

SEQ ID NO: 128 - protein, 736 aa, Adeno-associated virus
  source 1..736: note = AAV3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 129 - protein, 734 aa, Adeno-associated virus
  source 1..734: note = AAV4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 130 - protein, 736 aa, Adeno-associated virus
  source 1..736: note = AAV6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 131 - protein, 737 aa, Adeno-associated virus
  source 1..737: note = AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 132 - protein, 736 aa, Adeno-associated virus
  source 1..736: note = AAVhu31
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 133 - protein, 738 aa, Adeno-associated virus
  source 1..738: note = AAVrh10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 134 - protein, 12 aa, Synthetic construct
  source 1..12: note = Muscle-homing peptide M12
RRQPPRSISSHP

SEQ ID NO: 135 - protein, 7 aa, Synthetic construct
  source 1..7: note = Muscle-specific peptide MSP
ASSLNIA

SEQ ID NO: 136 - protein, 27 aa, Synthetic construct
  source 1..27: note = Muscle-homing peptide
AGSTSAGSAAGSSGDRRQPPRSISSHP

SEQ ID NO: 137 - protein, 9 aa, Synthetic construct
  source 1..9: note = Muscle-homing peptide
CYAIGSFDC

SEQ ID NO: 138 - protein, 6 aa, Synthetic construct
  source 1..6: note = Muscle-homing peptide
SGASAV

SEQ ID NO: 139 - protein, 6 aa, Synthetic construct
  source 1..6: note = Muscle-homing peptide
  VARIANT 5: note = X = any amino acid
GRSGXR

SEQ ID NO: 140 - protein, 7 aa, Synthetic construct
  source 1..7: note = RGD peptide
SLRSPPS

SEQ ID NO: 141 - protein, 7 aa, Synthetic construct
  source 1..7: note = RGD peptide
RGDLGLS

SEQ ID NO: 142 - protein, 9 aa, Synthetic construct
  source 1..9: note = 4C-RGD peptide
CDCRGDCFC

SEQ ID NO: 143 - protein, 12 aa, Synthetic construct
  source 1..12: note = 4C-RGD peptide
CDCRGDCFCGLS

SEQ ID NO: 144 - protein, 7 aa, Synthetic construct
  source 1..7: note = RGD peptide
RGDLTTP

SEQ ID NO: 145 - protein, 7 aa, Synthetic construct
  source 1..7: note = RGD peptide
RGDLSTP

SEQ ID NO: 146 - protein, 137 aa, Adeno-associated virus
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAK

SEQ ID NO: 147 - DNA, 411 bp, Adeno-associated virus
  source 1..411: mol_type = genomic DNA; note = AAVhu32 VP1u
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaag

SEQ ID NO: 148 - protein, 65 aa, Adeno-associated virus
  source 1..65: note = AAVhu32 VP1/2s (amino acids 138-202)
TAPGKKRPVEQSPQEPDSSAGIGKSGSQPAKKKLNFGQTGDTESVPDPQPIGEPPAAPSGVGSLT

SEQ ID NO: 149 - DNA, 195 bp, Adeno-associated virus
  source 1..195: mol_type = genomic DNA; note = AAVhu32 VP1/2s
acggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttaca

SEQ ID NO: 150 - protein, 7 aa, Synthetic construct
  source 1..7: note = Muscle-homing peptide
  VARIANT 7: note = X = any amino acid
DFSGIAX

SEQ ID NO: 151 - protein, 7 aa, Synthetic construct
  source 1..7: note = RGD peptide
RGDLRVS